(19) United States
(12) Patent Application Publication
(10) Pub. No.: US 2025/0066323 A1
(43) Pub. Date: Feb. 27, 2025

(54) INHIBITORS OF HUMAN RESPIRATORY SYNCYTIAL VIRUS AND METAPNEUMOVIRUS

(71) Applicant: Merck Sharp & Dohme LLC, Rahway, NJ (US)

(72) Inventors: Brett R. Ambler, Philadelphia, PA (US); Douglas C. Beshore, Lower Gwynedd, PA (US); Christopher James Bungard, Lansdale, PA (US); Weizhe Dong, Lansdale, PA (US); Danielle M. Hurzy, Garnet Valley, PA (US); Peter J. Manley, Harleysville, PA (US); Vanessa L. Rada, II, Glenside, PA (US); Kelly-Ann S. Schlegel, Fleetwood, PA (US); Linda M. Suen-Lai, Malvern, PA (US); Mahdieh Yazdani, Blue Bell, PA (US)

(73) Assignee: Merck Sharp & Dohme LLC, Rahway, NJ (US)

(21) Appl. No.: 18/805,625

(22) Filed: Aug. 15, 2024

Related U.S. Application Data
(60) Provisional application No. 63/675,434, filed on Jul. 25, 2024.
(60) Provisional application No. 63/520,246, filed on Aug. 17, 2023.

(51) Int. Cl. C07D 401/12; A61K 31/4355; A61K 31/4439; A61K 31/506; A61P 31/14; C07D 401/14; C07D 413/14; C07D 417/14; C07D 491/048

(52) U.S. Cl. CPC C07D 401/12; A61K 31/4355; A61K 31/4439; A61K 31/506; A61P 31/14; C07D 401/14; C07D 413/14; C07D 417/14; C07D 491/048

(57) ABSTRACT

The present disclosure is directed to compounds of Formula I:

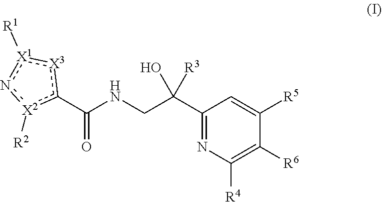

and their use as anti-viral agents for inhibition of the replication of hRSV and hMPV and treatment and prophylaxis of hRSV and hMPV infection.

### CROSS-REFERENCE TO RELATED APPLICATIONS

This application claims the benefit of U.S. Provisional Patent Application Ser. No. 63/520,246 filed Aug. 17, 2023 and U.S. Provisional Patent Application Ser. No. 63/675,434 filed Jul. 25, 2024, the entire contents of which are incorporated by reference herein.

### FIELD OF THE INVENTION

The present invention relates to therapeutic compounds useful for the inhibition of respiratory syncytial virus replication and metapneumovirus replication. The therapeutic compounds may be used in the treatment or prevention of respiratory syncytial virus infection and metapneumovirus infection.

### BACKGROUND OF THE INVENTION

Paramyxoviruses are enveloped negative-strand RNA viruses that are significant human and animal pathogens. Human Respiratory Syncytial Virus (hRSV, RSV) belongs to the family Paramyxoviridae, subfamily Pneumovirinae. Two subtypes, type A and type B, have been identified and are a major cause of severe and sometimes even fatal respiratory infections in children less than 6 months of age. Adults with underlying diseases, such as COPD, asthma, cancer, immunocompromised status, including HIV or post transplantation, are also at risk of developing severe RSV infections. Fifteen percent of annual hospitalizations in adults over 50 years of age are due to acute respiratory infections caused by RSV. In the United States, RSV causes more than 100,000 hospitalizations annually and is estimated to cause 160,000 deaths globally each year. Other viral family members, including human metapneumovirus (hMPV) and human Parainfluenza Virus (hPIV), are also responsible for acute respiratory illness similar to hRSV.

The RSV genome is a single-stranded negative-sense RNA molecule of approximately 15 kb, which encodes for 11 proteins. Two of these proteins are the main surface glycoproteins of the virion. These are the attachment (G) protein, which mediates virus binding to cells, and the fusion (F) protein, which promotes both fusion of the viral and cell membranes at the initial stages of the infectious cycle and fusion of the membrane of infected cells with those of adjacent cells to form characteristic syncytia. Four of the polypeptides, together with the viral RNA genome, form the RSV ribonucleoprotein (RNP) complex. These proteins are the nucleocapsid (N) protein, phosphoprotein (P), RNA polymerase (L) protein, and transcription factor M2-1, which are also each required for the transcription and replication of the viral genome and the subject of drug discovery research.

After hRSV, hMPV is the second most common cause of lower respiratory infection in young children. hMPV is responsible for 5-40% of respiratory tract infections in hospitalized and outpatient children. In healthy adults, hMPV generally results in mild respiratory tract infections; However, adults 70 years old and older, immunocompromised individuals and, people with comorbidities such as asthma and chronic obstructive pulmonary disease (COPD) are at higher risk for more serious disease and hospitalization as a result of hMPV infection. The hMPV genome is approximately 13 kb and the organization is similar to hRSV. hMPV genome RNA replication and mRNA transcription relies on the hMPV L-protein polymerase, which is highly homologous with the hRSV L-protein polymerase.

Currently there are few options available for prophylaxis and treatment of RSV infections. There is no marketed vaccine for RSV. A clinical trial with a formalin-inactivated virus was associated with increased disease severity in infants upon infection with RSV (see Kim et al., American Journal of Epidemiology, 89:422-434 (1969)). The monoclonal antibody palivizumab (Synagis®) is approved for prophylactic use but has limited efficacy and its use is limited to high-risk infants as a result of high cost. Ribavarin (ViraZole®), a guanosine nucleoside analog broad-spectrum antiviral is approved as an inhaled treatment for RSV infection in infants, but clear efficacy data is lacking (Fearns et al., 2016 Antiviral Research, 134:63-76). In addition, the teratogenic potential of ribavirin raises significant risks for caregivers. The standard of care currently for RSV-infected patients is palliative and includes supplemental oxygen and intravenous fluids.

There continues to be a need for anti-viral agents with pharmacokinetic properties suitable for a significant number of patients in the affected population. The present invention provides novel replication inhibitors of hRSV and hMPV useful for the inhibition of respiratory syncytial virus replication and metapneumovirus replication for addressing this need.

### SUMMARY OF THE INVENTION

The present disclosure is directed to compounds of Formula I and embodiments thereof for use as anti-viral agents for inhibition of the replication of hRSV and hMPV and the treatment and prophylaxis of hRSV and hMPV infection. Compositions and methods of use comprising the compounds of this disclosure are also provided.

In some aspects, the disclosure provides biaryl amide compounds containing a flexible linker adjacent to the amide bond. In some aspects, these compounds comprise a hydrophobic ring core and hydrophilic substituent containing a substituted amine moiety.

All structural Formulas, embodiments and classes thereof described herein include the pharmaceutically acceptable salts of the compounds defined therein. Reference to the compounds of this invention as those of a specific formula or embodiment, e.g., Formula I, II, or III, or embodiments thereof, is intended to encompass the specific compound or compounds falling within the scope of the formula or embodiment, including salts thereof, particularly pharmaceutically acceptable salts, solvates (including hydrates) of such compounds and solvated salt forms thereof, where such forms are possible, unless specified otherwise.

The present invention includes each of the Examples described herein, and pharmaceutically acceptable salts thereof. The invention also encompasses pharmaceutical compositions comprising an effective amount of a compound of the invention or a pharmaceutically acceptable salt thereof, and a pharmaceutically acceptable carrier.

### DETAILED DESCRIPTION OF THE INVENTION

The present disclosure is directed to compounds of Formula I

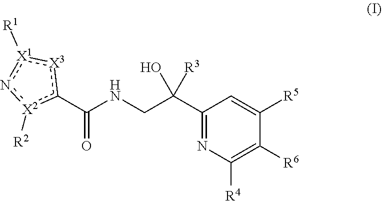

or a pharmaceutically acceptable salt thereof, wherein:
R¹ is selected from:
   (a) —OC₁₋₃ alkyl,
   (b) a 5-member heteroaryl ring comprised of three carbon atoms and (i) two of N, (ii) N and S, or (iii) N and O, and
   (c) a 6-member heteroaryl ring comprised of four carbon atoms and two of N;
X¹ is independently selected from C and N;
X² is selected from C, S and N,
   wherein X² is C when X¹ is N, or when X³ is S;
X³ is selected from C, S and N,
   wherein X³ is C when X² is S or N, or when X¹ is N;
R² is selected from:
   (a) —H,
   (b) —C₁₋₃alkyl,
   (c) —C₃₋₅cycloalkyl, and
   (d) —OC₁₋₃alkyl;
R³ is selected from:
   (a) —C₁₋₃alkyl,
   (b) —C₃₋₅cycloalkyl,
   (c) —CF₃,
   (d) —CF₂H, and
   (e) —H;
R⁴ is selected from:
(1) phenyl substituted with 1 to 3 substituents independently selected at each occurrence from:
   (a) halo, and
   (b) —C₁₋₃alkyl, or
(2),

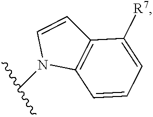

wherein R⁷ is selected from —H and halo, or
(3)

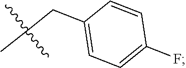 [SMILES: *Cc1ccc(F)cc1]

R⁵ is selected from:
   (1) a —C₁₋₅alkyl, unsubstituted or substituted with (i) —NH₂, (ii) —(C═O)NH₂, or (iii) —OH,
   (2) pyrrolidinyl, unsubstituted or substituted with —C₁₋₃ alkyl; and
R⁶ is selected from:
   (1) —H, and
   (2) —O—, wherein R⁶ and any alkyl moiety of R⁵ are taken together with their intervening atoms to form a cyclic group.

Further provided are compounds of Formula I, or a pharmaceutically acceptable salt thereof, wherein:
R¹ is selected from:
ethoxy,

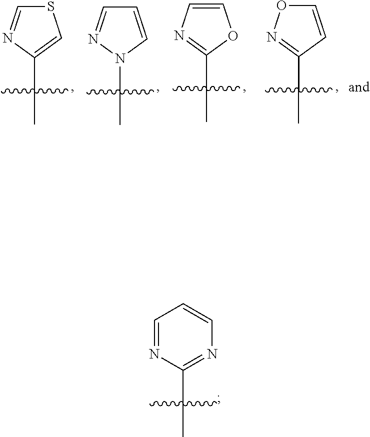

X¹ and X³ are C;
X² is N;
R² is methyl;
R³ is methyl;
R⁴ is phenyl substituted with 1 or 2 substituents independently selected at each occurrence from fluoro and chloro; and
R⁵ is a —C₃alkyl substituted with —NH₂; and
R⁶ is —H.

In Embodiment 1 of this disclosure are compounds of Formula I, or a pharmaceutically acceptable salt thereof, wherein X¹ is C and X² is N. In a class of Embodiment 1 are compounds of Formula I, or a pharmaceutically acceptable salt thereof, wherein X³ is C.

In Embodiment 2 of this disclosure are compounds of Formula I and Embodiment 1, or a pharmaceutically acceptable salt thereof, wherein R¹ is selected from ethoxy,

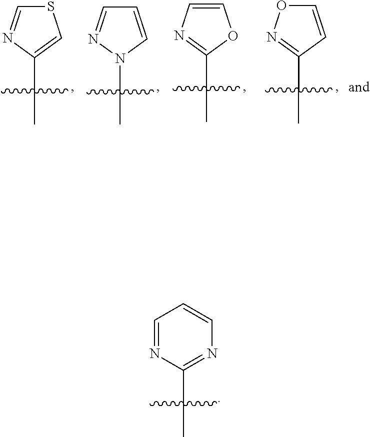

In a first class thereof, R¹ is

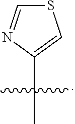 [SMILES: *c1cscn1]

In a second class thereof, R¹ is ethoxy. In a third class thereof, R¹ is

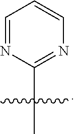 [SMILES: *c1ncccn1]

In Embodiment 3 of this disclosure are compounds of Formula I, and each of Embodiments 1 and 2, or a pharmaceutically acceptable salt thereof, wherein R² is methyl or ethyl. In a first class of Embodiment 3, R² is methyl. In a second class of Embodiment 3, R² is cyclopentanyl.

In a sub-class of the first class of Embodiment 3, R³ is methyl. In a second sub-class of the first class of Embodiment 3, R³ is cyclopentanyl. In a third sub-class of the first class of Embodiment 3, R³ is CF₃. In a fourth sub-class of the first class of Embodiment 3, R³ is —H.

In Embodiment 4 of this disclosure are compounds of Formula I, and each of Embodiments 1-3, or a pharmaceutically acceptable salt thereof, wherein R⁵ is a —C₃alkyl substituted with —NH₂. In a sub-class of Embodiment 4, R⁵ is 2-aminopropan-2-yl (or, isopropylaminyl).

In Embodiment 5 of this disclosure are compounds of Formula I, and each of Embodiments 1-4, or a pharmaceutically acceptable salt thereof, wherein R⁴ is a phenyl substituted with 1 or 2 substituents independently selected at each occurrence from methyl, fluoro and chloro. In a first class of Embodiment 5, R⁴ is a phenyl substituted with at least one fluoro group. In a sub-embodiment of the first class, R⁴ is

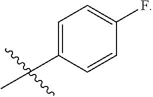 [SMILES: *c1ccc(F)cc1]

In a second class of Embodiment 5, R⁴ is a phenyl substituted with a chloro group. In a third class of Embodiment 5, R⁴ is a phenyl substituted with two or three fluoro groups. In a sub-class of the first and second classes of Embodiment 5, R⁴ is a phenyl substituted with a fluoro group and a chloro group.

In some aspects of the disclosed compounds, provided herein is a compound is of Formula II:

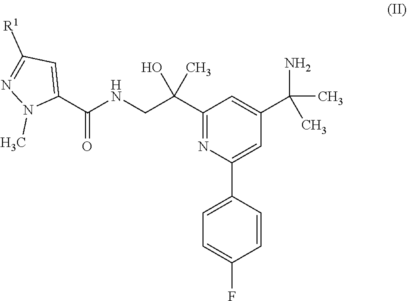

or a pharmaceutically acceptable salt thereof, wherein R¹ is defined as above.

In Embodiment 6 of this disclosure are compounds of Formula I, or a pharmaceutically acceptable salt thereof, wherein X¹ is C and X² is C. In a class of Embodiment 6 are compounds of Formula I, or a pharmaceutically acceptable salt thereof, wherein X³ is S. In some sub-embodiments of Embodiment 6, R² is methyl or cyclopentanyl. In some sub-embodiments of Embodiment 6, R³ is methyl or cyclopentanyl.

In Embodiment 7 of this disclosure are compounds of Formula I, or a pharmaceutically acceptable salt thereof, wherein X¹ is C and X² is S (and thus X³ is C).

In some aspects of the disclosed compounds, provided herein is a compound is of Formula III:

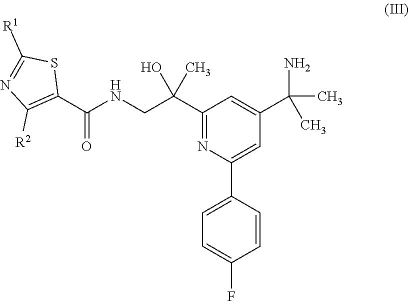

or a pharmaceutically acceptable salt thereof, wherein R¹, R² and R³ are defined as above.

In Embodiment 8 of this disclosure are compounds of Formula I, or a pharmaceutically acceptable salt thereof, wherein R⁵ and R⁶ are taken together with their intervening atoms to form a cyclic group. In a class of Embodiment 8, R⁵ is 2-aminopropan-2-yl, and R⁶ is —O—. In a second class of Embodiment 8, R⁵ and R⁶ are taken together with their intervening atoms to form a 5-membered cyclic group.

In Embodiment 9 of this disclosure are compounds of Formula I, and each of Embodiments 1-3, or a pharmaceutically acceptable salt thereof, wherein R⁵ is a —C₁₋₄alkyl, unsubstituted or substituted with —NH₂.

In Embodiment 10 of this disclosure are compounds of Formula I, and each of Embodiments 1-3, wherein R⁵ is a —C₁₋₅alkyl that is substituted with an amide group (i.e., —(C═O)NH₂)).

Reference to the compounds of Formula I herein encompasses the compounds of Formula II and III, and all embodiments, classes and sub-classes thereof and includes the compounds of the Examples herein. The compounds of Formula I encompass neutral compounds or salts thereof when such salts are possible, including pharmaceutically acceptable salts.

### Definitions

The term “e.g.” means “for example.” When the terms “e.g.,” or “for example” are used herein, the example(s) recited are intended to be illustrative and are not intended to be an exhaustive list of all relevant examples.

As used herein, “alkyl” refers to both branched- and straight-chain saturated aliphatic hydrocarbon groups having the specified number of carbon atoms in a specified range. For example the term “C₁₋₄alkyl” means linear or branched chain alkyl groups, including all possible isomers, having 1, 2, 3 or 4 carbon atoms, and includes n-, iso-, sec- and tert-butyl (butyl, i-butyl, s-butyl, t-butyl, collectively “C₄alkyl”; Bu=butyl), n- and i-propyl (propyl, i-propyl, collectively “C₃alkyl”; Pr=propyl), ethyl (Et) and methyl (Me). “C₁₋₃alkyl” has 1, 2 or 3 carbon atoms and includes each of n-propyl, i-propyl, ethyl and methyl.

“Cycloalkyl” refers to a cyclized alkyl ring having the indicated number of carbon atoms in a specified range. Thus, for example, “C₃₋₅cycloalkyl” includes each of cyclopropyl, cyclobutyl, and cyclopentyl.

“Halo” or “halogen” refers to chloro, fluoro, bromo or iodo. Chloro, fluoro and bromo are a class of halogens of interest, and more particularly fluoro and chloro.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present disclosure are limited to stable compounds embraced by Formula I and its embodiments. For example, certain moieties as defined in Formula I may be unsubstituted or substituted, and the latter is intended to encompass substitution patterns (i.e., number and kind of substituents) that are chemically possible for the moiety and that result in a stable compound.

This disclosure includes individual diastereomers, particularly epimers, i.e., compounds having the same chemical formula but which differ in the spatial arrangement around a single atom. This disclosure also includes mixtures of diastereomers, particularly mixtures of epimers, in all ratios. This disclosure encompasses compounds of Formula I having either the (R) or (S) stereo-configuration at an asymmetric center and at any additional asymmetric centers that may be present in a compound of Formula I, as well as stereo-isomeric mixtures thereof. Embodiments of this disclosure also include a mixture of enantiomers enriched with 51% or more of one of the enantiomers, including for example 60% or more, 70% or more, 80% or more, or 90% or more of one enantiomer. A single epimer is preferred. An individual or single enantiomer refers to an enantiomer obtained by chiral synthesis and/or using generally known separation and purification techniques, and which may be 100% of one enantiomer or may contain small amounts (e.g., 10% or less) of the opposite enantiomer. Thus, individual enantiomers are a subject of this disclosure in pure form, both as levorotatory and as dextrorotatory antipodes, in the form of racemates and in the form of mixtures of the two enantiomers in all ratios. In the case of a cis/trans isomerism this disclosure includes both the cis form and the trans form as well as mixtures of these forms in all ratios.

The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis. The present disclosure includes all such isomers, as well as salts, solvates (which includes hydrates). and solvated salts of such racemates, enantiomers, diastereomers and tautomers and mixtures thereof.

As would be recognized by one of ordinary skill in the art, certain compounds of the present disclosure may be able to exist as tautomers. All tautomeric forms of such compounds, whether isolated individually or in mixtures, are within the scope of the present disclosure. For example, in instances where an oxo (═O) substituent is permitted on an aromatic heterocyclic ring (also referred to as a heteroaromatic or heteroaryl ring) and keto-enol tautomerism is possible, it is understood that the substituent might in fact be present, in whole or in part, in the —OH form.

The atoms in a compound of Formula I may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of Formula I; for example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of Formula I can be prepared without undue experimentation by conventional techniques well known to those skilled in the art or by processes analogous to those described in the Schemes and Examples herein using appropriate isotopically-enriched reagents and/or intermediates.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). When the compounds of Formula I contain one or more acidic groups or basic groups, the invention includes the corresponding pharmaceutically acceptable salts.

The compounds of Formula I, and pharmaceutically acceptable salts thereof, which contain one or more basic groups, i.e., groups which can be protonated, can be used according to the invention in the form of their acid addition salts with inorganic or organic acids as, for example but not limited to, salts with hydrogen chloride, hydrogen fluoride, hydrogen bromide, trifluoroacetic acid (trifluoroacetate), phosphoric acid, sulfuric acid, nitric acid, benzenesulfonic acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, etc. In some embodiments, one or more NH₂ groups (e.g., a single NH₂ group, or two NH₂ groups) of any of the compounds of Formula I is protonated in a salt form. In some embodiments, one or more NH₂ groups (e.g., a single NH₂ group) of any of the compounds of Formula I is protonated in a salt form with trifluoroacetic acid (trifluoroacetate). In some embodiments, one or more NH₂ groups (e.g., a single NH₂ group) of any of the compounds of Formula I is protonated in a salt form with hydrogen chloride. If the compounds of Formula I simultaneously contain acidic and basic groups in the molecule the invention also includes, in addition to the salt forms mentioned, inner salts or betaines (zwitterions). Salts can be obtained from the compounds of Formula I by customary methods which are known to the person skilled in the art, for example by combination with an organic or inorganic acid or base in a solvent or dispersant, or by anion exchange or cation exchange from other salts. The present disclosure also includes all salts of the compounds of Formula I which, owing to low physiological compatibility, are not directly suitable for use in pharmaceuticals but which can be used, for example, as intermediates for chemical reactions or for the preparation of pharmaceutically acceptable salts.

The present disclosure encompasses any composition comprised of a compound of Formula I or a compound that is a salt thereof, including for example but not limited to, a composition comprised of said compound associated together with one or more additional molecular and/or ionic component(s) which may be referred to as a “co-crystal.” The term “co-crystal” as used herein refers to a solid phase (which may or may not be crystalline) wherein two or more different molecular and/or ionic components (generally in a stoichiometric ratio) are held together by non-ionic interactions including but not limited to hydrogen-bonding, dipole-dipole interactions, dipole-quadrupole interactions or dispersion forces (van der Waals). There is no proton transfer between the dissimilar components and the solid phase is neither a simple salt nor a solvate. A discussion of co-crystals can be found, e.g., in Aitipamula et al., Crystal Growth and Design, 2012, 12 (5), pp. 2147-2152.

Furthermore, compounds of the present disclosure may exist in amorphous form and/or one or more crystalline forms, and as such all amorphous and crystalline forms and mixtures thereof of the compounds of Formula I and salts thereof are intended to be included within the scope of the present disclosure. In addition, some of the compounds of the present disclosure may form solvates with water (i.e., a hydrate) or common organic solvents. Such solvates and hydrates, particularly the pharmaceutically acceptable solvates and hydrates, of the compounds of this disclosure are likewise encompassed within the scope of the compounds defined by Formula I and the pharmaceutically acceptable salts thereof, along with un-solvated and anhydrous forms of such compounds.

Accordingly, the compounds of Formula I or salts thereof including pharmaceutically acceptable salts thereof, embodiments thereof and specific compounds described and claimed herein, encompass all possible stereoisomers, tautomers, physical forms (e.g., amorphous and crystalline forms), co-crystal forms, solvate and hydrate forms, and any combination of the foregoing forms where such forms are possible.

Another embodiment of the present disclosure is a compound of Formula I wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound of Formula I or its salt (e.g., the product isolated from a reaction mixture affording the compound or salt) consists of the compound or salt. The level of purity of the compounds and salts can be determined using a standard method of analysis such as, high performance liquid chromatography, and/or mass spectrometry or NMR techniques. If more than one method of analysis is employed and the methods provide experimentally significant differences in the level of purity determined, then the method providing the highest purity level governs. A compound or salt of 100% purity is one which is free of detectable impurities as determined by a standard method of analysis. With respect to a compound of the invention which has one or more asymmetric centers and can occur as mixtures of stereoisomers, a substantially pure compound can be either a substantially pure mixture of the stereoisomers or a substantially pure individual stereoisomer.

The compounds of Formula I herein, and pharmaceutically acceptable salts thereof, are useful for the inhibition of respiratory syncytial virus replication and metapneumovirus replication. Thus, the compounds of Formula I and pharmaceutically acceptable salts thereof are useful for:
(i) a method for the treatment of respiratory syncytial virus infection in a human subject in need thereof which comprises administering to the human subject an effective amount of the compound according to Formula I, or a pharmaceutically acceptable salt thereof;
(ii) a method for the prophylaxis of respiratory syncytial virus infection in a human subject in need thereof which comprises administering to the human subject an effective amount of the compound according to Formula I, or a pharmaceutically acceptable salt thereof;
(iii) a method for the treatment of metapneumovirus infection in a human subject in need thereof which comprises administering to the human subject an effective amount of the compound according to Formula I, or a pharmaceutically acceptable salt thereof; and/or
(iv) a method for the prophylaxis of metapneumovirus infection in a human subject in need thereof which comprises administering to the human subject an effective amount of the compound according to Formula I, or a pharmaceutically acceptable salt thereof.

In some aspects, provided herein are uses of any of the disclosed compounds in therapy. Further provided herein are uses of any of the disclosed compounds as a medicament, or in the manufacture of a medicament, for the treatment of respiratory syncytial virus infection, prophylaxis of respiratory syncytial virus infection, treatment of metapneumovirus infection, prophylaxis of metapneumovirus infection, or inhibition of the replication of hRSV or hMPV.

Additional embodiments of the present disclosure include the following:
(a) a pharmaceutical composition comprising an effective amount of a compound of Formula I or a pharmaceutically acceptable salt thereof, and a pharmaceutically acceptable carrier, and
(b) a pharmaceutical composition which comprises the product prepared by combining (e.g., mixing) an effective amount of a compound of Formula I or a pharmaceutically acceptable salt thereof, and a pharmaceutically acceptable carrier.

Additional embodiments of the present disclosure include each of the pharmaceutical compositions, methods and uses set forth in the preceding paragraphs, wherein the compound of Formula I or its salt employed therein in substantially pure. With respect to a pharmaceutical composition comprising a compound of Formula I or its salt and a pharmaceutically acceptable carrier and optionally one or more excipients, it is understood that the term “substantially pure” is in reference to a compound of Formula I or its salt per se.

The term “administration” and variants thereof (e.g., “administering” a compound) in reference to a compound of Formula I means providing the compound to the individual in need of treatment or prophylaxis and includes both self-administration and administration to the patient by another person or any other means.

As used herein, the term “composition” is intended to encompass a product comprising the specified ingredients, as well as any product which results from combining the specified ingredients. Ingredients suitable for inclusion in a pharmaceutical composition are pharmaceutically acceptable ingredients, which means the ingredients must be compatible with each other and not deleterious to the recipient thereof.

The term “subject” as used herein refers to an animal, such as a human, that is the object of treatment, observation or experiment. In various embodiments of the present disclosure, a “subject” encompasses a mammalian animal. In some embodiments, the subject encompasses a domesticated or companion animal, or an experimental animal model. In some embodiments, the subject is a rodent, such as a mouse or rat. In some embodiments, the subject is a primate. In some embodiments, the subject is a non-human primate, such as a macaque. In some embodiments, the subject is a human.

The term “human subject” or “patient” as used herein refers to a human (or “person”) who has been the object of treatment, observation or experiment. Patients to be treated with an RSV inhibitor (RSV-i) and/or an MPV inhibitor (MPV-i) agent include but are not limited to, patients who have been infected with RSV and/or MPV. Patients to be treated with an RSV-i and/or an MPV-i agent also include, but are not limited to, those using an RSV-i and/or an MPV-i agent for prophylaxis of RSV and/or MPV infection or for post-exposure prophylaxis after being potentially exposed to RSV and/or MPV to prevent or reduce the severity of symptoms of virus-associated disease or condition.

“Prophylaxis” includes each of (i) pre-exposure prophylaxis (PrEP), i.e., using a compound of Formula I, or a pharmaceutically acceptable salt thereof, to prevent hRSV and/or hMPV infection in a person who is not infected with hRSV and/or hMPV, and (ii) post-exposure prophylaxis (PEP), i.e., using a compound of Formula I, or a pharmaceutically acceptable salt thereof, after being exposed or potentially exposed to hRSV and/or hMPV to prevent or reduce the severity of symptoms of virus-associated disease or condition. In some embodiments, a compound of Formula I, or a pharmaceutically acceptable salt thereof, is administered for PEP in patients exposed to RSV to minimize the risk of severe RSV disease or symptoms thereof.

The term “effective amount” as used herein means the amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented.

When the active compound (i.e., active ingredient) is administered as the salt, references to the amount of active ingredient are to the free form (i.e., the non-salt form) of the compound.

In the methods of the present disclosure, the disclosed compounds, and salts thereof, can be administered by means that produce contact of the active agent with the agent's site of action. They can be administered by conventional means available for use in conjunction with pharmaceuticals, either as individual therapeutic agents or with other therapeutic agents the patient may be in need of. The compound can be administered itself, but typically is administered with a pharmaceutical carrier selected on the basis of the chosen route of administration and standard pharmaceutical practice. The compounds of the invention can, for example, be administered orally (e.g., via tablet or capsule), parenterally (including subcutaneous injection; intravenous, intramuscular or intrasternal injection; or infusion techniques), by inhalation spray, or rectally, in the form of a unit dosage of a pharmaceutical composition containing an effective amount of the compound and conventional non-toxic pharmaceutically acceptable carriers, adjuvants and vehicles. The compound could also be administered via an implantable drug delivery device adapted to provide an effective amount of the compound or a pharmaceutical composition of the compound over an extended period of time.

### Formulations

Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Liquid preparations suitable for oral administration (e.g., suspensions, syrups, elixirs and the like) can be prepared according to techniques known in the art and can employ any of the usual media such as water, glycols, oils, alcohols and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Implantable compositions can be prepared according to methods known in the art wherein the carrier comprises the active chemical ingredient with polymers and suitable excipients, or utilizing an implantable device for drug delivery. Further description of methods suitable for use in preparing pharmaceutical compositions for use in the present disclosure and of ingredients suitable for use in said compositions is provided in Remington—The Science and Practice of Pharmacy, 22nd Edition, published by Pharmaceutical Press and Philadelphia College of Pharmacy at University of the Sciences, 2012, ISBN 978 0 85711-062-6 and prior editions.

Formulations of compounds of Formula I that result in drug supersaturation and/or rapid dissolution may be utilized to facilitate oral drug absorption. Formulation approaches to cause drug supersaturation and/or rapid dissolution include, but are not limited to, nanoparticulate systems, amorphous systems, solid solutions, solid dispersions, and lipid systems. Such formulation approaches and techniques for preparing them are known in the art. For example, solid dispersions can be prepared using excipients and processes as described in reviews (e.g., A. T. M. Serajuddin, J Pharm Sci, 88:10, pp. 1058-1066 (1999)). Nanoparticulate systems based on both attrition and direct synthesis have also been described in reviews such as Wu et al. (F. Kesisoglou, S. Panmai, Y. Wu, Advanced Drug Delivery Reviews, 59:7 pp. 631-644 (2007)).

The compounds of Formula I may be administered in a dosage range of, e.g., 1 to 20 mg/kg, or 1 to 10 mg/kg, or about 5 mg/kg of mammal (e.g., human) body weight per day, or at other time intervals as appropriate, in a single dose or in divided doses. The compounds of Formula I may be administered in a dosage range of 0.001 to 2000 mg per day in a single dose or in divided doses. Examples of dosage ranges are 0.01 to 1500 mg per day, or 0.1 to 1000 mg per day, administered orally or via other routes of administration in a single dose or in divided doses.

For oral (e.g., tablets or capsules) or other routes of administration, the dosage units may contain 100 mg to 1500 mg of the active ingredient for the symptomatic adjustment of the dosage to the patient to be treated. Furthermore, the compound may be formulated in oral formulations for immediate or modified release such as extended or controlled release. When the compound of Formula I is administered as a salt, reference to an amount of the compound in milligrams or grams is based on the free form (i.e., the non-salt form) of the compound.

Daily administration can be via any suitable route of administration but is preferably via oral administration and can be a single dose or more than one dose at staggered times (divided daily doses) within each 24-hour period. Each dose may be administered using one or multiple dosage units as appropriate.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, the effect of other drugs the patient is taking while using a RSV-i or a MPV-i compound described herein, the severity of the particular condition, and the host undergoing therapy. In some cases, depending on the potency of the compound or the individual response, it may be necessary to deviate upwards or downwards from the given dose. The amount and frequency of administration will be regulated according to the judgment of the attending clinician considering such factors.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals.

It is understood that a chiral or stereogenic center in any of the disclosed compounds may exist in the (S) or (R) stereo-configuration, or as a mixture of both. Within the provided molecular structures of these compounds, each bond drawn as a straight line from a chiral center encompasses each of the (R) and (S) stereoisomers, as well as mixtures thereof, unless otherwise noted. For compounds in the Examples that contain a chiral center, stereoisomer mixtures may have been separated, providing one or both of an isomer 1 (the faster eluting isomer) and an isomer 2 (the slower eluting isomer), based on their observed elution order resulting from the separation as performed at some time during the synthesis described in the Example, or that of an intermediate of the compound. Elution time and/or order of separated isomers may not be the same if performed under conditions different from those employed in the relevant Example. Absolute stereochemistry (R or S) of the chiral center in each of isomer “1” and/or isomer “2” in the Examples was not determined. The designations “1” and “2” solely refer to elution order resulting from the purification conditions as performed. This nomenclature extends to diastereomers (which may be referred to herein as “diastereoisomers”) of the disclosed compounds. Depending on the number of stereogenic centers, the numbering system may be “1”, “2”, “3”, “4”, etc., providing multiple isomers and are numbered based on their observed elution order resulting from the separation as performed at some time during the synthesis described in the Example, or that of an intermediate of the compound. Elution time and/or order of separated isomers may not be the same if performed under conditions different than those employed in the relevant Example.

### GENERAL PROCEDURES

The following reaction schemes and Examples illustrate methods which may be employed for the synthesis of the compounds of structural Formula I described in this invention. These reaction schemes and Examples are provided to illustrate the invention and are not to be construed as limiting the invention in any manner. All substituents are as defined above unless indicated otherwise. Several strategies based upon synthetic transformations known in the literature of organic synthesis may be employed for the preparation of the compounds of structural Formula I.

The compounds of the present disclosure can be prepared according to the procedures of the following Examples, using appropriate materials. The compounds illustrated in the examples are not, however, to be construed as forming the only genus that is considered as the invention. The Examples further illustrate details for the preparation of the compounds of the present disclosure. Those skilled in the art will readily understand that known variations of protecting groups, as well as of the conditions and processes of the following preparative procedures, can be used to prepare these compounds. It is also understood that whenever a chemical reagent such as a boronic acid or a boronate is not commercially available, such a chemical reagent can be readily prepared following one of numerous methods described in the literature.

Reactions sensitive to moisture or air were performed under nitrogen or argon using anhydrous solvents and reagents. The progress of reactions was determined by either liquid chromatography-mass spectrometry (LC-MS) or analytical thin layer chromatography (TLC) usually performed with glass-backed TLC plates (Merck KGaA), silica gel 60 F₂₅₄. All temperatures are degrees Celsius unless otherwise noted. Mass spectra (MS) were measured either by electrospray ionization mass spectroscopy (ESI) or by atmospheric pressure chemical ionization mass spectroscopy.

In general, compounds of Formula (I) can be prepared by amide coupling of appropriately functionalized carboxylic acids of Formula (IV) and amines of Formula (V). Acids of Formula (IV) and amines of Formula (V) are commercially available or may be synthesized from appropriate intermediates. Other synthetic methods to prepare compounds of Formula (I) are presented for the exemplification of compounds. Further, compounds of Formula (I) can be prepared by reaction procedures generally known in the art.

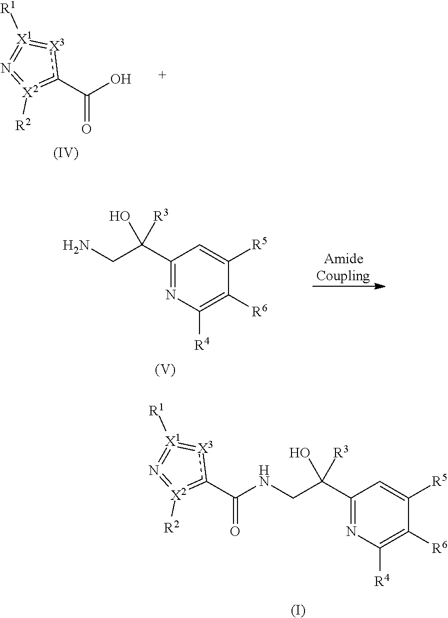

Analytical LC-MS was commonly performed on a Waters SQD single quadrupole mass spectrometer with electrospray ionization in positive ion detection mode (mass range set at 150-900 daltons, data collected in centroid mode and scan time set to 0.2 seconds) and a Waters Acquity UPLC system (binary solvent manager, sample manager, and TUV). The column used was a Waters Acquity BEH C18 1×50 mm, 1.7 μm, heated to 50° C. The mobile phases used were modified with either acidic or basic additives. The acidic mobile phase consisted of water (with 0.1% TFA modifier) for Solvent A and 100% MeCN for Solvent B. A two-minute run was established at a flow rate of 0.3 mL/min with initial conditions of 95% Solvent A and ramping up to 99% Solvent B at 1.60 minutes and holding at 99% Solvent B for 0.40 minutes. The injection volume was 0.5 μL using partial loop needle overfill injection mode. The TUV monitored wavelength 215 or 254 nm with a sampling rate of 20 points/second, normal filter constant and absorbance data mode. The basic mobile phase consisted of water (with 0.05% NH₄OH modifier) for solvent A and 100% MeCN for solvent B. A two-minute run was established at a flow rate of 0.3 mL/min with initial conditions of 99% Solvent A and ramping up to 99% Solvent B at 1.90 minutes and holding at 99% Solvent B for 0.10 minutes. A five-minute run was established at a flow rate of 0.3 mL/min with initial conditions of 95% Solvent A and ramping up to 99% Solvent B at 4.90 minutes and holding at 99% Solvent B for 0.10 minutes. For both methods, the injection volume was 5.0 μL using Partial Loop Needle Overfill Injection mode. The TUV monitored wavelength 215 nm with a sampling rate of 20 points/second, normal filter constant and absorbance data mode. Alternatively, a commonly used system consisted of a Waters ZQ™ platform with electrospray ionization in positive ion detection mode with an Agilent 1100 series HPLC with autosampler. The column was commonly a Waters Xterra MS C18, 3.0×50 mm, 5 μm or a Waters Acquity UPLC® BEH C18 1.0×50 mm, 1.7 m. The flow rate was 1 mL/min, and the injection volume was 10 μL. UV detection was in the range 210-400 nm. The mobile phase consisted of solvent A (water with 0.05% TFA modifier) and solvent B (MeCN with 0.05% TFA modifier) with a gradient of 100% solvent A for 0.7 min changing to 100% solvent B over 3.75 min, maintained for 1.1 min, then reverting to 100% solvent A over 0.2 min.

Preparative reverse-phase chromatography was generally carried out on a Teledyne ISCO ACCQPrep HP125 or HP150 apparatus equipped with UV and ELSD detectors. The UV detector typically monitored wavelengths of 215 and 254 nm. The column was commonly one of the following: Waters XBridge Prep C18 OBD 5 μm 30×150 mm, Waters XBridge Prep C18 OBD 5 μm 30×250 mm, Waters XBridge Prep C18 OBD 5 μm 50×250 mm, Waters SunFire Prep C18 OBD 5 μm 30×150 mm, Waters SunFire Prep C18 OBD 10 μm 30×150 mm, Waters SunFire Prep C18 OBD 5 μm 50×250 mm, Waters SunFire Prep C18 OBD 10 μm 50×250 mm, or Phenomenex Luna Prep C18 5 μm 50×250 mm. The mobile phases consisted of mixtures of MeCN (with 0.1% TFA modifier) and water (with 0.1% TFA modifier). Alternatively, a commonly used system was a Waters Chromatography Workstation configured with an LCMS system consisting of: Waters ZQ™ single quad MS system with Electrospray Ionization, Waters 2525 Gradient Pump, Waters 2767 Injector/Collector, Waters 996 PDA Detector. MS conditions were: 150-750 amu, positive electrospray, collection triggered by MS. Columns used were commonly a Waters SunFire C18 5 μm 30×150 mm, a Boston Green ODS 5 μm 150×30 mm, or a YMC-Actus Triart C18 5 μm 150×30 mm column. The mobile phases consisted of mixtures of MeCN (10-100%) in water (with 0.1% TFA modifier). Flow rates were maintained at 50 mL/min, and the UV detection range was 210-400 nm. An additional preparative HPLC system used was a Gilson Workstation consisting of: Gilson GX-281 Injector/Collector, Gilson UV/VIS-155 Detector, Gilson 333 and 334 Pumps, and either a Phenomenex Gemini-NX C18 5 μm 50×250 mm column, a Waters XBridge Prep C18 OBD 5 μm 30×250 mm, or a Welch Xtimate C18 5 μm 150×25 mm. The mobile phases consisted of mixtures of acetonitrile (0-75%) in water containing 5 mM (NH₄)HCO₃. Flow rates were maintained at 50 mL/min for the Waters XBridge column, 90 mL/min for the Phenomenex Gemini column, and 25 mL/min for the Welch Xtimate column. The UV detection range was 210-400 nm. Mobile phase gradients were optimized for the individual compounds.

Silica gel chromatography was typically performed using an ISCO CombiFlash Rf apparatus, a Biotage® Flash Chromatography apparatus (Dyax Corp.), or an ISCO CombiFlash® Companion XL apparatus on silica gel (60 Å pore size) in pre-packed RediSep Rf, RediSep Rf Gold, or SepaFlash columns. Mobile phases generally consisted of mixtures of hexanes, petroleum ether, or DCM with EtOAc, 3:1 EtOAc:EtOH, or MeOH. Mobile phase gradients were optimized for the individual compounds.

Chiral chromatography was typically performed by supercritical fluid chromatography with a column chosen from one of the following: CHIRAL ART Amylose-C NEO (YMC), ChiralCel® OD-3 (Daicel), ChiralPak® AD (Daicel), ChiralPak® AD-3, ChiralPak® AD-H, ChiralPak® AY-3, ChiralPak® AS, ChiralPak® AS-3, ChiralPak® AS-H, ChiralPak® IG, ChiralPak® IG-3, ChiralPak® IB-N, ChiralPak® OD-H, ChiralCel® OJ, ChiralCel® OJ-3, ChiralPak® OJ-H, ChiralPak® IC-3, Phenomenex-Cellulose-2, or (S,S)Whelk-O1. Mobile phases consisted of mixtures of CO₂, n-heptane, or hexane with MeOH, EtOH, or IPA. In some cases, a modifier of 0.05-0.1% isopropylamine, DEA or NH₄OH was used. Mobile phase gradients were optimized for the individual compounds. Pressure was typically maintained at 100 bar, and flow rates ranged from 50-200 mL/min. UV monitoring was generally carried out at 220 or 205 nM.

¹H NMR data were typically acquired using a Bruker NEO 500 MHz NMR spectrometer equipped with a room temperature 5 mm BBF iProbe, a Bruker Avance NEO 400 MHz NMR spectrometer equipped with a Bruker PI HR-BBO400S1-BBF/H/D-5.0-Z SP probe, or a Bruker Avance ITT 500 MHz NMR spectrometer equipped with a Bruker 5 mm PABBO probe. Chemical shift values are reported in delta (6) units, parts per million (ppm). Chemical shifts for ¹H NMR spectra are given relative to signals for residual non-deuterated solvent (CDCl₃ referenced at δ 7.26 ppm; DMSO-d⁶ referenced at δ 2.50 ppm and CD₃OD referenced at δ 3.31 ppm). Multiplets are reported by the following abbreviations: s=singlet, d=doublet, t=triplet, q=quartet, dd=doublet of doublets, m=multiplet or overlap of non-equivalent resonances. Coupling constants (J) are reported in Hertz (Hz). When compounds appear as mixtures of rotamers by NMR, spectral data corresponding to the major species observed in solution are reported.

### Abbreviations

| | |
|---|---|
| aq. | aqueous |
| DCM | dichloromethane |
| DCE | 1,2-dichloroethane |
| DEA | N,N-diethylamine |
| DIPEA | N,N-diisopropyl-N-ethylamine |
| DMA | N,N-dimethylacetamide |
| DME | 1,2-dimethoxyethane |
| DMF | N,N-dimethylformamide |
| DMSO | dimethyl sulfoxide |
| Et | ethyl |
| Et₂O | diethyl ether |
| EtOAc | ethyl acetate |
| EtOH | ethanol |
| h | hour(s) |
| HATU | 1-[bis(dimethylamino)methylene]-1H-1,2,3- |
| | triazolo[4,5-b]pyridinium 3-oxid hexafluorophosphate |
| HPLC | high-performance liquid chromatography |
| IPA | 2-propanol |
| Me | methyl |
| MeCN | acetonitrile |
| MeOH | methanol |
| MTBE | methyl tert-butyl ether |
| min | minute(s) |
| NMP | 1-methyl-2-pyrrolidinone |
| Ph | phenyl |
| PPA | polyphosphoric acid |
| rt | room temperature |
| SFC | supercritical fluid chromatography |
| 2-PrOH | isopropanol |
| TFA | trifluoroacetic acid |
| THF | tetrahydrofuran |
| TLC | thin-layer chromatography |

### Procedures

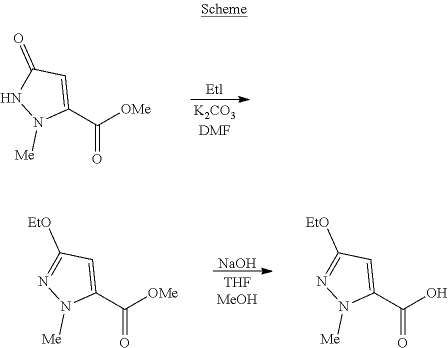 [SMILES: COC(=O)c1cc(=O)[nH]n1C ; CCOc1cc(C(=O)OC)n(C)n1 ; "NaOH" ; CCOc1cc(C(=O)O)n(C)n1]

### Intermediate A-01

### 3-ethoxy-1-methyl-1H-pyrazole-5-carboxylic acid

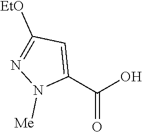 [SMILES: CCOc1cc(C(=O)O)n(C)n1]

### Step 1: methyl 3-ethoxy-1-methyl-1H-pyrazole-5-carboxylate

A mixture of methyl 2-methyl-5-oxo-2,5-dihydro-1H-pyrazole-3-carboxylate (0.20 g, 1.3 mmol), potassium carbonate (0.53 g, 3.8 mmol), iodoethane (0.21 mL, 2.6 mmol), and DMF (6.0 mL) was stirred at room temperature for 2 h. EtOAc was added and the mixture was washed with water (3×) and brine. The organic solution was dried with anhydrous magnesium sulfate, filtered, and concentrated in vacuo. The material was purified by silica gel chromatography (0-25% EtOAc/hexanes) to afford the title compound.

### Step 2: 3-ethoxy-1-methyl-1H-pyrazole-5-carboxylic acid

A mixture of methyl 3-ethoxy-1-methyl-1H-pyrazole-5-carboxylate (0.19 g, 1.0 mmol), THF (2.5 mL), methanol (2.5 mL), and aqueous sodium hydroxide (1.0 M, 1.5 mL, 1.5 mmol) was stirred at room temperature for 1.25 h. The mixture was concentrated and acidified with hydrochloric acid (1 M). The mixture was filtered and the filter cake was washed with water and dried to afford the title compound.

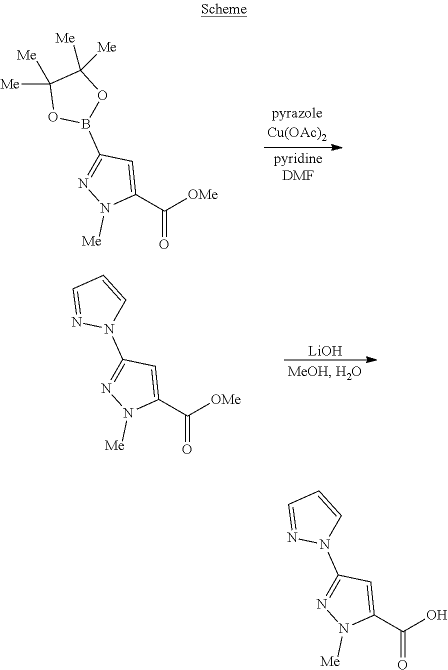

### Intermediate A-02

### 1′-methyl-1′H-[1,3′-bipyrazole]-5′-carboxylic acid

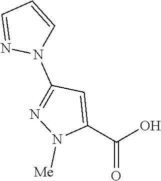 [SMILES: Cn1nc(-n2cccn2)cc1C(=O)O]

### Step 1: methyl 1′-methyl-1′H-[1,3′-bipyrazole]-5′-carboxylate

A mixture of methyl 1-methyl-3-(4,4,5,5-tetramethyl-1,3,2-dioxaborolan-2-yl)-1H-pyrazole-5-carboxylate (1.0 g, 3.8 mmol), 1H-pyrazole (0.34 g, 5.0 mmol), copper(II) acetate monohydrate (0.75 g, 3.8 mmol), DMF (0.030 L), and pyridine (1.5 mL, 19 mmol) was stirred at 80° C. under an atmosphere of O₂ for 12 h. The mixture was cooled to room temperature, diluted with water (15 mL), and extracted with EtOAc (20 mL×3). The combined organic extracts were washed with brine (20 mL), dried with anhydrous sodium sulfate, filtered, and concentrated in vacuo. The residue was purified by silica gel chromatography (0-25% EtOAc/petroleum ether) to afford the title compound.

### Step 2: 1′-methyl-1′H-[1,3′-bipyrazole]-5′-carboxylic acid

A mixture of methyl 1′-methyl-1′H-[1,3′-bipyrazole]-5′-carboxylate (0.46 g, 2.2 mmol), THF (5.0 mL), water (5.0 mL), and lithium hydroxide (0.27 g, 11 mmol) was stirred at 25° C. for 2 h. The mixture was partially concentrated and water (5 mL) was added. The mixture was acidified with hydrochloric acid (1 M) to pH=3 and extracted with EtOAc (10 mL×3). The combined organic extracts were dried with anhydrous sodium sulfate, filtered, and concentrated in vacuo to afford the title compound.

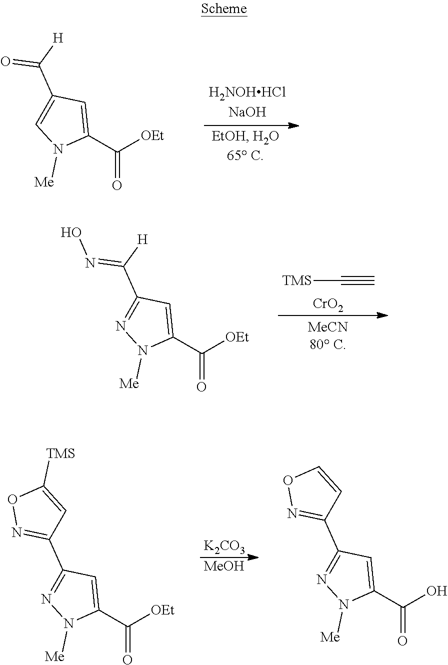

### Intermediate A-03

### 3-(isoxazol-3-yl)-1-methyl-1H-pyrazole-5-carboxylic acid

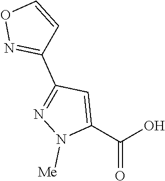 [SMILES: Cn1nc(-c2ccon2)cc1C(=O)O]

### Step 1: ethyl 3-((hydroxyimino)methyl)-1-methyl-1H-pyrazole-5-carboxylate

A mixture of ethyl 3-formyl-1-methyl-1H-pyrazole-5-carboxylate (0.50 g, 2.7 mmol), hydroxylamine hydrochloride (0.23 g, 3.3 mmol), sodium hydroxide (0.13 g, 3.3 mmol), water (3.0 mL), and ethanol (6.1 mL) was heated at 65° C. for 1 h. The mixture was cooled to room temperature, diluted with water, and extracted with EtOAc (2×). The combined organic extracts were washed with brine, dried with magnesium sulfate, filtered, and concentrated in vacuo to afford the title compound.

### Step 2: ethyl 1-methyl-3-(5-(trimethylsilyl)isoxazol-3-yl)-1H-pyrazole-5-carboxylate

A mixture of ethyl 3-((hydroxyimino)methyl)-1-methyl-1H-pyrazole-5-carboxylate (2.1 g, 11 mmol), chromium dioxide (8.9 g, 0.11 mol), trimethylsilylacetylene (4.6 mL, 32 mmol), and acetonitrile (0.050 L) was stirred at 80° C. for 72 h. The mixture was cooled to room temperature, filtered through a pad of celite, and eluted with EtOAc. The filtrate was concentrated in vacuo and the residue was purified by silica gel chromatography (0-20% EtOAc/hexanes) to afford the title compound.

### Step 3: 3-(isoxazol-3-yl)-1-methyl-1H-pyrazole-5-carboxylic acid

A mixture of ethyl 1-methyl-3-(5-(trimethylsilyl)isoxazol-3-yl)-1H-pyrazole-5-carboxylate (1.3 g, 4.4 mmol), potassium carbonate (0.92 g, 6.7 mmol), and methanol (22 mL) was stirred at room temperature for 16 h. The mixture was diluted with water and washed with EtOAc. The aqueous mixture was acidified with hydrochloric acid (1 M) and extracted with EtOAc (2×). The combined organic extracts were washed with brine, dried with anhydrous magnesium sulfate, filtered, and concentrated in vacuo to afford the title compound.

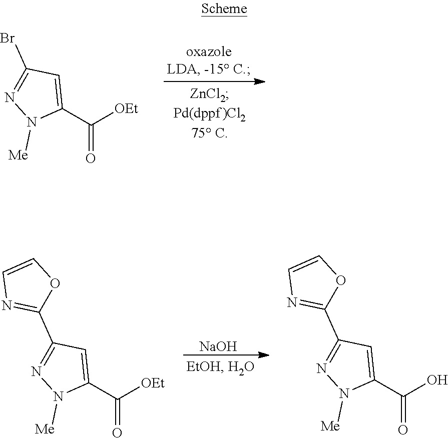 [SMILES: CCOC(=O)c1cc(Br)nn1C ; CCOC(=O)c1cc(-c2ncco2)nn1C ; "NaOH" ; Cn1nc(-c2ncco2)cc1C(=O)O]

### Intermediate A-04

### 1-methyl-3-(oxazol-2-yl)-1H-pyrazole-5-carboxylic acid

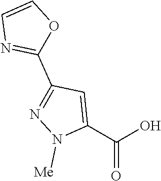 [SMILES: Cn1nc(-c2ncco2)cc1C(=O)O]

### Step 1: ethyl 1-methyl-3-(oxazol-2-yl)-1H-pyrazole-5-carboxylate

A solution of lithium bis(trimethylsilyl)amide (1.0 M in THF, 8.6 mL, 8.6 mmol) was added dropwise over a 15 min period to a −15° C. solution of oxazole (0.42 mL, 6.4 mmol) in THF (21 mL). The mixture was stirred at −15° C. for 30 min. A solution of zinc chloride (0.50 M in THF, 28 mL, 14 mmol) was added and the mixture was stirred at −15° C. for 15 min. The mixture was warmed to room temperature and stirred for 30 min, to which was then added to a mixture of ethyl 3-bromo-1-methyl-1H-pyrazole-5-carboxylate (1.0 g, 4.3 mmol) and [1,1′-bis(diphenylphosphino)ferrocene]dichloropalladium(II) (0.16 g, 0.22 mmol). The mixture was heated at 75° C. for 12 h, cooled to room temperature, and treated with ammonium chloride (aqueous saturated, 50 mL) and EtOAc (75 mL). The mixture was washed with water (75 mL), and brine (75 mL). The organic solution was dried with anhydrous magnesium sulfate, filtered, and concentrated in vacuo. The residue was purified by silica gel chromatography (0-15% (25% EtOH/EtOAc)/hexanes) to afford the title compound.

### Step 2: 1-methyl-3-(oxazol-2-yl)-1H-pyrazole-5-carboxylic acid

A mixture of ethyl 1-methyl-3-(oxazol-2-yl)-1H-pyrazole-5-carboxylate (0.85 g, 3.8 mmol), sodium hydroxide (1.0 M aqueous solution, 5.8 mL, 5.8 mmol), EtOH (9.6 mL), and THF (9.6 mL) was stirred at room temperature for 4 h. The mixture was concentrated and an aqueous solution of sodium hydroxide (0.05 M, 75 mL) was added and then washed with DCM (50 mL×3). The aqueous solution was acidified (pH=1) with hydrochloric acid (1 M) and then stirred at room temperature for 12 h, filtered, and the filter cake was washed with water (10 mL×3). The filter cake was purified by reverse phase HPLC (0-90% MeCN/water containing 0.1% TFA) to afford the title compound.

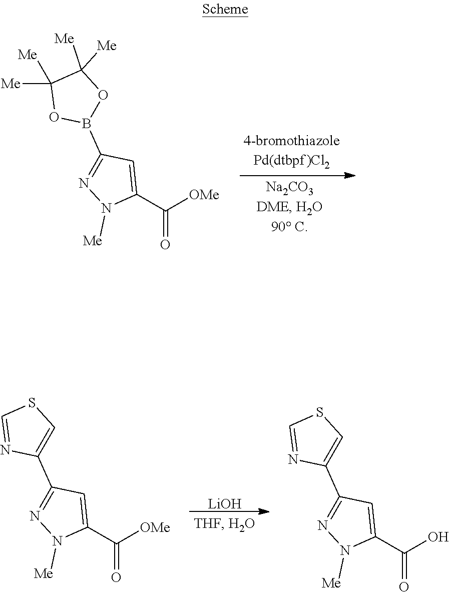 [SMILES: COC(=O)c1cc(B2OC(C)(C)C(C)(C)O2)nn1C ; COC(=O)c1cc(-c2cscn2)nn1C ; "LiOH" ; Cn1nc(-c2cscn2)cc1C(=O)O]

### Intermediate A-05

### 1-methyl-3-(thiazol-4-yl)-1H-pyrazole-5-carboxylic acid

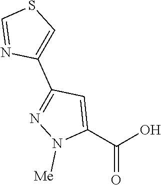 [SMILES: Cn1nc(-c2cscn2)cc1C(=O)O]

### Step 1: methyl 1-methyl-3-(thiazol-4-yl)-1H-pyrazole-5-carboxylate

A mixture of methyl 1-methyl-3-(4,4,5,5-tetramethyl-1,3,2-dioxaborolan-2-yl)-1H-pyrazole-5-carboxylate (2.0 g, 7.5 mmol), 4-bromothiazole (1.2 g, 7.5 mmol), [1,1′-bis(di-tert-butylphosphino)ferrocene]dichloropalladium(II) (0.25 g, 0.38 mmol), sodium carbonate (2.4 g, 23 mmol), DME (0.040 L), and water (4.0 mL) was stirred at 90° C. for 12 h. The mixture was diluted with water (15 mL) and extracted with EtOAc (30 mL×3). The combined organic extracts were washed with brine (20 mL×2), dried with anhydrous sodium sulfate, filtered, and concentrated in vacuo. The residue was purified by silica gel chromatography (20-35% EtOAc/petroleum ether) to afford the title compound.

### Step 2: 1-methyl-3-(thiazol-4-yl)-1H-pyrazole-5-carboxylic acid

A mixture of methyl 1-methyl-3-(thiazol-4-yl)-1H-pyrazole-5-carboxylate (1.2 g, 5.4 mmol), lithium hydroxide (0.64 g, 27 mmol), THF (0.010 L), and water (0.010 L) was stirred at room temperature for 0.5 h. The mixture was concentrated, diluted with water, and washed with DCM (10 mL×2). The aqueous solution was acidified (pH=4) with hydrochloric acid (1 M) and concentrated in vacuo. MeOH (10 mL) and DCM (10 mL) were added, and the mixture was stirred at 25° C. for 1 h. The mixture was filtered and the filtrate was concentrated in vacuo to afford the title compound.

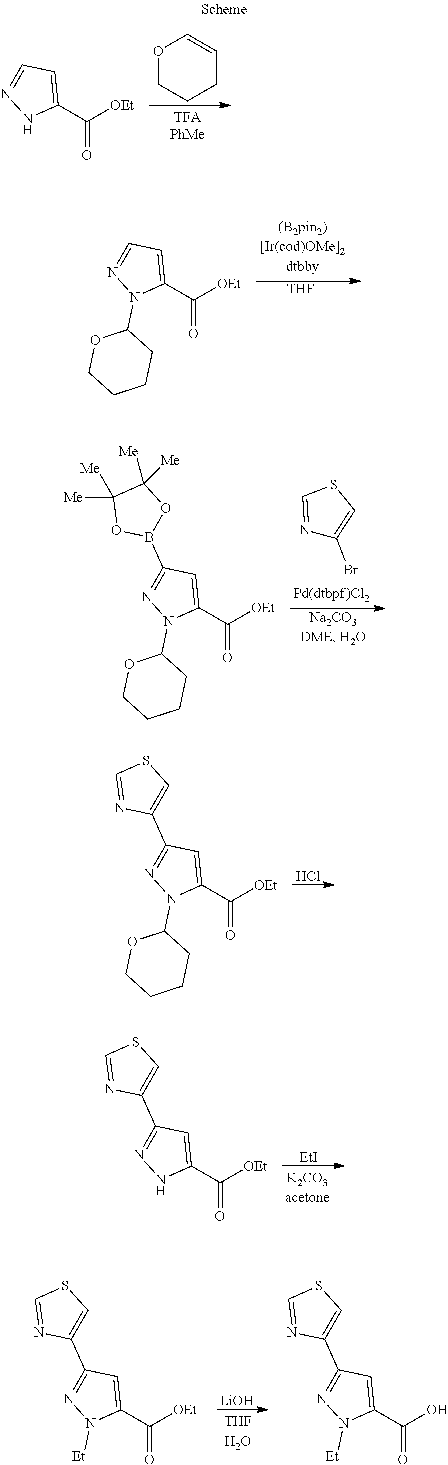

### Intermediate A-06

### 1-ethyl-3-(thiazol-4-yl)-1H-pyrazole-5-carboxylic acid

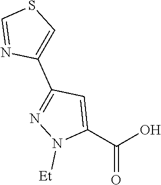 [SMILES: CCn1nc(-c2cscn2)cc1C(=O)O]

### Step 1: ethyl 1-(tetrahydro-2H-pyran-2-yl)-1H-pyrazole-5-carboxylate

A mixture of ethyl 1H-pyrazole-5-carboxylate (5.0 g, 36 mmol), 3,4-dihydro-2H-pyran (3.2 g, 38 mmol), TFA (0.027 mL, 0.36 mmol), and PhMe (0.030 L) was stirred at 80° C. for 2 h. The mixture was concentrated in vacuo. Water (30 mL) was added, and the mixture was extracted with EtOAc (30 mL×3). The combined organic extracts were dried with anhydrous sodium sulfate, filtered, and concentrated in vacuo. The residue was purified by silica gel chromatography (0-60% EtOAc/petroleum ether) to afford the title compound.

### Step 2: ethyl 1-(tetrahydro-2H-pyran-2-yl)-3-(4,4,5,5-tetramethyl-1,3,2-dioxaborolan-2-yl)-1H-pyrazole-5-carboxylate

A mixture of ethyl 1-(tetrahydro-2H-pyran-2-yl)-1H-pyrazole-5-carboxylate (4.8 g, 21 mmol), 4,4,4′,4′,5,5,5′,5′-octamethyl-2,2′-bi(1,3,2-dioxaborolane) (5.4 g, 21 mmol), 4,4′-di-tert-butyl-2,2′-bipyridine (0.57 g, 2.1 mmol), (1,5-cyclooctadiene)(methoxy)iridium(I) dimer (0.71 g, 1.1 mmol), and THF (0.080 L) was stirred under an atmosphere of nitrogen at 80° C. for 12 h. The mixture was cooled to ambient temperature, water (20 mL) was added, and then extracted with EtOAc (30 mL×3). The combined organic extracts were washed with brine (20 mL×3), dried with anhydrous sodium sulfate, filtered, concentrated in vacuo. The residue was purified by preparative HPLC (35% MeCN/water) to afford the title compound.

### Step 3: ethyl 1-(tetrahydro-2H-pyran-2-yl)-3-(thiazol-4-yl)-1H-pyrazole-5-carboxylate

A mixture of ethyl 1-(tetrahydro-2H-pyran-2-yl)-3-(4,4,5,5-tetramethyl-1,3,2-dioxaborolan-2-yl)-1H-pyrazole-5-carboxylate (1.0 g, 2.9 mmol), 4-bromothiazole (0.70 g, 4.3 mmol), [1,1′-bis(di-tert-butylphosphino)ferrocene]dichloropalladium(II) (0.19 g, 0.29 mmol), sodium carbonate (0.91 g, 8.6 mmol), DME (0.040 L) and water (4.0 mL) was stirred at 100° C. for 12 h. The mixture was cooled to ambient temperature, water (30 mL) was added, and then extracted with EtOAc (30 mL×3). The combined organic extracts were washed with brine (30 mL×3), dried with anhydrous sodium sulfate, filtered, and concentrated in vacuo. The material was purified by silica gel chromatography (0-50% EtOAc/petroleum ether) to afford the title compound.

### Step 4: ethyl 3-(thiazol-4-yl)-1H-pyrazole-5-carboxylate

A solution of ethyl 1-(tetrahydro-2H-pyran-2-yl)-3-(thiazol-4-yl)-1H-pyrazole-5-carboxylate (0.85 g, 2.8 mmol) in hydrochloric acid (37% aqueous, 2.0 mL) was stirred at 80° C. for 10 min. The mixture was cooled to ambient temperature and concentrated in vacuo to afford the title compound.

### Step 5: ethyl 1-ethyl-3-(thiazol-4-yl)-1H-pyrazole-5-carboxylate

A mixture of ethyl 3-(thiazol-4-yl)-1H-pyrazole-5-carboxylate (0.52 g, 2.3 mmol), K₂CO₃ (0.97 g, 7.0 mmol), iodoethane (0.90 mL, 12 mmol), and acetone (0.030 L) was heated under an atmosphere of N₂ at 70° C. for 19 h. The mixture was cooled to room temperature, filtered, and concentrated in vacuo. EtOAc (50 mL) was added and the mixture was washed with water (50 mL), hydrochloric acid (1 M, 50 mL), and brine (50 mL). The organic extracts were concentrated in vacuo and the residue was purified by silica gel chromatography (0-50% EtOAc/petroleum ether) to afford the title compound.

### Step 6: 1-ethyl-3-(thiazol-4-yl)-1H-pyrazole-5-carboxylic acid

A mixture of ethyl 1-ethyl-3-(thiazol-4-yl)-1H-pyrazole-5-carboxylate (0.030 g, 0.12 mmol), lithium hydroxide (8.6 mg, 0.36 mmol), THF (2.0 mL), and water (2.0 mL) was stirred at room temperature for 2 h. The mixture was partially concentrated, diluted with water, and washed with DCM (10 mL×2). The aqueous solution was acidified (pH=4) with hydrochloric acid (1 M) and the mixture was concentrated in vacuo. A mixture of MeOH and DCM (1:5, 20 mL) was added to the residue and the mixture was stirred at room temperature for 1 h. The mixture was filtered and the filtrate was concentrated to afford the title compound.

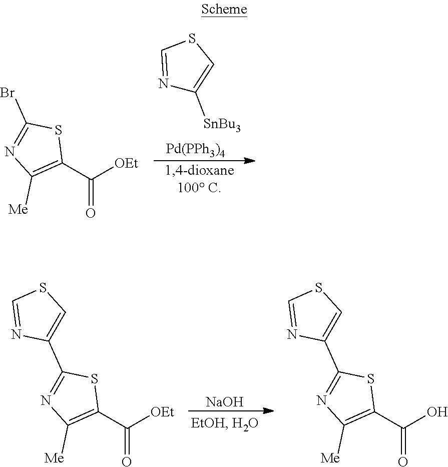

### Intermediate A-07

### 4-methyl-[2,4′-bithiazole]-5-carboxylic acid

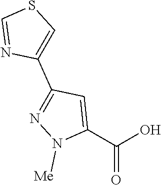 [SMILES: Cn1nc(-c2cscn2)cc1C(=O)O]

### Step 1: ethyl 4-methyl-[2,4′-bithiazole]-5-carboxylate

A mixture of ethyl 2-bromo-4-methylthiazole-5-carboxylate (0.15 g, 0.60 mmol), 4-(tributylstannyl)thiazole (0.22 g, 0.60 mmol), tetrakis(triphenylphosphine)palladium(0) (69 mg, 0.060 mmol), and 1,4-dioxane (3.0 mL) was stirred at 100° C. for 16 h. The mixture was cooled to room temperature and filtered through celite. The filter pad was washed with EtOAc and the filtrate was concentrated in vacuo. The residue was purified by silica gel chromatography (0-30% (25% EtOH/EtOAc)/hexanes) to afford the title compound.

### Step 2: 4-methyl-[2,4′-bithiazole]-5-carboxylic acid

An aqueous solution of sodium hydroxide (1.0 M, 0.77 mL, 0.77 mmol) was added to a mixture of ethyl 4-methyl-[2,4′-bithiazole]-5-carboxylate (0.13 g, 0.51 mmol), ethanol (1.0 mL), and water (1.0 mL). The mixture was stirred at room temperature for 1 h and then concentrated in vacuo. Methanol and hydrochloric acid (1 M) were added and the mixture was stirred for 1 h and then concentrated in vacuo to afford the title compound.

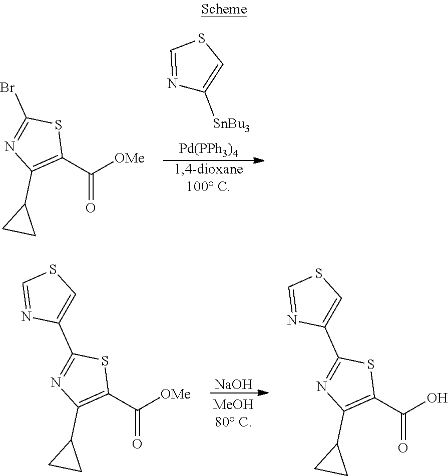

### Intermediate A-08

### 4-cyclopropyl-[2,4′-bithiazole]-5-carboxylic acid

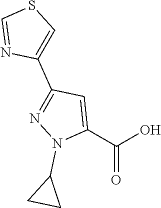 [SMILES: O=C(O)c1cc(-c2cscn2)nn1C1CC1]

### Step 1: methyl 4-cyclopropyl-[2,4′-bithiazole]-5-carboxylate

A mixture of methyl 2-bromo-4-cyclopropylthiazole-5-carboxylate (0.10 g, 0.38 mmol), 4-(tributylstannyl)thiazole (0.14 g, 0.38 mmol), tetrakis(triphenylphosphine)palladium(0) (44 mg, 0.038 mmol), and 1,4-dioxane (1.9 mL) was stirred at 100° C. for 2 h. The mixture was cooled to room temperature and filtered through celite. The filter pad was washed with EtOAc and the filtrate was concentrated in vacuo. The residue was purified by silica gel chromatography (0-30% (25% EtOH/EtOAc)/hexanes) to afford the title compound.

### Step 2: 4-cyclopropyl-[2,4′-bithiazole]-5-carboxylic acid

An aqueous solution of sodium hydroxide (1.0 M, 0.28 mL, 0.28 mmol) was added to a mixture of methyl 4-cyclopropyl-[2,4′-bithiazole]-5-carboxylate (49 mg, 0.18 mmol) and methanol (0.92 mL). The mixture was stirred at room temperature for 1 h and then the temperature was increased to 80° C. for 15 min. The mixture was cooled to room temperature and concentrated in vacuo. Methanol and hydrochloric acid (1 M, aqueous) were added, after which the mixture was stirred and then concentrated in vacuo to afford the title compound.

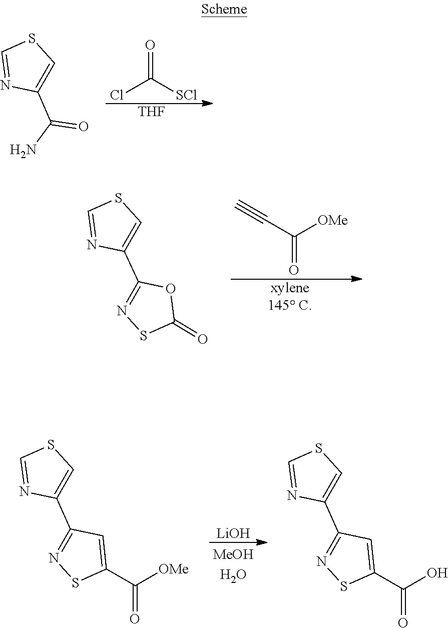

### Intermediate A-09

### 3-(thiazol-4-yl)isothiazole-5-carboxylic acid

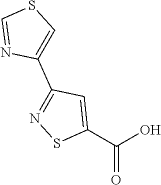 [SMILES: O=C(O)c1cc(-c2cscn2)ns1]

### Step 1: 5-(thiazol-4-yl)-1,3,4-oxathiazol-2-one

Carbonochloridic hypochlorous thioanhydride (0.60 mL, 7.0 mmol) was added to a 0° C. mixture of thiazole-4-carboxamide (0.50 g, 3.9 mmol) and THF (8.0 mL). The mixture was warmed to 25° C. and stirred for 12 h. The mixture was concentrated in vacuo to afford the title compound.

### Step 2: methyl 3-(thiazol-4-yl)isothiazole-5-carboxylate

A mixture of 5-(thiazol-4-yl)-1,3,4-oxathiazol-2-one (0.30 g, 1.6 mmol), methyl propiolate (2.0 mL, 32 mmol), and xylene (5.0 mL) was stirred at 145° C. for 3 days. The mixture cooled to ambient temperature, concentrated in vacuo, and then purified by preparative TLC (25% EtOAc/petroleum ether) to afford the title compound.

### Step 3: 3-(thiazol-4-yl)isothiazole-5-carboxylic acid

A mixture of methyl 3-(thiazol-4-yl)isothiazole-5-carboxylate (0.13 g, 0.55 mmol), lithium hydroxide (27 mg, 1.1 mmol), MeOH (3.0 mL), and water (3.0 mL) was stirred at 25° C. for 2 h. The mixture was partially concentrated in vacuo, diluted with water, and washed with DCM (5 mL×2). The aqueous solution was acidified (pH=4) with hydrochloric acid (1 M, aqueous) and then extracted with EtOAc (10 mL×3). The combined organic extracts were washed with brine (10 mL), dried with anhydrous sodium sulfate, filtered, and concentrated in vacuo to afford the title compound.

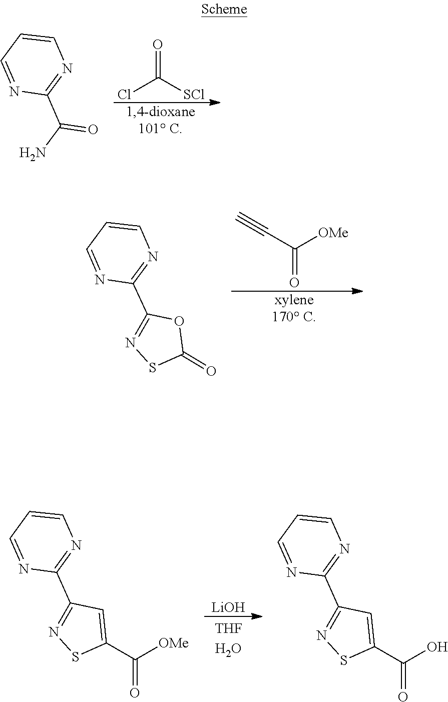

### Intermediate A-10

### 3-(pyrimidin-2-yl)isothiazole-5-carboxylic acid

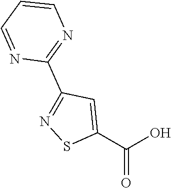 [SMILES: O=C(O)c1cc(-c2ncccn2)ns1]

### Step 1: 5-(pyrimidin-2-yl)-1,3,4-oxathiazol-2-one

Carbonochloridic hypochlorous thioanhydride (1.4 mL, 16 mmol) was added to a 0° C. mixture of pyrimidine-2-carboxamide (2.0 g, 16 mmol) and 1,4-dioxane (0.030 L). The mixture was stirred at 101° C. for 12 h, cooled to room temperature, and then concentrated in vacuo to afford the title compound.

### Step 2: methyl 3-(pyrimidin-2-yl)isothiazole-5-carboxylate

A mixture of 5-(pyrimidin-2-yl)-1,3,4-oxathiazol-2-one (0.60 g, 3.3 mmol), methyl propiolate (1.2 mL, 0.020 mol), and xylene (0.010 L) was stirred at 170° C. for 4 h. The mixture was cooled to ambient temperature, concentrated in vacuo, and the residue was purified by preparative TLC (50% EtOAc/petroleum ether) to afford the title compound.

### Step 3: 3-(pyrimidin-2-yl)isothiazole-5-carboxylic acid

A mixture of methyl 3-(pyrimidin-2-yl)isothiazole-5-carboxylate (0.14 g, 0.63 mmol), lithium hydroxide (76 mg, 3.2 mmol), THF (3.0 mL), and water (3.0 mL) was stirred at 25° C. for 0.5 h. The mixture was partially concentrated and then acidified (pH=6) with hydrochloric acid (1 M, aqueous). The mixture was filtered and the filter cake was dried to afford the title compound.

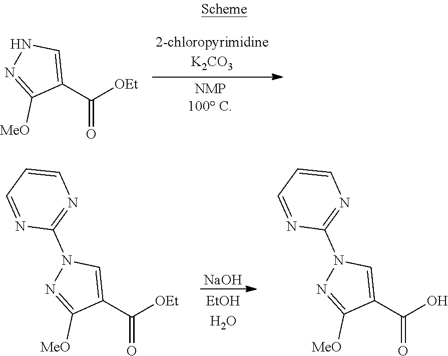

### Intermediate A-11

### 3-methoxy-1-(pyrimidin-2-yl)-1H-pyrazole-4-carboxylic acid

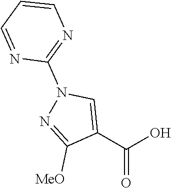 [SMILES: COc1nn(-c2ncccn2)cc1C(=O)O]

### Step 1: ethyl 3-methoxy-1-(pyrimidin-2-yl)-1H-pyrazole-4-carboxylate

A mixture of ethyl 3-methoxy-1H-pyrazole-4-carboxylate (0.010 kg, 59 mmol), 2-chloropyrimidine (0.010 kg, 88 mmol), potassium carbonate (16 g, 0.12 mol), and NMP (0.18 L) was stirred at 100° C. for 16 h. The mixture was cooled to room temperature and filtered. The filtrate was diluted with water (500 mL) and extracted with EtOAc (500 mL×3). The combined organic extracts were washed with brine, dried with anhydrous sodium sulfate, filtered, and concentrated in vacuo to afford the title compound.

### Step 2: 3-methoxy-1-(pyrimidin-2-yl)-1H-pyrazole-4-carboxylic acid

A mixture of ethyl 3-methoxy-1-(pyrimidin-2-yl)-1H-pyrazole-4-carboxylate (6.0 g, 24 mmol), ethanol (0.32 L), water (0.16 L), and sodium hydroxide (1.9 g, 48 mmol) was stirred for 16 h at room temperature. The mixture was partially concentrated in vacuo, water (30 mL) was added, and the pH was adjusted (pH=3) with hydrochloric acid (6 M). The mixture was filtered, and the filter cake was dried to afford the title compound.

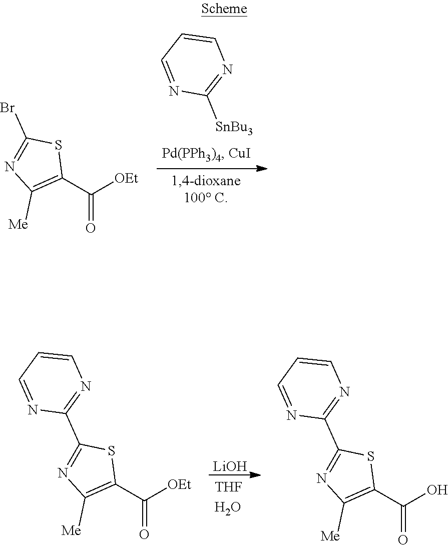

### Intermediate A-12

### 4-methyl-2-(pyrimidin-2-yl)thiazole-5-carboxylic acid

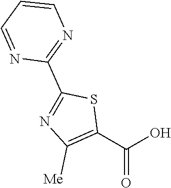 [SMILES: Cc1nc(-c2ncccn2)sc1C(=O)O]

### Step 1: ethyl 4-methyl-2-(pyrimidin-2-yl)thiazole-5-carboxylate

A mixture of ethyl 2-bromo-4-methylthiazole-5-carboxylate (1.8 g, 7.2 mmol), 2-(tributylstannyl)pyrimidine (3.5 g, 9.5 mmol), tetrakis(triphenylphosphine)palladium(0) (0.83 g, 0.72 mmol), copper(I) iodide (0.34 g, 1.8 mmol), and 1,4-dioxane (36 mL) was heated to 100° C. for 16 h. The mixture was cooled to room temperature and concentrated in vacuo. The residue was diluted with DCM, filtered through celite, and the filtrate was concentrated in vacuo. The residue was purified by silica gel chromatography (0-50% (25% EtOH/EtOAc)/heptane) to afford the title compound.

### Step 2: 4-methyl-2-(pyrimidin-2-yl)thiazole-5-carboxylic acid

A mixture of ethyl 4-methyl-2-(pyrimidin-2-yl)thiazole-5-carboxylate (1.6 g, 6.6 mmol), THF (25 mL), and aqueous lithium hydroxide (2.0 M, 0.010 L, 0.020 mol) was stirred at 25° C. for 3 h. Water and EtOAc were added, and the mixture was filtered. The aqueous layer from the filtrate was washed with EtOAc (2×). The aqueous mixture was then acidified, filtered, and the filter cake was dried to afford the title compound.

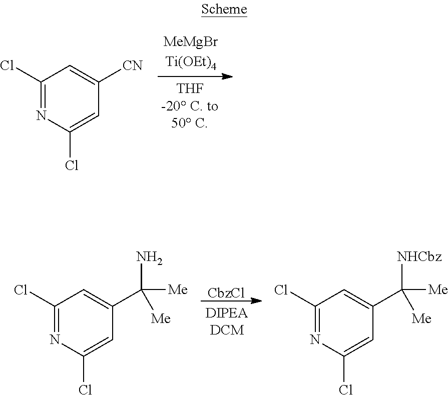

Intermediate B-01 benzyl (2-(2,6-dichloropyridin-4-yl)propan-2-yl)carbamate

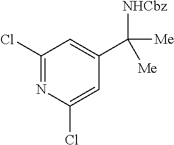 [SMILES: CC(C)(NC(=O)OCc1ccccc1)c1cc(Cl)nc(Cl)c1]

### Step 1: 2-(2,6-dichloropyridin-4-yl)propan-2-amine

A mixture of 2,6-dichloropyridine-4-carbonitrile (0.20 kg, 1.2 mol) and THF (3.0 L) was cooled to −20° C. A solution of methylmagnesium bromide (3.0 M in diethyl ether, 1.2 L, 3.5 mol) was added and the mixture was stirred for 2 h. Titanium(IV) ethoxide was added and the mixture was warmed to 50° C. and stirred for 2 h. The mixture was cooled to room temperature and THF (2 L) and water (1 L) were added. The mixture was filtered, and the filtrate concentrated in vacuo. The mixture was acidified (pH=1) with hydrochloric acid (6 M) and washed with EtOAc (200 mL×2). The pH of the aqueous mixture was adjusted (pH=9) with NaHCO₃. The mixture was filtered and the filter cake was dried to afford the title compound.

### Step 2: benzyl (2-(2,6-dichloropyridin-4-yl)propan-2-yl)carbamate

Benzyl chloroformate (92 g, 0.54 mol) was added to a 0° C. mixture of 2-(2,6-dichloropyridin-4-yl)propan-2-amine (0.10 kg, 0.49 mol), DIPEA (94 g, 0.73 mol), and DCM (1.5 L). The mixture was warmed to 25° C. and stirred for 10 h. Water (1 L) was added, and the mixture was extracted with DCM (300 mL×3). The combined organic extracts were dried with anhydrous sodium sulfate, filtered, and concentrated in vacuo. The residue was re-crystallized from MTBE to afford the title compound.

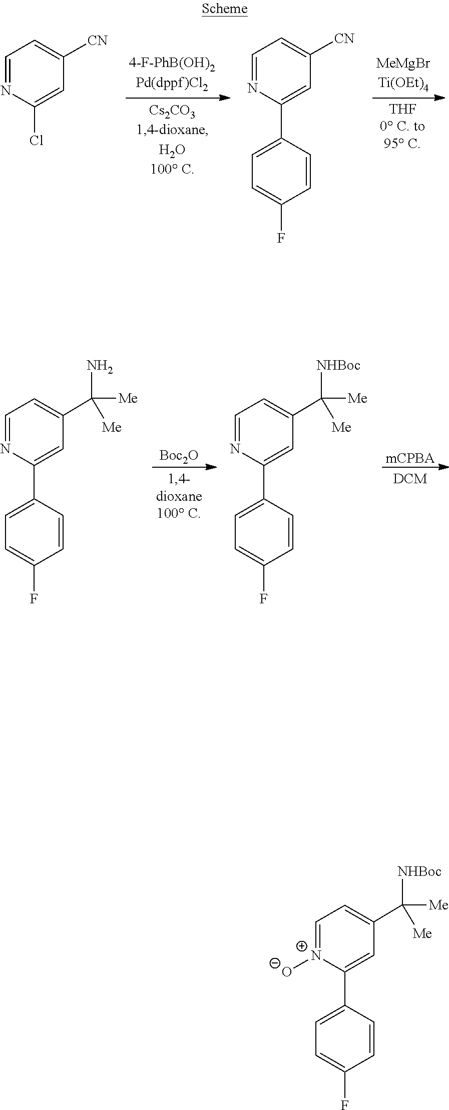

### Intermediate B-02

### 4-(2-((tert-butoxycarbonyl)amino)propan-2-yl)-2-(4-fluorophenyl)pyridine 1-oxide

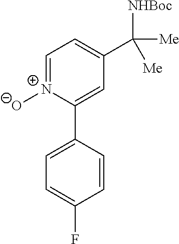 [SMILES: CC(C)(C)OC(=O)NC(C)(C)c1cc[n+]([O-])c(-c2ccc(F)cc2)c1]

### Step 1: 2-(4-fluorophenyl)isonicotinonitrile

A mixture of 2-chloropyridine-4-carbonitrile (0.090 kg, 0.65 mol), 4-fluorophenylboronic acid (0.090 kg, 0.65 mol), [1,1′-bis(diphenylphosphino)ferrocene]dichloropalladium(II) (24 g, 33 mmol), cesium carbonate (0.63 kg, 1.9 mol), 1,4-dioxane (0.67 L), and water (0.33 L) was stirred at 100° C. for 3 h. The mixture was cooled to 0° C. and a water/ice mixture (2 L) was added. The mixture was filtered, and the filtrate was extracted with EtOAc (500 mL×3). The combined organic extracts were dried with anhydrous sodium sulfate, filtered, and concentrated in vacuo. The residue was recrystallized from petroleum ether to afford the title compound.

### Step 2: 2-(2-(4-fluorophenyl)pyridin-4-yl)propan-2-amine

To a mixture of 2-(4-fluorophenyl)isonicotinonitrile (0.10 kg, 0.50 mol) and THF (1.5 L) at 0° C. under an atmosphere of N₂ was added a solution of methylmagnesium bromide (3.0 M in diethyl ether, 0.50 L, 1.5 mol). The mixture was warmed to room temperature and stirred for 0.5 h. Titanium(IV) ethoxide (0.14 kg, 0.50 mmol) was added and the mixture was then heated at 95° C. for 3 h. The mixture was cooled to ambient temperature and treated with a water/ice mixture (3 L). The mixture was filtered and the filtrate was extracted with EtOAc (500 mL×3). The combined organic extracts were dried with anhydrous sodium sulfate, filtered, and concentrated in vacuo. The residue was purified by silica gel chromatography (80% EtOAc/petroleum ether) to afford the title compound.

### Step 3: tert-butyl (2-(2-(4-fluorophenyl)pyridin-4-yl)propan-2-yl)carbamate

A mixture of di-tert-butyl dicarbonate (0.14 kg, 0.64 mol), 2-(2-(4-fluorophenyl)pyridin-4-yl)propan-2-amine (0.10 kg, 0.43 mol), and 1,4-dioxane (2.0 L) was stirred at 100° C. for 1 h. The mixture was cooled to room temperature and then concentrated in vacuo. The residue was purified by silica gel chromatography (10% EtOAc/petroleum ether) to afford the title compound.

### Step 4: 4-(2-((tert-butoxycarbonyl)amino)propan-2-yl)-2-(4-fluorophenyl)pyridine 1-oxide

A mixture of 3-chloroperoxybenzoic acid (68 g, 0.39 mol), tert-butyl (2-(2-(4-fluorophenyl)pyridin-4-yl)propan-2-yl)carbamate (65 g, 0.20 mol), and DCM (1.0 L) was stirred at room temperature for 16 h. A water/ice mixture (500 mL) was added and resulting mixture was washed with sodium carbonate (aqueous saturated, 500 mL×3). The organic layer was dried with anhydrous sodium sulfate, filtered, and concentrated in vacuo. The residue was recrystallized from MTBE to afford the title compound.

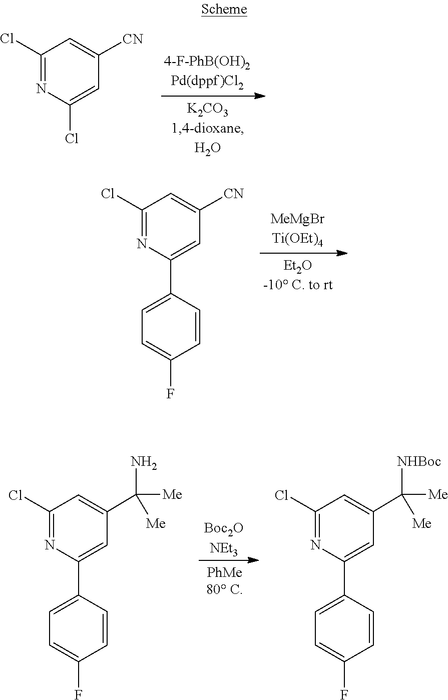 [SMILES: "Boc2ONEt3" ; N#Cc1cc(Cl)nc(Cl)c1 ; N#Cc1cc(Cl)nc(-c2ccc(F)cc2)c1 ; CC(C)(N)c1cc(Cl)nc(-c2ccc(F)cc2)c1 ; CC(C)(C)OC(=O)NC(C)(C)c1cc(Cl)nc(-c2ccc(F)cc2)c1]

### Intermediate B-03

### tert-butyl (2-(2-chloro-6-(4-fluorophenyl)pyridin-4-yl)propan-2-yl)carbamate

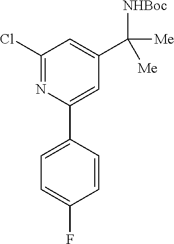 [SMILES: CC(C)(C)OC(=O)NC(C)(C)c1cc(Cl)nc(-c2ccc(F)cc2)c1]

### Step 1: 2-chloro-6-(4-fluorophenyl)isonicotinonitrile

A 0° C. vessel under an atmosphere of N₂ was charged with 2,6-dichloropyridine-4-carbonitrile (0.14 kg, 0.81 mol), 4-fluorophenylboronic acid (0.11 kg, 0.81 mol), potassium carbonate (0.22 kg, 1.6 mol), 1,4-dioxane (0.70 L), water (0.35 L), and [1,1′-bis(diphenylphosphino)ferrocene]dichloropalladium(II) (1:1) complex with dichloromethane (13 g, 16 mmol). The mixture was warmed to room temperature and stirred for 5 h. Water (2 L) was added and the mixture was extracted with EtOAc (3 L×3). The combined organic extracts were washed with brine (5 L×2), dried with anhydrous sodium sulfate, filtered, and concentrated in vacuo. The residue was purified by preparative HPLC (C-18; 0-100% MeCN/water containing 0.05% TFA) to afford the title compound.

### Step 2: 2-(2-chloro-6-(4-fluorophenyl)pyridin-4-yl)propan-2-amine

A mixture of 2-chloro-6-(4-fluorophenyl)pyridine-4-carbonitrile (0.090 kg, 0.39 mol) and diethyl ether (1.8 L) was cooled to −10° C. under an atmosphere of N₂. A solution of methylmagnesium bromide (3.0 M in diethyl ether, 0.39 L, 1.2 mol) was added and the mixture was stirred at −10° C. for 1.5 h. The mixture was warmed to 0° C. and titanium(IV) ethoxide (0.11 kg, 0.39 mmol) was added. The mixture was then warmed to room temperature and stirred for an additional 16 h. An aqueous solution of NaOH (1 L) and DCM (2 L) were added and the mixture was filtered. The filtrate was extracted with DCM (3 L×3) and the combined organic extracts were dried with anhydrous sodium sulfate, filtered, and concentrated in vacuo. The residue was purified by preparative HPLC (C-18; 0-100% MeCN/water) to afford the title compound.

### Step 3: tert-butyl (2-(2-chloro-6-(4-fluorophenyl)pyridin-4-yl)propan-2-yl)carbamate

Di-tert-butyl dicarbonate (89 g, 0.41 mol) was added to a 0° C. mixture of 2-(2-chloro-6-(4-fluorophenyl)pyridin-4-yl)propan-2-amine (0.090 kg, 0.34 mol), triethylamine (0.10 kg, 1.0 mol), and toluene (1.8 L). The mixture was then heated at 80° C. for 2 h, cooled to 0° C., and then treated with water (2 L). The mixture was extracted with EtOAc (2 L×3) and the combined organic extracts were dried with anhydrous sodium sulfate, filtered, and concentrated in vacuo. The residue was purified by silica gel chromatography (10% EtOAc/petroleum ether) to afford the title compound.

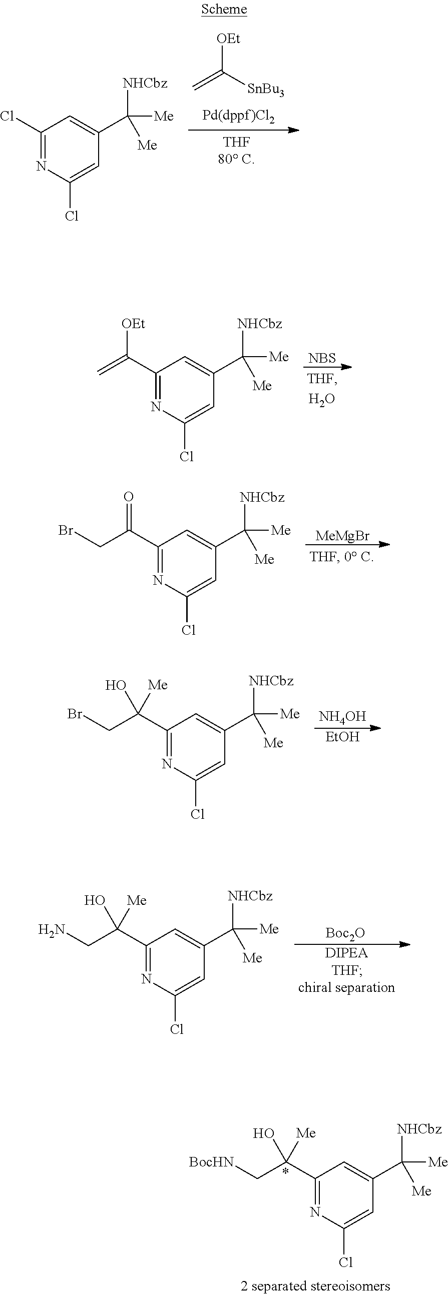

### Intermediate C-01-enantiomer-1 and Intermediate C-01-enantiomer-2

### tert-butyl (2-(4-(2-(((benzyloxy)carbonyl)amino)propan-2-yl)-6-chloropyridin-2-yl)-2-hydroxypropyl)carbamate (enantiomer-1 and enantiomer-2)

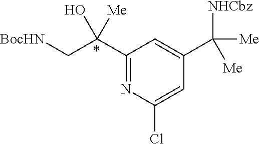 [SMILES: CC(C)(C)OC(=O)NCC(C)(O)c1cc(C(C)(C)NC(=O)OCc2ccccc2)cc(Cl)n1]

### Step 1: benzyl (2-(2-chloro-6-(1-ethoxyvinyl)pyridin-4-yl)propan-2-yl)carbamate

A mixture of benzyl (2-(2,6-dichloropyridin-4-yl)propan-2-yl)carbamate (Intermediate B-01, 0.090 kg, 0.27 mol), [1,1′-bis(diphenylphosphino)ferrocene]dichloropalladium(II) (9.7 g, 13 mmol), THF (2.0 L), and tributyl(1-ethoxyethenyl)stannane (0.11 kg, 0.32 mol) was stirred at 80° C. for 10 h. The mixture was cooled to ambient temperature, filtered, and the filtrate was concentrated in vacuo. The residue was purified by silica gel chromatography (5-20% EtOAc/petroleum ether) to afford the title compound.

### Step 2: benzyl (2-(2-(2-bromoacetyl)-6-chloropyridin-4-yl)propan-2-yl)carbamate

N-Bromosuccinimide (46 g, 0.26 mol) was added portion wise to a 0° C. mixture of benzyl (2-(2-chloro-6-(1-ethoxyvinyl)pyridin-4-yl)propan-2-yl)carbamate (0.080 kg, 0.21 mol), THF (1.0 L), and water (0.25 L). The mixture was warmed to room temperature and stirred for 2 h. Water (1 L) was added and the mixture was extracted with EtOAc (300 mL×3). The combined organic extracts were dried with anhydrous sodium sulfate, filtered, and concentrated in vacuo. The material was purified by silica gel chromatography (5% EtOAc/petroleum ether) to afford the title compound.

### Step 3: benzyl (2-(2-(1-bromo-2-hydroxypropan-2-yl)-6-chloropyridin-4-yl)propan-2-yl)carbamate

A solution of methylmagnesium bromide (3.0 M in diethyl ether, 62 mL, 0.18 mol) was added dropwise to a 0° C. solution of benzyl (2-(2-(2-bromoacetyl)-6-chloropyridin-4-yl)propan-2-yl)carbamate (65 g, 0.15 mol) in THF (1.0 L). After stirring for 2 h at 0° C., an aqueous solution of ammonium chloride (saturated, 1 L) was added, and the mixture was extracted with EtOAc (300 mL×3). The combined organic extracts were dried with anhydrous sodium sulfate, filtered, and concentrated in vacuo. The residue was purified by silica gel chromatography (5-25% EtOAc/petroleum ether) to afford the title compound.

### Step 4: benzyl (2-(2-(1-amino-2-hydroxypropan-2-yl)-6-chloropyridin-4-yl)propan-2-yl)carbamate

A mixture of benzyl (2-(2-(1-bromo-2-hydroxypropan-2-yl)-6-chloropyridin-4-yl)propan-2-yl)carbamate (0.040 kg, 91 mmol), ethanol (0.50 L), and ammonium hydroxide (0.040 L) was stirred at room temperature for 4 h. The mixture was concentrated in vacuo, diluted with water, and extracted with EtOAc (200 mL×3). The combined organic extracts were dried with anhydrous sodium sulfate, filtered, and concentrated in vacuo to afford the title compound.

### Step 5: tert-butyl (2-(4-(2-(((benzyloxy)carbonyl)amino)propan-2-yl)-6-chloropyridin-2-yl)-2-hydroxypropyl)carbamate (enantiomer-1 and enantiomer-2)

Di-tert-butyl dicarbonate (28 g, 0.13 mol) was added to a 0° C. mixture of benzyl (2-(2-(1-amino-2-hydroxypropan-2-yl)-6-chloropyridin-4-yl)propan-2-yl)carbamate (0.040 kg, 0.11 mol), DIPEA (21 g, 0.16 mol), and THF (0.50 L). The mixture was warmed to room temperature and stirred for 2 h. Water (200 mL) was added and the mixture was extracted with EtOAc (200 mL×3). The combined organic extracts were dried with anhydrous sodium sulfate, filtered, and concentrated in vacuo. The residue was purified by silica gel chromatography (5-25% EtOAc/petroleum ether). The material was further purified by prep-SFC (CHIRAL ART Amylose-C NEO, 3×25 cm; CO₂:2-PrOH) to afford the resolved enantiomers of the title compounds. The first eluting isomer was designated Intermediate C-01-enantiomer-1 and the second eluting isomer was designated Intermediate C-01-enantiomer-2.

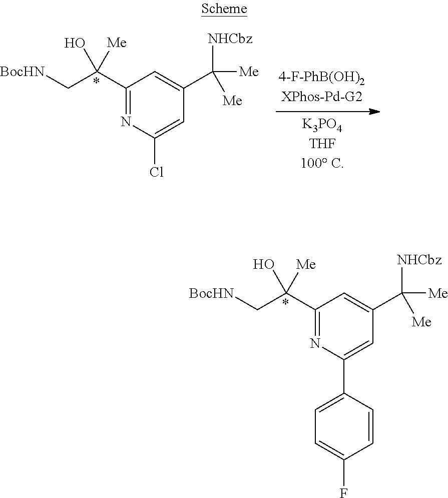 [SMILES: CC(C)(C)OC(=O)NCC(C)(O)c1cc(C(C)(C)NC(=O)OCc2ccccc2)cc(Cl)n1 ; CC(C)(C)OC(=O)NCC(C)(O)c1cc(C(C)(C)NC(=O)OCc2ccccc2)cc(-c2ccc(F)cc2)n1]

### Intermediate C-02-enantiomer-1

### tert-butyl (2-(4-(2-(((benzyloxy)carbonyl)amino)propan-2-yl)-6-(4-fluorophenyl)pyridin-2-yl)-2-hydroxypropyl)carbamate (enantiomer-1)

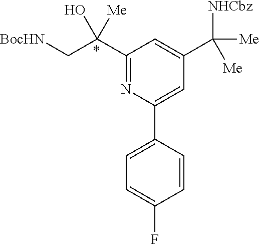 [SMILES: CC(C)(C)OC(=O)NCC(C)(O)c1cc(C(C)(C)NC(=O)OCc2ccccc2)cc(-c2ccc(F)cc2)n1]

A mixture of tert-butyl (2-(4-(2-(((benzyloxy)carbonyl)amino)propan-2-yl)-6-chloropyridin-2-yl)-2-hydroxypropyl)carbamate (Intermediate C-01-enantiomer-1, 0.20 g, 0.42 mmol), (4-fluorophenyl)boronic acid (0.070 g, 0.50 mmol), chloro(2-dicyclohexylphosphino-2′,4′,6′-triisopropyl-1,1′-biphenyl)[2-(2′-amino-1,1′-biphenyl)]palladium(II) (33 mg, 0.042 mmol), an aqueous solution of potassium phosphate tribasic (1.0 M, 0.84 mL, 0.84 mmol), and THF (2.1 mL) was stirred at 100° C. for 1.25 h. The mixture was cooled to room temperature, filtered through celite, and the filter pad was washed with EtOAc. The filtrate was concentrated in vacuo and the residue was purified by silica gel chromatography (0-50% EtOAc/hexanes) to afford the title compound.

### Intermediate C-02-enantiomer-2

### tert-butyl (2-(4-(2-(((benzyloxy)carbonyl)amino)propan-2-yl)-6-(4-fluorophenyl)pyridin-2-yl)-2-hydroxypropyl)carbamate (enantiomer-2)

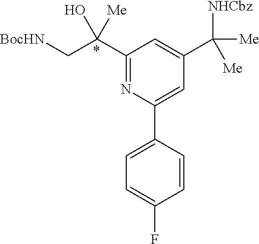 [SMILES: CC(C)(C)OC(=O)NCC(C)(O)c1cc(C(C)(C)NC(=O)OCc2ccccc2)cc(-c2ccc(F)cc2)n1]

A mixture of tert-butyl (2-(4-(2-(((benzyloxy)carbonyl)amino)propan-2-yl)-6-chloropyridin-2-yl)-2-hydroxypropyl)carbamate (Intermediate C-01-enantiomer-2, 5.0 g, 0.010 mol), (4-fluorophenyl)boronic acid (1.8 g, 13 mmol), chloro(2-dicyclohexylphosphino-2′,4′,6′-triisopropyl-1,1′-biphenyl)[2-(2′-amino-1,1′-biphenyl)]palladium(II) (0.82 g, 1.0 mmol), an aqueous solution of potassium phosphate tribasic (1.0 M, 21 mL, 21 mmol), and THF (52 mL) was stirred at 100° C. for 2 h. The mixture was cooled to room temperature, diluted with water, and extracted with EtOAc. The combined organic extracts were dried with anhydrous magnesium sulfate, filtered, and concentrated in vacuo. The residue was purified by silica gel chromatography (0-100% (20% EtOH/EtOAc)/hexanes) to afford the title compound.

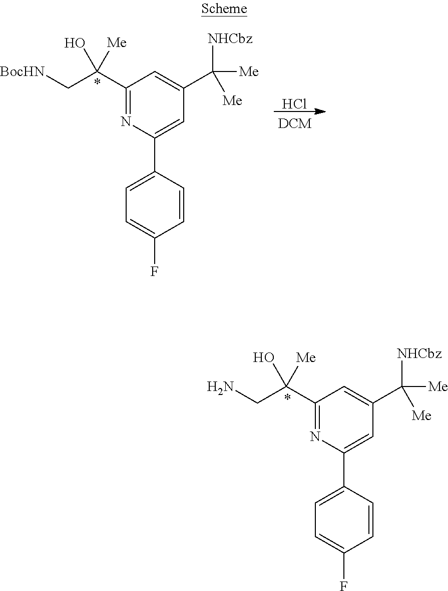 [SMILES: CC(C)(C)OC(=O)NCC(C)(O)c1cc(C(C)(C)NC(=O)OCc2ccccc2)cc(-c2ccc(F)cc2)n1 ; Cl ; CC(O)(CN)c1cc(C(C)(C)NC(=O)OCc2ccccc2)cc(-c2ccc(F)cc2)n1]

### Intermediate C-03-enantiomer-1

### benzyl (2-(2-(1-amino-2-hydroxypropan-2-yl)-6-(4-fluorophenyl)pyridin-4-yl)propan-2-yl)carbamate (enantiomer-1)

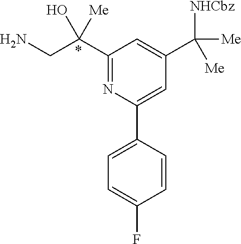 [SMILES: CC(O)(CN)c1cc(C(C)(C)NC(=O)OCc2ccccc2)cc(-c2ccc(F)cc2)n1]

A mixture of tert-butyl (2-(4-(2-(((benzyloxy)carbonyl)amino)propan-2-yl)-6-(4-fluorophenyl)pyridin-2-yl)-2-hydroxypropyl)carbamate (Intermediate C-02-enantiomer-1, 0.12 g, 0.22 mmol), DCM (1.0 mL), and HCl (4.0 M in 1,4-dioxane, 0.27 mL, 1.1 mmol) was stirred at room temperature for 18 h. Additional HCl (4.0 M in 1,4-dioxane, 0.27 mL, 1.1 mmol) was added and the mixture was stirred an additional 2 h. The mixture was concentrated in vacuo to afford the title compound.

### Intermediate C-03-enantiomer-2

### benzyl (2-(2-(1-amino-2-hydroxypropan-2-yl)-6-(4-fluorophenyl)pyridin-4-yl)propan-2-yl)carbamate (enantiomer-2)

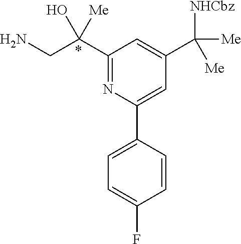 [SMILES: CC(O)(CN)c1cc(C(C)(C)NC(=O)OCc2ccccc2)cc(-c2ccc(F)cc2)n1]

A mixture of tert-butyl (2-(4-(2-(((benzyloxy)carbonyl)amino)propan-2-yl)-6-(4-fluorophenyl)pyridin-2-yl)-2-hydroxypropyl)carbamate (Intermediate C-02-enantiomer-2, 0.13 g, 0.24 mmol), DCM (1.0 mL), and HCl (4.0 M in 1,4-dioxane, 0.30 mL, 1.2 mmol) was stirred at room temperature for 18 h. Additional HCl (4.0 M in 1,4-dioxane, 0.27 mL, 1.1 mmol) was added and the mixture was stirred an additional 2 h. The mixture was concentrated in vacuo to afford the title compound.

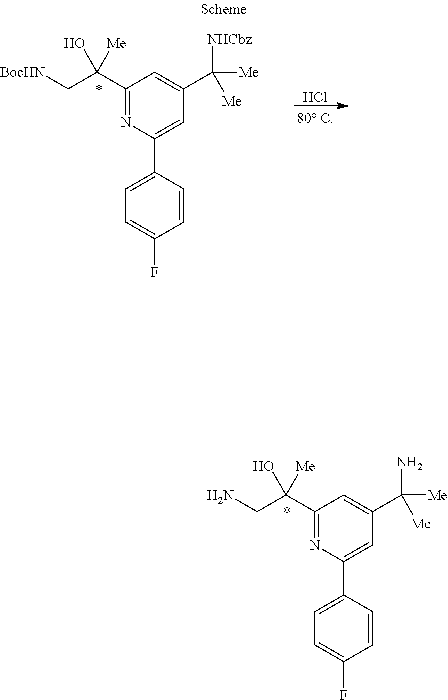 [SMILES: CC(C)(C)OC(=O)NCC(C)(O)c1cc(C(C)(C)NC(=O)OCc2ccccc2)cc(-c2ccc(F)cc2)n1 ; Cl ; CC(C)(N)c1cc(-c2ccc(F)cc2)nc(C(C)(O)CN)c1]

### Intermediate C-04-enantiomer-2

### 1-amino-2-(4-(2-aminopropan-2-yl)-6-(4-fluorophenyl)pyridin-2-yl)propan-2-ol (enantiomer-2)

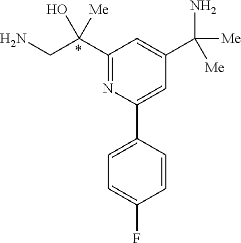 [SMILES: CC(C)(N)c1cc(-c2ccc(F)cc2)nc(C(C)(O)CN)c1]

A mixture of tert-butyl (2-(4-(2-(((benzyloxy)carbonyl)amino)propan-2-yl)-6-(4-fluorophenyl)pyridin-2-yl)-2-hydroxypropyl)carbamate (Intermediate C-02-enantiomer-2, 3.7 g, 6.8 mmol) and hydrochloric acid (12 M aqueous, 68 mL) was stirred at 80° C. for 10 min. The mixture was cooled to room temperature, diluted with water, and passed through a solid-phase extraction column (ISOLUTE® SCX-2; propylsulfonic acid bonded sorbent). The column was washed with MeOH, which was discarded. The column was then eluted with methanolic ammonia (2.0 M), and the eluent was concentrated in vacuo. DCM (100 mL) and HCl (4.0 M in 1,4-dioxane) were added, and the mixture was stirred for 15 min. The mixture was concentrated in vacuo to afford the title compound.

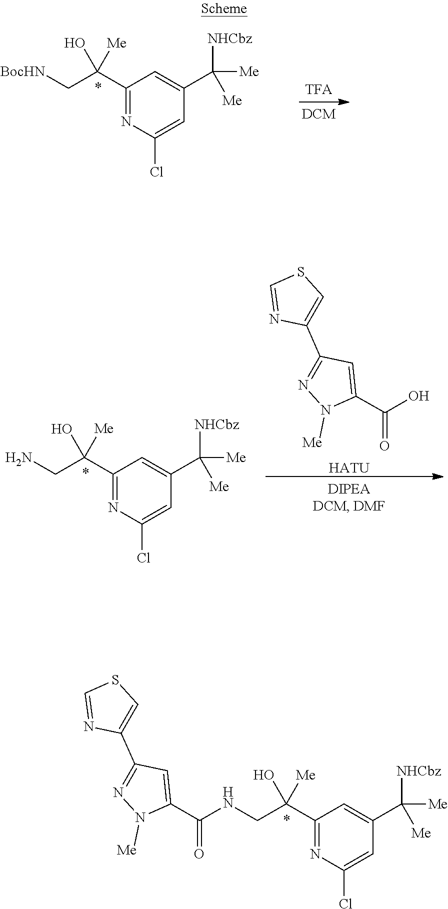

### Intermediate C-05-enantiomer-2

### benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (enantiomer-2)

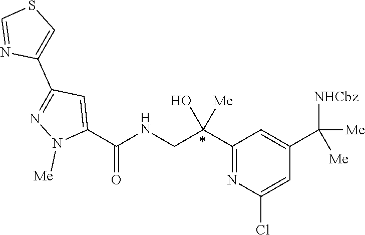 [SMILES: Cn1nc(-c2cscn2)cc1C(=O)NCC(C)(O)c1cc(C(C)(C)NC(=O)OCc2ccccc2)cc(Cl)n1]

### Step 1: benzyl (2-(2-(1-amino-2-hydroxypropan-2-yl)-6-chloropyridin-4-yl)propan-2-yl)carbamate (enantiomer-2)

A mixture of tert-butyl (2-(4-(2-(((benzyloxy)carbonyl)amino)propan-2-yl)-6-chloropyridin-2-yl)-2-hydroxypropyl)carbamate (Intermediate C-01-enantiomer-2, 0.57 g, 1.2 mmol), DCM (9.0 mL), and TFA (3.0 mL) was stirred at room temperature for 4 h. The mixture was poured into aqueous NaOH (1.0 M, 75 mL) and extracted with DCM (50 mL×3). The combined organic extracts were dried with anhydrous magnesium sulfate, filtered, and concentrated in vacuo to afford the title compound.

### Step 2: benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (enantiomer-2)

A mixture of benzyl (2-(2-(1-amino-2-hydroxypropan-2-yl)-6-chloropyridin-4-yl)propan-2-yl)carbamate (enantiomer-2, 0.45 g, 1.2 mmol), 1-methyl-3-(thiazol-4-yl)-1H-pyrazole-5-carboxylic acid (Intermediate A-05, 0.25 g, 1.2 mmol), HATU (0.50 g, 1.3 mmol), DIPEA (0.63 mL, 3.6 mmol), DMF (0.60 mL), and DCM (11 mL) was stirred at room temperature for 16 h. Brine (50 mL) was added and the mixture was extracted with DCM (50 mL×3). The combined organic extracts were dried with magnesium sulfate, filtered, and concentrated in vacuo. The residue was purified by silica gel chromatography (0-50% (25% EtOH/EtOAc)/hexanes) to afford the title compound.

### Intermediate C-06-enantiomer-2

### benzyl (2-(2-chloro-6-(2-hydroxy-1-(1′-methyl-1′H-[1,3′-bipyrazole]-5′-carboxamido)propan-2-yl)pyridin-4-yl)propan-2-yl)carbamate (enantiomer-2)

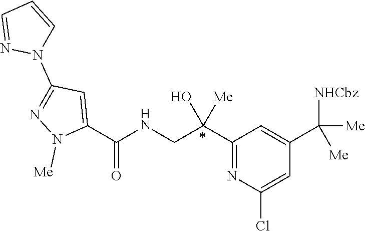 [SMILES: Cn1nc(-n2cccn2)cc1C(=O)NCC(C)(O)c1cc(C(C)(C)NC(=O)OCc2ccccc2)cc(Cl)n1]

A mixture of benzyl (2-(2-(1-amino-2-hydroxypropan-2-yl)-6-chloropyridin-4-yl)propan-2-yl)carbamate (enantiomer-2, 0.40 g, 1.1 mmol), 1′-methyl-1′H-[1,3′-bipyrazole]-5′-carboxylic acid (Intermediate A-02, 0.21 g, 1.1 mmol), HATU (0.42 g, 1.1 mmol), DIPEA (0.55 mL, 3.1 mmol), and DCM (11 mL) was stirred at room temperature for 4 h. EtOAc (50 mL) was added and the mixture was washed with water (50 mL) and brine (50 mL). The combined organic extracts were dried with magnesium sulfate, filtered, and concentrated in vacuo. The residue was purified by silica gel chromatography (0-20% (25% EtOH/EtOAc)/hexanes) to afford the title compound.

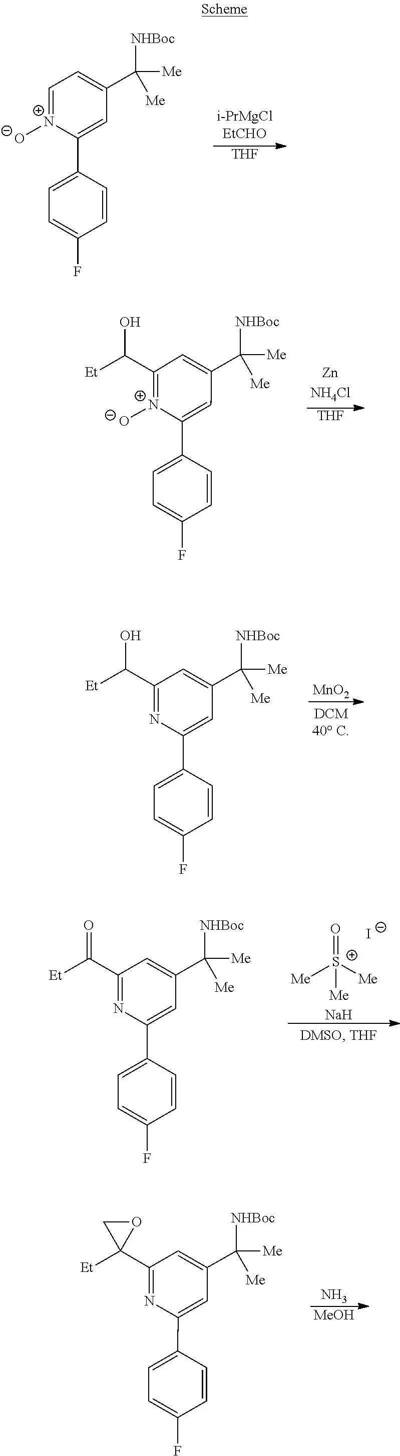

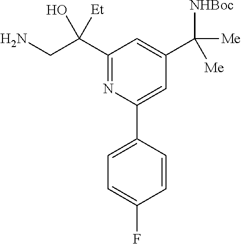 [SMILES: CCC(O)(CN)c1cc(C(C)(C)NC(=O)OC(C)(C)C)cc(-c2ccc(F)cc2)n1]

### Intermediate D-01

### tert-butyl (2-(2-(1-amino-2-hydroxybutan-2-yl)-6-(4-fluorophenyl)pyridin-4-yl)propan-2-yl)carbamate

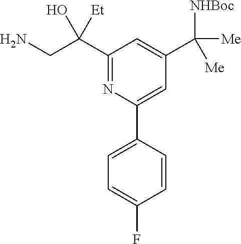 [SMILES: CCC(O)(CN)c1cc(C(C)(C)NC(=O)OC(C)(C)C)cc(-c2ccc(F)cc2)n1]

### Step 1: 4-(2-((tert-butoxycarbonyl)amino)propan-2-yl)-2-(4-fluorophenyl)-6-(1-hydroxypropyl)pyridine 1-oxide

A solution of isopropylmagnesium chloride (1.6 mL, 3.2 mmol) was added to a −78° C. solution of 4-(2-((tert-butoxycarbonyl)amino)propan-2-yl)-2-(4-fluorophenyl)pyridine 1-oxide (Intermediate B-02, 0.50 g, 1.4 mmol) in THF (15 mL) under an atmosphere of N₂. The mixture was warmed to −30° C. and stirred for 4 h. The mixture was then cooled to −78° C., propionaldehyde (1.7 g, 29 mmol) was added, and then was warmed to 25° C. and stirred for 12 h. Ammonium chloride (aqueous saturate, 20 mL) was added and the mixture was extracted with EtOAc (20 mL×3). The combined organic extracts were dried with anhydrous sodium sulfate, filtered, and concentrated in vacuo. The residue was purified by silica gel chromatography (0-30% EtOAc/petroleum ether) to afford the title compound.

### Step 2: tert-butyl (2-(2-(4-fluorophenyl)-6-(1-hydroxypropyl)pyridin-4-yl)propan-2-yl)carbamate

Zinc (0.86 g, 13 mmol) and a saturated aqueous solution of ammonium chloride (0.020 L) was added to a solution of 4-(2-((tert-butoxycarbonyl)amino)propan-2-yl)-2-(4-fluorophenyl)-6-(1-hydroxypropyl)pyridine 1-oxide (0.53 g, 1.3 mmol) in THF (15 mL). The mixture was stirred at 25° C. for 12 h. The mixture was filtered, and the filtrate was concentrated to afford the title compound.

### Step 3: tert-butyl (2-(2-(4-fluorophenyl)-6-propionylpyridin-4-yl)propan-2-yl)carbamate

A mixture of tert-butyl (2-(2-(4-fluorophenyl)-6-(1-hydroxypropyl)pyridin-4-yl)propan-2-yl)carbamate (0.50 g, 1.3 mmol), manganese(IV) oxide (1.1 g, 13 mmol), and DCM (0.010 L) was stirred at 40° C. for 24 h. The mixture was cooled to room temperature, filtered, and the filtrate was concentrated in vacuo. The residue was purified by silica gel chromatography (0-30% EtOAc/petroleum ether) to afford the title compound.

### Step 4: tert-butyl (2-(2-(2-ethyloxiran-2-yl)-6-(4-fluorophenyl)pyridin-4-yl)propan-2-yl)carbamate

Sodium hydride (60 wt % dispersion in mineral oil, 0.070 g, 1.8 mmol) was washed with pentane. THF (3.0 mL) and DMSO (3.0 mL) were added, and the mixture was stirred for 5 min. Trimethylsulfoxonium iodide (0.39 g, 1.8 mmol) was added and the mixture was stirred at 25° C. for 1 h. The mixture was then cooled to 0° C. and a solution of tert-butyl (2-(2-(4-fluorophenyl)-6-propionylpyridin-4-yl)propan-2-yl)carbamate (0.34 g, 0.88 mmol) in THF (4.0 mL) was added. The mixture was then warmed to 25° C. and stirred for 12 h. A saturated aqueous solution of ammonium chloride (8 mL) was added and the mixture was extracted with EtOAc (15 mL×3). The combined organic extracts were dried with anhydrous sodium sulfate, filtered, and concentrated in vacuo to afford the title compound.

### Step 5: tert-butyl (2-(2-(1-amino-2-hydroxybutan-2-yl)-6-(4-fluorophenyl)pyridin-4-yl)propan-2-yl)carbamate

A mixture of tert-butyl (2-(2-(2-ethyloxiran-2-yl)-6-(4-fluorophenyl)pyridin-4-yl)propan-2-yl)carbamate (0.35 g, 0.87 mmol) and methanolic ammonia (7.0 M, 0.020 L) was stirred at 25° C. for 24 h. The mixture was concentrated in vacuo and the residue was purified by preparative TLC (10% MeOH/DCM) to afford the title compound.

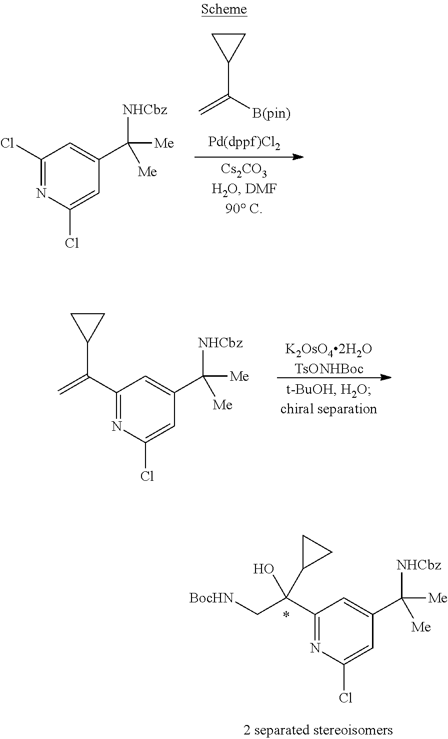

### Intermediate E-01-enantiomer-1 and Intermediate E-01-enantiomer-2

### tert-butyl (2-(4-(2-(((benzyloxy)carbonyl)amino)propan-2-yl)-6-chloropyridin-2-yl)-2-cyclopropyl-2-hydroxyethyl)carbamate (enantiomer-1 and enantiomer-2)

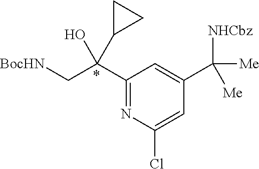 [SMILES: CC(C)(C)OC(=O)NCC(O)(c1cc(C(C)(C)NC(=O)OCc2ccccc2)cc(Cl)n1)C1CC1]

### Step 1: benzyl (2-(2-chloro-6-(1-cyclopropylvinyl)pyridin-4-yl)propan-2-yl)carbamate

A mixture of benzyl (2-(2,6-dichloropyridin-4-yl)propan-2-yl)carbamate (Intermediate B-01, 0.030 kg, 88 mmol), [1,1′-bis(diphenylphosphino)ferrocene]dichloropalladium(II) (6.5 g, 8.8 mmol), 2-(1-cyclopropylvinyl)-4,4,5,5-tetramethyl-1,3,2-dioxaborolane (19 g, 93 mmol), DMF (0.60 L), and an aqueous cesium carbonate solution (6.0 M, 45 mL, 0.27 mol) was stirred at 90° C. for 16 h under an atmosphere of N₂. The mixture was cooled to 40° C. and an aqueous solution of lithium chloride (5 wt %, 1.2 L) and EtOAc (200 mL) were added. The mixture was filtered through a pad of celite, the pad was washed with EtOAc (200 mL×3), and the filtrate was extracted with EtOAc (200 mL×3). The combined organic extracts were dried with anhydrous sodium sulfate, filtered, and concentrated in vacuo. The residue was purified by silica gel chromatography (0-100% EtOAc/petroleum ether) to afford the title compound.

### Step 2: tert-butyl (2-(4-(2-(((benzyloxy)carbonyl)amino)propan-2-yl)-6-chloropyridin-2-yl)-2-cyclopropyl-2-hydroxyethyl)carbamate (enantiomer-1 and enantiomer-2)

Potassium osmate(VI) dihydrate (1.9 g, 5.0 mmol) was added to a mixture of benzyl (2-(2-chloro-6-(1-cyclopropylvinyl)pyridin-4-yl)propan-2-yl)carbamate (11 g, 0.030 mol), tert-butyl (tosyloxy)carbamate (13 g, 45 mmol), tert-butanol (99 mL), and water (33 mL). The mixture was stirred at 25° C. for 12 h. A saturated aqueous solution of sodium sulfite (300 mL) was added, and the mixture was filtered through a pad of celite. The pad was washed with EtOAc (50 mL×2), and the filtrate was extracted with EtOAc (150 mL×3). The combined organic extracts were washed with brine (100 mL), dried with anhydrous sodium sulfate, filtered, and concentrated in vacuo. The residue was purified by silica gel chromatography (0-100% EtOAc/petroleum ether) and then further purified by SFC (DAICEL CHIRALPAK® AD, 250×50 mm, 10 μm; 20% 2-PrOH (with 0.1% NH₄OH)/CO₂) to afford the title compounds. The first eluting isomer was designated Intermediate E-01-enantiomer-1 and the second eluting isomer was designated Intermediate E-01-enantiomer-2.

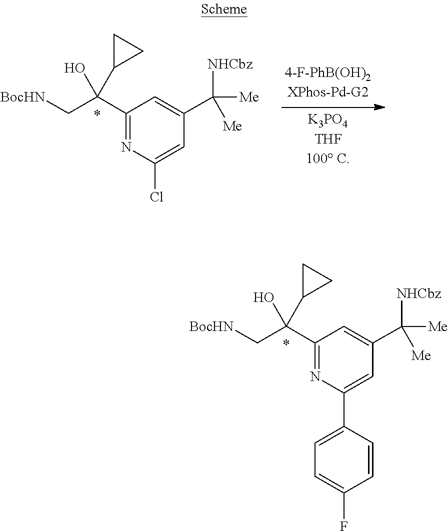 [SMILES: CC(C)(C)OC(=O)NCC(O)(c1cc(C(C)(C)NC(=O)OCc2ccccc2)cc(Cl)n1)C1CC1 ; CC(C)(C)OC(=O)NCC(O)(c1cc(C(C)(C)NC(=O)OCc2ccccc2)cc(-c2ccc(F)cc2)n1)C1CC1]

### Intermediate E-02-enantiomer-1

### tert-butyl (2-(4-(2-(((benzyloxy)carbonyl)amino)propan-2-yl)-6-(4-fluorophenyl)pyridin-2-yl)-2-cyclopropyl-2-hydroxyethyl)carbamate (enantiomer-1)

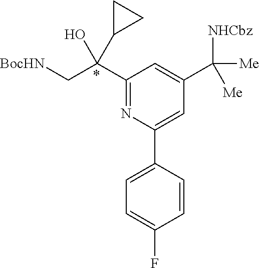 [SMILES: CC(C)(C)OC(=O)NCC(O)(c1cc(C(C)(C)NC(=O)OCc2ccccc2)cc(-c2ccc(F)cc2)n1)C1CC1]

A mixture of tert-butyl (2-(4-(2-(((benzyloxy)carbonyl)amino)propan-2-yl)-6-chloropyridin-2-yl)-2-cyclopropyl-2-hydroxyethyl)carbamate (Intermediate E-01-enantiomer-1, 75 mg, 0.15 mmol), (4-fluorophenyl)boronic acid (25 mg, 0.18 mmol), chloro(2-dicyclohexylphosphino-2′,4′,6′-triisopropyl-1,1′-biphenyl)[2-(2′-amino-1,1′-biphenyl)]palladium(II) (12 mg, 0.015 mmol), aqueous potassium phosphate tribasic (1.0 M, 0.30 mL, 0.30 mmol), and THF (0.74 mL) was stirred at 100° C. for 2 h. The mixture was cooled to room temperature, diluted with water, and extracted with EtOAc. The organic extract was dried with anhydrous magnesium sulfate, filtered, and concentrated in vacuo. The residue was purified by silica gel chromatography (0-100% EtOAc/hexanes) to afford the title compound.

### Intermediate E-02-enantiomer-2

### tert-butyl (2-(4-(2-(((benzyloxy)carbonyl)amino)propan-2-yl)-6-(4-fluorophenyl)pyridin-2-yl)-2-cyclopropyl-2-hydroxyethyl)carbamate (enantiomer-2)

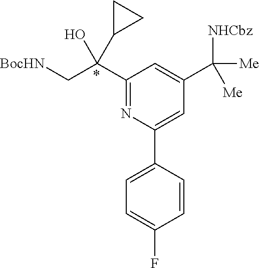 [SMILES: CC(C)(C)OC(=O)NCC(O)(c1cc(C(C)(C)NC(=O)OCc2ccccc2)cc(-c2ccc(F)cc2)n1)C1CC1]

A mixture of tert-butyl (2-(4-(2-(((benzyloxy)carbonyl)amino)propan-2-yl)-6-chloropyridin-2-yl)-2-cyclopropyl-2-hydroxyethyl)carbamate (Intermediate E-01-enantiomer-2, 2.6 g, 5.1 mmol), (4-fluorophenyl)boronic acid (0.86 g, 6.1 mmol), chloro(2-dicyclohexylphosphino-2′,4′,6′-triisopropyl-1,1′-biphenyl)[2-(2′-amino-1,1′-biphenyl)]palladium(II) (0.40 g, 0.51 mmol), aqueous potassium phosphate tribasic (1.0 M, 0.010 L, 0.010 mol), and THF (26 mL) was stirred at 100° C. for 2 h. The mixture was cooled to room temperature, diluted with water, and extracted with EtOAc. The organic extract was dried with anhydrous magnesium sulfate, filtered, and concentrated in vacuo. The residue was purified by silica gel chromatography (0-100% (20% EtOH/EtOAc)/hexanes) to afford the title compound.

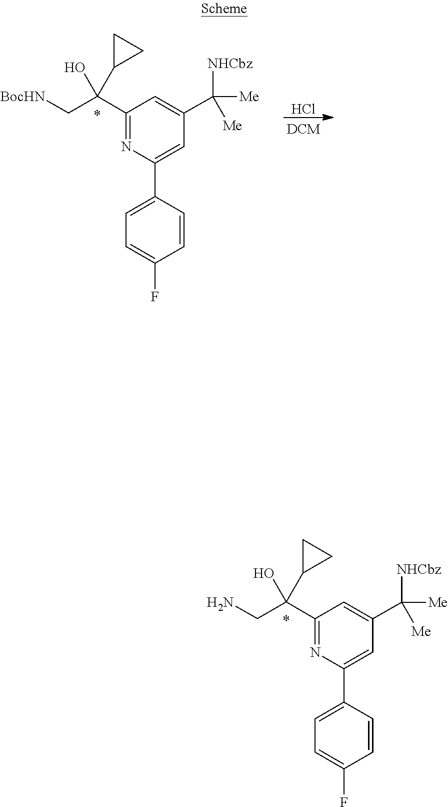 [SMILES: CC(C)(C)OC(=O)NCC(O)(c1cc(C(C)(C)NC(=O)OCc2ccccc2)cc(-c2ccc(F)cc2)n1)C1CC1 ; Cl ; CC(C)(NC(=O)OCc1ccccc1)c1cc(-c2ccc(F)cc2)nc(C(O)(CN)C2CC2)c1]

### Intermediate E-03-enantiomer-1

### benzyl (2-(2-(2-amino-1-cyclopropyl-1-hydroxyethyl)-6-(4-fluorophenyl)pyridin-4-yl)propan-2-yl)carbamate (enantiomer-1)

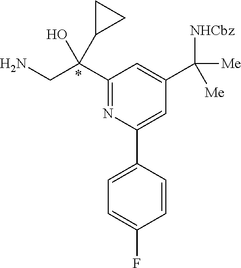 [SMILES: CC(C)(NC(=O)OCc1ccccc1)c1cc(-c2ccc(F)cc2)nc(C(O)(CN)C2CC2)c1]

A mixture of tert-butyl (2-(4-(2-(((benzyloxy)carbonyl)amino)propan-2-yl)-6-(4-fluorophenyl)pyridin-2-yl)-2-cyclopropyl-2-hydroxyethyl)carbamate (Intermediate E-02-enantiomer-1, 44 mg, 0.078 mmol), DCM (0.78 mL), and HCl (4.0 M in 1,4-dioxane, 0.39 mL, 1.6 mmol) was stirred at room temperature for 18 h. The mixture was concentrated in vacuo and residue was triturated (Et₂O) to afford the title compound.

### Intermediate E-03-enantiomer-2

### benzyl (2-(2-(2-amino-1-cyclopropyl-1-hydroxyethyl)-6-(4-fluorophenyl)pyridin-4-yl)propan-2-yl)carbamate (enantiomer-2)

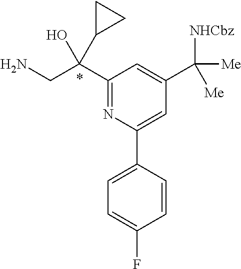 [SMILES: CC(C)(NC(=O)OCc1ccccc1)c1cc(-c2ccc(F)cc2)nc(C(O)(CN)C2CC2)c1]

A mixture of tert-butyl (2-(4-(2-(((benzyloxy)carbonyl)amino)propan-2-yl)-6-(4-fluorophenyl)pyridin-2-yl)-2-cyclopropyl-2-hydroxyethyl)carbamate (Intermediate E-02-enantiomer-2, 37 mg, 0.066 mmol), DCM (0.66 mL), and HCl (4.0 M in 1,4-dioxane, 0.33 mL, 1.3 mmol) was stirred at room temperature for 18 h. The mixture was concentrated in vacuo and the residue was triturated (Et₂O) to afford the title compound.

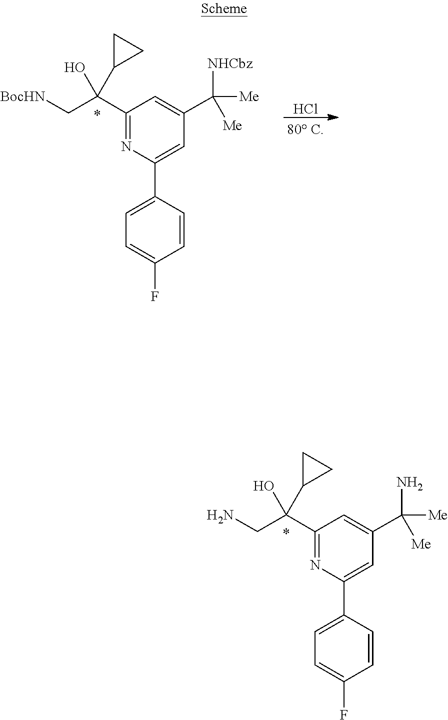 [SMILES: CC(C)(C)OC(=O)NCC(O)(c1cc(C(C)(C)NC(=O)OCc2ccccc2)cc(-c2ccc(F)cc2)n1)C1CC1 ; Cl ; CC(C)(N)c1cc(-c2ccc(F)cc2)nc(C(O)(CN)C2CC2)c1]

### Intermediate E-04-enantiomer-2

### 2-amino-1-(4-(2-aminopropan-2-yl)-6-(4-fluorophenyl)pyridin-2-yl)-1-cyclopropylethan-1-ol (enantiomer-2)

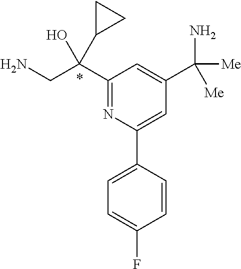 [SMILES: CC(C)(N)c1cc(-c2ccc(F)cc2)nc(C(O)(CN)C2CC2)c1]

A mixture of tert-butyl (2-(4-(2-(((benzyloxy)carbonyl)amino)propan-2-yl)-6-(4-fluorophenyl)pyridin-2-yl)-2-cyclopropyl-2-hydroxyethyl)carbamate (Intermediate E-02-enantiomer-2, 2.0 g, 3.6 mmol) and hydrochloric acid (12 M aqueous, 36 mL) was stirred at 80° C. for 10 min. The mixture was cooled to room temperature, diluted with water, and loaded onto a solid-phase extraction column (ISOLUTE® SCX-2; propylsulfonic acid bonded sorbent). The column was washed with MeOH, which was discarded. The column was eluted with methanolic ammonia (2.0 M) and the eluent was concentrated in vacuo. DCM (50 mL) and HCl (4.0 M in 1,4-dioxane, 4.4 mL) were added and the mixture was stirred for 15 min and then concentrated in vacuo to afford the title compound.

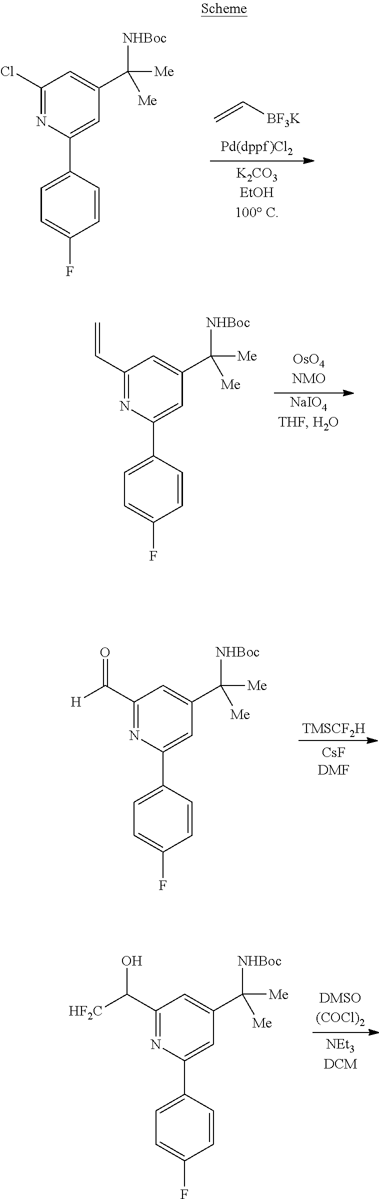

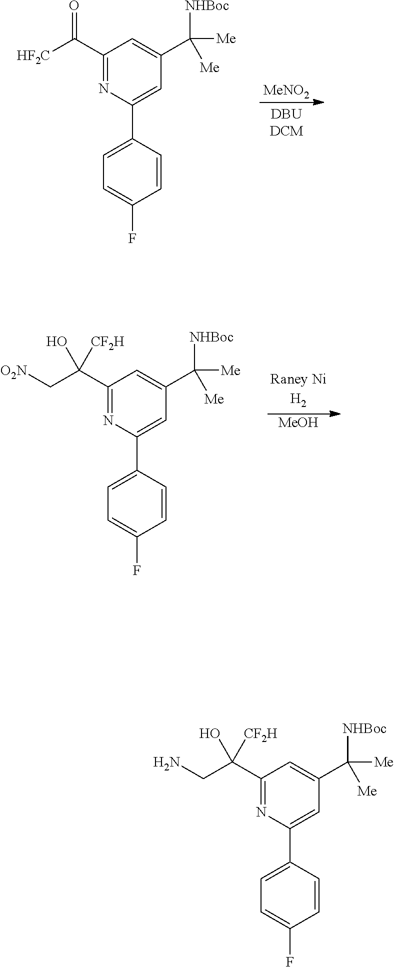

### Intermediate-F-01

### tert-butyl (2-(2-(3-amino-1,1-difluoro-2-hydroxypropan-2-yl)-6-(4-fluorophenyl)pyridin-4-yl)propan-2-yl)carbamate

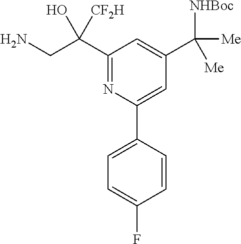 [SMILES: CC(C)(C)OC(=O)NC(C)(C)c1cc(-c2ccc(F)cc2)nc(C(O)(CN)C(F)F)c1]

### Step 1: tert-butyl (2-(2-(4-fluorophenyl)-6-vinylpyridin-4-yl)propan-2-yl)carbamate

A mixture of tert-butyl (2-(2-chloro-6-(4-fluorophenyl)pyridin-4-yl)propan-2-yl)carbamate (Intermediate B-03, 2.0 g, 5.5 mmol), potassium vinyltrifluoroborate (0.88 g, 6.6 mmol), potassium carbonate (1.1 g, 8.2 mmol), [1,1′-bis(diphenylphosphino)ferrocene]dichloropalladium(II) (0.40 g, 0.55 mmol), and EtOH (25 mL) was stirred at 100° C. for 12 h. The mixture was cooled to room temperature, water was added, and the mixture was extracted with EtOAc (3×). The combined organic extracts were washed with brine (2×), dried with anhydrous sodium sulfate, filtered, and concentrated in vacuo. The residue was purified by silica gel chromatography (0-25% EtOAc/petroleum ether) to afford the title compound.

### Step 2: tert-butyl (2-(2-(4-fluorophenyl)-6-formylpyridin-4-yl)propan-2-yl)carbamate

Potassium osmate(VI) dihydrate (21 mg, 0.056 mmol) was added to a mixture of tert-butyl (2-(2-(4-fluorophenyl)-6-vinylpyridin-4-yl)propan-2-yl)carbamate (0.10 g, 0.28 mmol), 4-methylmorpholine N-oxide (66 mg, 0.56 mmol), and THF (4.0 mL). The mixture was stirred for 1 h at 25° C., sodium periodate (0.18 g, 0.84 mmol) was added, and then stirred for 12 h at 25° C. Aqueous sodium sulfite (saturated, 30 mL) was added and the mixture was extracted with EtOAc (30 mL×3). The combined organic extracts were dried with anhydrous sodium sulfate, filtered, and concentrated in vacuo. The material was purified by silica gel chromatography (0-5% EtOAc/petroleum ether) to afford the title compound.

### Step 3: tert-butyl (2-(2-(2,2-difluoro-1-hydroxyethyl)-6-(4-fluorophenyl)pyridin-4-yl)propan-2-yl)carbamate

(Difluoromethyl)trimethylsilane (0.35 mg, 2.8 mmol) and cesium fluoride (0.42 g, 2.8 mmol) were added to a 0° C. mixture of tert-butyl (2-(2-(4-fluorophenyl)-6-formylpyridin-4-yl)propan-2-yl)carbamate (0.50 g, 1.4 mmol) and DMF (0.020 L). The mixture was warmed to 25° C. and stirred for 12 h. The mixture was diluted with water and extracted with EtOAc (2×). The combined organic extracts were washed with brine (2×), dried with anhydrous sodium sulfate, filtered, and concentrated in vacuo. The residue was purified by silica gel chromatography (0-25% EtOAc/petroleum ether) to afford the title compound.

### Step 4: tert-butyl (2-(2-(2,2-difluoroacetyl)-6-(4-fluorophenyl)pyridin-4-yl)propan-2-yl)carbamate

DMSO (0.36 mL, 5.1 mmol) was added to a −78° C. solution of oxalyl chloride (0.21 mL, 2.5 mmol) in DCM (15 mL) and the mixture was stirred for 15 min. A solution of tert-butyl (2-(2-(2,2-difluoro-1-hydroxyethyl)-6-(4-fluorophenyl)pyridin-4-yl)propan-2-yl)carbamate (0.26 g, 0.63 mmol) in DCM (5.0 mL) was added and the mixture was stirred for 30 min. Triethylamine (1.1 mL, 7.6 mmol) was added and the mixture was warmed to 0° C. and stirred for 30 min. Saturated aqueous ammonium chloride was added and the mixture was extracted with DCM (3×). The combined organic extracts were washed with brine, dried with anhydrous sodium sulfate, filtered, and concentrated in vacuo to afford the title compound.

### Step 5: tert-butyl (2-(2-(1,1-difluoro-2-hydroxy-3-nitropropan-2-yl)-6-(4-fluorophenyl)pyridin-4-yl)propan-2-yl)carbamate

Nitromethane (65 mg, 1.1 mmol) and 1,8-diazabicyclo[5.4.0]undec-7-ene (0.11 mL, 0.74 mmol) were added to a mixture of tert-butyl (2-(2-(2,2-difluoroacetyl)-6-(4-fluorophenyl)pyridin-4-yl)propan-2-yl)carbamate (0.27 g, 0.53 mmol) and DCM (0.010 L). The mixture was stirred at 25° C. for 1 h. Water was added and the mixture was extracted with DCM (3×). The combined organic extracts were dried with anhydrous sodium sulfate, filtered, and concentrated in vacuo. The residue was purified by preparative TLC (25% EtOAc/petroleum ether) to afford the title compound.

### Step 6: tert-butyl (2-(2-(3-amino-1,1-difluoro-2-hydroxypropan-2-yl)-6-(4-fluorophenyl)pyridin-4-yl)propan-2-yl)carbamate

Raney®-nickel (28 mg, 0.47 mmol) was added to a mixture of tert-butyl (2-(2-(1,1-difluoro-2-hydroxy-3-nitropropan-2-yl)-6-(4-fluorophenyl)pyridin-4-yl)propan-2-yl)carbamate (0.22 g, 0.47 mmol) and MeOH (0.020 L) under an atmosphere of N₂. The mixture was placed under an atmosphere of H2 and stirred at 25° C. for 1 h. The mixture was filtered and the filtrate was concentrated in vacuo. The residue was purified by preparative TLC (EtOAc) to afford the title compound.

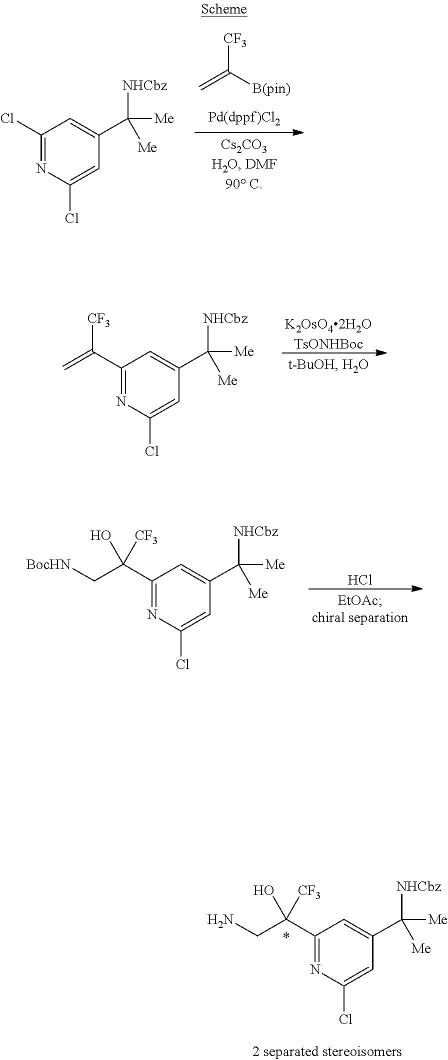

### Intermediate G-01-enantiomer-1 and Intermediate G-01-enantiomer-2

### benzyl (2-(2-(3-amino-1,1,1-trifluoro-2-hydroxypropan-2-yl)-6-chloropyridin-4-yl)propan-2-yl)carbamate (enantiomer-1 and enantiomer-2)

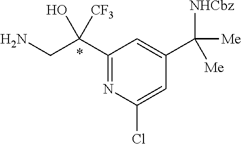 [SMILES: CC(C)(NC(=O)OCc1ccccc1)c1cc(Cl)nc(C(O)(CN)C(F)(F)F)c1]

### Step 1: benzyl (2-(2-chloro-6-(3,3,3-trifluoroprop-1-en-2-yl)pyridin-4-yl)propan-2-yl)carbamate

A mixture of benzyl (2-(2,6-dichloropyridin-4-yl)propan-2-yl)carbamate (Intermediate B-01, 22 g, 65 mmol), [1,1′-bis(diphenylphosphino)ferrocene]dichloropalladium(II) (6.5 g, 8.8 mmol), 4,4,5,5-tetramethyl-2-(3,3,3-trifluoroprop-1-en-2-yl)-1,3,2-dioxaborolane (14 g, 65 mmol), DME (0.44 L), and an aqueous cesium carbonate solution (6.0 M, 33 mL, 0.20 mol) was stirred at 85° C. for 2 h under an atmosphere of Argon. The mixture was cooled to 5-10° C. and an ice-water mixture (300 mL) was added. The mixture was extracted with EtOAc (200 mL×2) and the combined organic extracts were concentrated in vacuo. The residue was purified by silica gel chromatography (5-100% EtOAc/petroleum ether) to afford the title compound.

### Step 2: tert-butyl (2-(4-(2-(((benzyloxy)carbonyl)amino)propan-2-yl)-6-chloropyridin-2-yl)-3,3,3-trifluoro-2-hydroxypropyl)carbamate

Potassium osmate(VI) dihydrate (1.2 g, 3.2 mmol) was added to a mixture of benzyl (2-(2-chloro-6-(3,3,3-trifluoroprop-1-en-2-yl)pyridin-4-yl)propan-2-yl)carbamate (7.0 g, 18 mmol), tert-butyl (tosyloxy)carbamate (9.1 g, 32 mmol), tert-butanol (47 mL), and water (16 mL). The mixture was stirred at 25° C. for 12 h and then poured into an ice/water mixture (50 mL) and extracted with DCM (30 mL×2). The combined organic extracts were concentrated in vacuo and purified by silica gel chromatography (5-100% EtOAc/petroleum ether) to afford the title compound.

### Step 3: benzyl (2-(2-(3-amino-1,1,1-trifluoro-2-hydroxypropan-2-yl)-6-chloropyridin-4-yl)propan-2-yl)carbamate (enantiomer-1 and enantiomer-2)

HCl (4.0 M in EtOAc, 0.24 L, 0.96 mol) was added to a 0° C. mixture of tert-butyl (2-(4-(2-(((benzyloxy)carbonyl)amino)propan-2-yl)-6-chloropyridin-2-yl)-3,3,3-trifluoro-2-hydroxypropyl)carbamate (12 g, 23 mmol) and EtOAc (0.12 L). The mixture was warmed to ambient temperature and stirred for 12 h. The mixture was concentrated in vacuo and EtOAc (50 mL) and ice/water (50 mL) were added. The pH of the mixture was adjusted to pH 7-8 with sodium carbonate (aqueous saturated) and the mixture was extracted with EtOAc (50 mL×2). The combined organic extracts were concentrated and purified by SFC (Chiralcel® OJ, 250×50 mm, 10 μm; 17% EtOH (0.1% isopropylamine modifier)/n-heptane) to afford the title compounds. The first eluting isomer was designated Intermediate G-01-enantiomer-1 and the second eluting isomer was designated Intermediate G-01-enantiomer-2.

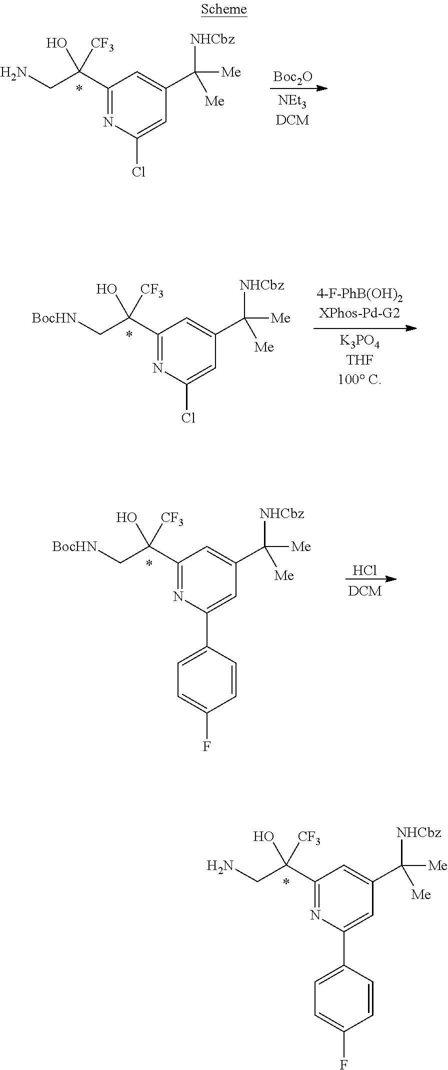 [SMILES: "Boc2O" ; CC(C)(NC(=O)OCc1ccccc1)c1cc(Cl)nc(C(O)(CN)C(F)(F)F)c1 ; CC(C)(C)OC(=O)NCC(O)(c1cc(C(C)(C)NC(=O)OCc2ccccc2)cc(Cl)n1)C(F)(F)F ; CC(C)(C)OC(=O)NCC(O)(c1cc(C(C)(C)NC(=O)OCc2ccccc2)cc(-c2ccc(F)cc2)n1)C(F)(F)F ; Cl ; CC(C)(NC(=O)OCc1ccccc1)c1cc(-c2ccc(F)cc2)nc(C(O)(CN)C(F)(F)F)c1]

### Intermediate G-02-enantiomer-1

### 3-amino-2-(4-(2-aminopropan-2-yl)-6-(4-fluorophenyl)pyridin-2-yl)-1,1,1-trifluoropropan-2-ol (enantiomer-1)

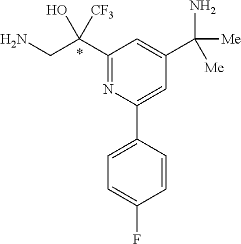 [SMILES: CC(C)(N)c1cc(-c2ccc(F)cc2)nc(C(O)(CN)C(F)(F)F)c1]

### Step 1: tert-butyl (2-(4-(2-(((benzyloxy)carbonyl)amino)propan-2-yl)-6-chloropyridin-2-yl)-3,3,3-trifluoro-2-hydroxypropyl)carbamate (enantiomer-1)

A mixture of benzyl (2-(2-(3-amino-1,1,1-trifluoro-2-hydroxypropan-2-yl)-6-chloropyridin-4-yl)propan-2-yl)carbamate (Intermediate-G-01-enantiomer-1, 2.3 g, 5.3 mmol), di-tert-butyl dicarbonate (1.2 g, 5.6 mmol), triethylamine (2.2 mL, 16 mmol), and DCM (27 mL) was stirred at room temperature for 2 h. The mixture was concentrated in vacuo and the residue was purified by silica gel chromatography (0-100% EtOAc/hexanes) to afford the title compound.

### Step 2: tert-butyl (2-(4-(2-aminopropan-2-yl)-6-(4-fluorophenyl)pyridin-2-yl)-3,3,3-trifluoro-2-hydroxypropyl)carbamate (enantiomer-1)

A mixture of tert-butyl (2-(4-(2-(((benzyloxy)carbonyl)amino)propan-2-yl)-6-chloropyridin-2-yl)-3,3,3-trifluoro-2-hydroxypropyl)carbamate (enantiomer-1, 2.8 g, 5.3 mmol), (4-fluorophenyl)boronic acid (0.88 g, 6.3 mmol), chloro(2-dicyclohexylphosphino-2′,4′,6′-triisopropyl-1,1′-biphenyl)[2-(2′-amino-1,1′-biphenyl)]palladium(II) (0.41 g, 0.53 mmol), aqueous potassium phosphate tribasic (1.0 M, 11 mL, 11 mmol), and THF (26 mL) was stirred at 100° C. for 2 h. The mixture was cooled to room temperature, diluted with water, and extracted with EtOAc (3×). The combined organic extracts were dried with anhydrous magnesium sulfate, filtered, and concentrated in vacuo. The residue was purified by silica gel chromatography (0-100% (20% EtOH/EtOAc)/hexanes) to afford the title compound.

### Step 3: 3-amino-2-(4-(2-aminopropan-2-yl)-6-(4-fluorophenyl)pyridin-2-yl)-1,1,1-trifluoropropan-2-ol (enantiomer-1)

A mixture of tert-butyl (2-(4-(2-aminopropan-2-yl)-6-(4-fluorophenyl)pyridin-2-yl)-3,3,3-trifluoro-2-hydroxypropyl)carbamate (enantiomer-1, 0.91 g, 2.0 mmol), HCl (4.0 M in 1,4-dioxane, 0.010 L, 0.010 mol), and DCM (0.020 L) was stirred at room temperature for 16 h. The mixture was concentrated in vacuo to afford the title compound.

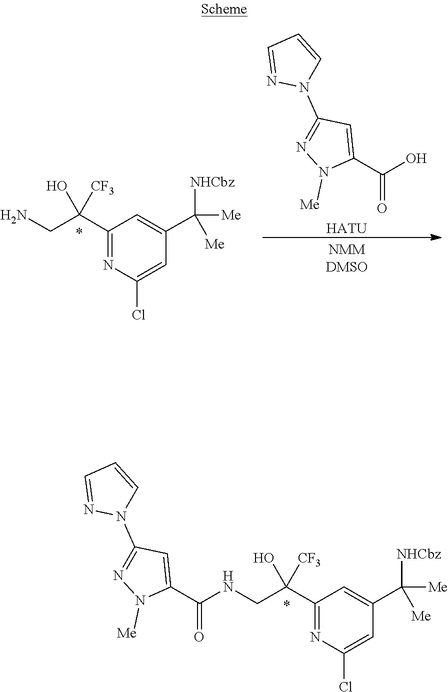

### Intermediate G-03-enantiomer-1

### benzyl (2-(2-chloro-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (enantiomer-1)

A mixture of benzyl (2-(2-(3-amino-1,1,1-trifluoro-2-hydroxypropan-2-yl)-6-chloropyridin-4-yl)propan-2-yl)carbamate (Intermediate G-01-enantiomer-1, 0.050 g, 0.12 mmol), 1′-methyl-1′H-[1,3′-bipyrazole]-5′-carboxylic acid (Intermediate A-02, 25 mg, 0.13 mmol), HATU (53 mg, 0.14 mmol), N-methylmorpholine (0.038 mL, 0.35 mmol), and DMSO (0.58 mL) was stirred at 25° C. for 18 h. EtOAc (30 mL) was added and the mixture was washed with a saturated aqueous solution of sodium bicarbonate (30 mL). The combined organic extracts were dried with magnesium sulfate, filtered, and concentrated in vacuo. The residue was purified by silica gel chromatography (0-100% EtOAc/hexanes) to afford the title compound.

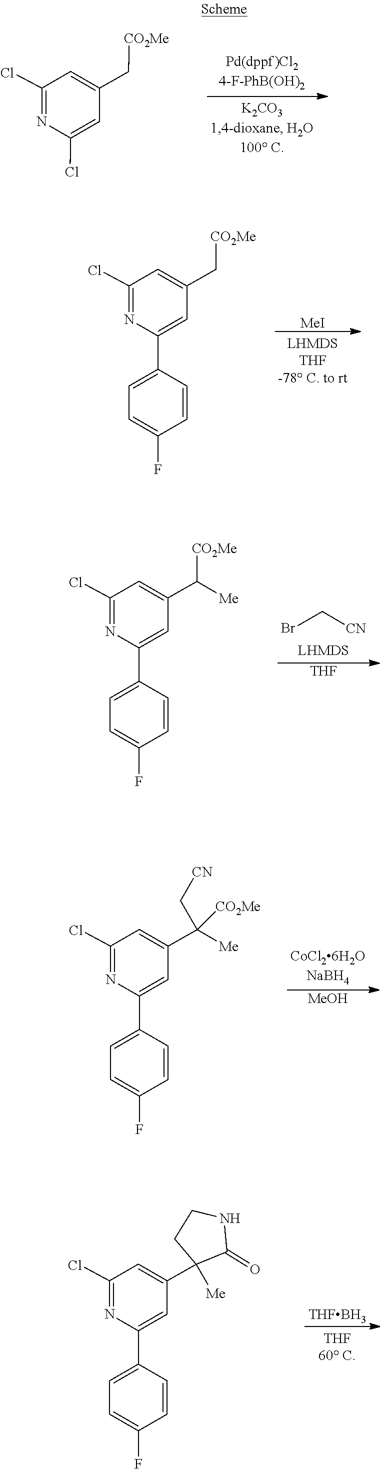

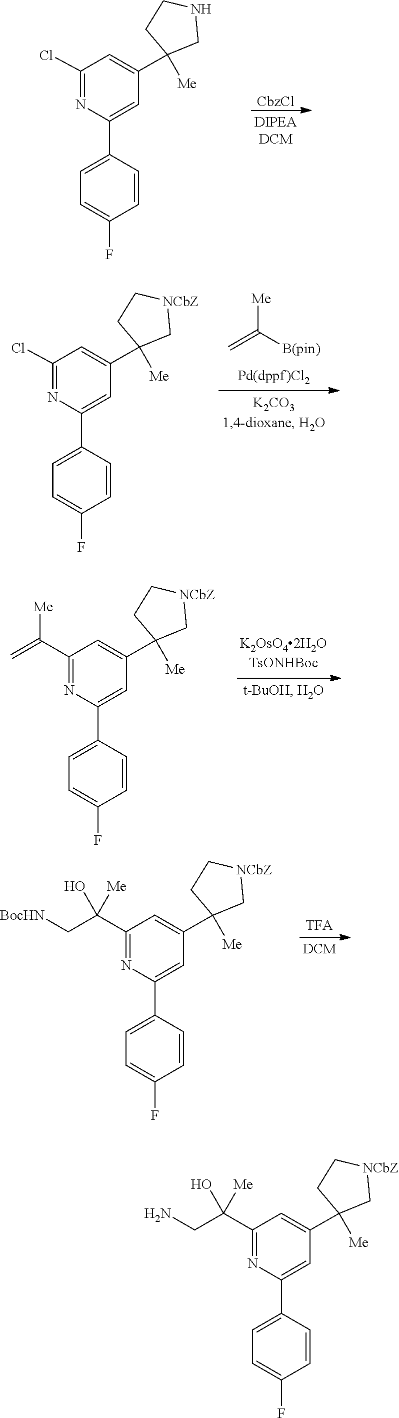

### Intermediate H-01

### benzyl 3-(2-(1-amino-2-hydroxypropan-2-yl)-6-(4-fluorophenyl)pyridin-4-yl)-3-methylpyrrolidine-1-carboxylate

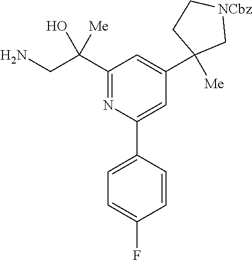 [SMILES: CC(O)(CN)c1cc(C2(C)CCN(C(=O)OCc3ccccc3)C2)cc(-c2ccc(F)cc2)n1]

### Step 1: methyl 2-(2-chloro-6-(4-fluorophenyl)pyridin-4-yl)acetate

Under an atmosphere of nitrogen, a mixture of methyl 2-(2,6-dichloropyridin-4-yl)acetate (0.030 kg, 0.14 mol), (4-fluorophenyl)boronic acid (19 g, 0.14 mol), [1,1′-bis(diphenylphosphino)ferrocene]dichloropalladium(II) (5.0 g, 6.8 mmol), potassium carbonate (38 g, 0.27 mol), 1,4-dioxane (0.40 L), and water (0.040 L) was stirred at 100° C. for 2 h. The mixture was cooled to room temperature, filtered, and concentrated in vacuo. The residue was purified by silica gel chromatography (0-8% EtOAc/petroleum ether) to afford the title compound.

### Step 2: methyl 2-(2-chloro-6-(4-fluorophenyl)pyridin-4-yl)propanoate

A solution of lithium bis(trimethylsilyl)amide (1.0 M in THF, 0.060 L, 0.060 mol) was added to a −78° C. solution of methyl 2-(2-chloro-6-(4-fluorophenyl)pyridin-4-yl)acetate (14 g, 0.050 mol) in THF (0.15 L) under an atmosphere of N₂. The mixture was stirred for 0.5 h and iodomethane (6.3 mL, 0.10 mol) was added dropwise. The mixture was warmed to room temperature and stirred for an additional 2 h. Water (100 mL) was added and the mixture was extracted with EtOAc (100 mL×3). The combined organic extracts were washed with brine (100 mL), dried with anhydrous sodium sulfate, and concentrated under reduced pressure. The residue was purified by silica gel chromatography (0-5% EtOAc/petroleum ether) to afford the title compound.

### Step 3: methyl 2-(2-chloro-6-(4-fluorophenyl)pyridin-4-yl)-3-cyano-2-methylpropanoate

A solution of lithium bis(trimethylsilyl)amide (1.0 M in THF, 6.6 mL, 6.6 mmol) was added to a −78° C. mixture of methyl 2-(2-chloro-6-(4-fluorophenyl)pyridin-4-yl)propanoate (1.5 g, 5.1 mmol) and THF (0.030 L). After stirring for 1 h, 2-bromoacetonitrile (0.80 g, 6.6 mmol) was added. The mixture was warmed to 25° C. and then stirred for an additional 2 h. Water (20 mL) was added, and the mixture was extracted with EtOAc (15 mL×3). The combined organic extracts were washed with brine (20 mL), dried with anhydrous sodium sulfate, filtered, and concentrated in vacuo. The residue was purified by silica gel chromatography (0-10% EtOAc/petroleum ether) to afford the title compound.

### Step 4: 3-(2-chloro-6-(4-fluorophenyl)pyridin-4-yl)-3-methylpyrrolidin-2-one

A mixture of methyl 2-(2-chloro-6-(4-fluorophenyl)pyridin-4-yl)-3-cyano-2-methylpropanoate (1.5 g, 4.5 mmol), cobalt(II) chloride hexahydrate (1.1 g, 4.5 mmol), and MeOH (0.030 L) was cooled to 0° C. and stirred for 5 min. Sodium borohydride (1.0 g, 27 mmol) was added portion wise over a 5 min period. The mixture was warmed to room temperature and stirred for 16 h. An aqueous solution of ammonium chloride (20 mL) was added and the mixture was extracted with EtOAc (20 mL×3). The combined organic extracts were dried with anhydrous sodium sulfate, filtered, and concentrated in vacuo. The residue was purified by silica gel chromatography (0-35% EtOAc/petroleum ether) to afford the title compound.

### Step 5: 2-chloro-6-(4-fluorophenyl)-4-(3-methylpyrrolidin-3-yl)pyridine

A borane·THF complex solution (1.0 M in THF, 0.020 L, 0.020 mol) was added to a 0° C. mixture of 3-(2-chloro-6-(4-fluorophenyl)pyridin-4-yl)-3-methylpyrrolidin-2-one (0.60 g, 2.0 mmol) and THF (0.020 L). The mixture was heated at 60° C. for 12 h and then cooled to 0° C. and MeOH (15 mL) was added. The mixture was concentrated in vacuo to afford the title compound.

### Step 6: benzyl 3-(2-chloro-6-(4-fluorophenyl)pyridin-4-yl)-3-methylpyrrolidine-1-carboxylate

Benzyl chloroformate (0.39 mL, 2.8 mmol) was added to a mixture of 2-chloro-6-(4-fluorophenyl)-4-(3-methylpyrrolidin-3-yl)pyridine (0.50 g, 1.4 mmol), DIPEA (0.72 mL, 4.1 mmol), and DCM (15 mL). The mixture was stirred at 25° C. for 1 h. Water (10 mL) was added and the mixture was extracted with EtOAc (15 mL×3). The combined organic extracts were dried with anhydrous sodium sulfate, filtered, and concentrated in vacuo. The residue was purified by silica gel chromatography (0-25% EtOAc/petroleum ether) to afford the title compound.

### Step 7: benzyl 3-(2-(4-fluorophenyl)-6-(prop-1-en-2-yl)pyridin-4-yl)-3-methylpyrrolidine-1-carboxylate

A mixture of 4,4,5,5-tetramethyl-2-(prop-1-en-2-yl)-1,3,2-dioxaborolane (0.20 g, 1.2 mmol), benzyl 3-(2-chloro-6-(4-fluorophenyl)pyridin-4-yl)-3-methylpyrrolidine-1-carboxylate (0.50 g, 1.2 mmol), potassium carbonate (0.33 g, 2.4 mmol) and [1,1′-bis(diphenylphosphino)ferrocene]dichloropalladium(II) (43 mg, 0.059 mmol), and 1,4-dioxane (0.010 L) and water (1.0 mL) was heated at 100° C. for 12 h under an atmosphere of N₂. The mixture was cooled to room temperature, filtered, and the filtrate was concentrated in vacuo. The residue was purified by silica gel chromatography (0-25% EtOAc/petroleum ether) to afford the title compound.

### Step 8: benzyl 3-(2-(1-((tert-butoxycarbonyl)amino)-2-hydroxypropan-2-yl)-6-(4-fluorophenyl)pyridin-4-yl)-3-methylpyrrolidine-1-carboxylate

Potassium osmate(VI) dihydrate (51 mg, 0.14 mmol) was added to a mixture of benzyl 3-(2-(4-fluorophenyl)-6-(prop-1-en-2-yl)pyridin-4-yl)-3-methylpyrrolidine-1-carboxylate (0.30 g, 0.70 mmol), tert-butyl (tosyloxy)carbamate (0.35 mg, 1.2 mmol), tert-butanol (12 mL), and water (3.0 mL). The mixture was stirred at 25° C. for 12 h. The mixture was poured into a water/ice mixture (10 mL). The mixture was extracted with EtOAc (20 mL×2). The combined organic extracts were washed with brine (20 mL), dried with anhydrous sodium sulfate, filtered, and concentrated in vacuo. The residue was purified by silica gel chromatography (0-50% EtOAc/petroleum ether) to afford the title compound.

### Step 9: benzyl 3-(2-(1-amino-2-hydroxypropan-2-yl)-6-(4-fluorophenyl)pyridin-4-yl)-3-methylpyrrolidine-1-carboxylate

A mixture of benzyl 3-(2-(1-((tert-butoxycarbonyl)amino)-2-hydroxypropan-2-yl)-6-(4-fluorophenyl)pyridin-4-yl)-3-methylpyrrolidine-1-carboxylate (0.16 mg, 0.28 mmol), DCM (5.0 mL) and TFA (2.0 mL) was stirred at 25° C. for 0.5 h. The mixture was concentrated in vacuo to afford the title compound.

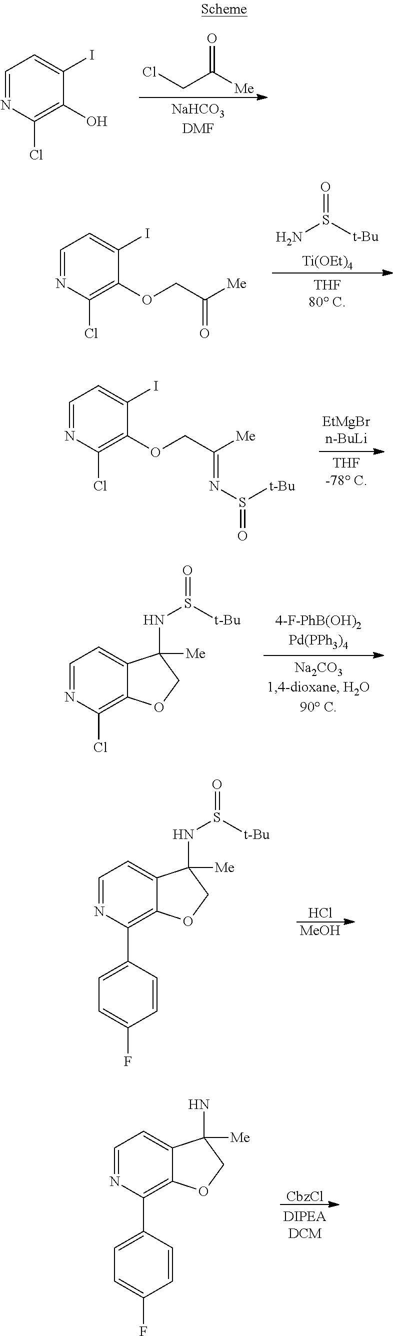

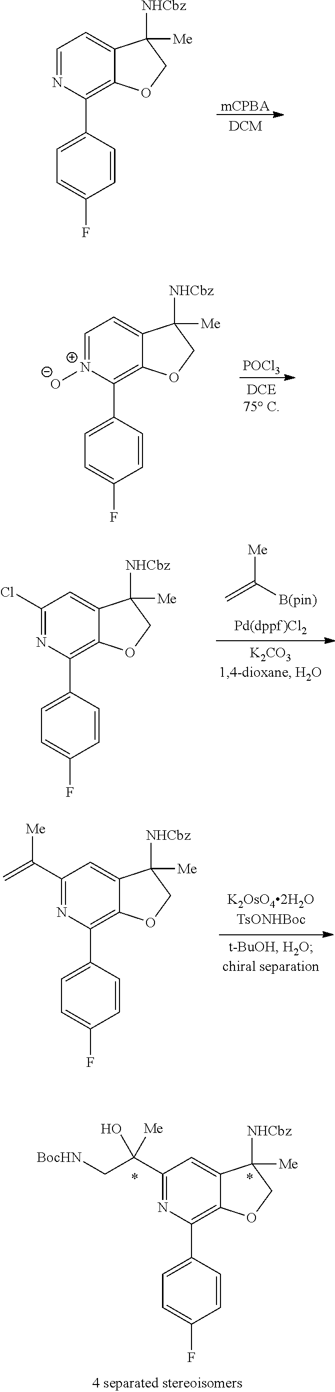

### Intermediate I-01-diastereomer-1, Intermediate I-01-diastereomer-2, Intermediate I-01-diastereomer-3, and Intermediate I-01-diastereomer-4

### tert-butyl (2-(3-(((benzyloxy)carbonyl)amino)-7-(4-fluorophenyl)-3-methyl-2,3-dihydrofuro[2,3-c]pyridin-5-yl)-2-hydroxypropyl)carbamate (diastereoisomer-1, diastereoisomer-2, diastereoisomer-3, and diastereoisomer-4)

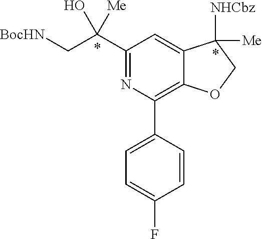 [SMILES: CC(C)(C)OC(=O)NCC(C)(O)c1cc2c(c(-c3ccc(F)cc3)n1)OCC2(C)NC(=O)OCc1ccccc1]

### Step 1: 1-((2-chloro-4-iodopyridin-3-yl)oxy)propan-2-one

A mixture of 2-chloro-4-iodopyridin-3-ol (7.5 g, 26 mmol), sodium bicarbonate (7.6 g, 0.090 mol), chloroacetone (5.2 mL, 64 mmol), and DMF (0.060 L) was stirred at 25° C. for 12 h. The mixture was filtered, and the filtrate was concentrated in vacuo. DCM (40 mL) was added, the mixture was filtered, and the filter cake was dried in vacuo to afford the title compound.

### Step 2: N-1-((2-chloro-4-iodopyridin-3-yl)oxy)propan-2-ylidene)-2-methylpropane-2-sulfinamide

A mixture of 1-((2-chloro-4-iodopyridin-3-yl)oxy)propan-2-one (11 g, 35 mmol), 2-methylpropane-2-sulfinamide (6.4 g, 53 mmol), tetraethoxytitanium (0.020 kg, 87 mmol), and THF (0.12 L) was stirred at 80° C. for 6 h. EtOAc (100 mL) and ice/water (100 mL) were added and the mixture was filtered. The filtrate was extracted with EtOAc (150 mL×3). The combined organic extracts were washed with brine (80 mL×2), dried with anhydrous sodium sulfate, filtered, and concentrated in vacuo. The residue was purified by silica gel chromatography (0-50% EtOAc/petroleum ether) to afford the title compound.

### Step 3: N-7-chloro-3-methyl-2,3-dihydrofuro[2,3-c]pyridin-3-yl)-2-methylpropane-2-sulfinamide

A solution of n-butyllithium (2.5 M in hexane, 8.7 mL, 22 mmol) was added to a 0° C. mixture of ethylmagnesium bromide (3.0 M in Et₂O/2-methyltetrahydrofuran, 3.7 mL, 11 mmol) and THF (0.040 L). The mixture was stirred for 10 min and then cooled to −78° C. A solution of N-1-((2-chloro-4-iodopyridin-3-yl)oxy)propan-2-ylidene)-2-methylpropane-2-sulfinamide (4.1 g, 9.9 mmol) in THF (0.010 L) was added dropwise and the mixture was stirred for 1 h. Water (30 mL) was added and the mixture was extracted with EtOAc (50 mL×3). The combined organic extracts were washed with brine (30 mL×2), dried with anhydrous sodium sulfate, filtered, and concentrated in vacuo. The residue was purified by silica gel chromatography (0-70% EtOAc/petroleum ether) to afford the title compound.

### Step 4: N-7-(4-fluorophenyl)-3-methyl-2,3-dihydrofuro[2,3-c]pyridin-3-yl)-2-methylpropane-2-sulfinamide

A mixture of N-7-chloro-3-methyl-2,3-dihydrofuro[2,3-c]pyridin-3-yl)-2-methylpropane-2-sulfinamide (1.5 g, 5.2 mmol), sodium carbonate (1.7 g, 16 mmol), (4-fluorophenyl)boronic acid (1.5 g, 0.010 mol), tetrakis(triphenylphosphine)palladium(0) (0.60 g, 0.52 mmol), 1,4-dioxane (0.020 L), and water (4.0 mL) was stirred at 90° C. for 12 h. The mixture was cooled to room temperature, water was added, and the mixture was extracted with EtOAc (50 mL×3). The combined organic extracts were washed with brine (80 mL×2), dried with anhydrous sodium sulfate, filtered, and concentrated in vacuo. The residue was purified by silica gel chromatography (0-70% EtOAc/petroleum ether) to afford the title compound.

### Step 5: 7-(4-fluorophenyl)-3-methyl-2,3-dihydrofuro[2,3-c]pyridin-3-amine

A mixture of N-7-(4-fluorophenyl)-3-methyl-2,3-dihydrofuro[2,3-c]pyridin-3-yl)-2-methylpropane-2-sulfinamide (1.9 g, 4.4 mmol), methanolic HCl (4.0 M, 5 mL), and methanol (0.020 L) was stirred at 20° C. for 1 h. The mixture was concentrated in vacuo to afford the title compound.

### Step 6: benzyl (7-(4-fluorophenyl)-3-methyl-2,3-dihydrofuro[2,3-c]pyridin-3-yl)carbamate

Benzyl chloroformate (0.87 mL, 6.1 mmol) was added to a 0° C. mixture of 7-(4-fluorophenyl)-3-methyl-2,3-dihydrofuro[2,3-c]pyridin-3-amine (2.0 g, 4.1 mmol), DIPEA (3.6 mL, 0.020 mol), and DCM (0.030 L). The ice bath was removed and the mixture was then stirred at 20° C. for 12 h. The mixture was concentrated in vacuo and the residue was purified by silica gel chromatography (0-30% EtOAc/petroleum ether) to afford the title compound.

### Step 7: 3-(((benzyloxy)carbonyl)amino)-7-(4-fluorophenyl)-3-methyl-2,3-dihydrofuro[2,3-c]pyridine 6-oxide

A mixture of benzyl (7-(4-fluorophenyl)-3-methyl-2,3-dihydrofuro[2,3-c]pyridin-3-yl)carbamate (1.4 g, 3.7 mmol), 3-chloroperbenzoic acid (1.9 g, 9.3 mmol), and DCM (25 mL) was stirred at 25° C. for 12 h. Sodium sulfite (aqueous saturated, 15 mL) and sodium carbonate (aqueous saturated, 15 mL) were added and the mixture was extracted with DCM (50 mL×3). The combined organic extracts were washed with brine (40 mL), dried with anhydrous sodium sulfate, filtered, and concentrated in vacuo. The residue was purified by silica gel chromatography (0-100% EtOAc/petroleum ether) to afford the title compound.

### Step 8: benzyl (5-chloro-7-(4-fluorophenyl)-3-methyl-2,3-dihydrofuro[2,3-c]pyridin-3-yl)carbamate

A mixture of 3-(((benzyloxy)carbonyl)amino)-7-(4-fluorophenyl)-3-methyl-2,3-dihydrofuro[2,3-c]pyridine 6-oxide (1.0 g, 2.5 mmol), phosphorus(V) oxychloride (3.6 mL, 38 mmol), and DCE (0.020 L) was stirred at 75° C. for 16 h. The mixture was cooled to room temperature, water (30 mL) was added, and the mixture was cooled to 0° C. Sodium bicarbonate (aqueous saturated) was added until the mixture was pH 8. The mixture was extracted with DCM (30 mL×3). The combined organic extracts were washed with brine (20 mL), dried with anhydrous sodium sulfate, filtered, and concentrated in vacuo. The residue was purified by silica gel chromatography (0-20% EtOAc/petroleum ether) to afford the title compound.

### Step 9: benzyl (7-(4-fluorophenyl)-3-methyl-5-(prop-1-en-2-yl)-2,3-dihydrofuro[2,3-c]pyridin-3-yl)carbamate

A mixture of benzyl (5-chloro-7-(4-fluorophenyl)-3-methyl-2,3-dihydrofuro[2,3-c]pyridin-3-yl)carbamate (0.45 g, 1.1 mmol), 4,4,5,5-tetramethyl-2-(prop-1-en-2-yl)-1,3,2-dioxaborolane (0.37 g, 2.2 mmol), [1,1′-bis(diphenylphosphino)ferrocene]dichloropalladium(II) (0.080 g, 0.11 mmol), potassium carbonate (0.45 g, 3.3 mmol), 1,4-dioxane (0.010 L), and water (1.0 mL) was stirred at 100° C. for 12 h. The mixture was cooled to room temperature, filtered, and concentrated in vacuo. The residue was purified by silica gel chromatography (0-20% EtOAc/petroleum ether) to afford the title compound.

### Step 10: tert-butyl (2-(3-(((benzyloxy)carbonyl)amino)-7-(4-fluorophenyl)-3-methyl-2,3-dihydrofuro[2,3-c]pyridin-5-yl)-2-hydroxypropyl)carbamate (diastereomer-1, diastereomer-2, diastereomer-3, and diastereomer-4)

Potassium osmate(VI) dihydrate (76 mg, 0.21 mmol) was added to a mixture of benzyl (7-(4-fluorophenyl)-3-methyl-5-(prop-1-en-2-yl)-2,3-dihydrofuro[2,3-c]pyridin-3-yl)carbamate (0.43 g, 1.0 mmol), tert-butyl (tosyloxy)carbamate (0.52 g, 1.8 mmol), tert-butanol (12 mL), and water (4.0 mL). The mixture was stirred at 25° C. for 12 h and then poured into an ice/water mixture (20 mL) and extracted with DCM (30 mL×2). The combined organic extracts were washed with brine (20 mL×3), dried with anhydrous sodium sulfate, filtered, and concentrated in vacuo. The residue was purified by silica gel chromatography (5-100% EtOAc/petroleum ether). The mixture of 4 stereoisomers was further purified by SFC (ChiralPak AD-3, 150×4.6 mm, 3 μm; 5-40% MeOH (with 0.05% diethylamine)/CO₂). The first eluting isomer was designated diastereomer-1, the second eluting isomer was designated diastereomer-2, the third eluting isomer was designated diastereomer-3, and the fourth eluting isomer was designated diastereomer-4.

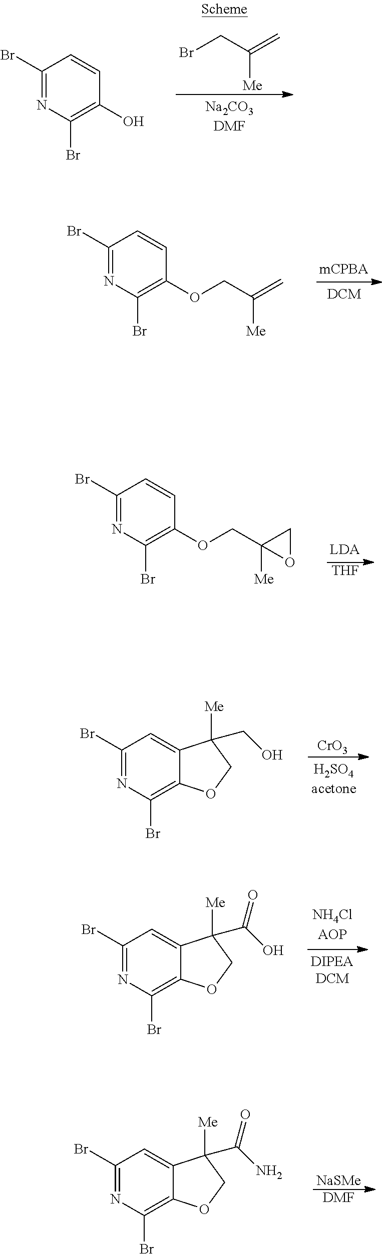

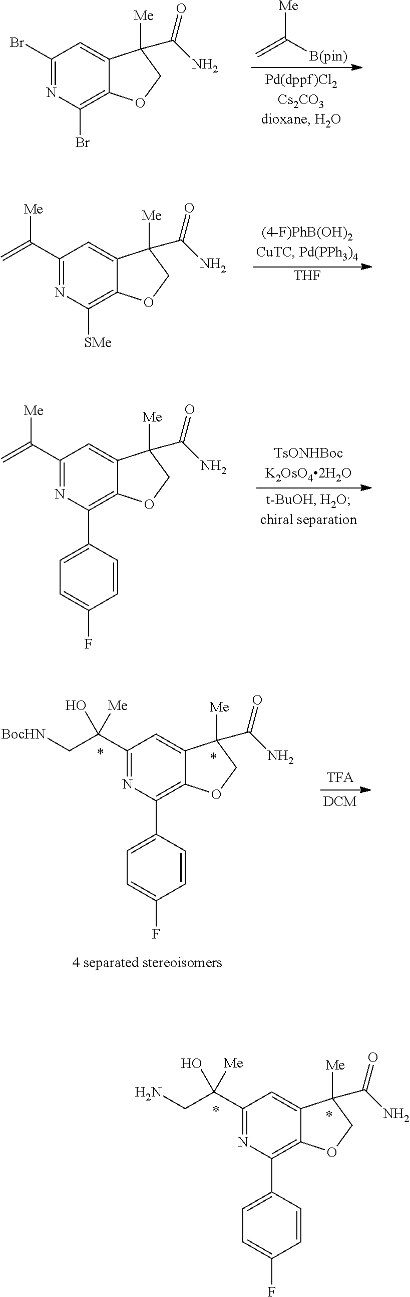

### Intermediate J-01-diastereomer-1, Intermediate J-01-diastereomer-2, Intermediate J-01-diastereomer-3, and Intermediate J-01-diastereomer-4

### 5-(1-amino-2-hydroxypropan-2-yl)-7-(4-fluorophenyl)-3-methyl-2,3-dihydrofuro[2,3-c]pyridine-3-carboxamide (diastereoisomer-1, diastereoisomer-2, diastereoisomer-3, and diastereoisomer-4)

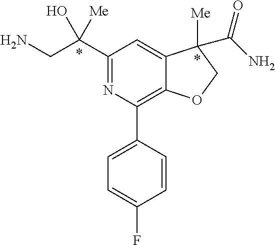 [SMILES: CC(O)(CN)c1cc2c(c(-c3ccc(F)cc3)n1)OCC2(C)C(N)=O]

### Step 1: 2,6-dibromo-3-((2-methylallyl)oxy)pyridine

A mixture of 2,6-dibromopyridin-3-ol (0.030 kg, 0.12 mol), sodium carbonate (25 g, 0.24 mol), DMF (0.50 L), and 3-bromo-2-methylprop-1-ene (32 g, 0.24 mol) was stirred at 25° C. for 12 h. A saturated aqueous solution of sodium chloride (1 L) was added and the mixture was extracted with EtOAc (600 mL×2). The combined organic extracts were dried with anhydrous sodium sulfate, filtered, and concentrated in vacuo. The residue was purified by silica gel chromatography (0-30% EtOAc/petroleum ether) to afford the title compound.

### Step 2: 2,6-dibromo-3-((2-methyloxiran-2-yl)methoxy)pyridine

A mixture of 2,6-dibromo-3-((2-methylallyl)oxy)pyridine (35 g, 0.10 mol), 3-chloroperbenzoic acid (85 wt %, 25 g, 0.12 mol), and DCM (0.60 L) was stirred at 25° C. for 12 h. A saturated aqueous solution of sodium sulfite (100 mL) was added and the mixture was stirred for 30 min. A saturated aqueous solution of sodium bicarbonate (240 mL) was added and the mixture was stirred for 30 min. The mixture was extracted with DCM (500 mL×2). The combined organic extracts were dried with anhydrous sodium sulfate, filtered, and concentrated in vacuo. The residue was purified by silica gel chromatography (0-30% EtOAc/petroleum ether) to afford the title compound.

### Step 3: (5,7-dibromo-3-methyl-2,3-dihydrofuro[2,3-c]pyridin-3-yl)methanol

A solution of 2,6-dibromo-3-((2-methyloxiran-2-yl)methoxy)pyridine (15 g, 46 mmol) in THF (0.050 L) was mixed with a THF solution of lithium diisopropyl amide (2.0 M, 0.030 L, 0.060 mol) in a 0° C. flow cell. The resulting mixture was warmed to 25° C. and water (150 mL) was added. The mixture was extracted with EtOAc (100 mL×3). The combined organic extracts were dried with anhydrous sodium sulfate, filtered, and concentrated in vacuo. The residue was purified by silica gel chromatography (0-30% EtOAc/petroleum ether) to afford the title compound.

### Step 4: 5,7-dibromo-3-methyl-2,3-dihydrofuro[2,3-c]pyridine-3-carboxylic acid

A mixture of (5,7-dibromo-3-methyl-2,3-dihydrofuro[2,3-c]pyridin-3-yl)methanol (0.010 kg, 31 mmol) and acetone (0.16 L) was stirred at 25° C. A solution of chromium trioxide in sulfuric acid (2.0 M, 23 mL, 46 mmol) was added and the mixture was stirred for 1 h. 2-Propanol (50 mL) and water (400 mL) were added and the mixture was extracted with EtOAc (200 mL×3). The pH of the aqueous phase was adjusted to ˜4 and the mixture was extracted with EtOAc (150 mL×3). The combined organic extracts were dried with anhydrous sodium sulfate, filtered, and concentrated in vacuo to afford the title compound.

### Step 5: 5,7-dibromo-3-methyl-2,3-dihydrofuro[2,3-c]pyridine-3-carboxamide

A mixture of 5,7-dibromo-3-methyl-2,3-dihydrofuro[2,3-c]pyridine-3-carboxylic acid (7.0 g, 21 mmol), ((3H-[1,2,3]triazolo[4,5-b]pyridin-3-yl)oxy)tris(dimethylamino)phosphonium hexafluorophosphate(V) (14 g, 31 mmol), DCM (0.12 L), and DIPEA (11 mL, 62 mmol) was stirred at 25° C. for 10 min. Ammonium chloride (2.2 g, 42 mmol) was added and the mixture was stirred at 25° C. for 1 h. Water (200 mL) was added and the mixture was extracted with DCM (125 mL×3). The combined organic extracts were dried with anhydrous sodium sulfate, filtered, and concentrated in vacuo. The residue was purified by silica gel chromatography (0-30% EtOAc/petroleum ether) to afford the title compound.

### Step 6: 5-bromo-3-methyl-7-(methylthio)-2,3-dihydrofuro[2,3-c]pyridine-3-carboxamide

A mixture of 5,7-dibromo-3-methyl-2,3-dihydrofuro[2,3-c]pyridine-3-carboxamide (7.1 g, 21 mmol), sodium methanethiolate (3.0 g, 43 mmol), and DMF (0.12 L) was stirred at 25° C. for 16 h. A saturated aqueous solution of ammonium chloride (500 mL) was added and the mixture was extracted with EtOAc (250 mL×3). The combined organic extracts were dried with anhydrous sodium sulfate, filtered, and concentrated in vacuo. The residue was purified by silica gel chromatography (0-30% EtOAc/petroleum ether) to afford the title compound.

### Step 7: 3-methyl-7-(methylthio)-5-(prop-1-en-2-yl)-2,3-dihydrofuro[2,3-c]pyridine-3-carboxamide

A mixture of 5-bromo-3-methyl-7-(methylthio)-2,3-dihydrofuro[2,3-c]pyridine-3-carboxamide (1.0 g, 3.3 mmol), 4,4,5,5-tetramethyl-2-(prop-1-en-2-yl)-1,3,2-dioxaborolane (0.67 g, 4.0 mmol), potassium carbonate (0.91 g, 6.6 mmol), [1,1′-bis(diphenylphosphino)ferrocene]dichloropalladium(II) (0.15 g, 0.20 mmol), 1,4-dioxane (15 mL), and water (3.0 mL) was placed under an atmosphere of N₂ and stirred in a 60° C. heating block for 12 h. The mixture was cooled to room temperature, filtered, diluted with water (25 mL), and extracted with EtOAc (15 mL×3). The combined organic extracts were washed with brine (20 mL), dried with anhydrous sodium sulfate, filtered, and concentrated in vacuo. The residue was purified by silica gel chromatography (0-30% EtOAc/petroleum ether) to afford the title compound.

### Step 8: 7-(4-fluorophenyl)-3-methyl-5-(prop-1-en-2-yl)-2,3-dihydrofuro[2,3-c]pyridine-3-carboxamide

A mixture of 3-methyl-7-(methylthio)-5-(prop-1-en-2-yl)-2,3-dihydrofuro[2,3-c]pyridine-3-carboxamide (1.0 g, 2.9 mmol), (4-fluorophenyl)boronic acid (0.65 g, 4.7 mmol), copper(I) thiophene-2-carboxylate (1.1 g, 5.8 mmol), tetrakis(triphenylphosphine)palladium(0) (0.17 g, 0.15 mmol), and THF (15 mL) was placed under an atmosphere of N₂ and stirred in a 60° C. heating block for 12 h. The mixture was cooled to room temperature, diluted with 8% ammonium hydroxide (50 mL), and extracted with EtOAc (50 mL×3). The combined organic extracts were dried with anhydrous sodium sulfate, filtered, and concentrated in vacuo. The residue was purified by preparative HPLC (25-45% MeCN/water (0.1% TFA)) to afford the title compound.

### Step 9: tert-butyl (2-(3-carbamoyl-7-(4-fluorophenyl)-3-methyl-2,3-dihydrofuro[2,3-c]pyridin-5-yl)-2-hydroxypropyl)carbamate (diastereoisomer-1, diastereoisomer-2, diastereoisomer-3, and diastereoisomer-4)

Tert-butyl (tosyloxy)carbamate (0.38 g, 1.3 mmol) and potassium osmate(VI) dihydrate (61 mg, 0.17 mmol) were added to a mixture of 7-(4-fluorophenyl)-3-methyl-5-(prop-1-en-2-yl)-2,3-dihydrofuro[2,3-c]pyridine-3-carboxamide (0.26 g, 0.83 mmol), tert-butanol (6.0 mL) and water (2.0 mL). The mixture was stirred in a 40° C. heating block for 4 h. The mixture was cooled to room temperature and filtered. An aqueous solution of sodium sulfite (2 M, 20 mL) was added and the mixture was extracted with EtOAc (15 mL×3). The combined organic extracts were dried with anhydrous sodium sulfate, filtered, and concentrated in vacuo. The residue was purified by preparative HPLC (C18; 50-70% MeCN/water (0.1% TFA)) to afford racemic diastereomer pairs. The first racemic mixture eluted from HPLC was purified by SFC (Chiralcel® OD-3, 100×4.6 mm, 3 μm; 5-40% EtOH (with 0.05% diethylamine)/CO₂) to afford the title compounds. The first eluting isomer was designated diastereomer-1 and the second eluting isomer was designated diastereomer-2. The second racemic mixture eluted from HPLC was purified by SFC ((S,S)Whelk-01, 100×4.6 mm, 5 μm; 5-40% EtOH (with 0.05% diethylamine)/CO₂) to afford the title compounds. The first eluting isomer was designated diastereomer-3 and the second eluting isomer was designated diastereomer-4.

### Step 10-A: 5-(1-amino-2-hydroxypropan-2-yl)-7-(4-fluorophenyl)-3-methyl-2,3-dihydrofuro[2,3-c]pyridine-3-carboxamide (diastereoisomer-1)

A mixture of tert-butyl (2-(3-carbamoyl-7-(4-fluorophenyl)-3-methyl-2,3-dihydrofuro[2,3-c]pyridin-5-yl)-2-hydroxypropyl)carbamate (diastereomer-1) (47 mg, 0.11 mmol), DCM (1.0 mL), and TFA (0.30 mL) was stirred at 25° C. for 30 min. The mixture was concentrated to afford the title compound.

### Step 10-B: 5-(1-amino-2-hydroxypropan-2-yl)-7-(4-fluorophenyl)-3-methyl-2,3-dihydrofuro[2,3-c]pyridine-3-carboxamide (diastereoisomer-2)

A mixture of tert-butyl (2-(3-carbamoyl-7-(4-fluorophenyl)-3-methyl-2,3-dihydrofuro[2,3-c]pyridin-5-yl)-2-hydroxypropyl)carbamate (diastereomer-2) (49 mg, 0.11 mmol), DCM (1.0 mL), and TFA (0.30 mL) was stirred at 25° C. for 30 min. The mixture was concentrated to afford the title compound.

### Step 10-C: 5-(1-amino-2-hydroxypropan-2-yl)-7-(4-fluorophenyl)-3-methyl-2,3-dihydrofuro[2,3-c]pyridine-3-carboxamide (diastereoisomer-3)

A mixture of tert-butyl (2-(3-carbamoyl-7-(4-fluorophenyl)-3-methyl-2,3-dihydrofuro[2,3-c]pyridin-5-yl)-2-hydroxypropyl)carbamate (diastereomer-3) (46 mg, 0.10 mmol), DCM (1.0 mL), and TFA (0.30 mL) was stirred at 25° C. for 30 min. The mixture was concentrated to afford the title compound.

### Step 10-D: 5-(1-amino-2-hydroxypropan-2-yl)-7-(4-fluorophenyl)-3-methyl-2,3-dihydrofuro[2,3-c]pyridine-3-carboxamide (diastereoisomer-4)

A mixture of tert-butyl (2-(3-carbamoyl-7-(4-fluorophenyl)-3-methyl-2,3-dihydrofuro[2,3-c]pyridin-5-yl)-2-hydroxypropyl)carbamate (diastereomer-4) (47 mg, 0.11 mmol), DCM (1.0 mL), and TFA (0.30 mL) was stirred at 25° C. for 30 min. The mixture was concentrated to afford the title compound.

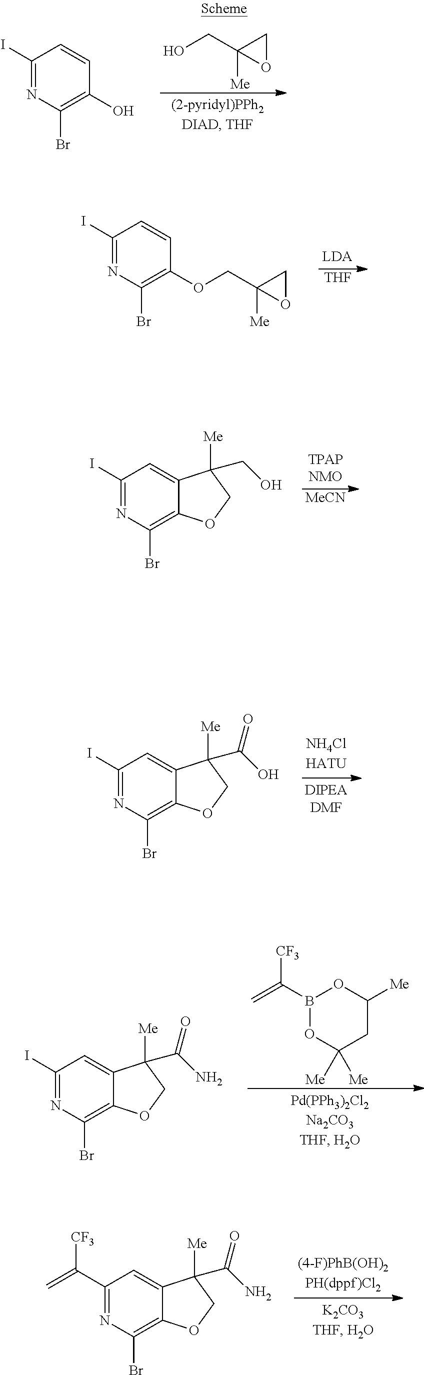

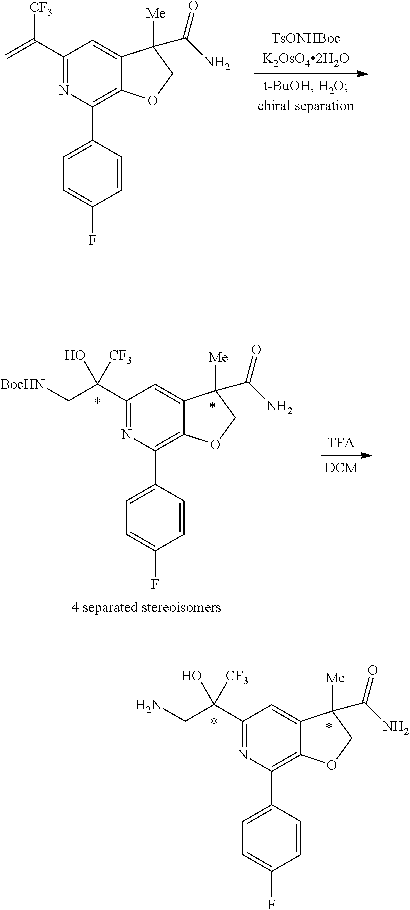 [SMILES: C=C(c1cc2c(c(-c3ccc(F)cc3)n1)OCC2(C)C(N)=O)C(F)(F)F ; CC(C)(C)OC(=O)NCC(O)(c1cc2c(c(-c3ccc(F)cc3)n1)OCC2(C)C(N)=O)C(F)(F)F ; CC1(C(N)=O)COc2c1cc(C(O)(CN)C(F)(F)F)nc2-c1ccc(F)cc1]

### Intermediate K-01-diastereomer-2

### 5-(3-amino-1,1,1-trifluoro-2-hydroxypropan-2-yl)-7-(4-fluorophenyl)-3-methyl-2,3-dihydrofuro[2,3-c]pyridine-3-carboxamide (diastereoisomer-2)

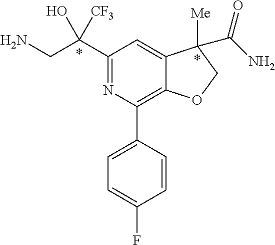 [SMILES: CC1(C(N)=O)COc2c1cc(C(O)(CN)C(F)(F)F)nc2-c1ccc(F)cc1]

### Step 1: 2-bromo-6-iodo-3-((2-methyloxiran-2-yl)methoxy)pyridine

A mixture of 2-bromo-6-iodopyridin-3-ol (2.5 g, 8.3 mmol), diphenyl-2-pyridylphosphine (4.4 g, 17 mmol), and THF (0.030 L) was cooled in a 0° C. ice bath. (2-Methyloxiran-2-yl)methanol (1.5 g, 17 mmol) and diisopropyl azodicarboxylate (3.3 mL, 17 mmol) were added and the mixture was warmed to 25° C. and stirred for 12 h. The mixture was concentrated and purified by silica gel chromatography (0-20% EtOAc/petroleum ether) to afford the title compound.

### Step 2: (7-bromo-5-iodo-3-methyl-2,3-dihydrofuro[2,3-c]pyridin-3-yl)methanol

A mixture of 2-bromo-6-iodo-3-((2-methyloxiran-2-yl)methoxy)pyridine (1.9 g, 5.1 mmol) and THF (0.020 L) was cooled in a 0° C. ice bath. A solution of lithium diisopropylamide in THF (2.0 M, 4.1 mL, 8.2 mmol) was added and the mixture was warmed to 25° C. and stirred for 12 h. A saturated aqueous solution of ammonium chloride (10 mL) was added and the mixture was extracted with EtOAc (20 mL×3). The combined organic extracts were washed with brine (50 mL), dried with anhydrous sodium sulfate, filtered, and concentrated in vacuo. The residue was purified by silica gel chromatography (0-30% EtOAc/petroleum ether) to afford the title compound.

### Step 3: 7-bromo-5-iodo-3-methyl-2,3-dihydrofuro[2,3-c]pyridine-3-carboxylic acid

A mixture of (7-bromo-5-iodo-3-methyl-2,3-dihydrofuro[2,3-c]pyridin-3-yl)methanol (0.57 g, 1.5 mmol), 4-methylmorpholine N-oxide (2.1 g, 15 mmol), acetonitrile (0.010 L), and tetrapropylammonium perruthenate (54 mg, 0.15 mmol) was stirred at 25° C. for 12 h. 2-Propanol (8 mL) and water (10 mL) were added and the mixture was extracted with EtOAc (25 mL×6). The combined organic extracts were dried with anhydrous sodium sulfate, filtered, and concentrated in vacuo to afford the title compound.

### Step 4: 7-bromo-5-iodo-3-methyl-2,3-dihydrofuro[2,3-c]pyridine-3-carboxamide

A mixture of 7-bromo-5-iodo-3-methyl-2,3-dihydrofuro[2,3-c]pyridine-3-carboxylic acid (0.71 g, 1.3 mmol), HATU (0.99 g, 2.6 mmol), DIPEA (0.70 mL, 3.9 mmol), and DMF (0.010 L) was stirred at 25° C. for 10 min. Ammonium chloride (0.14 g, 2.6 mmol) was added and the mixture was stirred for 1 h at 25° C. Water (10 mL) was added and the mixture was extracted with EtOAc (30 mL×3). The combined organic extracts were washed with brine (30 mL), dried with anhydrous sodium sulfate, filtered, and concentrated in vacuo. The residue was purified by silica gel chromatography (0-100% EtOAc/petroleum ether) to afford the title compound.

### Step 5: 7-bromo-3-methyl-5-(3,3,3-trifluoroprop-1-en-2-yl)-2,3-dihydrofuro[2,3-c]pyridine-3-carboxamide

A mixture of 7-bromo-5-iodo-3-methyl-2,3-dihydrofuro[2,3-c]pyridine-3-carboxamide (0.46 g, 1.2 mmol), 4,4,6-trimethyl-2-[1-(trifluoromethyl)ethenyl]-1,3,2-dioxaborinane (0.54 g, 2.4 mmol), potassium carbonate (0.50 g, 3.6 mmol), [1,1′-bis(diphenylphosphino)ferrocene]dichloropalladium(II) (0.18 g, 0.24 mmol), 1,4-dioxane (0.010 L), and water (1.0 mL) was placed under an atmosphere of N₂ and stirred in a 90° C. heating block for 1 h. The mixture was cooled to room temperature, diluted with water (10 mL), and extracted with EtOAc (15 mL×3). The combined organic extracts were washed with brine (15 mL×2), dried with anhydrous sodium sulfate, filtered, and concentrated in vacuo. The residue was purified by silica gel chromatography (0-30% EtOAc/petroleum ether) to afford the title compound.

### Step 6: 7-(4-fluorophenyl)-3-methyl-5-(3,3,3-trifluoroprop-1-en-2-yl)-2,3-dihydrofuro[2,3-c]pyridine-3-carboxamide

A mixture of 7-bromo-3-methyl-5-(3,3,3-trifluoroprop-1-en-2-yl)-2,3-dihydrofuro[2,3-c]pyridine-3-carboxamide (0.36 g, 0.95 mmol), 4-fluorophenylboronic acid (0.20 g, 1.4 mmol) sodium carbonate (0.25 g, 2.4 mmol), bis(triphenylphosphine)palladium(II) dichloride (67 mg, 0.095 mmol), THF (8.0 mL), and water (2.0 mL) was placed under an atmosphere of N₂ and stirred in a 70° C. heating block for 1 h. The mixture was cooled to room temperature, diluted with water (10 mL), and extracted with EtOAc (15 mL×3). The combined organic extracts were washed with brine (15 mL×2), dried with anhydrous sodium sulfate, filtered, and concentrated in vacuo. The residue was purified by silica gel chromatography (0-30% EtOAc/petroleum ether) to afford the title compound.

### Step 7: tert-butyl (2-(3-carbamoyl-7-(4-fluorophenyl)-3-methyl-2,3-dihydrofuro[2,3-c]pyridin-5-yl)-3,3,3-trifluoro-2-hydroxypropyl)carbamate (diastereomer-2)

Tert-butyl (tosyloxy)carbamate (0.50 g, 1.7 mmol) and potassium osmate(VI) dihydrate (73 mg, 0.20 mmol) were added to a mixture of 7-(4-fluorophenyl)-3-methyl-5-(3,3,3-trifluoroprop-1-en-2-yl)-2,3-dihydrofuro[2,3-c]pyridine-3-carboxamide (0.36 g, 0.99 mmol), tert-butanol (15 mL) and water (5.0 mL). The mixture was stirred at 25° C. for 12 h. The mixture was poured into a ice/water mix (20 mL) and extracted with DCM (30 mL×2). The combined organic extracts were washed with brine (20 mL×3), dried with anhydrous sodium sulfate, filtered, and concentrated in vacuo. The residue was purified by silica gel chromatography (0-50% EtOAc/petroleum ether). The first eluting material was further purified by SFC (CHIRALPAK AD-3, 150×4.6 mm, 3 μm; 5-40% 2-PrOH (with 0.05% NH₄OH)/CO₂) and the second eluting isomer was designated diastereomer-2 of the title compound.

### Step 8: 5-(3-amino-1,1,1-trifluoro-2-hydroxypropan-2-yl)-7-(4-fluorophenyl)-3-methyl-2,3-dihydrofuro[2,3-c]pyridine-3-carboxamide (diastereomer-2)

A mixture of tert-butyl (2-(3-carbamoyl-7-(4-fluorophenyl)-3-methyl-2,3-dihydrofuro[2,3-c]pyridin-5-yl)-3,3,3-trifluoro-2-hydroxypropyl)carbamate (diastereomer-2) (67 mg, 0.13 mmol), DCM (4.0 mL), and TFA (1.0 mL) was stirred at 25° C. for 1 h. The mixture was concentrated to afford the title compound.

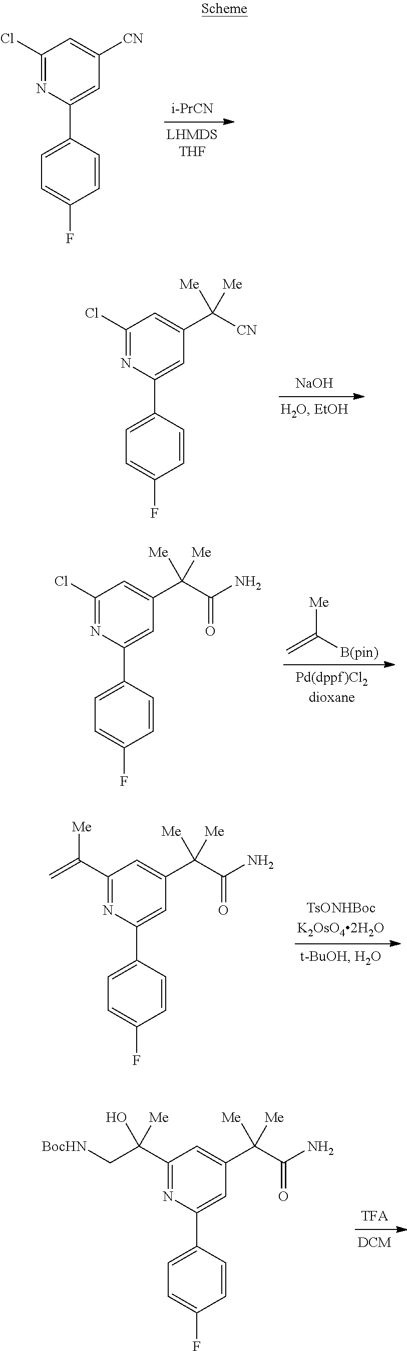

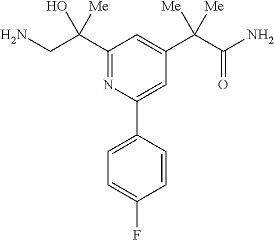 [SMILES: CC(O)(CN)c1cc(C(C)(C)C(N)=O)cc(-c2ccc(F)cc2)n1]

### Intermediate L-01

### 2-(2-(1-amino-2-hydroxypropan-2-yl)-6-(4-fluorophenyl)pyridin-4-yl)-2-methylpropanamide

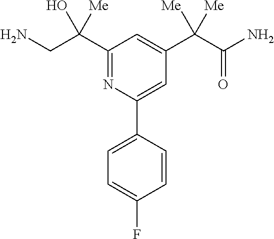 [SMILES: CC(O)(CN)c1cc(C(C)(C)C(N)=O)cc(-c2ccc(F)cc2)n1]

### Step 1: 2-(2-chloro-6-(4-fluorophenyl)pyridin-4-yl)-2-methylpropanenitrile

A mixture of 2-chloro-6-(4-fluorophenyl)isonicotinonitrile (1.0 g, 4.3 mmol), isobutyronitrile (0.39 g, 5.6 mmol), and THF (0.020 L) was cooled in a −78° C. dry ice/acetone bath. A THF solution of lithium diisopropylamide (1.0 M, 4.3 mL, 4.3 mmol) was added and the mixture was warmed to 25° C. and stirred for 2 h. Water (20 mL) was added and the mixture was extracted with EtOAc (15 mL×3). The combined organic extracts were washed with brine (20 mL), dried with anhydrous sodium sulfate, filtered, and concentrated in vacuo. The residue was purified by silica gel chromatography (0-15% EtOAc/petroleum ether) to afford the title compound.

### Step 2: 2-(2-chloro-6-(4-fluorophenyl)pyridin-4-yl)-2-methylpropanamide

A mixture of 2-(2-chloro-6-(4-fluorophenyl)pyridin-4-yl)-2-methylpropanenitrile (0.60 g, 2.2 mmol), ethanol (0.010 L), and an aqueous solution of NaOH (2.0 M, 3.3 mL, 6.6 mmol) was stirred in an 80° C. heating block for 4 h. The mixture was cooled to room temperature, water (20 mL) was added, and the mixture was extracted with EtOAc (15 mL×3). The combined organic extracts were washed with brine (20 mL), dried with anhydrous sodium sulfate, filtered, and concentrated in vacuo. The residue was purified by silica gel chromatography (0-20% EtOAc/petroleum ether) to afford the title compound.

### Step 3: 2-(2-(4-fluorophenyl)-6-(prop-1-en-2-yl)pyridin-4-yl)-2-methylpropanamide

A mixture of 2-(2-chloro-6-(4-fluorophenyl)pyridin-4-yl)-2-methylpropanamide (0.25 g, 0.85 mmol), 4,4,5,5-tetramethyl-2-(prop-1-en-2-yl)-1,3,2-dioxaborolane (0.14 g, 0.85 mmol), potassium carbonate (0.24 g, 1.7 mmol), [1,1′-bis(diphenylphosphino)ferrocene]dichloropalladium(II) (31 mg, 0.043 mmol), 1,4-dioxane (0.010 L), and water (1.0 mL) was placed under a N₂ atmosphere and stirred in a 100° C. heating block for 12 h. The mixture was cooled to room temperature, filtered, concentrated, and purified by silica gel chromatography (0-25% EtOAc/petroleum ether) to afford the title compound.

### Step 4: tert-butyl (2-(4-(1-amino-2-methyl-1-oxopropan-2-yl)-6-(4-fluorophenyl)pyridin-2-yl)-2-hydroxypropyl)carbamate

Tert-butyl (tosyloxy)carbamate (0.22 g, 0.76 mmol) and potassium osmate(VI) dihydrate (32 mg, 0.087 mmol) were added to a mixture of 2-(2-(4-fluorophenyl)-6-(prop-1-en-2-yl)pyridin-4-yl)-2-methylpropanamide (0.13 g, 0.44 mmol), tert-butanol (6.0 mL) and water (2.0 mL). The mixture was stirred at 25° C. for 12 h. The mixture was poured into an ice/water mix (10 mL) and extracted with DCM (20 mL×2). The combined organic extracts were washed with brine (20 mL), dried with anhydrous sodium sulfate, filtered, and concentrated in vacuo. The residue was purified by silica gel chromatography (0-50% EtOAc/petroleum ether) to afford the title compound.

### Step 5: 2-(2-(1-amino-2-hydroxypropan-2-yl)-6-(4-fluorophenyl)pyridin-4-yl)-2-methylpropanamide

A mixture of tert-butyl (2-(4-(1-amino-2-methyl-1-oxopropan-2-yl)-6-(4-fluorophenyl)pyridin-2-yl)-2-hydroxypropyl)carbamate (0.10 g, 0.23 mmol), DCM (5.0 mL), and TFA (2.0 mL) was stirred at 25° C. for 30 min. The mixture was concentrated to afford the title compound.

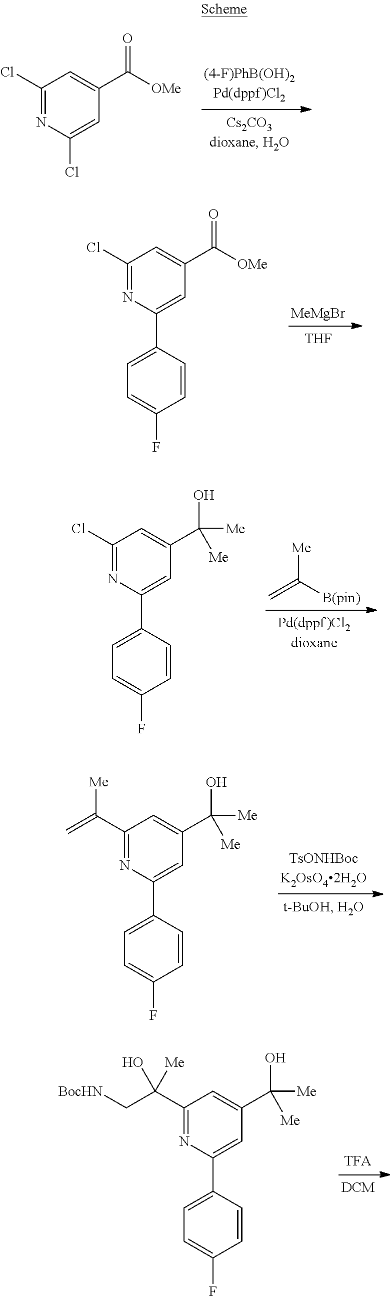

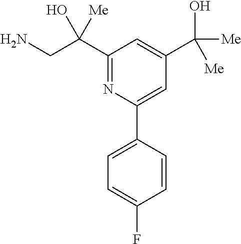 [SMILES: CC(C)(O)c1cc(-c2ccc(F)cc2)nc(C(C)(O)CN)c1]

### Intermediate M-01

### 1-amino-2-(6-(4-fluorophenyl)-4-(2-hydroxypropan-2-yl)pyridin-2-yl)propan-2-ol

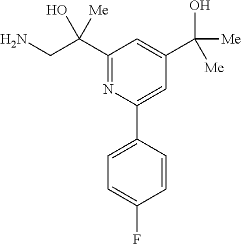 [SMILES: CC(C)(O)c1cc(-c2ccc(F)cc2)nc(C(C)(O)CN)c1]

### Step 1: methyl 2-chloro-6-(4-fluorophenyl)isonicotinate

A mixture of methyl 2,6-dichloroisonicotinate (0.010 kg, 49 mmol), 4-fluorophenylboronic acid (4.1 g, 29 mmol), cesium carbonate (47 g, 0.15 mol), bis(triphenylphosphine)palladium(II) dichloride (1.8 g, 2.4 mmol), 1,4-dioxane (0.20 L), and water (0.010 L) was stirred at 25° C. for 12 h. The mixture was filtered and the filtrate was extracted with EtOAc (100 mL×3). The combined organic extracts were washed with brine (50 mL×2), dried with anhydrous sodium sulfate, filtered, and concentrated in vacuo. The residue was purified by silica gel chromatography (0-10% EtOAc/petroleum ether) to afford the title compound.

### Step 2: 2-(2-chloro-6-(4-fluorophenyl)pyridin-4-yl)propan-2-ol

A mixture of methyl 2-chloro-6-(4-fluorophenyl)isonicotinate (0.010 kg, 38 mmol) and THF (0.20 L) was cooled in a 0° C. ice bath. A THF solution of methylmagnesium bromide (3.0M, 63 mL, 0.19 mol) was added, the ice bath was removed, and the mixture was stirred at 25° C. for 2 h. The mixture was cooled in a 0° C. ice bath and a saturated aqueous solution of ammonium chloride (10 mL) was added. The mixture was extracted with EtOAc (20 mL×3) and the combined organic extracts were washed with brine (10 mL), dried with anhydrous sodium sulfate, filtered, and concentrated in vacuo. The residue was purified by silica gel chromatography (0-10% EtOAc/petroleum ether) to afford the title compound.

### Step 3: 2-(2-(4-fluorophenyl)-6-(prop-1-en-2-yl)pyridin-4-yl)propan-2-ol

A mixture of 2-(2-chloro-6-(4-fluorophenyl)pyridin-4-yl)propan-2-ol (2.4 g, 8.9 mmol), 4,4,5,5-tetramethyl-2-(prop-1-en-2-yl)-1,3,2-dioxaborolane (1.5 g, 8.9 mmol), potassium carbonate (2.6 g, 19 mmol), [1,1′-bis(diphenylphosphino)ferrocene]dichloropalladium(II) (0.52 g, 0.71 mmol), 1,4-dioxane (0.050 L), and water (5.0 mL) was placed under a N₂ atmosphere and stirred in a 110° C. heating block for 1 h. The mixture was cooled to room temperature, filtered, concentrated, and purified by silica gel chromatography (0-100% EtOAc/petroleum ether) to afford the title compound.

### Step 4: tert-butyl (2-(6-(4-fluorophenyl)-4-(2-hydroxypropan-2-yl)pyridin-2-yl)-2-hydroxypropyl)carbamate

Tert-butyl (tosyloxy)carbamate (2.1 g, 7.2 mmol) and potassium osmate(VI) dihydrate (0.35 g, 0.96 mmol) were added to a mixture of 2-(2-(4-fluorophenyl)-6-(prop-1-en-2-yl)pyridin-4-yl)propan-2-ol (1.3 g, 4.8 mmol), tert-butanol (0.020 L), acetonitrile (0.010 L), and water (0.010 L). The mixture was stirred at 25° C. for 12 h. The mixture was filtered and purified by silica gel chromatography (0-100% EtOAc/petroleum ether) to afford the title compound.

### Step 5: 1-amino-2-(6-(4-fluorophenyl)-4-(2-hydroxypropan-2-yl)pyridin-2-yl)propan-2-ol

A mixture of tert-butyl (2-(6-(4-fluorophenyl)-4-(2-hydroxypropan-2-yl)pyridin-2-yl)-2-hydroxypropyl)carbamate (1.0 g, 2.5 mmol), DCM (0.010 L), and TFA (3.0 mL) was stirred at 25° C. for 1 h. The mixture was concentrated to afford the title compound.

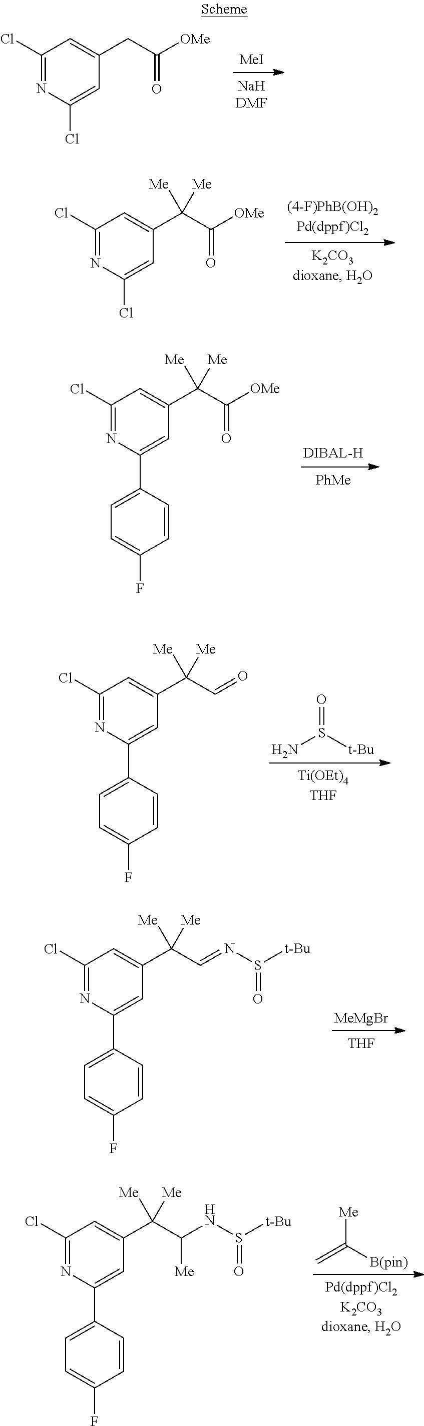

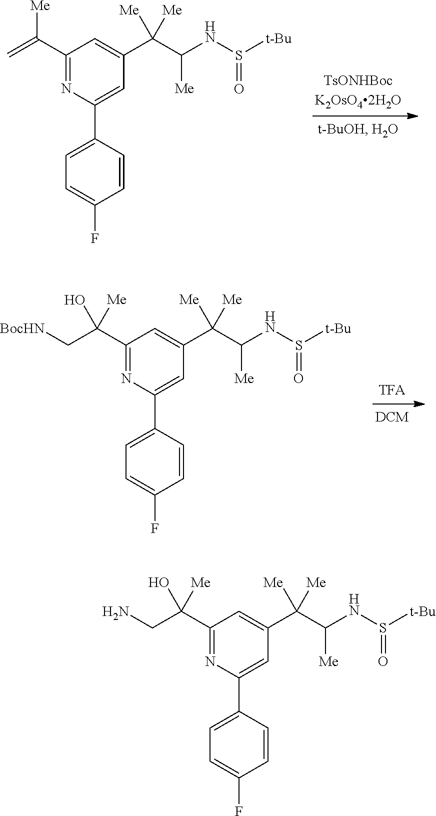 [SMILES: C=C(C)c1cc(C(C)(C)C(C)NS(=O)C(C)(C)C)cc(-c2ccc(F)cc2)n1 ; CC(NS(=O)C(C)(C)C)C(C)(C)c1cc(-c2ccc(F)cc2)nc(C(C)(O)CNC(=O)OC(C)(C)C)c1 ; CC(NS(=O)C(C)(C)C)C(C)(C)c1cc(-c2ccc(F)cc2)nc(C(C)(O)CN)c1]

### Intermediate N-01

### N-(3-(2-(1-amino-2-hydroxypropan-2-yl)-6-(4-fluorophenyl)pyridin-4-yl)-3-methylbutan-2-yl)-2-methylpropane-2-sulfinamide

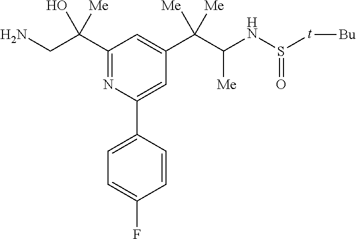

### Step 1: methyl 2-(2,6-dichloropyridin-4-yl)-2-methylpropanoate

A mixture of methyl 2-(2,6-dichloropyridin-4-yl)acetate (5.0 g, 23 mmol), DMF (0.070 L), sodium hydride (60 wt % dispersion in mineral oil, 5.5 g, 0.14 mol), and iodomethane (16 g, 0.11 mol) was stirred at 25° C. for 2 h. Hydrochloric acid (1 M, 100 mL) was added and the mixture was extracted with EtOAc (125 mL×3). The combined organic extracts were washed with brine (500 mL×2), dried with anhydrous sodium sulfate, filtered, and concentrated in vacuo. The residue was purified by silica gel chromatography (0-30% EtOAc/petroleum ether) to afford the title compound.

### Step 2: methyl 2-(2-chloro-6-(4-fluorophenyl)pyridin-4-yl)-2-methylpropanoate

A mixture of methyl 2-(2,6-dichloropyridin-4-yl)-2-methylpropanoate (7.2 g, 29 mmol), 4-fluorophenylboronic acid (4.1 g, 29 mmol), potassium carbonate (8.0 g, 58 mmol), bis(triphenylphosphine)palladium(II) dichloride (1.1 g, 1.5 mmol), 1,4-dioxane (0.12 L), and water (12 mL) was stirred at 25° C. for 12 h. The mixture was filtered, diluted with water (200 mL), and extracted with EtOAc (200 mL×3). The combined organic extracts were washed with brine (400 mL), dried with anhydrous sodium sulfate, filtered, and concentrated in vacuo. The residue was purified by reverse phase chromatography (C18, 20-35 M, 0-95% MeCN/water (0.5% TFA)) to afford the title compound.

### Step 3: 2-(2-chloro-6-(4-fluorophenyl)pyridin-4-yl)-2-methylpropanal

A mixture of methyl 2-(2-chloro-6-(4-fluorophenyl)pyridin-4-yl)-2-methylpropanoate (3.1 g, 0.010 mol) and DCM (0.040 L) was cooled in a −78° C. dry ice/acetone bath. A solution of diisobutyl hydride in toluene (1.0 M, 2.5 mL, 2.5 mmol) was added and the mixture was stirred for 2 h. A saturated aqueous solution of ammonium chloride (50 mL) was added and the mixture was extracted with DCM (100 mL×3). The combined organic extracts were washed with brine (200 mL), dried with anhydrous sodium sulfate, filtered, and concentrated in vacuo. The residue was purified by preparative thin-layer chromatography (25% EtOAc/petroleum ether) to afford the title compound.

### Step 4: N-(2-(2-chloro-6-(4-fluorophenyl)pyridin-4-yl)-2-methylpropylidene)-2-methylpropane-2-sulfinamide

A mixture of 2-(2-chloro-6-(4-fluorophenyl)pyridin-4-yl)-2-methylpropanal (0.10 g, 0.36 mmol), 2-methyl-2-propanesulfinamide (79 mg, 0.65 mmol), THF (4.0 mL), and titanium(IV) ethoxide (0.25 g, 1.1 mmol) was stirred in a 60° C. heating block for 4 h. The mixture was cooled to room temperature and concentrated. Water (8 mL) was added and the mixture was extracted with EtOAc (3 mL×3). The combined organic extracts were washed with brine (5 mL×2), dried with anhydrous sodium sulfate, filtered, and concentrated in vacuo. The residue was purified by preparative thin-layer chromatography (25% EtOAc/petroleum ether) to afford the title compound.

### Step 5: N-(3-(2-chloro-6-(4-fluorophenyl)pyridin-4-yl)-3-methylbutan-2-yl)-2-methylpropane-2-sulfinamide

A mixture of N-(2-(2-chloro-6-(4-fluorophenyl)pyridin-4-yl)-2-methylpropylidene)-2-methylpropane-2-sulfinamide (0.12 g, 0.32 mmol) and THF (2.0 mL) was cooled in a 0° C. ice/water bath. A THF solution of methylmagnesium bromide (3.0 M, 0.30 mL, 0.90 mmol) was added and the mixture was stirred for 2 h. A saturated aqueous solution of ammonium chloride (6 mL) was added and the mixture was extracted with EtOAc (3 mL×3). The combined organic extracts were washed with brine (5 mL×2), dried with anhydrous sodium sulfate, filtered, and concentrated in vacuo. The residue was purified by preparative thin-layer chromatography (50% EtOAc/petroleum ether) to afford the title compound.

### Step 6: N-(3-(2-(4-fluorophenyl)-6-(prop-1-en-2-yl)pyridin-4-yl)-3-methylbutan-2-yl)-2-methylpropane-2-sulfinamide

A mixture of N-(3-(2-chloro-6-(4-fluorophenyl)pyridin-4-yl)-3-methylbutan-2-yl)-2-methylpropane-2-sulfinamide (0.060 g, 0.15 mmol), 4,4,5,5-tetramethyl-2-(prop-1-en-2-yl)-1,3,2-dioxaborolane (38 mg, 0.23 mmol), potassium carbonate (63 mg, 0.45 mmol), [1,1′-bis(diphenylphosphino)ferrocene]dichloropalladium(II) (22 mg, 0.030 mmol), 1,4-dioxane (1.0 mL), and water (0.20 mL) was placed under a N₂ atmosphere and stirred in an 80° C. heating block for 2 h. The mixture was cooled to room temperature, filtered, and concentrated. The mixture was diluted with water (3 mL) and extracted with EtOAc (1.5 mL×3). The combined organic extracts were washed with brine (2 mL), dried with anhydrous sodium sulfate, filtered, and concentrated in vacuo. The residue was purified by preparative thin-layer chromatography (50% EtOAc/petroleum ether) to afford the title compound.

### Step 7: tert-butyl (2-(4-(3-((tert-butylsulfinyl)amino)-2-methylbutan-2-yl)-6-(4-fluorophenyl)pyridin-2-yl)-2-hydroxypropyl)carbamate

Tert-butyl (tosyloxy)carbamate (0.39 g, 1.3 mmol) and potassium osmate(VI) dihydrate (0.050 g, 0.13 mmol) were added to a mixture of N-(3-(2-(4-fluorophenyl)-6-(prop-1-en-2-yl)pyridin-4-yl)-3-methylbutan-2-yl)-2-methylpropane-2-sulfinamide (0.27 g, 0.67 mmol), tert-butanol (6.0 mL), and water (2.0 mL). The mixture was stirred at 25° C. for 12 h. The mixture was filtered and water (10 mL) was added to the filtrate. The mixture was extracted with EtOAc (10 mL×3) and the combined organic extracts were washed with brine (20 mL×2), dried with anhydrous sodium sulfate, filtered, and concentrated in vacuo. The residue was purified by preparative HPLC (C18; 46-66% MeCN/water (0.1% TFA)) to afford the title compound.

### Step 8: N-(3-(2-(1-amino-2-hydroxypropan-2-yl)-6-(4-fluorophenyl)pyridin-4-yl)-3-methylbutan-2-yl)-2-methylpropane-2-sulfinamide

A mixture of tert-butyl (2-(4-(3-((tert-butylsulfinyl)amino)-2-methylbutan-2-yl)-6-(4-fluorophenyl)pyridin-2-yl)-2-hydroxypropyl)carbamate (0.18 g, 0.34 mmol), DCM (3.0 mL), and TFA (0.30 mL) was stirred at 25° C. for 2 h. The mixture was concentrated to afford the title compound.

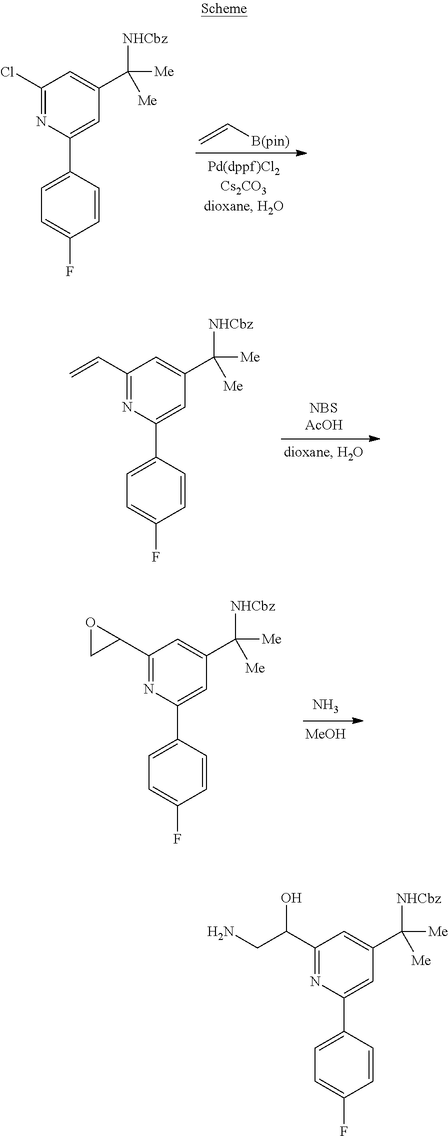

### Intermediate 0-01

### benzyl (2-(2-(2-amino-1-hydroxyethyl)-6-(4-fluorophenyl)pyridin-4-yl)propan-2-yl)carbamate

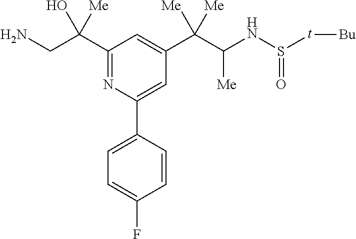

### Step 1: benzyl (2-(2-(4-fluorophenyl)-6-vinylpyridin-4-yl)propan-2-yl)carbamate

A mixture of benzyl (2-(2-chloro-6-(4-fluorophenyl)pyridin-4-yl)propan-2-yl)carbamate (0.30 g, 0.75 mmol), 4,4,5,5-tetramethyl-2-vinyl-1,3,2-dioxaborolane (0.17 g, 1.1 mmol), cesium carbonate (0.31 g, 2.3 mmol), [1,1′-bis(diphenylphosphino)ferrocene]dichloropalladium(II) (0.11 g, 0.15 mmol), 1,4-dioxane (4.0 mL), and water (0.40 mL) was placed under a N₂ atmosphere and stirred in an 80° C. heating block for 2 h. The mixture was cooled to room temperature, filtered, and concentrated. The mixture was diluted with water (50 mL) and extracted with EtOAc (30 mL×3). The combined organic extracts were washed with brine (50 mL), dried with anhydrous sodium sulfate, filtered, and concentrated in vacuo. The residue was purified by silica gel chromatography (15% EtOAc/petroleum ether) to afford the title compound.

### Step 2: benzyl (2-(2-(4-fluorophenyl)-6-(oxiran-2-yl)pyridin-4-yl)propan-2-yl)carbamate

A mixture of benzyl (2-(2-(4-fluorophenyl)-6-vinylpyridin-4-yl)propan-2-yl)carbamate (0.030 g, 0.077 mmol), 1,4-dioxane (0.30 mL), water (0.90 mL), acetic acid (4.4 μL, 0.077 mmol), and N-bromosuccinimide (14 mg, 0.077 mmol) was stirred at 25° C. for 1 h. The mixture was filtered and concentrated. The mixture was diluted with water (20 mL) and extracted with EtOAc (30 mL×3). The combined organic extracts were washed with brine (50 mL), dried with anhydrous sodium sulfate, filtered, and concentrated in vacuo. The residue was purified by preparative thin-layer chromatography (25% EtOAc/petroleum ether) to afford the title compound.

### Step 3: benzyl (2-(2-(2-amino-1-hydroxyethyl)-6-(4-fluorophenyl)pyridin-4-yl)propan-2-yl)carbamate

A mixture of benzyl (2-(2-(4-fluorophenyl)-6-(oxiran-2-yl)pyridin-4-yl)propan-2-yl)carbamate (0.060 g, 0.15 mmol) and methanolic ammonia (7.0 N, 3.0 mL) was stirred at 25° C. for 12 h. The mixture was filtered and concentrated to afford the title compound.

### EXAMPLES

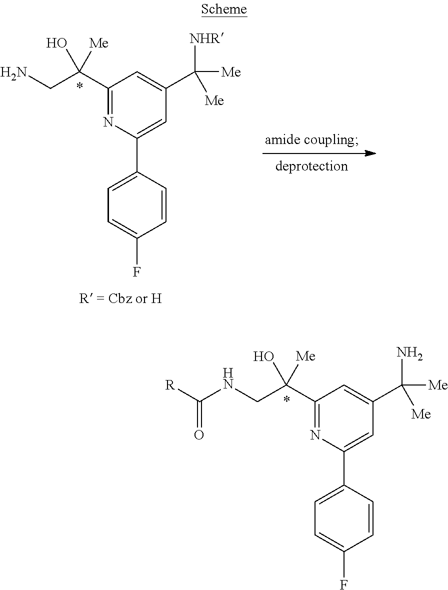

### Example 01-A

### N-2-(4-(2-aminopropan-2-yl)-6-(4-fluorophenyl)pyridin-2-yl)-2-hydroxypropyl)-3-ethoxy-1-methyl-1H-pyrazole-5-carboxamide (enantiomer-1)

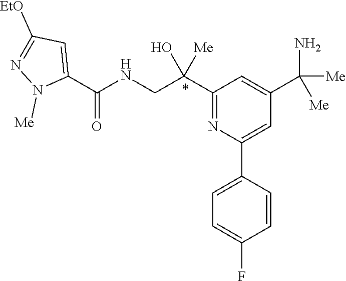 [SMILES: CCOc1cc(C(=O)NCC(C)(O)c2cc(C(C)(C)N)cc(-c3ccc(F)cc3)n2)n(C)n1]

A mixture of 3-ethoxy-1-methyl-1H-pyrazole-5-carboxylic acid (Intermediate A-01, 0.010 g, 0.059 mmol), HATU (27 mg, 0.071 mmol), and DCM (0.50 mL) was stirred at room temperature for 10 min. Benzyl (2-(2-(1-amino-2-hydroxypropan-2-yl)-6-(4-fluorophenyl)pyridin-4-yl)propan-2-yl)carbamate (Intermediate C-03-enantiomer-1, 0.030 g, 0.059 mmol) and triethylamine (0.025 mL, 0.18 mmol) were added and the mixture was stirred for 1 h. Palladium(II) chloride (3.1 mg, 0.018 mmol), triethylamine (0.025 mL, 0.18 mmol), and triethylsilane (0.047 mL, 0.29 mmol) were added and the mixture was stirred for 30 min. The mixture was concentrated and the residue was purified by preparative HPLC (C-18; 5-95% MeCN (with 0.1% TFA)/water (with 0.1% TFA)) to afford the title compound. MS m/z=(M+H)⁺: calculated 456.2, observed 456.3. ¹H NMR (500 MHz, CD₃OD) δ 8.17-8.11 (m, 2H), 7.78 (d, J=1.7 Hz, 1H), 7.75 (d, J=1.7 Hz, 1H), 7.24-7.18 (m, 2H), 6.00-5.99 (m, 1H), 4.03 (q, J=7.1 Hz, 2H), 3.91-3.85 (m, 1H), 3.75 (s, 3H), 3.74-3.68 (m, 1H), 1.77 (s, 3H), 1.76 (s, 3H), 1.66 (s, 3H), 1.31 (t, J=7.0 Hz, 3H) ppm.

### Example 01-B

### N-2-(4-(2-aminopropan-2-yl)-6-(4-fluorophenyl)pyridin-2-yl)-2-hydroxypropyl)-3-ethoxy-1-methyl-1H-pyrazole-5-carboxamide (enantiomer-2)

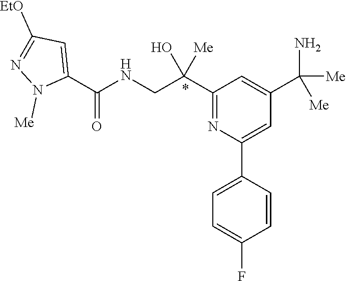 [SMILES: CCOc1cc(C(=O)NCC(C)(O)c2cc(C(C)(C)N)cc(-c3ccc(F)cc3)n2)n(C)n1]

A mixture of 3-ethoxy-1-methyl-1H-pyrazole-5-carboxylic acid (Intermediate A-01, 0.010 g, 0.059 mmol), HATU (27 mg, 0.071 mmol), and DCM (0.50 mL) was stirred at room temperature for 10 min. Benzyl (2-(2-(1-amino-2-hydroxypropan-2-yl)-6-(4-fluorophenyl)pyridin-4-yl)propan-2-yl)carbamate (Intermediate C-03-enantiomer-2, 0.030 g, 0.059 mmol) and triethylamine (0.025 mL, 0.18 mmol) were added and the mixture was stirred for 1 h. Palladium(II) chloride (3.1 mg, 0.018 mmol), triethylamine (0.025 mL, 0.18 mmol), and triethylsilane (0.047 mL, 0.29 mmol) were added and the mixture was stirred for 30 min. The mixture was concentrated in vacuo and the residue was purified by preparative HPLC (C-18; 5-95% MeCN (with 0.1% TFA)/water (with 0.1% TFA)) to afford the title compound. MS m/z=(M+H)⁺: calculated 456.2, observed 456.3. ¹H NMR (500 MHz, CD₃OD) δ 8.17-8.10 (m, 2H), 7.78 (d, J=1.7 Hz, 1H), 7.75 (d, J=1.7 Hz, 1H), 7.25-7.17 (m, 2H), 6.00-5.99 (m, 1H), 4.03 (q, J=7.1 Hz, 2H), 3.90-3.84 (m, 1H), 3.75 (s, 3H), 3.74-3.68 (m, 1H), 1.77 (s, 3H), 1.76 (s, 3H), 1.66 (s, 3H), 1.31 (t, J=7.0 Hz, 3H) ppm.

### Example 02-B

### N-2-(4-(2-aminopropan-2-yl)-6-(4-fluorophenyl)pyridin-2-yl)-2-hydroxypropyl)-1′-methyl-1′H-[1,3′-bipyrazole]-5′-carboxamide (enantiomer-2)

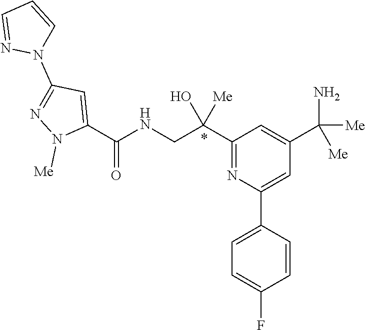 [SMILES: Cn1nc(-n2cccn2)cc1C(=O)NCC(C)(O)c1cc(C(C)(C)N)cc(-c2ccc(F)cc2)n1]

### Step 1: benzyl (2-(2-(4-fluorophenyl)-6-(2-hydroxy-1-(1′-methyl-1′H-[1,3′-bipyrazole]-5′-carboxamido)propan-2-yl)pyridin-4-yl)propan-2-yl)carbamate (enantiomer-2)

A mixture of 1′-methyl-1′H-[1,3′-bipyrazole]-5′-carboxylic acid (Intermediate A-02, 44 mg, 0.23 mmol), benzyl (2-(2-(1-amino-2-hydroxypropan-2-yl)-6-(4-fluorophenyl)pyridin-4-yl)propan-2-yl)carbamate (Intermediate C-03-enantiomer-2, 0.10 g, 0.23 mmol), (benzotriazol-1-yloxy)tripyrrolidinophosphonium hexafluorophosphate (0.14 g, 0.27 mmol), DIPEA (0.12 mL, 0.69 mmol), and DMF (8.0 mL) was stirred at 25° C. for 1 h. Water (10 mL) was added and the mixture was extracted with EtOAc (15 mL×3). The combined organic extracts were washed with brine (15 mL), dried with anhydrous sodium sulfate, filtered, and concentrated in vacuo. The residue was purified by preparative TLC (33% EtOAc/petroleum ether) to afford the title compound.

### Step 2: N-2-(4-(2-aminopropan-2-yl)-6-(4-fluorophenyl)pyridin-2-yl)-2-hydroxypropyl)-1′-methyl-1′H-[1,3′-bipyrazole]-5′-carboxamide (enantiomer-2)

A mixture of benzyl (2-(2-(4-fluorophenyl)-6-(2-hydroxy-1-(1′-methyl-1′H-[1,3′-bipyrazole]-5′-carboxamido)propan-2-yl)pyridin-4-yl)propan-2-yl)carbamate (enantiomer-2, 98 mg, 0.16 mmol) and hydrochloric acid (12 M aqueous, 1.0 mL) was stirred at 80° C. for 10 min. The mixture was cooled to room temperature and the pH was adjusted to ˜8 with ammonium hydroxide. The mixture was purified by preparative HPLC (C-18; 35-65% MeCN/water (10 mM ammonium bicarbonate)) to afford the title compound. MS m/z=(M+H)⁺: calculated 478.2, observed 478.2. ¹H NMR (500 MHz, CD₃OD) δ 8.15-8.10 (m, 2H), 8.09 (d, J=2.4 Hz, 1H), 7.86 (d, J=1.2 Hz, 1H), 7.74 (d, J=1.2 Hz, 1H), 7.68 (d, J=1.6 Hz, 1H), 7.16 (t, J=8.8 Hz, 2H), 6.77 (s, 1H), 6.47 (t, J=2.0 Hz, 1H), 3.96-3.90 (m, 4H), 3.73 (d, J=13.6 Hz, 1H), 1.66 (s, 3H), 1.55-1.45 (m, 6H) ppm.

### Example 03-B

### N-2-(4-(2-aminopropan-2-yl)-6-(4-fluorophenyl)pyridin-2-yl)-2-hydroxypropyl)-3-(isoxazol-3-yl)-1-methyl-1H-pyrazole-5-carboxamide (enantiomer-2)

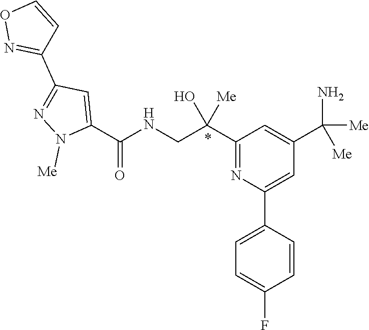 [SMILES: Cn1nc(-c2ccon2)cc1C(=O)NCC(C)(O)c1cc(C(C)(C)N)cc(-c2ccc(F)cc2)n1]

### Step 1: benzyl (2-(2-(4-fluorophenyl)-6-(2-hydroxy-1-(3-(isoxazol-3-yl)-1-methyl-1H-pyrazole-5-carboxamido)propan-2-yl)pyridin-4-yl)propan-2-yl)carbamate (enantiomer-2)

HATU (0.59 g, 1.6 mmol) was added to a mixture of 3-(isoxazol-3-yl)-1-methyl-1H-pyrazole-5-carboxylic acid (Intermediate A-03, 0.25 g, 1.3 mmol), DIPEA (0.70 mL, 3.9 mmol), and DMF (0.010 L). The mixture was stirred for 5 min at 25° C. Benzyl (2-(2-(1-amino-2-hydroxypropan-2-yl)-6-(4-fluorophenyl)pyridin-4-yl)propan-2-yl)carbamate (Intermediate C-03-enantiomer-2, 0.57 g, 1.3 mmol) was added and the mixture was stirred for 0.5 h. Water (10 mL) was added and the mixture was extracted with EtOAc (20 mL×3). The combined organic extracts were washed with brine (10 mL), dried with anhydrous sodium sulfate, filtered, and concentrated in vacuo. The residue was purified by preparative TLC (67% EtOAc/petroleum ether) to afford the title compound.

### Step 2: N-2-(4-(2-aminopropan-2-yl)-6-(4-fluorophenyl)pyridin-2-yl)-2-hydroxypropyl)-3-(isoxazol-3-yl)-1-methyl-1H-pyrazole-5-carboxamide (enantiomer-2)

A mixture of benzyl (2-(2-(4-fluorophenyl)-6-(2-hydroxy-1-(3-(isoxazol-3-yl)-1-methyl-1H-pyrazole-5-carboxamido)propan-2-yl)pyridin-4-yl)propan-2-yl)carbamate (enantiomer-2, 15 mg, 0.024 mmol) and hydrochloric acid (12 M aqueous, 0.50 mL) was stirred at 80° C. for 10 min. The mixture was cooled to room temperature and the pH was adjusted to ˜7 with ammonium hydroxide. The mixture was purified by preparative HPLC (C-18; 31-61% MeCN/water (10 mM ammonium bicarbonate)) to afford the title compound. MS m/z=(M+H)⁺: calculated 479.2, observed 479.2. ¹H NMR (500 MHz, CD₃OD) δ 8.69 (d, J=1.5 Hz, 1H), 8.12 (dd, J=9.0, 5.5 Hz, 2H), 7.86 (d, J=1.5 Hz, 1H), 7.74 (d, J=1.5 Hz, 1H), 7.16 (t, J=9 Hz, 2H), 7.07 (s, 1H), 6.78 (d, J=1.5 Hz, 1H), 4.02 (s, 3H), 3.93 (d, J=13.5 Hz, 1H), 3.74 (d, J=13.5 Hz, 1H), 1.66 (s, 3H), 1.56-1.48 (m, 6H) ppm.

### Example 04-B

### N-2-(4-(2-aminopropan-2-yl)-6-(4-fluorophenyl)pyridin-2-yl)-2-hydroxypropyl)-1-methyl-3-(oxazol-2-yl)-1H-pyrazole-5-carboxamide (enantiomer-2)

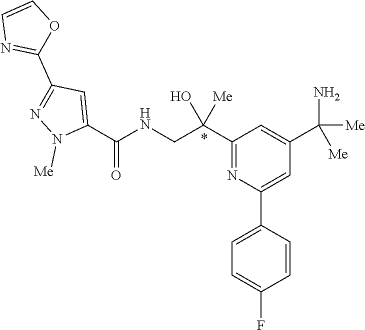 [SMILES: Cn1nc(-c2ncco2)cc1C(=O)NCC(C)(O)c1cc(C(C)(C)N)cc(-c2ccc(F)cc2)n1]

### Step 1: benzyl (2-(2-(4-fluorophenyl)-6-(2-hydroxy-1-(1-methyl-3-(oxazol-2-yl)-1H-pyrazole-5-carboxamido)propan-2-yl)pyridin-4-yl)propan-2-yl)carbamate (enantiomer-2)

A mixture of 1-methyl-3-(oxazol-2-yl)-1H-pyrazole-5-carboxylic acid (Intermediate A-04, 0.030 g, 0.16 mmol), benzyl (2-(2-(1-amino-2-hydroxypropan-2-yl)-6-(4-fluorophenyl)pyridin-4-yl)propan-2-yl)carbamate (Intermediate C-03-enantiomer-2, 68 mg, 0.16 mmol), 1-propanephosphonic anhydride (50 wt % in EtOAc, 0.30 g, 0.47 mmol), DIPEA (0.082 mL, 0.47 mmol), and DMF (5.0 mL) was stirred at 25° C. for 0.5 h. Water (5 mL) was added and the mixture was extracted with EtOAc (5 mL×3). The combined organic extracts were washed with brine (5 mL), dried with anhydrous sodium sulfate, filtered, and concentrated in vacuo. The material was purified by preparative TLC (EtOAc) to afford the title compound.

### Step 2: N-2-(4-(2-aminopropan-2-yl)-6-(4-fluorophenyl)pyridin-2-yl)-2-hydroxypropyl)-1-methyl-3-(oxazol-2-yl)-1H-pyrazole-5-carboxamide (enantiomer-2)

A mixture of benzyl (2-(2-(4-fluorophenyl)-6-(2-hydroxy-1-(1-methyl-3-(oxazol-2-yl)-1H-pyrazole-5-carboxamido)propan-2-yl)pyridin-4-yl)propan-2-yl)carbamate (enantiomer-2, 0.060 g, 0.098 mmol) and hydrochloric acid (12 M aqueous, 1.0 mL) was stirred at 80° C. for 10 min. The mixture was cooled to room temperature and purified by preparative HPLC (C-18; 20-50% MeCN/water (with 0.1% TFA)) to afford the title compound. MS m/z=(M+H)⁺: calculated 479.2, observed 479.2. ¹H NMR (400 MHz, CD₃OD) δ 8.14 (dd, J=8.8, 5.4 Hz, 2H), 7.96 (s, 1H), 7.81-7.76 (m, 2H), 7.28 (s, 1H), 7.19 (t, J=8.7 Hz, 2H), 7.13 (s, 1H), 4.01 (s, 3H), 3.95-3.91 (m, 1H), 3.81-3.74 (m, 1H), 1.78 (s, 6H), 1.71-1.66 (m, 3H) ppm.

### Example 05-B

### N-2-(4-(2-aminopropan-2-yl)-6-(4-fluorophenyl)pyridin-2-yl)-2-hydroxypropyl)-1-methyl-3-(thiazol-4-yl)-1H-pyrazole-5-carboxamide (enantiomer-2)

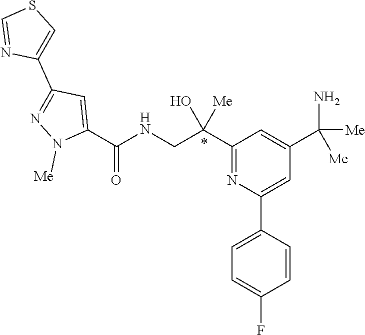 [SMILES: Cn1nc(-c2cscn2)cc1C(=O)NCC(C)(O)c1cc(C(C)(C)N)cc(-c2ccc(F)cc2)n1]

A mixture of 1-methyl-3-(thiazol-4-yl)-1H-pyrazole-5-carboxylic acid (Intermediate A-05, 11 mg, 0.051 mmol), HATU (33 mg, 0.087 mmol), and DMF (0.50 mL) was stirred at room temperature for 5 min. 1-Amino-2-(4-(2-aminopropan-2-yl)-6-(4-fluorophenyl)pyridin-2-yl)propan-2-ol (Intermediate C-04-enantiomer-2, 0.030 g, 0.073 mmol) and triethylamine (0.030 mL, 0.22 mmol) were added and the mixture was stirred for 30 min. The mixture was purified by preparative HPLC (C-18; 10-95% MeCN (with 0.1% TFA)/water (with 0.1% TFA)) to afford the title compound. MS m/z=(M+H)⁺: calculated 495.2, observed 495.2. ¹H NMR (500 MHz, CD₃OD) δ 9.05 (s, 1H), 8.17-8.10 (m, 2H), 7.81-7.76 (m, 3H), 7.22-7.14 (m, 2H), 7.02 (s, 1H), 3.98 (s, 3H), 3.94 (d, J=13.5 Hz, 1H), 3.76 (d, J=13.6 Hz, 1H), 1.77 (s, 6H), 1.69 (s, 3H) ppm.

### Example 06-B

### N-2-(4-(2-aminopropan-2-yl)-6-(4-fluorophenyl)pyridin-2-yl)-2-hydroxypropyl)-1-ethyl-3-(thiazol-4-yl)-1H-pyrazole-5-carboxamide (enantiomer-2)

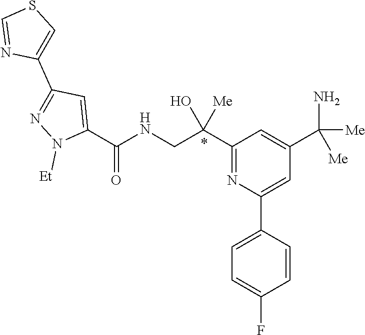 [SMILES: CCn1nc(-c2cscn2)cc1C(=O)NCC(C)(O)c1cc(C(C)(C)N)cc(-c2ccc(F)cc2)n1]

### Step 1: benzyl (2-(2-(1-(1-ethyl-3-(thiazol-4-yl)-1H-pyrazole-5-carboxamido)-2-hydroxypropan-2-yl)-6-(4-fluorophenyl)pyridin-4-yl)propan-2-yl)carbamate (enantiomer-2)

A mixture of 1-ethyl-3-(thiazol-4-yl)-1H-pyrazole-5-carboxylic acid (Intermediate A-06, 0.040 g, 0.18 mmol), benzyl (2-(2-(1-amino-2-hydroxypropan-2-yl)-6-(4-fluorophenyl)pyridin-4-yl)propan-2-yl)carbamate (Intermediate C-03-enantiomer-2, 78 mg, 0.18 mmol), 1-propanephosphonic anhydride (50 wt % in EtOAc, 0.34 g, 0.54 mmol), DIPEA (0.094 mL, 0.54 mmol), and DMF (3.0 mL) was stirred at 25° C. for 0.5 h. Water (5 mL) was added and the mixture was extracted with EtOAc (5 mL×3). The combined organic extracts were washed with brine (5 mL), dried with anhydrous sodium sulfate, filtered, and concentrated in vacuo. The residue was purified by preparative TLC (50% EtOAc/petroleum ether) to afford the title compound.

### Step 2: N-2-(4-(2-aminopropan-2-yl)-6-(4-fluorophenyl)pyridin-2-yl)-2-hydroxypropyl)-1-ethyl-3-(thiazol-4-yl)-1H-pyrazole-5-carboxamide (enantiomer-2)

A mixture of benzyl (2-(2-(1-(1-ethyl-3-(thiazol-4-yl)-1H-pyrazole-5-carboxamido)-2-hydroxypropan-2-yl)-6-(4-fluorophenyl)pyridin-4-yl)propan-2-yl)carbamate (enantiomer-2, 65 mg, 0.10 mmol) and hydrochloric acid (12 M aqueous, 1.0 mL) was stirred at 80° C. for 10 min. The mixture was cooled to room temperature and purified by preparative HPLC (C-18; 23-53% MeCN/water (with 0.1% TFA)) to afford the title compound. MS m/z=(M+H)⁺: calculated 509.2, observed 509.2. ¹H NMR (400 MHz, CD₃OD) δ 9.05 (d, J=1.5 Hz, 1H), 8.18-8.11 (m, 2H), 7.80 (d, J=3.4 Hz, 3H), 7.17 (t, J=8.7 Hz, 2H), 7.01 (s, 1H), 4.47-4.37 (m, 2H), 3.97 (d, J=13.7 Hz, 1H), 3.76 (d, J=13.4 Hz, 1H), 1.77 (s, 6H), 1.69 (s, 3H), 1.30 (t, J=7.1 Hz, 3H) ppm.

### Example 07-B

### N-2-(4-(2-aminopropan-2-yl)-6-(4-fluorophenyl)pyridin-2-yl)-2-hydroxypropyl)-4-methyl-[2,4′-bithiazole]-5-carboxamide (enantiomer-2)

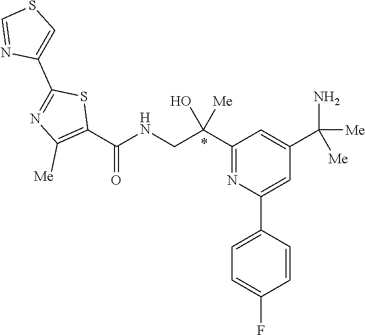 [SMILES: Cc1nc(-c2cscn2)sc1C(=O)NCC(C)(O)c1cc(C(C)(C)N)cc(-c2ccc(F)cc2)n1]

### Step 1: benzyl (2-(2-(4-fluorophenyl)-6-(2-hydroxy-1-(4-methyl-[2,4′-bithiazole]-5-carboxamido)propan-2-yl)pyridin-4-yl)propan-2-yl)carbamate (enantiomer-2)

A mixture of 4-methyl-[2,4′-bithiazole]-5-carboxylic acid (Intermediate A-07, 0.010 g, 0.046 mmol), benzyl (2-(2-(1-amino-2-hydroxypropan-2-yl)-6-(4-fluorophenyl)pyridin-4-yl)propan-2-yl)carbamate (Intermediate C-03-enantiomer-2, 0.020 g, 0.046 mmol), HATU (17 mg, 0.046 mmol), DIPEA (0.024 mL, 0.14 mmol), and DCM (0.23 mL) was stirred at room temperature for 30 min. The mixture was purified by silica gel chromatography (0-60% (25% EtOH/EtOAc)/hexanes) to afford the title compound.

### Step 2: N-2-(4-(2-aminopropan-2-yl)-6-(4-fluorophenyl)pyridin-2-yl)-2-hydroxypropyl)-4-methyl-[2,4′-bithiazole]-5-carboxamide (enantiomer-2)

A mixture of benzyl (2-(2-(4-fluorophenyl)-6-(2-hydroxy-1-(4-methyl-[2,4′-bithiazole]-5-carboxamido)propan-2-yl)pyridin-4-yl)propan-2-yl)carbamate (enantiomer-2, 48 mg, 0.074 mmol) and hydrochloric acid (12 M aqueous, 1.0 mL) was stirred at 80° C. for 10 min. The mixture was concentrated and purified by preparative HPLC (C-18; 5-70% MeCN (with 0.1% TFA)/water (with 0.1% TFA)) to afford the title compound. MS m/z=(M+H)⁺: calculated 512.2, observed 512.1. ¹H NMR (500 MHz, CDCl₃) δ 8.82 (s, 1H), 8.04-7.96 (m, 3H), 7.76 (s, 1H), 7.57 (s, 1H), 7.19-7.13 (m, 2H), 6.33 (s, 1H), 5.71 (s, 1H), 4.09-4.01 (m, 1H), 3.77-3.69 (m, 1H), 2.55 (s, 3H), 1.62 (s, 3H), 1.52 (s, 6H) ppm.

### Example 08-B

### N-2-(4-(2-aminopropan-2-yl)-6-(4-fluorophenyl)pyridin-2-yl)-2-hydroxypropyl)-4-cyclopropyl-[2,4′-bithiazole]-5-carboxamide (enantiomer-2)

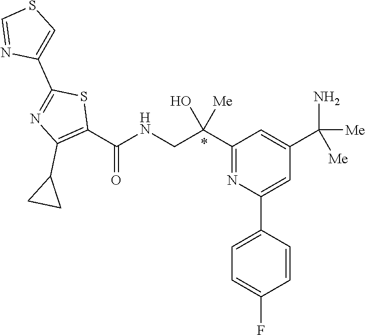 [SMILES: CC(C)(N)c1cc(-c2ccc(F)cc2)nc(C(C)(O)CNC(=O)c2sc(-c3cscn3)nc2C2CC2)c1]

A mixture of 4-cyclopropyl-[2,4′-bithiazole]-5-carboxylic acid (Intermediate A-08, 2.0 mg, 8.0 μmol), 1-amino-2-(4-(2-aminopropan-2-yl)-6-(4-fluorophenyl)pyridin-2-yl)propan-2-ol (Intermediate C-04-enantiomer-2, 4.8 mg, 16 μmol) HATU (3.0 mg, 8.0 μmol), DIPEA (4.1 μL, 24 μmol) and DMF (1.0 mL) was stirred at room temperature for 30 min. The mixture was purified by preparative HPLC (C-18; 10-95% MeCN (with 0.05% TFA)/water (with 0.05% TFA)) to afford the title compound. MS m/z=(M+H)⁺: calculated 538.2, observed 538.3. ¹H NMR (500 MHz, DMSO-d6) δ 9.20 (d, J=1.9 Hz, 1H), 8.59 (s, 3H), 8.26 (d, J=1.9 Hz, 1H), 8.22-8.17 (m, 2H), 7.97 (t, J=5.9 Hz, 1H), 7.89 (d, J=1.3 Hz, 1H), 7.76 (d, J=1.4 Hz, 1H), 7.39-7.32 (m, 2H), 3.82-3.71 (m, 2H), 2.55-2.51 (m, 1H), 1.66 (s, 6H), 1.56 (s, 3H), 0.98-0.92 (m, 2H), 0.91-0.85 (m, 2H) ppm.

### Example 09-B

### N-2-(4-(2-aminopropan-2-yl)-6-(4-fluorophenyl)pyridin-2-yl)-2-hydroxypropyl)-3-(thiazol-4-yl)isothiazole-5-carboxamide (enantiomer-2)

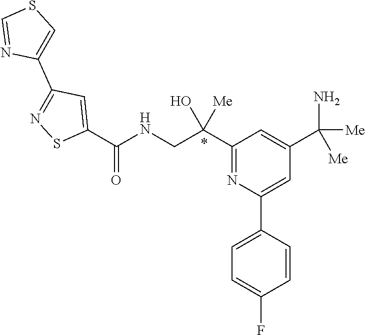 [SMILES: CC(C)(N)c1cc(-c2ccc(F)cc2)nc(C(C)(O)CNC(=O)c2cc(-c3cscn3)ns2)c1]

### Step 1: benzyl (2-(2-(4-fluorophenyl)-6-(2-hydroxy-1-(3-(thiazol-4-yl)isothiazole-5-carboxamido)propan-2-yl)pyridin-4-yl)propan-2-yl)carbamate (enantiomer-2)

A mixture of 3-(thiazol-4-yl)isothiazole-5-carboxylic acid (Intermediate A-09, 0.030 g, 0.14 mmol), benzyl (2-(2-(1-amino-2-hydroxypropan-2-yl)-6-(4-fluorophenyl)pyridin-4-yl)propan-2-yl)carbamate (Intermediate C-03-enantiomer-2, 68 mg, 0.16 mmol), 1-propanephosphonic anhydride (50 wt % in EtOAc, 0.18 g, 0.28 mmol), DIPEA (0.074 mL, 0.42 mmol), and DMF (4.0 mL) was stirred at 25° C. for 0.5 h. Water (5 mL) was added and the mixture was extracted with EtOAc (5 mL×3). The combined organic solution was washed with brine (10 mL), dried with anhydrous sodium sulfate, filtered, and concentrated. The material was purified by prep-TLC (EtOAc) to afford the title compound.

### Step 2: N-2-(4-(2-aminopropan-2-yl)-6-(4-fluorophenyl)pyridin-2-yl)-2-hydroxypropyl)-3-(thiazol-4-yl)isothiazole-5-carboxamide (enantiomer-2)

A mixture of benzyl (2-(2-(4-fluorophenyl)-6-(2-hydroxy-1-(3-(thiazol-4-yl)isothiazole-5-carboxamido)propan-2-yl)pyridin-4-yl)propan-2-yl)carbamate (enantiomer-2, 25 mg, 0.040 mmol) and hydrochloric acid (12 M aqueous, 1.0 mL) was stirred at 80° C. for 10 min. The mixture was cooled to room temperature and purified by prep-HPLC (C-18; 30-50% MeCN/water (with 0.1% TFA modifier)) to afford the title compound. MS m/z=(M+H)⁺: calculated 498.1, observed 498.1. ¹H NMR (400 MHz, CD₃OD) δ 9.09 (s, 1H), 8.16-8.10 (m, 4H), 7.78 (q, J=1.7 Hz, 2H), 7.18-7.12 (m, 2H), 3.96-3.92 (m, 1H), 3.83-3.78 (m, 1H), 1.80-1.72 (m, 6H), 1.71 (s, 3H) ppm.

### Example 10-B

### N-2-(4-(2-aminopropan-2-yl)-6-(4-fluorophenyl)pyridin-2-yl)-2-hydroxypropyl)-3-(pyrimidin-2-yl)isothiazole-5-carboxamide (enantiomer-2)

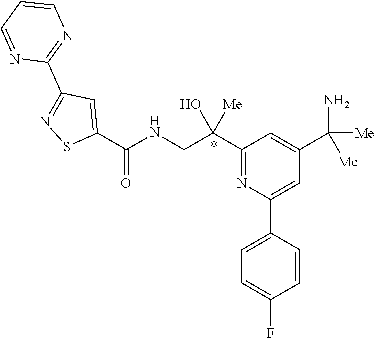 [SMILES: CC(C)(N)c1cc(-c2ccc(F)cc2)nc(C(C)(O)CNC(=O)c2cc(-c3ncccn3)ns2)c1]

### Step 1: benzyl (2-(2-(4-fluorophenyl)-6-(2-hydroxy-1-(3-(pyrimidin-2-yl)isothiazole-5-carboxamido)propan-2-yl)pyridin-4-yl)propan-2-yl)carbamate (enantiomer-2)

A mixture of 3-(pyrimidin-2-yl)isothiazole-5-carboxylic acid (Intermediate A-10, 0.030 g, 0.15 mmol), benzyl (2-(2-(1-amino-2-hydroxypropan-2-yl)-6-(4-fluorophenyl)pyridin-4-yl)propan-2-yl)carbamate (Intermediate C-03-enantiomer-2, 63 mg, 0.15 mmol), 1-propanephosphonic anhydride (50 wt % in EtOAc, 0.14 g, 0.22 mmol), DIPEA (0.076 mL, 0.43 mmol), and DMF (5.0 mL) was stirred at 25° C. for 1 h. Water (10 mL) was added and the mixture was extracted with EtOAc (10 mL×3). The combined organic extracts were dried with anhydrous sodium sulfate, filtered, and concentrated in vacuo. The residue was purified by preparative TLC (50% EtOAc/petroleum ether) to afford the title compound.

### Step 2: N-2-(4-(2-aminopropan-2-yl)-6-(4-fluorophenyl)pyridin-2-yl)-2-hydroxypropyl)-3-(pyrimidin-2-yl)isothiazole-5-carboxamide (enantiomer-2)

A mixture of benzyl (2-(2-(4-fluorophenyl)-6-(2-hydroxy-1-(3-(pyrimidin-2-yl)isothiazole-5-carboxamido)propan-2-yl)pyridin-4-yl)propan-2-yl)carbamate (enantiomer-2, 0.060 g, 0.096 mmol) and hydrochloric acid (12 M aqueous, 2.0 mL) was stirred at 80° C. for 10 min. The mixture was cooled to room temperature, diluted with MeOH (2 mL), and filtered. The filtrate was concentrated in vacuo and the residue was purified by preparative HPLC (C-18; 15-35% MeCN/water (with 0.05% HCl)) to afford the title compound. MS m/z=(M+H)⁺: calculated 493.3, observed 493.2. ¹H NMR (500 MHz, CD₃OD) δ 8.96 (d, J=5.0 Hz, 2H), 8.52 (s, 1H), 8.22-8.05 (m, 2H), 7.87 (d, J=2.5 Hz, 2H), 7.58 (t, J=5.0 Hz, 1H), 7.20 (t, J=9.0 Hz, 2H), 4.00 (d, J=14.0 Hz, 1H), 3.82 (d, J=13.4 Hz, 1H), 1.80 (s, 6H), 1.76 (s, 3H) ppm.

### Example 11-B

### N-2-(4-(2-aminopropan-2-yl)-6-(4-fluorophenyl)pyridin-2-yl)-2-hydroxypropyl)-3-methoxy-1-(pyrimidin-2-yl)-1H-pyrazole-4-carboxamide (enantiomer-2)

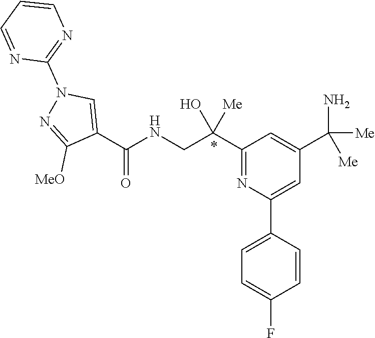 [SMILES: COc1nn(-c2ncccn2)cc1C(=O)NCC(C)(O)c1cc(C(C)(C)N)cc(-c2ccc(F)cc2)n1]

A mixture of 3-methoxy-1-(pyrimidin-2-yl)-1H-pyrazole-4-carboxylic acid (Intermediate A-11, 25 mg, 0.11 mmol), 1-amino-2-(4-(2-aminopropan-2-yl)-6-(4-fluorophenyl)pyridin-2-yl)propan-2-ol (Intermediate C-04-enantiomer-2, 47 mg, 0.11 mmol), HATU (43 mg, 0.11 mmol), DIPEA (79 μL, 0.45 mmol), and DMF (1.1 mL) was stirred at room temperature for 16 h. The mixture was purified by preparative HPLC (C-18; 10-100% MeCN (with 0.1% TFA)/water (with 0.1% TFA)) to afford the title compound. MS m/z=(M+H)⁺: calculated 506.2, observed 506.4. ¹H NMR (500 MHz, DMSO-d6) δ 8.84 (d, J=4.8 Hz, 2H), 8.76 (s, 1H), 8.57 (s, 3H), 8.22 (dd, J=8.6, 5.6 Hz, 2H), 7.94 (s, 1H), 7.76 (s, 1H), 7.49-7.42 (m, 2H), 7.39 (t, J=8.8 Hz, 2H), 5.85 (s, 1H), 3.86 (s, 3H), 3.80-3.71 (m, 2H), 1.68-1.64 (m, 6H), 1.54 (s, 3H) ppm.

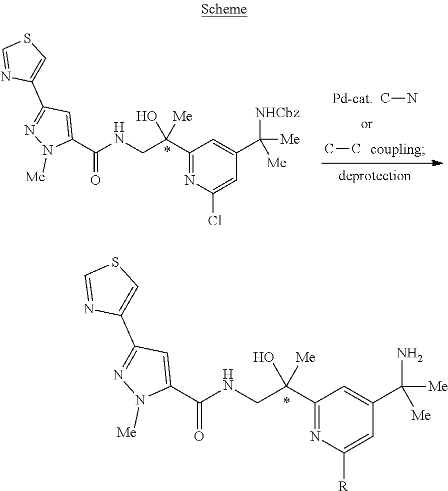

### Example 12-B

### N-2-(4-(2-aminopropan-2-yl)-6-(4-fluoro-1H-indol-1-yl)pyridin-2-yl)-2-hydroxypropyl)-1-methyl-3-(thiazol-4-yl)-1H-pyrazole-5-carboxamide (enantiomer-2)

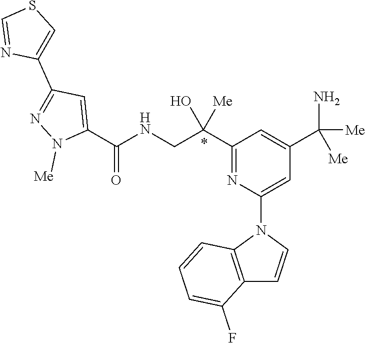 [SMILES: Cn1nc(-c2cscn2)cc1C(=O)NCC(C)(O)c1cc(C(C)(C)N)cc(-n2ccc3c(F)cccc32)n1]

A mixture of benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (Intermediate C-05-enantiomer-2, 75 mg, 0.13 mmol), 4-fluoroindole (27 mg, 0.20 mmol), tris(dibenzylideneacetone)dipalladium(0) (6.0 mg, 6.6 μmol), 5-(di-tert-butylphosphaneyl)-1′,3′,5′-triphenyl-1′H-1,4′-bipyrazole (6.7 mg, 13 μmol), potassium phosphate tribasic (84 mg, 0.40 mmol), and 1,4-dioxane (1.3 mL) was stirred at 100° C. for 16 h. The mixture was cooled to room temperature, filtered through a pad of celite, and the pad was washed with EtOAc (1 mL×3). The filtrate was concentrated in vacuo and aqueous hydrochloric acid (12 M, 1.3 mL) was added. The mixture was stirred at 80° C. for 10 min. The mixture was cooled to room temperature, diluted with water, and the pH was adjusted to >12 with aqueous NaOH (1.0 M). The mixture was extracted with DCM (15 mL×3) and the combined organic extracts were dried with anhydrous magnesium sulfate, filtered, and concentrated in vacuo. The residue was purified by preparative HPLC (C-18; 10-100% MeCN (with 0.1% TFA)/water (with 0.1% TFA)). The appropriate fractions were loaded onto a solid-phase extraction column (ISOLUTE® SCX-2; propylsulfonic acid bonded sorbent), the column was washed with MeOH, which was discarded. The column was eluted with methanolic ammonia (2.0 M) and the eluent was concentrated in vacuo to afford the title compound. MS m/z=(M+H)⁺: calculated 534.2, observed 534.5. ¹H NMR (500 MHz, DMSO-d6) δ 9.15 (d, J=2.0 Hz, 1H), 8.39 (t, J=6.1 Hz, 1H), 8.20 (d, J=8.5 Hz, 1H), 8.10 (d, J=3.5 Hz, 1H), 7.88 (d, J=2.0 Hz, 1H), 7.75 (d, J=1.3 Hz, 1H), 7.72 (d, J=1.3 Hz, 1H), 7.25 (s, 1H), 7.25-7.20 (m, 1H), 6.96 (dd, J=10.3, 7.9 Hz, 1H), 6.83 (d, J=3.0 Hz, 1H), 5.63 (s, 1H), 3.98 (s, 3H), 3.74 (dd, J=13.3, 5.8 Hz, 1H), 3.69 (dd, J=13.3, 6.4 Hz, 1H), 1.58 (s, 3H), 1.44 (s, 3H), 1.42 (s, 3H) ppm.

### Example 13-B

### N-2-(4-(2-aminopropan-2-yl)-6-(4-chlorophenyl)pyridin-2-yl)-2-hydroxypropyl)-1-methyl-3-(thiazol-4-yl)-1H-pyrazole-5-carboxamide (enantiomer-2)

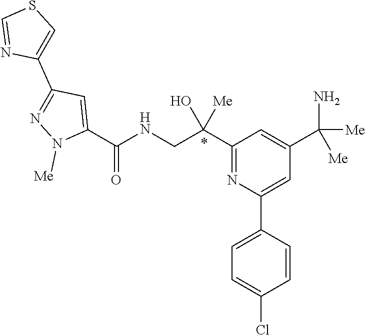 [SMILES: Cn1nc(-c2cscn2)cc1C(=O)NCC(C)(O)c1cc(C(C)(C)N)cc(-c2ccc(Cl)cc2)n1]

A mixture of benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (Intermediate C-05-enantiomer-2, 0.090 g, 0.16 mmol), (4-chlorophenyl)boronic acid (0.030 g, 0.19 mmol), tetrakis(triphenylphosphine)palladium(0) (9.1 mg, 7.9 μmol), potassium phosphate tribasic (0.050 g, 0.24 mmol), and 1,4-dioxane (1.6 mL) was stirred at 100° C. for 16 h. The mixture was cooled to room temperature and filtered through a pad of celite, which was washed with EtOAc (1 mL×3). The filtrate was concentrated in vacuo and aqueous hydrochloric acid (12 M, 1.6 mL) was added. The mixture was stirred at 80° C. for 10 min, cooled to room temperature, diluted with water, and the pH was adjusted to >12 with aqueous NaOH (1.0 M). The mixture was extracted with DCM (15 mL×3) and the combined organic extracts were dried with anhydrous magnesium sulfate, filtered, and concentrated in vacuo. The residue was purified by preparative HPLC (C-18; 10-100% MeCN (with 0.1% TFA)/water (with 0.1% TFA)). The appropriate fractions were loaded onto a solid-phase extraction column (ISOLUTE® SCX-2; propylsulfonic acid bonded sorbent). The column was washed with MeOH, which was discarded. The column was eluted with methanolic ammonia (2.0 M) and the eluent was concentrated in vacuo to afford the title compound. MS m/z=(M+H)⁺: calculated 511.2, observed 511.5. ¹H NMR (500 MHz, DMSO-d6) δ 9.16 (d, J=1.8 Hz, 1H), 8.35 (t, J=5.9 Hz, 1H), 8.16 (d, J=8.5 Hz, 2H), 7.97 (s, 1H), 7.89 (d, J=1.8 Hz, 1H), 7.79 (s, 1H), 7.54 (d, J=8.5 Hz, 2H), 7.25 (s, 1H), 5.59 (s, 1H), 4.00 (s, 3H), 3.75 (dd, J=13.2, 5.8 Hz, 1H), 3.63 (dd, J=13.3, 6.2 Hz, 1H), 1.54 (s, 3H), 1.41-1.37 (m, 6H) ppm.

### Example 14-B

### N-2-(4-(2-aminopropan-2-yl)-6-(p-tolyl)pyridin-2-yl)-2-hydroxypropyl)-1-methyl-3-(thiazol-4-yl)-1H-pyrazole-5-carboxamide (enantiomer-2)

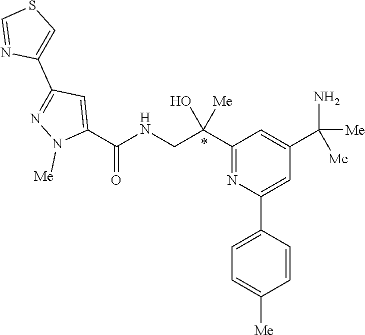 [SMILES: Cc1ccc(-c2cc(C(C)(C)N)cc(C(C)(O)CNC(=O)c3cc(-c4cscn4)nn3C)n2)cc1]

A mixture of benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (Intermediate C-05-enantiomer-2, 0.070 g, 0.12 mmol), p-tolylboronic acid (0.020 g, 0.15 mmol), chloro(2-dicyclohexylphosphino-2′,4′,6′-triisopropyl-1,1′-biphenyl)[2-(2′-amino-1,1′-biphenyl)]palladium(II) (4.8 mg, 6.2 μmol), potassium phosphate tribasic (39 mg, 0.19 mmol), and 1,4-dioxane (1.2 mL) was stirred at 100° C. for 16 h. The mixture was cooled to room temperature and filtered through a pad of celite, which was washed with EtOAc (1 mL×3). The filtrate was concentrated in vacuo and aqueous hydrochloric acid (12 M, 1.2 mL) was added. The mixture was stirred at 80° C. for 10 min, cooled to room temperature, diluted with water, and the pH was adjusted to >12 with aqueous NaOH (1.0 M). The mixture was extracted with DCM (15 mL×3) and the combined organic extracts were dried with anhydrous magnesium sulfate, filtered, and concentrated in vacuo. The residue was purified by preparative HPLC (C-18; 10-100% MeCN (with 0.1% TFA)/water (with 0.1% TFA)). The appropriate fractions were loaded onto a solid-phase extraction column (ISOLUTE® SCX-2; propylsulfonic acid bonded sorbent). The column was washed with MeOH, which was discarded. The column was then eluted with methanolic ammonia (2.0 M) and the eluent was concentrated in vacuo to afford the title compound. MS m/z=(M+H)⁺: calculated 491.2, observed 491.5. ¹H NMR (500 MHz, DMSO-d6) δ 9.17 (d, J=1.9 Hz, 1H), 8.35 (t, J=6.0 Hz, 1H), 8.01 (d, J=8.1 Hz, 2H), 7.91 (s, 1H), 7.88 (d, J=1.8 Hz, 1H), 7.74 (s, 1H), 7.29 (d, J=8.0 Hz, 2H), 7.25 (s, 1H), 5.57 (s, 1H), 4.01 (s, 3H), 3.76 (dd, J=13.3, 5.8 Hz, 1H), 3.62 (dd, J=13.2, 6.2 Hz, 1H), 2.35 (s, 3H), 1.53 (s, 3H), 1.42-1.38 (m, 6H) ppm.

### Example 15-B

### N-2-(4-(2-aminopropan-2-yl)-6-(3,4-difluorophenyl)pyridin-2-yl)-2-hydroxypropyl)-1-methyl-3-(thiazol-4-yl)-1H-pyrazole-5-carboxamide (enantiomer-2)

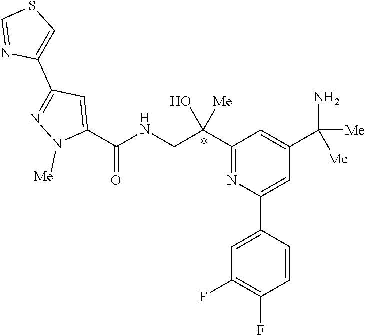 [SMILES: Cn1nc(-c2cscn2)cc1C(=O)NCC(C)(O)c1cc(C(C)(C)N)cc(-c2ccc(F)c(F)c2)n1]

A mixture of benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (Intermediate C-05-enantiomer-2, 0.090 g, 0.16 mmol), (3,4-difluorophenyl)boronic acid (0.030 g, 0.19 mmol), chloro(2-dicyclohexylphosphino-2′,4′,6′-triisopropyl-1,1′-biphenyl)[2-(2′-amino-1,1′-biphenyl)]palladium(II) (6.2 mg, 7.9 μmol), potassium phosphate tribasic (0.050 g, 0.24 mmol), and 1,4-dioxane (1.6 mL) was stirred at 100° C. for 16 h. The mixture was cooled to room temperature and filtered through a pad of celite, which was washed with EtOAc (1 mL×3). The filtrate was concentrated in vacuo and aqueous hydrochloric acid (12 M, 1.6 mL) was added. The mixture was stirred at 80° C. for 10 min, cooled to room temperature, diluted with water, and the pH was adjusted to >12 with aqueous NaOH (1.0 M). The mixture was extracted with DCM (15 mL×3) and the combined organic extracts were dried with anhydrous magnesium sulfate, filtered, and concentrated in vacuo. The residue was purified by preparative HPLC (C-18; 10-100% MeCN (with 0.1% TFA)/water (with 0.1% TFA)). The appropriate fractions were loaded onto a solid-phase extraction column (ISOLUTE® SCX-2; propylsulfonic acid bonded sorbent) and washed with MeOH, which was discarded. The column was then eluted with methanolic ammonia (2.0 M) and the eluent was concentrated in vacuo to afford the title compound. MS m/z=(M+H)⁺: calculated 513.2, observed 513.6. ¹H NMR (500 MHz, DMSO-d6) δ 9.15 (d, J=1.9 Hz, 1H), 8.33 (t, J=5.9 Hz, 1H), 8.23-8.15 (m, 1H), 8.04-7.99 (m, 1H), 7.98 (s, 1H), 7.88 (d, J=1.9 Hz, 1H), 7.80 (s, 1H), 7.54 (q, J=8.6 Hz, 1H), 7.22 (s, 1H), 5.59 (s, 1H), 3.99 (s, 3H), 3.73 (dd, J=13.2, 5.7 Hz, 1H), 3.67 (dd, J=13.3, 6.3 Hz, 1H), 1.53 (s, 3H), 1.43-1.34 (m, 6H) ppm.

### Example 16-B

### N-2-(4-(2-aminopropan-2-yl)-6-(3-chloro-4-fluorophenyl)pyridin-2-yl)-2-hydroxypropyl)-1-methyl-3-(thiazol-4-yl)-1H-pyrazole-5-carboxamide (enantiomer-2)

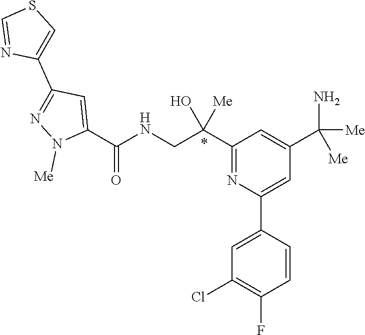 [SMILES: Cn1nc(-c2cscn2)cc1C(=O)NCC(C)(O)c1cc(C(C)(C)N)cc(-c2ccc(F)c(Cl)c2)n1]

A mixture of benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (Intermediate C-05-enantiomer-2, 62 mg, 0.11 mmol), (3-chloro-4-fluorophenyl)boronic acid (23 mg, 0.13 mmol), chloro(2-dicyclohexylphosphino-2′,4′,6′-triisopropyl-1,1′-biphenyl)[2-(2′-amino-1,1′-biphenyl)]palladium(II) (4.3 mg, 5.5 μmol), potassium phosphate tribasic (35 mg, 0.16 mmol), and 1,4-dioxane (1.1 mL) was stirred at 100° C. for 16 h. The mixture was cooled to room temperature and filtered through a pad of celite, which was washed with EtOAc (1 mL×3). The filtrate was concentrated in vacuo and aqueous hydrochloric acid (12 M, 1.1 mL) was added. The mixture was stirred at 80° C. for 10 min, cooled to room temperature, diluted with water, and the pH was adjusted to >12 with aqueous NaOH (1.0 M). The mixture was extracted with DCM (15 mL×3) and the combined organic extracts were dried with anhydrous magnesium sulfate, filtered, and concentrated in vacuo. The residue was purified by preparative HPLC (C-18; 10-100% MeCN (with 0.1% TFA)/water (with 0.1% TFA)). The appropriate fractions were loaded onto a solid-phase extraction column (ISOLUTE® SCX-2; propylsulfonic acid bonded sorbent). The column was washed with MeOH, the compound was eluted with methanolic ammonia (2.0 M), and the eluent was concentrated in vacuo to afford the title compound. MS m/z=(M+H)⁺: calculated 529.2, observed 529.5. ¹H NMR (500 MHz, DMSO-d6) δ 9.15 (d, J=1.9 Hz, 1H), 8.37-8.30 (m, 2H), 8.20-8.13 (m, 1H), 7.99 (s, 1H), 7.88 (d, J=1.9 Hz, 1H), 7.80 (s, 1H), 7.53 (t, J=9.0 Hz, 1H), 7.23 (s, 1H), 5.60 (s, 1H), 3.99 (s, 3H), 3.73 (dd, J=13.2, 5.7 Hz, 1H), 3.67 (dd, J=13.3, 6.4 Hz, 1H), 1.54 (s, 3H), 1.43-1.38 (m, 6H) ppm.

### Example 17-B

### N-2-(4-(2-aminopropan-2-yl)-6-(4-chloro-3-fluorophenyl)pyridin-2-yl)-2-hydroxypropyl)-1-methyl-3-(thiazol-4-yl)-1H-pyrazole-5-carboxamide (enantiomer-2)

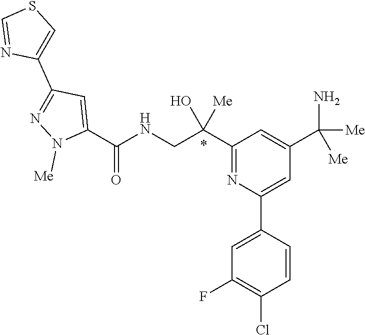 [SMILES: Cn1nc(-c2cscn2)cc1C(=O)NCC(C)(O)c1cc(C(C)(C)N)cc(-c2ccc(Cl)c(F)c2)n1]

A mixture of benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (Intermediate C-05-enantiomer-2, 0.090 g, 0.16 mmol), (4-chloro-3-fluorophenyl)boronic acid (33 mg, 0.19 mmol), tetrakis(triphenylphosphine)palladium(0) (9.1 mg, 7.9 μmol), potassium phosphate tribasic (0.050 g, 0.24 mmol), and 1,4-dioxane (1.6 mL) was stirred at 100° C. for 16 h. The mixture was cooled to room temperature, filtered through celite, and the filter pad was washed with EtOAc (1 mL×3). The filtrate was concentrated in vacuo and aqueous hydrochloric acid (12 M, 1.6 mL) was added. The mixture was stirred at 80° C. for 10 min, cooled to room temperature, diluted with water, and the pH was adjusted to >12 with aqueous NaOH (1.0 M). The mixture was extracted with DCM (15 mL×3) and the combined organic extracts were dried with anhydrous magnesium sulfate, filtered, and concentrated in vacuo. The residue was purified by preparative HPLC (C-18; 10-100% MeCN (with 0.1% TFA)/water (with 0.1% TFA)). The appropriate fractions were loaded onto a solid-phase extraction column (ISOLUTE® SCX-2; propylsulfonic acid bonded sorbent) that was washed with MeOH, which was discarded. The column was then eluted with methanolic ammonia (2.0 M) and the eluent was concentrated in vacuo to afford the title compound. MS m/z=(M+H)⁺: calculated 529.2, observed 529.5. ¹H NMR (500 MHz, DMSO-d6) δ 9.15 (d, J=1.9 Hz, 1H), 8.33 (t, J=5.8 Hz, 1H), 8.18 (dd, J=11.1, 1.8 Hz, 1H), 8.06-8.00 (m, 2H), 7.88 (d, J=1.9 Hz, 1H), 7.82 (s, 1H), 7.69 (t, J=8.1 Hz, 1H), 7.23 (s, 1H), 5.60 (s, 1H), 3.99 (s, 3H), 3.73 (dd, J=13.3, 5.8 Hz, 1H), 3.67 (dd, J=13.3, 6.4 Hz, 1H), 1.54 (s, 3H), 1.42-1.37 (m, 6H) ppm.

### Example 18-B

### N-2-(4-(2-aminopropan-2-yl)-6-(2,4-difluorophenyl)pyridin-2-yl)-2-hydroxypropyl)-1-methyl-3-(thiazol-4-yl)-1H-pyrazole-5-carboxamide (enantiomer-2)

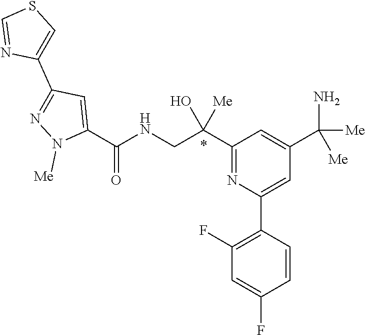 [SMILES: Cn1nc(-c2cscn2)cc1C(=O)NCC(C)(O)c1cc(C(C)(C)N)cc(-c2ccc(F)cc2F)n1]

A mixture of benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (Intermediate C-05-enantiomer-2, 67 mg, 0.12 mmol), (2,4-difluorophenyl)boronic acid (22 mg, 0.14 mmol), chloro(2-dicyclohexylphosphino-2′,4′,6′-triisopropyl-1,1′-biphenyl)[2-(2′-amino-1,1′-biphenyl)]palladium(II) (4.6 mg, 5.9 μmol), potassium phosphate tribasic (38 mg, 0.18 mmol), and 1,4-dioxane (1.2 mL) was stirred at 100° C. for 16 h. The mixture was cooled to room temperature, filtered through celite, and the filter pad was washed with EtOAc (1 mL×3). The filtrate was concentrated in vacuo and aqueous hydrochloric acid (12 M, 1.2 mL) was added. The mixture was stirred at 80° C. for 10 min, cooled to room temperature, diluted with water, and the pH was adjusted to >12 with aqueous NaOH (1.0 M). The mixture was extracted with DCM (15 mL×3) and the combined organic extracts were dried with anhydrous magnesium sulfate, filtered, and concentrated in vacuo. The residue was purified by preparative HPLC (C-18; 10-100% MeCN (with 0.1% TFA)/water (with 0.1% TFA)). The appropriate fractions were loaded onto a solid-phase extraction column (ISOLUTE® SCX-2; propylsulfonic acid bonded sorbent) that was washed with MeOH, which was discarded. The column was then eluted with methanolic ammonia (2.0 M) and the eluent was concentrated in vacuo to afford the title compound. MS m/z=(M+H)⁺: calculated 513.2, observed 513.5. ¹H NMR (500 MHz, DMSO-d6) δ 9.16 (d, J=1.9 Hz, 1H), 8.35 (t, J=6.1 Hz, 1H), 8.01 (q, J=8.9 Hz, 1H), 7.89 (d, J=1.9 Hz, 1H), 7.84 (d, J=1.3 Hz, 1H), 7.78 (s, 1H), 7.39-7.33 (m, 1H), 7.25-7.19 (m, 2H), 5.58 (s, 1H), 4.00 (s, 3H), 3.72 (dd, J=13.3, 5.7 Hz, 1H), 3.65 (dd, J=13.2, 6.4 Hz, 1H), 1.52 (s, 3H), 1.41-1.33 (m, 6H) ppm.

### Example 19-B

### N-2-(4-(2-aminopropan-2-yl)-6-(4-fluoro-2-methylphenyl)pyridin-2-yl)-2-hydroxypropyl)-1-methyl-3-(thiazol-4-yl)-1H-pyrazole-5-carboxamide (enantiomer-2)

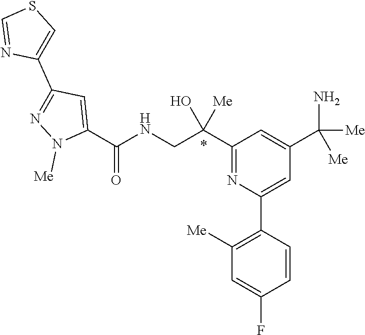 [SMILES: Cc1cc(F)ccc1-c1cc(C(C)(C)N)cc(C(C)(O)CNC(=O)c2cc(-c3cscn3)nn2C)n1]

A mixture of benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (Intermediate C-05-enantiomer-2, 64 mg, 0.11 mmol), (4-fluoro-2-methylphenyl)boronic acid (21 mg, 0.14 mmol), chloro(2-dicyclohexylphosphino-2′,4′,6′-triisopropyl-1,1′-biphenyl)[2-(2′-amino-1,1′-biphenyl)]palladium(II) (4.4 mg, 5.6 μmol), potassium phosphate tribasic (36 mg, 0.17 mmol), and 1,4-dioxane (1.1 mL) was stirred at 100° C. for 16 h. The mixture was cooled to room temperature, filtered through a pad of celite, which was washed with EtOAc (1 mL×3). The filtrate was concentrated in vacuo and was treated with aqueous hydrochloric acid (12 M, 1.1 mL). The mixture was stirred at 80° C. for 10 min, cooled to room temperature, diluted with water, and the pH was adjusted to >12 with aqueous NaOH (1.0 M). The mixture was extracted with DCM (15 mL×3) and the combined organic extracts were dried with anhydrous magnesium sulfate, filtered, and concentrated in vacuo. The residue was purified by preparative HPLC (C-18; 10-100% MeCN (with 0.1% TFA)/water (with 0.1% TFA)). The appropriate fractions were loaded onto a solid-phase extraction column (ISOLUTE® SCX-2; propylsulfonic acid bonded sorbent) that was washed with MeOH, which was discarded. The column was further eluted with methanolic ammonia (2.0 M) and the eluent was concentrated in vacuo to afford the title compound. MS m/z=(M+H)⁺: calculated 509.2, observed 509.5. ¹H NMR (500 MHz, DMSO-d6) δ 9.18-9.16 (m, 1H), 8.34 (t, J=5.9 Hz, 1H), 7.90-7.88 (m, 1H), 7.79 (s, 1H), 7.52 (s, 1H), 7.49 (dd, J=8.3, 6.3 Hz, 1H), 7.23 (s, 1H), 7.15 (dd, J=10.1, 2.4 Hz, 1H), 7.13-7.08 (m, 1H), 5.54 (s, 1H), 4.00 (s, 3H), 3.72 (dd, J=13.3, 5.7 Hz, 1H), 3.57 (dd, J=13.2, 6.2 Hz, 1H), 2.36 (s, 3H), 1.49 (s, 3H), 1.40-1.34 (m, 6H) ppm.

### Example 20-B

### N-2-(4-(2-aminopropan-2-yl)-6-(2,3,4-trifluorophenyl)pyridin-2-yl)-2-hydroxypropyl)-1-methyl-3-(thiazol-4-yl)-1H-pyrazole-5-carboxamide (enantiomer-2)

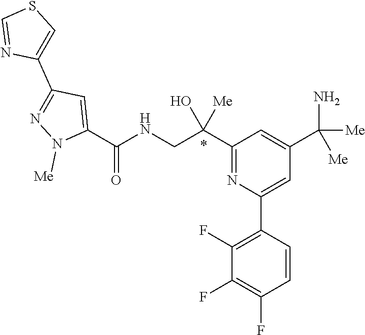 [SMILES: Cn1nc(-c2cscn2)cc1C(=O)NCC(C)(O)c1cc(C(C)(C)N)cc(-c2ccc(F)c(F)c2F)n1]

A mixture of benzyl (2-(2-chloro-6-(2-hydroxy-1-(1-methyl-3-(thiazol-4-yl)-1H-pyrazole-5-carboxamido)propan-2-yl)pyridin-4-yl)propan-2-yl)carbamate (Intermediate C-05-enantiomer-2, 62 mg, 0.11 mmol), (2,3,4-trifluorophenyl)boronic acid (23 mg, 0.13 mmol), chloro(2-dicyclohexylphosphino-2′,4′,6′-triisopropyl-1,1′-biphenyl)[2-(2′-amino-1,1′-biphenyl)]palladium(II) (4.3 mg, 5.5 μmol), potassium phosphate tribasic (35 mg, 0.16 mmol), and 1,4-dioxane (1.1 mL) was stirred at 100° C. for 16 h. The mixture was cooled to room temperature, filtered through celite, and the filter pad was washed with EtOAc (1 mL×3). The filtrate was concentrated in vacuo and aqueous hydrochloric acid (12 M, 1.1 mL) was added. The mixture was stirred at 80° C. for 10 min, cooled to room temperature, diluted with water, and the pH was adjusted to >12 with aqueous NaOH (1.0 M). The mixture was extracted with DCM (15 mL×3) and the combined organic extracts were dried with anhydrous magnesium sulfate, filtered, and concentrated in vacuo. The residue was purified by preparative HPLC (C-18; 10-100% MeCN (with 0.1% TFA)/water (with 0.1% TFA)). The appropriate fractions were loaded onto a solid-phase extraction column (ISOLUTE® SCX-2; propylsulfonic acid bonded sorbent) that was washed with MeOH, which was discarded. The column was further eluted with methanolic ammonia (2.0 M) and the eluent was concentrated in vacuo to afford the title compound. MS m/z=(M+H)⁺: calculated 531.2, observed 531.5. ¹H NMR (500 MHz, DMSO-d6) δ 9.15 (d, J=1.9 Hz, 1H), 8.35 (t, J=6.0 Hz, 1H), 7.89 (d, J=1.9 Hz, 1H), 7.88 (d, J=1.4 Hz, 1H), 7.84-7.77 (m, 2H), 7.44 (q, J=8.7, 8.1 Hz, 1H), 7.20 (s, 1H), 5.61 (s, 1H), 3.99 (s, 3H), 3.71 (dd, J=13.3, 5.8 Hz, 1H), 3.65 (dd, J=13.3, 6.4 Hz, 1H), 1.52 (s, 3H), 1.42-1.35 (m, 6H) ppm.

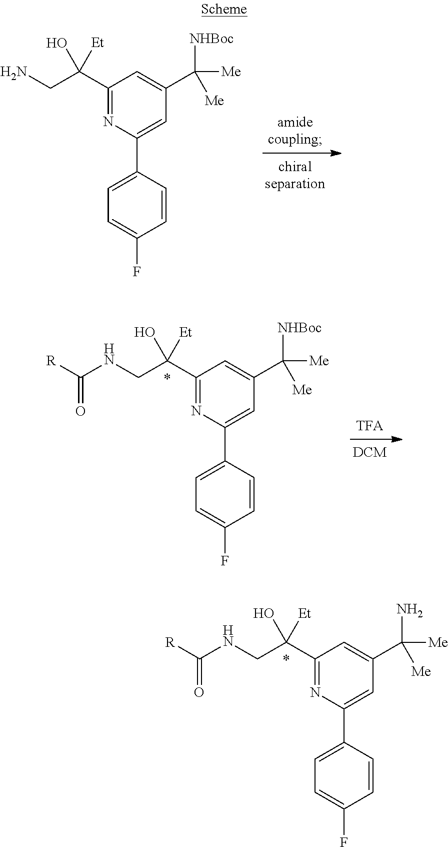

### Examples 21-A and 21-B

### N-2-(4-(2-aminopropan-2-yl)-6-(4-fluorophenyl)pyridin-2-yl)-2-hydroxybutyl)-1-methyl-3-(thiazol-4-yl)-1H-pyrazole-5-carboxamide (enantiomer-1 and enantiomer-2)

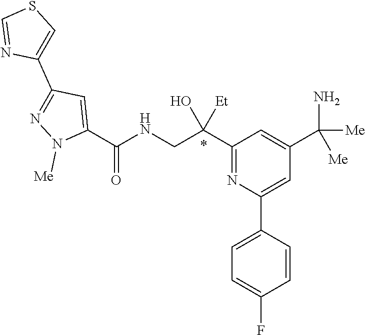 [SMILES: CCC(O)(CNC(=O)c1cc(-c2cscn2)nn1C)c1cc(C(C)(C)N)cc(-c2ccc(F)cc2)n1]

### Step 1: tert-butyl (2-(2-(4-fluorophenyl)-6-(2-hydroxy-1-(1-methyl-3-(thiazol-4-yl)-1H-pyrazole-5-carboxamido)butan-2-yl)pyridin-4-yl)propan-2-yl)carbamate (enantiomer-1 and enantiomer-2)

A mixture of tert-butyl (2-(2-(1-amino-2-hydroxybutan-2-yl)-6-(4-fluorophenyl)pyridin-4-yl)propan-2-yl)carbamate (Intermediate D-01, 0.13 g, 0.32 mmol), 1-methyl-3-(thiazol-4-yl)-1H-pyrazole-5-carboxylic acid (Intermediate A-05, 0.060 g, 0.29 mmol), 1-propanephosphonic anhydride (50 wt % in EtOAc, 0.18 g, 0.57 mmol), DIPEA (0.15 mL, 0.86 mmol), and DMF (5.0 mL) was stirred at 25° C. for 12 h. Water (5 mL) was added and the mixture was extracted with EtOAc (5 mL×3). The combined organic extracts were washed with brine (10 mL) and concentrated in vacuo. The residue was purified by preparative TLC (50% EtOAc/petroleum ether) and then further purified by SFC (Chiralpak IC-3, 150×4.6 mm, 3 μm; 40% 2-PrOH (with 0.05% diethylamine)/CO₂) to afford the title compounds. The first eluting isomer was designated enantiomer-1 and the second eluting isomer was designated enantiomer-2.

### Step 2-A: N-2-(4-(2-aminopropan-2-yl)-6-(4-fluorophenyl)pyridin-2-yl)-2-hydroxybutyl)-1-methyl-3-(thiazol-4-yl)-1H-pyrazole-5-carboxamide (enantiomer-1=EXAMPLE 21-A)

A mixture of tert-butyl (2-(2-(4-fluorophenyl)-6-(2-hydroxy-1-(1-methyl-3-(thiazol-4-yl)-1H-pyrazole-5-carboxamido)butan-2-yl)pyridin-4-yl)propan-2-yl)carbamate (enantiomer-1, 38 mg, 0.062 mmol), DCM (2.0 mL) and TFA (1.0 mL) was stirred at 25° C. for 0.5 h. The mixture was concentrated in vacuo and the residue was purified by preparative HPLC (C-18; 25-55% MeCN/water (with 0.1% TFA)) to afford the title compound. MS m/z=(M+H)⁺: calculated 509.2, observed 509.2. ¹H NMR (500 MHz, CD₃OD) δ 9.04 (d, J=2.4 Hz, 1H), 8.17-8.09 (m, 2H), 7.81-7.72 (m, 3H), 7.22-7.13 (m, 2H), 6.96 (s, 1H), 4.04 (d, J=13.4 Hz, 1H), 3.97 (s, 3H), 3.71 (d, J=13.4 Hz, 1H), 2.29-2.18 (m, 1H), 2.03-1.94 (m, 1H), 1.77 (s, 6H), 0.81 (t, J=7.3 Hz, 3H) ppm.

### Step 2-B: N-2-(4-(2-aminopropan-2-yl)-6-(4-fluorophenyl)pyridin-2-yl)-2-hydroxybutyl)-1-methyl-3-(thiazol-4-yl)-1H-pyrazole-5-carboxamide (enantiomer-2=EXAMPLE 21-B)

A mixture of tert-butyl (2-(2-(4-fluorophenyl)-6-(2-hydroxy-1-(1-methyl-3-(thiazol-4-yl)-1H-pyrazole-5-carboxamido)butan-2-yl)pyridin-4-yl)propan-2-yl)carbamate (enantiomer-2, 55 mg, 0.090 mmol), DCM (2.0 mL) and TFA (1.0 mL) was stirred at 25° C. for 0.5 h. The mixture was concentrated in vacuo and the residue was purified by preparative HPLC (C-18; 25-55% MeCN/water (with 0.1% TFA)) to afford the title compound. MS m/z=(M+H)⁺: calculated 509.2, observed 509.2. ¹H NMR (500 MHz, CD₃OD) δ 9.04 (d, J=1.8 Hz, 1H), 8.22-8.08 (m, 2H), 7.86-7.71 (m, 3H), 7.23-7.10 (m, 2H), 6.98-6.89 (m, 1H), 4.04 (d, J=13.4 Hz, 1H), 3.97 (s, 3H), 3.71 (d, J=13.4 Hz, 1H), 2.25 (d, J=7.3, 14.2 Hz, 1H), 2.03-1.95 (m, 1H), 1.77 (s, 6H), 0.81 (t, J=7.3 Hz, 3H) ppm.

### Example 22-A

### N-2-(4-(2-aminopropan-2-yl)-6-(4-fluorophenyl)pyridin-2-yl)-2-cyclopropyl-2-hydroxyethyl)-4-methyl-2-(pyrimidin-2-yl)thiazole-5-carboxamide(enantiomer-1)

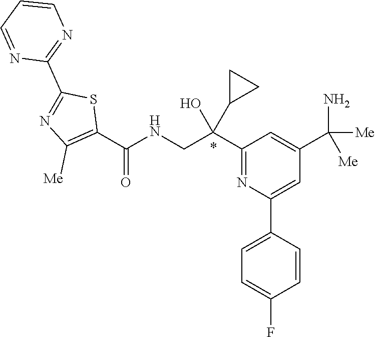 [SMILES: Cc1nc(-c2ncccn2)sc1C(=O)NCC(O)(c1cc(C(C)(C)N)cc(-c2ccc(F)cc2)n1)C1CC1]

### Step 1: benzyl (2-(2-(1-cyclopropyl-1-hydroxy-2-(4-methyl-2-(pyrimidin-2-yl)thiazole-5-carboxamido)ethyl)-6-(4-fluorophenyl)pyridin-4-yl)propan-2-yl)carbamate (enantiomer-1)

A mixture of benzyl (2-(2-(2-amino-1-cyclopropyl-1-hydroxyethyl)-6-(4-fluorophenyl)pyridin-4-yl)propan-2-yl)carbamate (Intermediate E-03-enantiomer-1, 37 mg, 0.068 mmol), 4-methyl-2-(pyrimidin-2-yl)thiazole-5-carboxylic acid (Intermediate A-12, 18 mg, 0.082 mmol), HATU (26 mg, 0.068 mmol), N-methylmorpholine (0.023 mL, 0.21 mmol), and DMSO (0.34 mL) was stirred at room temperature for 1 h. Aqueous sodium bicarbonate (saturated) was added and the mixture was extracted with EtOAc (3×). The combined organic extracts were dried with anhydrous magnesium sulfate, filtered, and concentrated in vacuo to afford the title compound.

### Step 2: N-2-(4-(2-aminopropan-2-yl)-6-(4-fluorophenyl)pyridin-2-yl)-2-cyclopropyl-2-hydroxyethyl)-4-methyl-2-(pyrimidin-2-yl)thiazole-5-carboxamide (enantiomer-1)

A mixture of benzyl (2-(2-(1-cyclopropyl-1-hydroxy-2-(4-methyl-2-(pyrimidin-2-yl)thiazole-5-carboxamido)ethyl)-6-(4-fluorophenyl)pyridin-4-yl)propan-2-yl)carbamate (enantiomer-1, 46 mg, 0.068 mmol) and aqueous hydrochloric acid (12 M, 0.68 mL) was stirred at 80° C. for 10 min. The mixture was cooled to room temperature, concentrated in vacuo, and the residue was purified by preparative HPLC (C-18; 5-95% MeCN (with 0.1% TFA)/water (with 0.1% TFA)). The appropriate fractions were loaded onto a solid-phase extraction column (ISOLUTE® SCX-2; propylsulfonic acid bonded sorbent) that was washed with MeOH, which was discarded. The column was further eluted with methanolic ammonia (2.0 M) and the eluent was concentrated in vacuo to afford the title compound. MS m/z=(M+H)⁺: calculated 533.2, observed 533.2. ¹H NMR (500 MHz, CDCl₃) δ 8.81 (d, J=4.9 Hz, 2H), 7.99-7.93 (m, 2H), 7.77-7.75 (m, 1H), 7.65-7.63 (m, 1H), 7.31 (t, J=4.9 Hz, 1H), 7.16 (t, J=8.6 Hz, 2H), 6.36-6.29 (m, 1H), 5.53 (s, 1H), 4.32 (dd, J=13.7, 7.7 Hz, 1H), 3.76 (dd, J=13.8, 3.8 Hz, 1H), 2.57 (s, 3H), 1.52 (s, 6H), 1.31-1.24 (m, 1H), 0.74-0.66 (m, 1H), 0.57-0.49 (m, 1H), 0.41-0.33 (m, 1H), 0.30-0.22 (m, 1H) ppm.

### Example 22-B

### N-2-(4-(2-aminopropan-2-yl)-6-(4-fluorophenyl)pyridin-2-yl)-2-cyclopropyl-2-hydroxyethyl)-4-methyl-2-(pyrimidin-2-yl)thiazole-5-carboxamide (enantiomer-2)

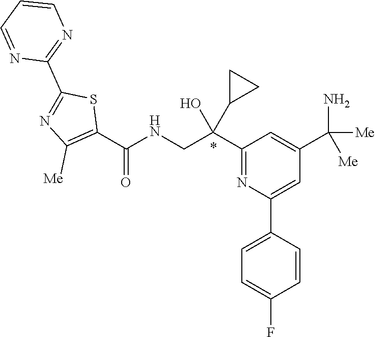 [SMILES: Cc1nc(-c2ncccn2)sc1C(=O)NCC(O)(c1cc(C(C)(C)N)cc(-c2ccc(F)cc2)n1)C1CC1]

### Step 1: benzyl (2-(2-(1-cyclopropyl-1-hydroxy-2-(4-methyl-2-(pyrimidin-2-yl)thiazole-5-carboxamido)ethyl)-6-(4-fluorophenyl)pyridin-4-yl)propan-2-yl)carbamate (enantiomer-2)

A mixture of benzyl (2-(2-(2-amino-1-cyclopropyl-1-hydroxyethyl)-6-(4-fluorophenyl)pyridin-4-yl)propan-2-yl)carbamate (Intermediate E-03-enantiomer-2, 35 mg, 0.066 mmol), 4-methyl-2-(pyrimidin-2-yl)thiazole-5-carboxylic acid (Intermediate A-12, 17 mg, 0.079 mmol), HATU (25 mg, 0.066 mmol), N-methylmorpholine (0.022 mL, 0.20 mmol), and DMSO (0.33 mL) was stirred at room temperature for 1 h. Aqueous sodium bicarbonate (saturated) was added and the mixture was extracted with EtOAc (3×). The combined organic extracts were dried with anhydrous magnesium sulfate, filtered, and concentrated in vacuo to afford the title compound.

### Step 2: N-2-(4-(2-aminopropan-2-yl)-6-(4-fluorophenyl)pyridin-2-yl)-2-cyclopropyl-2-hydroxyethyl)-4-methyl-2-(pyrimidin-2-yl)thiazole-5-carboxamide (enantiomer-2)

A mixture of benzyl (2-(2-(1-cyclopropyl-1-hydroxy-2-(4-methyl-2-(pyrimidin-2-yl)thiazole-5-carboxamido)ethyl)-6-(4-fluorophenyl)pyridin-4-yl)propan-2-yl)carbamate (enantiomer-2, 46 mg, 0.068 mmol) and aqueous hydrochloric acid (12 M, 0.68 mL) was stirred at 80° C. for 10 min. The mixture was cooled to room temperature, concentrated in vacuo, and the residue was purified by preparative HPLC (C-18; 5-95% MeCN (with 0.1% TFA)/water (with 0.1% TFA)). The appropriate fractions were loaded onto a solid-phase extraction column (ISOLUTE® SCX-2; propylsulfonic acid bonded sorbent) that was washed with MeOH, which was discarded. The column was further eluted with methanolic ammonia (2.0 M) and the eluent was concentrated in vacuo to afford the title compound. MS m/z=(M+H)⁺: calculated 533.2, observed 533.2. ¹H NMR (500 MHz, CDCl₃) δ 8.81 (d, J=4.8 Hz, 2H), 8.01-7.92 (m, 2H), 7.76 (s, 1H), 7.64 (s, 1H), 7.31 (t, J=4.9 Hz, 1H), 7.16 (t, J=8.2 Hz, 2H), 6.35-6.28 (m, 1H), 5.53 (s, 1H), 4.32 (dd, J=13.7, 7.7 Hz, 1H), 3.76 (dd, J=13.8, 3.3 Hz, 1H), 2.57 (s, 3H), 1.52 (s, 6H), 1.33-1.21 (m, 1H), 0.74-0.66 (m, 1H), 0.57-0.49 (m, 1H), 0.41-0.34 (m, 1H), 0.30-0.23 (m, 1H) ppm.

### Example 23-B

### N-2-(4-(2-aminopropan-2-yl)-6-(4-fluorophenyl)pyridin-2-yl)-2-cyclopropyl-2-hydroxyethyl)-1-methyl-3-(thiazol-4-yl)-1H-pyrazole-5-carboxamide (enantiomer-2)

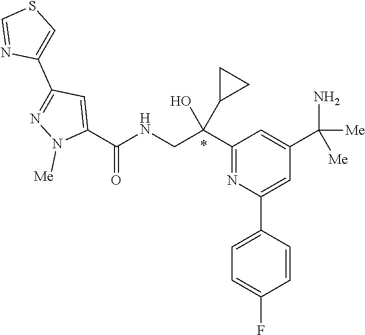 [SMILES: Cn1nc(-c2cscn2)cc1C(=O)NCC(O)(c1cc(C(C)(C)N)cc(-c2ccc(F)cc2)n1)C1CC1]

A mixture of 1-methyl-3-(thiazol-4-yl)-1H-pyrazole-5-carboxylic acid (Intermediate A-05, 33 mg, 0.16 mmol), 2-amino-1-(4-(2-aminopropan-2-yl)-6-(4-fluorophenyl)pyridin-2-yl)-1-cyclopropylethan-1-ol (Intermediate E-04-enantiomer-2, 0.10 g, 0.23 mmol), HATU (0.10 g, 0.27 mmol), triethylamine (0.095 mL, 0.68 mmol), and DMF (1.0 mL) was stirred at room temperature for 15 min. The mixture was purified by preparative HPLC (C-18; 10-95% MeCN (with 0.1% TFA)/water (with 0.1% TFA)). The appropriate fractions were extracted with EtOAc and the organic extracts were washed with aqueous sodium bicarbonate and brine, dried with anhydrous magnesium sulfate, filtered, and concentrated in vacuo to afford the title compound. MS m/z=(M+H)⁺: calculated 521.2, observed 521.2. ¹H NMR (500 MHz, DMSO-d6) δ 9.15 (d, J=1.9 Hz, 1H), 8.33 (t, J=5.6 Hz, 1H), 8.19 (dd, J=8.7, 5.6 Hz, 2H), 7.95-7.91 (m, 1H), 7.88 (d, J=1.9 Hz, 1H), 7.72-7.68 (m, 1H), 7.31 (t, J=8.8 Hz, 2H), 7.16 (s, 1H), 5.32 (s, 1H), 3.98 (s, 3H), 3.93 (dd, J=13.5, 6.4 Hz, 1H), 3.86 (dd, J=13.4, 5.3 Hz, 1H), 1.56-1.48 (m, 1H), 1.41 (s, 6H), 0.60-0.52 (m, 1H), 0.43-0.35 (m, 1H), 0.32-0.23 (m, 1H), 0.16-0.07 (m, 1H) ppm.

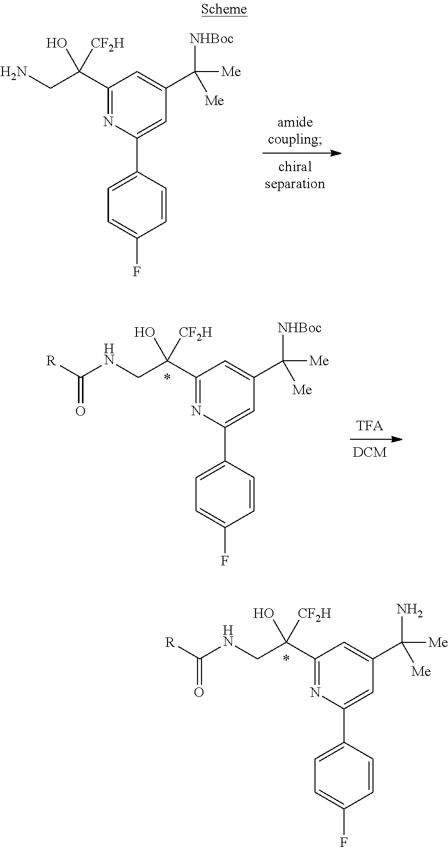

### Examples 24-A and 24-B

### N-2-(4-(2-aminopropan-2-yl)-6-(4-fluorophenyl)pyridin-2-yl)-3,3-difluoro-2-hydroxypropyl)-1-methyl-3-(thiazol-4-yl)-1H-pyrazole-5-carboxamide (enantiomer-1 and enantiomer-2)

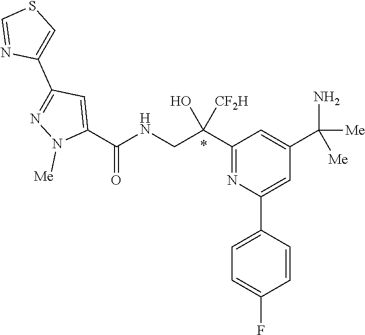 [SMILES: Cn1nc(-c2cscn2)cc1C(=O)NCC(O)(c1cc(C(C)(C)N)cc(-c2ccc(F)cc2)n1)C(F)F]

### Step 1: tert-butyl (2-(2-(1,1-difluoro-2-hydroxy-3-(1-methyl-3-(thiazol-4-yl)-1H-pyrazole-5-carboxamido)propan-2-yl)-6-(4-fluorophenyl)pyridin-4-yl)propan-2-yl)carbamate (enantiomer-1 and enantiomer-2)

A mixture of tert-butyl (2-(2-(3-amino-1,1-difluoro-2-hydroxypropan-2-yl)-6-(4-fluorophenyl)pyridin-4-yl)propan-2-yl)carbamate (Intermediate F-01, 0.080 g, 0.15 mmol), 1-methyl-3-(thiazol-4-yl)-1H-pyrazole-5-carboxylic acid (Intermediate A-05, 31 mg, 0.15 mmol), 1-propanephosphonic anhydride (50 wt % in EtOAc, 0.14 g, 0.22 mmol), DIPEA (0.076 mL, 0.44 mmol), and DMF (5.0 mL) was stirred at 25° C. for 1 h. Water (10 mL) was added and the mixture was extracted with EtOAc (10 mL×3). The combined organic extracts were washed with brine (10 mL×3), dried with anhydrous sodium sulfate, filtered, and concentrated in vacuo. The residue was purified by preparative TLC (50% EtOAc/petroleum ether), which was further purified by SFC (Chiralcel® OJ-3, 100×4.6 mm, 3 μm; 5-40% EtOH (with 0.05% diethylamine modifier)/CO₂) to afford the title compounds. The first eluting isomer was designated enantiomer-1 and the second eluting isomer was designated enantiomer-2.

### Step 2-A: N-2-(4-(2-aminopropan-2-yl)-6-(4-fluorophenyl)pyridin-2-yl)-3,3-difluoro-2-hydroxypropyl)-1-methyl-3-(thiazol-4-yl)-1H-pyrazole-5-carboxamide (enantiomer-1=EXAMPLE 24-A)

A mixture of tert-butyl (2-(2-(1,1-difluoro-2-hydroxy-3-(1-methyl-3-(thiazol-4-yl)-1H-pyrazole-5-carboxamido)propan-2-yl)-6-(4-fluorophenyl)pyridin-4-yl)propan-2-yl)carbamate (enantiomer-1, 36 mg, 0.057 mmol), DCM (5.0 mL) and TFA (1.0 mL) was stirred at 25° C. for 0.5 h. The mixture was concentrated in vacuo and the residue was purified by preparative HPLC (C-18; 20-40% MeCN/water (with 0.1% TFA)) to afford the title compound. MS m/z=(M+H)⁺: calculated 531.2, observed 531.1. ¹H NMR (400 MHz, CD₃OD) δ 9.05 (s, 1H), 8.17-8.04 (m, 2H), 7.91-7.72 (m, 3H), 7.16 (t, J=8.8 Hz, 2H), 6.97 (s, 1H), 6.79-6.37 (m, 1H), 4.21-4.09 (m, 1H), 3.93-3.88 (m, 4H), 1.76 (d, J=3.8 Hz, 6H) ppm.

### Step 2-B: N-2-(4-(2-aminopropan-2-yl)-6-(4-fluorophenyl)pyridin-2-yl)-3,3-difluoro-2-hydroxypropyl)-1-methyl-3-(thiazol-4-yl)-1H-pyrazole-5-carboxamide (enantiomer-2=EXAMPLE 24-B)

A mixture of tert-butyl (2-(2-(1,1-difluoro-2-hydroxy-3-(1-methyl-3-(thiazol-4-yl)-1H-pyrazole-5-carboxamido)propan-2-yl)-6-(4-fluorophenyl)pyridin-4-yl)propan-2-yl)carbamate (enantiomer-2, 0.040 g, 0.063 mmol), DCM (5.0 mL) and TFA (1.0 mL) was stirred at 25° C. for 0.5 h. The mixture was concentrated in vacuo and the residue was purified by preparative HPLC (C-18; 20-40% MeCN/water (with 0.1% TFA)) to afford the title compound. MS m/z=(M+H)⁺: calculated 531.2, observed 531.1. ¹H NMR (400 MHz, CD₃OD) δ 9.05 (s, 1H), 8.16-8.06 (m, 2H), 7.86-7.83 (m, 2H), 7.78 (d, J=1.5 Hz, 1H), 7.21-7.09 (m, 2H), 6.97 (s, 1H), 6.73-6.44 (m, 1H), 4.18-4.12 (m, 1H), 3.94-3.87 (m, 4H), 1.76 (d, J=4.3 Hz, 6H) ppm.

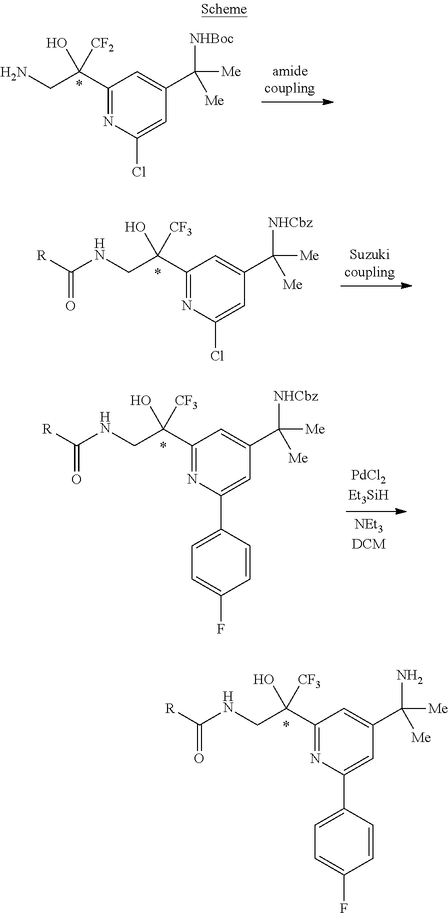

### Example 25-A

### N-2-(4-(2-aminopropan-2-yl)-6-(4-fluorophenyl)pyridin-2-yl)-3,3,3-trifluoro-2-hydroxypropyl)-4-methyl-2-(pyrimidin-2-yl)thiazole-5-carboxamide (enantiomer-1)

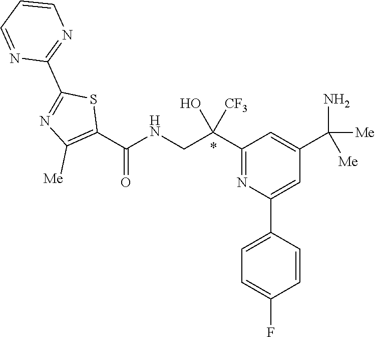 [SMILES: Cc1nc(-c2ncccn2)sc1C(=O)NCC(O)(c1cc(C(C)(C)N)cc(-c2ccc(F)cc2)n1)C(F)(F)F]

### Step 1: benzyl (2-(2-chloro-6-(1,1,1-trifluoro-2-hydroxy-3-(4-methyl-2-(pyrimidin-2-yl)thiazole-5-carboxamido)propan-2-yl)pyridin-4-yl)propan-2-yl)carbamate (enantiomer-1)

A mixture of benzyl (2-(2-(3-amino-1,1,1-trifluoro-2-hydroxypropan-2-yl)-6-chloropyridin-4-yl)propan-2-yl)carbamate (Intermediate G-01-enantiomer-1, 0.050 g, 0.12 mmol), 4-methyl-2-(pyrimidin-2-yl)thiazole-5-carboxylic acid (Intermediate A-12, 31 mg, 0.14 mmol), HATU (44 mg, 0.12 mmol), 4-methylmorpholine N-oxide (0.038 mL, 0.35 mmol), and DMSO (0.58 mL) was stirred at room temperature for 1 h. Aqueous sodium bicarbonate (saturated) was added and the mixture was extracted with EtOAc (3×). The combined organic extracts were dried with anhydrous magnesium sulfate, filtered, and concentrated in vacuo to afford the title compound.

### Step 2: benzyl (2-(2-(4-fluorophenyl)-6-(1,1,1-trifluoro-2-hydroxy-3-(4-methyl-2-(pyrimidin-2-yl)thiazole-5-carboxamido)propan-2-yl)pyridin-4-yl)propan-2-yl)carbamate (enantiomer-1)

A mixture of benzyl (2-(2-chloro-6-(1,1,1-trifluoro-2-hydroxy-3-(4-methyl-2-(pyrimidin-2-yl)thiazole-5-carboxamido)propan-2-yl)pyridin-4-yl)propan-2-yl)carbamate (enantiomer-1, 66 mg, 0.11 mmol), (4-fluorophenyl)boronic acid (18 mg, 0.13 mmol), chloro(2-dicyclohexylphosphino-2′,4′,6′-triisopropyl-1,1′-biphenyl)[2-(2′-amino-1,1′-biphenyl)]palladium(II) (8.2 mg, 0.010 mmol), aqueous potassium phosphate tribasic (1.0 M, 0.21 mL, 0.21 mmol), and THF (0.52 mL) was stirred at 100° C. for 2 h. The mixture was cooled to room temperature, water was added, and the mixture was extracted with EtOAc (3×). The combined organic extracts were dried with anhydrous magnesium sulfate, filtered, and concentrated in vacuo. The residue was purified by silica gel chromatography (0-100% EtOAc/hexanes) to afford the title compound.

### Step 3: N-2-(4-(2-aminopropan-2-yl)-6-(4-fluorophenyl)pyridin-2-yl)-3,3,3-trifluoro-2-hydroxypropyl)-4-methyl-2-(pyrimidin-2-yl)thiazole-5-carboxamide (enantiomer-1)

A mixture of benzyl (2-(2-(4-fluorophenyl)-6-(1,1,1-trifluoro-2-hydroxy-3-(4-methyl-2-(pyrimidin-2-yl)thiazole-5-carboxamido)propan-2-yl)pyridin-4-yl)propan-2-yl)carbamate (enantiomer-1, 73 mg, 0.11 mmol), palladium(II) chloride (5.6 mg, 0.032 mmol), triethylamine (0.059 mL, 0.42 mmol), triethylsilane (0.084 mL, 0.53 mmol), and DCM (0.53 mL) was stirred at room temperature for 30 min. The mixture was filtered through a pad of celite that was eluted with EtOH, and the filtrate was concentrated in vacuo. The residue was purified by preparative HPLC (C-18; 5-95% MeCN (with 0.1% TFA)/water (with 0.1% TFA)). The appropriate fractions were loaded onto a solid-phase extraction column (ISOLUTE® SCX-2; propylsulfonic acid bonded sorbent) that was washed with MeOH, which was discarded. The column was further eluted with methanolic ammonia (2.0 M) and the eluent was concentrated in vacuo to afford the title compound. MS m/z=(M+H)⁺: calculated 561.2, observed 561.1. ¹H NMR (500 MHz, CDCl₃) δ 8.81 (d, J=4.8 Hz, 2H), 8.01-7.92 (m, 2H), 7.91 (s, 1H), 7.78 (s, 1H), 7.32 (t, J=4.8 Hz, 1H), 7.17 (t, J=8.5 Hz, 2H), 6.74 (s, 1H), 6.22-6.13 (m, 1H), 4.76 (dd, J=13.9, 8.8 Hz, 1H), 3.87 (dd, J=14.1, 3.0 Hz, 1H), 2.56 (s, 3H), 1.52 (s, 6H) ppm.

### Example 25-B

### N-2-(4-(2-aminopropan-2-yl)-6-(4-fluorophenyl)pyridin-2-yl)-3,3,3-trifluoro-2-hydroxypropyl)-4-methyl-2-(pyrimidin-2-yl)thiazole-5-carboxamide (enantiomer-2)

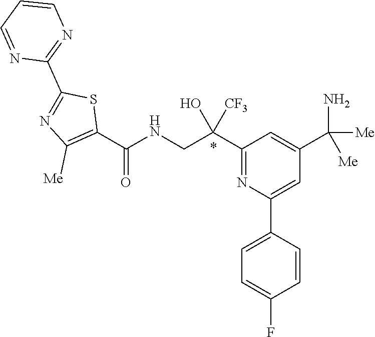 [SMILES: Cc1nc(-c2ncccn2)sc1C(=O)NCC(O)(c1cc(C(C)(C)N)cc(-c2ccc(F)cc2)n1)C(F)(F)F]

### Step 1: benzyl (2-(2-chloro-6-(1,1,1-trifluoro-2-hydroxy-3-(4-methyl-2-(pyrimidin-2-yl)thiazole-5-carboxamido)propan-2-yl)pyridin-4-yl)propan-2-yl)carbamate (enantiomer-2)

A mixture of benzyl (2-(2-(3-amino-1,1,1-trifluoro-2-hydroxypropan-2-yl)-6-chloropyridin-4-yl)propan-2-yl)carbamate (Intermediate G-01-enantiomer-2, 0.050 g, 0.12 mmol), 4-methyl-2-(pyrimidin-2-yl)thiazole-5-carboxylic acid (Intermediate A-12, 31 mg, 0.14 mmol), HATU (44 mg, 0.12 mmol), 4-methylmorpholine N-oxide (0.038 mL, 0.35 mmol), and DMSO (0.58 mL) was stirred at room temperature for 1 h. Aqueous sodium bicarbonate (saturated) was added and the mixture was extracted with EtOAc (3×). The combined organic extracts were dried with anhydrous magnesium sulfate, filtered, and concentrated in vacuo to afford the title compound.

### Step 2: benzyl (2-(2-(4-fluorophenyl)-6-(1,1,1-trifluoro-2-hydroxy-3-(4-methyl-2-(pyrimidin-2-yl)thiazole-5-carboxamido)propan-2-yl)pyridin-4-yl)propan-2-yl)carbamate (enantiomer-2)

A mixture of benzyl (2-(2-chloro-6-(1,1,1-trifluoro-2-hydroxy-3-(4-methyl-2-(pyrimidin-2-yl)thiazole-5-carboxamido)propan-2-yl)pyridin-4-yl)propan-2-yl)carbamate (enantiomer-2, 66 mg, 0.11 mmol), (4-fluorophenyl)boronic acid (18 mg, 0.13 mmol), chloro(2-dicyclohexylphosphino-2′,4′,6′-triisopropyl-1,1′-biphenyl)[2-(2′-amino-1,1′-biphenyl)]palladium(II) (8.2 mg, 0.010 mmol), aqueous potassium phosphate tribasic (1.0 M, 0.21 mL, 0.21 mmol), and THF (0.52 mL) was stirred at 100° C. for 2 h. The mixture was cooled to room temperature, water was added, and the mixture was extracted with EtOAc (3×). The combined organic extracts were dried with anhydrous magnesium sulfate, filtered, and concentrated in vacuo. The residue was purified by silica gel chromatography (0-100% EtOAc/hexanes) to afford the title compound.

### Step 3: N-2-(4-(2-aminopropan-2-yl)-6-(4-fluorophenyl)pyridin-2-yl)-3,3,3-trifluoro-2-hydroxypropyl)-4-methyl-2-(pyrimidin-2-yl)thiazole-5-carboxamide (enantiomer-2)

A mixture of benzyl (2-(2-(4-fluorophenyl)-6-(1,1,1-trifluoro-2-hydroxy-3-(4-methyl-2-(pyrimidin-2-yl)thiazole-5-carboxamido)propan-2-yl)pyridin-4-yl)propan-2-yl)carbamate (enantiomer-2, 66 mg, 0.095 mmol), palladium(II) chloride (5.1 mg, 0.029 mmol), triethylamine (0.053 mL, 0.38 mmol), triethylsilane (0.076 mL, 0.48 mmol), and DCM (0.48 mL) was stirred at room temperature for 30 min. The mixture was filtered through celite, the filter pad was eluted with EtOH, and the filtrate was concentrated in vacuo. The residue was purified by HPLC (C-18; 5-95% MeCN (with 0.1% TFA)/water (with 0.1% TFA)). The appropriate fractions were loaded onto a solid-phase extraction column (ISOLUTE® SCX-2; propylsulfonic acid bonded sorbent) that was washed with MeOH, which was discarded. The column was further eluted with methanolic ammonia (2.0 M) and the eluent was concentrated in vacuo to afford the title compound. MS m/z=(M+H)⁺: calculated 561.2, observed 561.1. ¹H NMR (500 MHz, CDCl₃) δ 8.81 (d, J=4.7 Hz, 2H), 8.01-7.92 (m, 2H), 7.91 (s, 1H), 7.78 (s, 1H), 7.32 (t, J=4.7 Hz, 1H), 7.17 (t, J=8.2 Hz, 2H), 6.74 (s, 1H), 6.17 (d, J=7.6 Hz, 1H), 4.76 (dd, J=13.9, 8.9 Hz, 1H), 3.87 (d, J=14.0 Hz, 1H), 2.56 (s, 3H), 1.52 (s, 6H) ppm.

### Example 26-A

### N-2-(4-(2-aminopropan-2-yl)-6-(4-fluorophenyl)pyridin-2-yl)-3,3,3-trifluoro-2-hydroxypropyl)-1-methyl-3-(thiazol-4-yl)-1H-pyrazole-5-carboxamide (enantiomer-1)

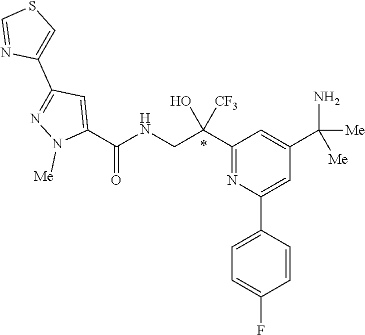 [SMILES: Cn1nc(-c2cscn2)cc1C(=O)NCC(O)(c1cc(C(C)(C)N)cc(-c2ccc(F)cc2)n1)C(F)(F)F]

A mixture of 1-methyl-3-(thiazol-4-yl)-1H-pyrazole-5-carboxylic acid (Intermediate A-05, 3.1 mg, 15 μmol), 3-amino-2-(4-(2-aminopropan-2-yl)-6-(4-fluorophenyl)pyridin-2-yl)-1,1,1-trifluoropropan-2-ol (Intermediate G-02-enantiomer-1, 0.010 g, 21 μmol) HATU (9.8 mg, 26 μmol), triethylamine (9.0 μL, 64 μmol) and DMF (0.5 mL) was stirred at room temperature for 30 min. The mixture was purified by preparative HPLC (C-18; 10-95% MeCN (with 0.05% TFA)/water (with 0.05% TFA)) to afford the title compound. MS m/z=(M+H)⁺: calculated 549.2, observed 549.2. ¹H NMR (500 MHz, DMSO-d6) δ 9.14 (d, J=1.9 Hz, 1H), 8.61 (s, 3H), 8.52 (t, J=6.1 Hz, 1H), 8.24 (dd, J=8.8, 5.6 Hz, 2H), 8.06-8.02 (m, 1H), 7.89 (d, J=1.9 Hz, 1H), 7.86 (s, 1H), 7.39 (t, J=8.8 Hz, 2H), 7.26 (s, 1H), 7.19 (s, 1H), 4.25 (dd, J=13.9, 6.5 Hz, 1H), 4.14 (dd, J=14.0, 5.7 Hz, 1H), 3.98 (s, 3H), 1.65 (s, 6H) ppm.

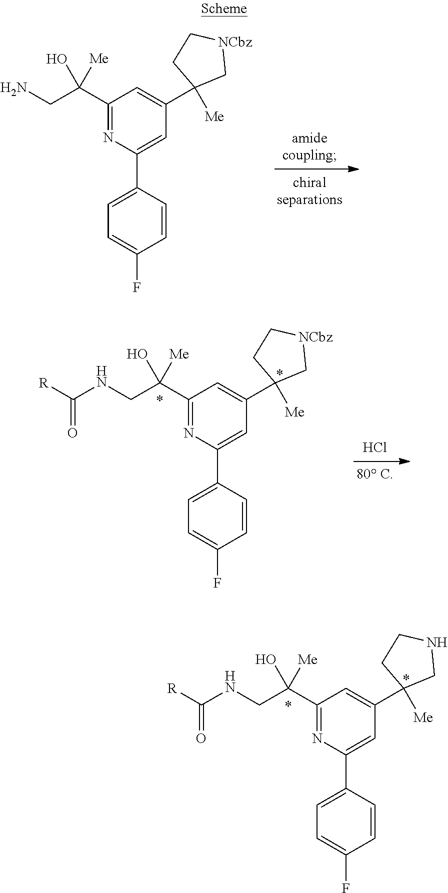

### Examples 27-A, 27-B, 27-C, and 27-D

### N-2-(6-(4-fluorophenyl)-4-(3-methylpyrrolidin-3-yl)pyridin-2-yl)-2-hydroxypropyl)-1-methyl-3-(thiazol-4-yl)-1H-pyrazole-5-carboxamide (diastereomer-1, diastereomer-2, diastereomer-3, and diastereomer-4)

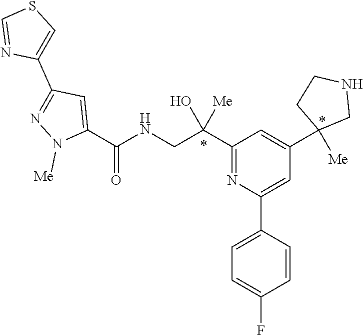 [SMILES: Cn1nc(-c2cscn2)cc1C(=O)NCC(C)(O)c1cc(C2(C)CCNC2)cc(-c2ccc(F)cc2)n1]

### Step 1: benzyl 3-(2-(4-fluorophenyl)-6-(2-hydroxy-1-(1-methyl-3-(thiazol-4-yl)-1H-pyrazole-5-carboxamido)propan-2-yl)pyridin-4-yl)-3-methylpyrrolidine-1-carboxylate (diastereomer-1, diastereomer-2, diastereomer-3, and diastereomer-4)

A mixture of 1-methyl-3-(thiazol-4-yl)-1H-pyrazole-5-carboxylic acid (Intermediate A-05, 43 mg, 0.21 mmol), benzyl 3-(2-(1-amino-2-hydroxypropan-2-yl)-6-(4-fluorophenyl)pyridin-4-yl)-3-methylpyrrolidine-1-carboxylate (Intermediate H-01, 0.12 g, 0.21 mmol), 1-propanephosphonic anhydride (50 wt % in EtOAc, 0.20 g, 0.31 mmol), DIPEA (0.11 mL, 0.62 mmol), and DMF (8.0 mL) was stirred at 25° C. for 1 h. Water (10 mL) was added and the mixture was extracted with EtOAc (15 mL×3). The combined organic extracts were washed with brine (10 mL), dried with anhydrous sodium sulfate, filtered, and concentrated in vacuo. The residue was purified by preparative TLC (50% EtOAc/hexanes) to afford the title compound that was furthered purified by SFC (DAICEL CHIRALPAK AD, 250×30 mm, 10 μm; 50% 2-PrOH (with 0.1% NH₄OH)/CO₂). The first eluting isomer was designated diastereomer-1 and the second eluting isomer was designated diastereomer-2. The third and fourth eluting isomers coeluted and were subjected to additional purification by SFC (DAICEL CHIRALPAK IG, 250×30 mm, 10 μm; 60% EtOH (with 0.1% NH₄OH)/CO₂). The first eluting isomer from the second purification was designated diastereomer-3 and the second eluting isomer from the second purification was designated diastereomer-4.

### Step 2-A: N-2-(6-(4-fluorophenyl)-4-(3-methylpyrrolidin-3-yl)pyridin-2-yl)-2-hydroxypropyl)-1-methyl-3-(thiazol-4-yl)-1H-pyrazole-5-carboxamide (diastereomer-1=EXAMPLE 27-A)

A mixture of benzyl 3-(2-(4-fluorophenyl)-6-(2-hydroxy-1-(1-methyl-3-(thiazol-4-yl)-1H-pyrazole-5-carboxamido)propan-2-yl)pyridin-4-yl)-3-methylpyrrolidine-1-carboxylate (diastereomer-1, 22 mg, 0.034 mmol) and hydrochloric acid (12 M aqueous, 2.0 mL) was stirred at 80° C. for 10 min. The mixture was cooled to room temperature, diluted with MeOH (2 mL), and filtered. The filtrate was concentrated in vacuo and the residue was purified by preparative HPLC (C-18; 15-35% MeCN/water (with 0.05% HCl)) to afford the title compound. MS m/z=(M+H)⁺: calculated 521.1, observed 521.1. ¹H NMR (500 MHz, CD₃OD) δ 9.65 (s, 1H), 8.34-8.12 (m, 3H), 8.06-7.96 (m, 2H), 7.41 (t, J=8.9 Hz, 2H), 7.33 (s, 1H), 4.22 (d, J=14.0 Hz, 1H), 4.04 (s, 3H), 3.85-3.71 (m, 3H), 3.65-3.52 (m, 2H), 2.67-2.45 (m, 2H), 1.91 (s, 3H), 1.59 (s, 3H) ppm.

### Step 2-B: N-2-(6-(4-fluorophenyl)-4-(3-methylpyrrolidin-3-yl)pyridin-2-yl)-2-hydroxypropyl)-1-methyl-3-(thiazol-4-yl)-1H-pyrazole-5-carboxamide (diastereomer-2=EXAMPLE 27-B)

A mixture of benzyl 3-(2-(4-fluorophenyl)-6-(2-hydroxy-1-(1-methyl-3-(thiazol-4-yl)-1H-pyrazole-5-carboxamido)propan-2-yl)pyridin-4-yl)-3-methylpyrrolidine-1-carboxylate (diastereomer-2, 0.020 g, 0.031 mmol) and hydrochloric acid (12 M aqueous, 2.0 mL) was stirred at 80° C. for 10 min. The mixture was cooled to room temperature, diluted with MeOH (2 mL), and filtered. The filtrate was concentrated in vacuo and the residue was purified by prep-HPLC (C-18; 15-35% MeCN/water (with 0.05% HCl)) to afford the title compound. MS m/z=(M+H)⁺: calculated 521.1, observed 521.1. ¹H NMR (CD₃OD, 500 MHz) δ 9.72 (s, 1H), 8.29-8.14 (m, 3H), 8.09-7.98 (m, 2H), 7.42 (t, J=8.5 Hz, 2H), 7.35 (s, 1H), 4.20 (d, J=14.0 Hz, 1H), 4.04 (s, 3H), 3.85-3.69 (m, 3H), 3.63-3.51 (m, 2H), 2.69-2.40 (m, 2H), 1.91 (s, 3H), 1.59 (s, 3H) ppm.

### Step 2-C: N-2-(6-(4-fluorophenyl)-4-(3-methylpyrrolidin-3-yl)pyridin-2-yl)-2-hydroxypropyl)-1-methyl-3-(thiazol-4-yl)-1H-pyrazole-5-carboxamide (diastereomer-3=EXAMPLE 27-C)

A mixture of benzyl 3-(2-(4-fluorophenyl)-6-(2-hydroxy-1-(1-methyl-3-(thiazol-4-yl)-1H-pyrazole-5-carboxamido)propan-2-yl)pyridin-4-yl)-3-methylpyrrolidine-1-carboxylate (diastereomer-3, 0.020 g, 0.031 mmol) and hydrochloric acid (12 M aqueous, 2.0 mL) was stirred at 80° C. for 10 min. The mixture was cooled to room temperature, diluted with MeOH (2 mL), and filtered. The filtrate was concentrated in vacuo and the residue was purified by prep-HPLC (C-18; 15-35% MeCN/water (with 0.05% HCl)) to afford the title compound. MS m/z=(M+H)⁺: calculated 521.1, observed 521.1. ¹H NMR (500 MHz, CD₃OD) δ 9.62 (s, 1H), 8.26-8.13 (m, 3H), 8.08-7.98 (m, 2H), 7.41 (t, J=8.5 Hz, 2H), 7.32 (s, 1H), 4.20 (d, J=14.0 Hz, 1H), 4.03 (s, 3H), 3.85-3.69 (m, 3H), 3.63-3.45 (m, 2H), 2.69-2.46 (m, 2H), 1.90 (s, 3H), 1.58 (s, 3H) ppm.

### Step 2-D: N-2-(6-(4-fluorophenyl)-4-(3-methylpyrrolidin-3-yl)pyridin-2-yl)-2-hydroxypropyl)-1-methyl-3-(thiazol-4-yl)-1H-pyrazole-5-carboxamide (diastereomer-4=EXAMPLE 27-D)

A mixture of benzyl 3-(2-(4-fluorophenyl)-6-(2-hydroxy-1-(1-methyl-3-(thiazol-4-yl)-1H-pyrazole-5-carboxamido)propan-2-yl)pyridin-4-yl)-3-methylpyrrolidine-1-carboxylate (diastereomer-4, 0.020 g, 0.031 mmol) and hydrochloric acid (12 M aqueous, 2.0 mL) was stirred at 80° C. for 10 min. The mixture was cooled to room temperature, diluted with MeOH (2 mL), and filtered. The filtrate was concentrated in vacuo and the residue was purified by prep-HPLC (C-18; 15-35% MeCN/water (with 0.05% HCl)) to afford the title compound. MS m/z=(M+H)⁺: calculated 521.1, observed 521.1. ¹H NMR (500 MHz, CD₃OD) δ 9.60 (s, 1H), 8.23-8.11 (m, 3H), 8.06-7.96 (m, 2H), 7.41 (t, J=8.9 Hz, 2H), 7.31 (s, 1H), 4.22 (d, J=14.0 Hz, 1H), 4.03 (s, 3H), 3.84-3.70 (m, 3H), 3.63-3.48 (m, 2H), 2.68-2.46 (m, 2H), 1.90 (s, 3H), 1.59 (s, 3H) ppm.

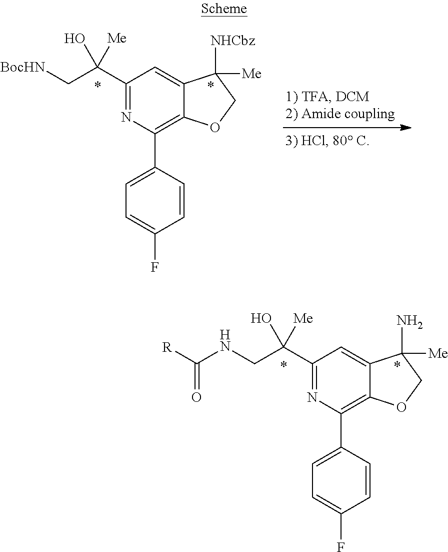

### Example 28-A

### N-2-(3-amino-7-(4-fluorophenyl)-3-methyl-2,3-dihydrofuro[2,3-c]pyridin-5-yl)-2-hydroxypropyl)-1-methyl-3-(thiazol-4-yl)-1H-pyrazole-5-carboxamide (diastereomer-1)

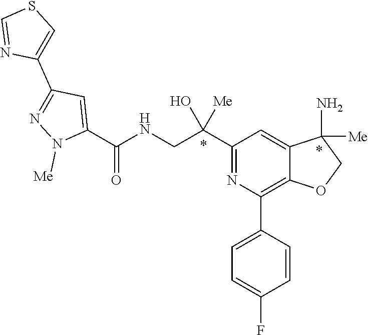 [SMILES: Cn1nc(-c2cscn2)cc1C(=O)NCC(C)(O)c1cc2c(c(-c3ccc(F)cc3)n1)OCC2(C)N]

### Step 1: benzyl (5-(1-amino-2-hydroxypropan-2-yl)-7-(4-fluorophenyl)-3-methyl-2,3-dihydrofuro[2,3-c]pyridin-3-yl)carbamate (diastereomer-1)

A mixture of tert-butyl (2-(3-(((benzyloxy)carbonyl)amino)-7-(4-fluorophenyl)-3-methyl-2,3-dihydrofuro[2,3-c]pyridin-5-yl)-2-hydroxypropyl)carbamate (Intermediate 1-01-diastereomer-1, 83 mg, 0.15 mmol), TFA (2.0 mL), and DCM (8.0 mL) was stirred at 25° C. for 1 h. The mixture was concentrated in vacuo to afford the title compound.

### Step 2: benzyl (7-(4-fluorophenyl)-5-(2-hydroxy-1-(1-methyl-3-(thiazol-4-yl)-1H-pyrazole-5-carboxamido)propan-2-yl)-3-methyl-2,3-dihydrofuro[2,3-c]pyridin-3-yl)carbamate (diastereomer-1)

A mixture of 1-methyl-3-(thiazol-4-yl)-1H-pyrazole-5-carboxylic acid (Intermediate A-05, 0.040 g, 0.19 mmol), HATU (83 mg, 0.22 mmol), DIPEA (0.13 mL, 0.73 mmol), and DMF (5.0 mL) was stirred at 25° C. for 10 min. A solution of benzyl (5-(1-amino-2-hydroxypropan-2-yl)-7-(4-fluorophenyl)-3-methyl-2,3-dihydrofuro[2,3-c]pyridin-3-yl)carbamate (diastereomer-1, 66 mg, 0.15 mmol) in DMF (3.0 mL) was added and the mixture was stirred for 1 h. Aqueous sodium bicarbonate (saturated, 10 mL) was added and the mixture was extracted with EtOAc (20 mL×3). The combined organic extracts were washed with brine (30 mL), dried with anhydrous sodium sulfate, filtered, and concentrated in vacuo. The residue was purified by silica gel chromatography (0-100% EtOAc/hexanes) to afford the title compound.

### Step 3: N-2-(3-amino-7-(4-fluorophenyl)-3-methyl-2,3-dihydrofuro[2,3-c]pyridin-5-yl)-2-hydroxypropyl)-1-methyl-3-(thiazol-4-yl)-1H-pyrazole-5-carboxamide (diastereomer-1)

A mixture of benzyl (7-(4-fluorophenyl)-5-(2-hydroxy-1-(1-methyl-3-(thiazol-4-yl)-1H-pyrazole-5-carboxamido)propan-2-yl)-3-methyl-2,3-dihydrofuro[2,3-c]pyridin-3-yl)carbamate (diastereomer-1, 35 mg, 0.054 mmol) and hydrochloric acid (12 M aqueous, 1.5 mL) was stirred at 80° C. for 10 min. The mixture was cooled to room temperature, concentrated in vacuo, and the pH was adjusted to ˜8 with ammonium hydroxide. The residue was purified by preparative HPLC (C-18; 32-62% MeCN/water (10 mM ammonium bicarbonate)) to afford the title compound. MS m/z=(M+H)⁺: calculated 509.2, observed 509.1. ¹H NMR (500 MHz, CD₃OD) δ 9.04 (d, J=2.0 Hz, 1H), 8.34-8.27 (m, 2H), 7.77 (d, J=2.0 Hz, 1H), 7.67 (s, 1H), 7.11 (t, J=8.8 Hz, 2H), 7.01 (s, 1H), 4.47-4.42 (m, 2H), 4.01 (s, 3H), 3.86 (d, J1.4 Hz, 1H), 3.73 (d, J=13.4 Hz, 1H), 1.65 (s, 3H), 1.53 (s, 3H) ppm.

### Example 28-B

### N-2-(3-amino-7-(4-fluorophenyl)-3-methyl-2,3-dihydrofuro[2,3-c]pyridin-5-yl)-2-hydroxypropyl)-1-methyl-3-(thiazol-4-yl)-1H-pyrazole-5-carboxamide (diastereomer-2)

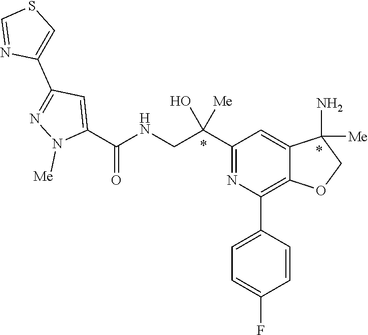 [SMILES: Cn1nc(-c2cscn2)cc1C(=O)NCC(C)(O)c1cc2c(c(-c3ccc(F)cc3)n1)OCC2(C)N]

### Step 1: benzyl (5-(1-amino-2-hydroxypropan-2-yl)-7-(4-fluorophenyl)-3-methyl-2,3-dihydrofuro[2,3-c]pyridin-3-yl)carbamate (diastereomer-2)

A mixture of tert-butyl (2-(3-(((benzyloxy)carbonyl)amino)-7-(4-fluorophenyl)-3-methyl-2,3-dihydrofuro[2,3-c]pyridin-5-yl)-2-hydroxypropyl)carbamate (Intermediate I-01-diastereomer-2, 84 mg, 0.15 mmol), TFA (2.0 mL), and DCM (8.0 mL) was stirred at 25° C. for 1 h. The mixture was concentrated in vacuo to afford the title compound.

### Step 2: benzyl (7-(4-fluorophenyl)-5-(2-hydroxy-1-(1-methyl-3-(thiazol-4-yl)-1H-pyrazole-5-carboxamido)propan-2-yl)-3-methyl-2,3-dihydrofuro[2,3-c]pyridin-3-yl)carbamate (diastereomer-2)

A mixture of 1-methyl-3-(thiazol-4-yl)-1H-pyrazole-5-carboxylic acid (Intermediate A-05, 35 mg, 0.17 mmol), HATU (86 mg, 0.23 mmol), DIPEA (0.13 mL, 0.75 mmol), and DMF (7.0 mL) was stirred at 25° C. for 10 min. A solution of benzyl (5-(1-amino-2-hydroxypropan-2-yl)-7-(4-fluorophenyl)-3-methyl-2,3-dihydrofuro[2,3-c]pyridin-3-yl)carbamate (diastereomer-2, 66 mg, 0.15 mmol) in DMF (3.0 mL) was added and the mixture was stirred for 1 h. Aqueous sodium bicarbonate (saturated, 10 mL) was added and the mixture was extracted with EtOAc (20 mL×3). The combined organic extracts were washed with brine (30 mL), dried with anhydrous sodium sulfate, filtered, and concentrated in vacuo. The residue was purified by silica gel chromatography (0-100% EtOAc/hexanes) to afford the title compound.

### Step 3: N-2-(3-amino-7-(4-fluorophenyl)-3-methyl-2,3-dihydrofuro[2,3-c]pyridin-5-yl)-2-hydroxypropyl)-1-methyl-3-(thiazol-4-yl)-1H-pyrazole-5-carboxamide (diastereomer-2)

A mixture of benzyl (7-(4-fluorophenyl)-5-(2-hydroxy-1-(1-methyl-3-(thiazol-4-yl)-1H-pyrazole-5-carboxamido)propan-2-yl)-3-methyl-2,3-dihydrofuro[2,3-c]pyridin-3-yl)carbamate (diastereomer-2, 73 mg, 0.11 mmol) and hydrochloric acid (12 M aqueous, 1.5 mL) was stirred at 80° C. for 10 min. The mixture was cooled to room temperature, concentrated in vacuo, and the pH was adjusted to ˜8 with ammonium hydroxide. The mixture was purified by preparative HPLC (C-18; 32-62% MeCN/water (10 mM ammonium bicarbonate)) to afford the title compound. MS m/z=(M+H)⁺: calculated 509.2, observed 509.1. ¹H NMR (500 MHz, CD₃OD) δ 9.03 (d, J=2.0 Hz, 1H), 8.35-8.26 (m, 2H), 7.77 (d, J=2.1 Hz, 1H), 7.67 (s, 1H), 7.15-7.07 (m, 2H), 7.06-6.95 (m, 1H), 4.48-4.42 (m, 2H), 4.01 (s, 3H), 3.87 (d, J=13.7 Hz, 1H), 3.73 (d, J=13.4 Hz, 1H), 1.65 (s, 3H), 1.56 (s, 3H) ppm.

### Example 28-C

### N-2-(3-amino-7-(4-fluorophenyl)-3-methyl-2,3-dihydrofuro[2,3-c]pyridin-5-yl)-2-hydroxypropyl)-1-methyl-3-(thiazol-4-yl)-1H-pyrazole-5-carboxamide (diastereomer-3)

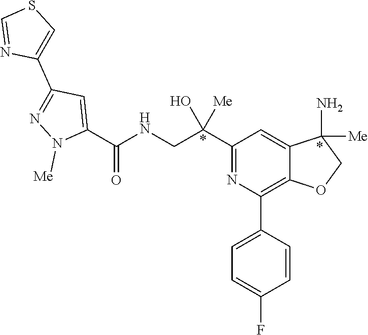 [SMILES: Cn1nc(-c2cscn2)cc1C(=O)NCC(C)(O)c1cc2c(c(-c3ccc(F)cc3)n1)OCC2(C)N]

### Step 1: benzyl (5-(1-amino-2-hydroxypropan-2-yl)-7-(4-fluorophenyl)-3-methyl-2,3-dihydrofuro[2,3-c]pyridin-3-yl)carbamate (diastereomer-3)

A mixture of tert-butyl (2-(3-(((benzyloxy)carbonyl)amino)-7-(4-fluorophenyl)-3-methyl-2,3-dihydrofuro[2,3-c]pyridin-5-yl)-2-hydroxypropyl)carbamate (Intermediate I-01-diastereomer-3, 93 mg, 0.17 mmol), TFA (2.0 mL), and DCM (8.0 mL) was stirred at 25° C. for 1 h. The mixture was concentrated in vacuo to afford the title compound.

### Step 2: benzyl (7-(4-fluorophenyl)-5-(2-hydroxy-1-(1-methyl-3-(thiazol-4-yl)-1H-pyrazole-5-carboxamido)propan-2-yl)-3-methyl-2,3-dihydrofuro[2,3-c]pyridin-3-yl)carbamate (diastereomer-3)

A mixture of 1-methyl-3-(thiazol-4-yl)-1H-pyrazole-5-carboxylic acid (Intermediate A-05, 38 mg, 0.18 mmol), HATU (95 mg, 0.25 mmol), DIPEA (0.15 mL, 0.83 mmol), and DMF (7.0 mL) was stirred at 25° C. for 10 min. A solution of benzyl (5-(1-amino-2-hydroxypropan-2-yl)-7-(4-fluorophenyl)-3-methyl-2,3-dihydrofuro[2,3-c]pyridin-3-yl)carbamate (diastereomer-3, 75 mg, 0.17 mmol) in DMF (3.0 mL) was added and the mixture was stirred for 1 h. Aqueous sodium bicarbonate (saturated, 10 mL) was added and the mixture was extracted with EtOAc (20 mL×3). The combined organic extracts were washed with brine (30 mL), dried with anhydrous sodium sulfate, filtered, and concentrated in vacuo. The residue was purified by silica gel chromatography (0-100% EtOAc/hexanes) to afford the title compound.

### Step 3: N-2-(3-amino-7-(4-fluorophenyl)-3-methyl-2,3-dihydrofuro[2,3-c]pyridin-5-yl)-2-hydroxypropyl)-1-methyl-3-(thiazol-4-yl)-1H-pyrazole-5-carboxamide (diastereomer-3)

A mixture of benzyl (7-(4-fluorophenyl)-5-(2-hydroxy-1-(1-methyl-3-(thiazol-4-yl)-1H-pyrazole-5-carboxamido)propan-2-yl)-3-methyl-2,3-dihydrofuro[2,3-c]pyridin-3-yl)carbamate (diastereomer-3, 81 mg, 0.13 mmol) and hydrochloric acid (12 M aqueous, 1.5 mL) was stirred at 80° C. for 10 min. The mixture was cooled to room temperature, concentrated in vacuo, and the pH was adjusted to ˜8 with ammonium hydroxide. The mixture was purified by preparative HPLC (C-18; 32-62% MeCN/water (10 mM ammonium bicarbonate)) to afford the title compound. MS m/z=(M+H)⁺: calculated 509.2, observed 509.1. ¹H NMR (500 MHz, CD₃OD) δ 9.03 (d, J=2.0 Hz, 1H), 8.36-8.25 (m, 2H), 7.77 (d, J=2.0 Hz, 1H), 7.67 (s, 1H), 7.14-7.09 (m, 2H), 7.01 (s, 1H), 4.47-4.41 (m, 2H), 4.01 (s, 3H), 3.86 (d, J=13.4 Hz, 1H), 3.73 (d, J=13.4 Hz, 1H), 1.65 (s, 3H), 1.52 (s, 3H) ppm.

### Example 28-D

### N-2-(3-amino-7-(4-fluorophenyl)-3-methyl-2,3-dihydrofuro[2,3-c]pyridin-5-yl)-2-hydroxypropyl)-1-methyl-3-(thiazol-4-yl)-1H-pyrazole-5-carboxamide (diastereomer-4)

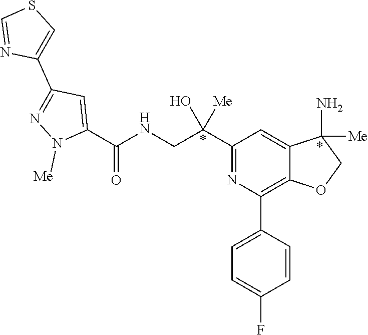 [SMILES: Cn1nc(-c2cscn2)cc1C(=O)NCC(C)(O)c1cc2c(c(-c3ccc(F)cc3)n1)OCC2(C)N]

### Step 1: benzyl (5-(1-amino-2-hydroxypropan-2-yl)-7-(4-fluorophenyl)-3-methyl-2,3-dihydrofuro[2,3-c]pyridin-3-yl)carbamate (diastereomer-4)

A mixture of tert-butyl (2-(3-(((benzyloxy)carbonyl)amino)-7-(4-fluorophenyl)-3-methyl-2,3-dihydrofuro[2,3-c]pyridin-5-yl)-2-hydroxypropyl)carbamate (Intermediate I-01-diastereomer-4, 96 mg, 0.17 mmol), TFA (2.0 mL), and DCM (8.0 mL) was stirred at 25° C. for 1 h. The mixture was concentrated in vacuo to afford the title compound.

### Step 2: benzyl (7-(4-fluorophenyl)-5-(2-hydroxy-1-(1-methyl-3-(thiazol-4-yl)-1H-pyrazole-5-carboxamido)propan-2-yl)-3-methyl-2,3-dihydrofuro[2,3-c]pyridin-3-yl)carbamate (diastereomer-4)

A mixture of 1-methyl-3-(thiazol-4-yl)-1H-pyrazole-5-carboxylic acid (Intermediate A-05, 39 mg, 0.19 mmol), HATU (97 mg, 0.26 mmol), DIPEA (0.15 mL, 0.85 mmol), and DMF (7.0 mL) was stirred at 25° C. for 10 min. A solution of benzyl (5-(1-amino-2-hydroxypropan-2-yl)-7-(4-fluorophenyl)-3-methyl-2,3-dihydrofuro[2,3-c]pyridin-3-yl)carbamate (diastereomer-4, 77 mg, 0.17 mmol) in DMF (3.0 mL) was added and the mixture was stirred for 1 h. Aqueous sodium bicarbonate (saturated, 10 mL) was added and the mixture was extracted with EtOAc (20 mL×3). The combined organic extracts were washed with brine (30 mL), dried with anhydrous sodium sulfate, filtered, and concentrated in vacuo. The residue was purified by silica gel chromatography (0-100% EtOAc/hexanes) to afford the title compound.

### Step 3: N-2-(3-amino-7-(4-fluorophenyl)-3-methyl-2,3-dihydrofuro[2,3-c]pyridin-5-yl)-2-hydroxypropyl)-1-methyl-3-(thiazol-4-yl)-1H-pyrazole-5-carboxamide (diastereomer-4)

A mixture of benzyl (7-(4-fluorophenyl)-5-(2-hydroxy-1-(1-methyl-3-(thiazol-4-yl)-1H-pyrazole-5-carboxamido)propan-2-yl)-3-methyl-2,3-dihydrofuro[2,3-c]pyridin-3-yl)carbamate (diastereomer-4, 93 mg, 0.13 mmol) and hydrochloric acid (12 M aqueous, 1.5 mL) was stirred at 80° C. for 10 min. The mixture was cooled to room temperature, concentrated in vacuo, and the pH was adjusted to ˜8 with ammonium hydroxide. The residue was purified by preparative HPLC (C-18; 32-62% MeCN/water (10 mM ammonium bicarbonate)) to afford the title compound. MS m/z=(M+H)⁺: calculated 509.2, observed 509.1. ¹H NMR (500 MHz, CD₃OD) δ 9.03 (d, J=2.1 Hz, 1H), 8.34-8.27 (m, 2H), 7.77 (d, J=2.0 Hz, 1H), 7.70-7.64 (m, 1H), 7.15-7.07 (m, 2H), 6.99 (s, 1H), 4.49-4.40 (m, 2H), 4.03-3.99 (m, 3H), 3.87 (d, J=13.4 Hz, 1H), 3.73 (d, J=13.6 Hz, 1H), 1.65 (s, 3H), 1.56 (s, 3H) ppm.

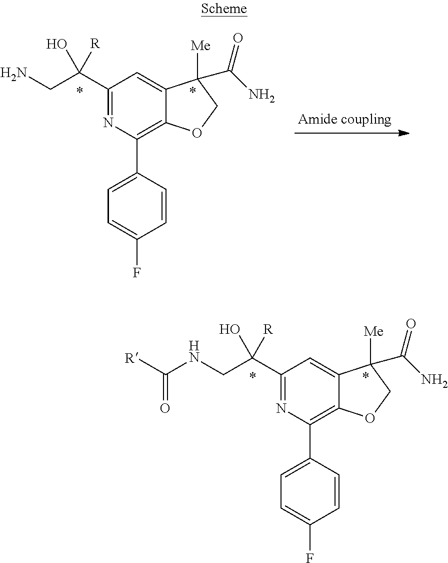

### Example 29-A

### 7-(4-fluorophenyl)-5-(2-hydroxy-1-(1′-methyl-1′H-[1,3′-bipyrazole]-5′-carboxamido)propan-2-yl)-3-methyl-2,3-dihydrofuro[2,3-c]pyridine-3-carboxamide (diastereomer-1)

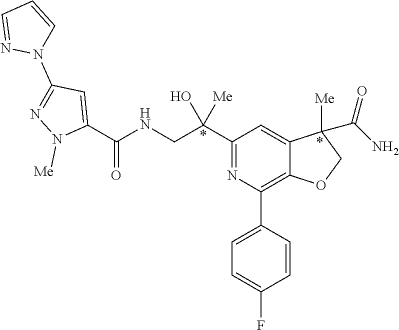 [SMILES: Cn1nc(-n2cccn2)cc1C(=O)NCC(C)(O)c1cc2c(c(-c3ccc(F)cc3)n1)OCC2(C)C(N)=O]

A mixture of 1′-methyl-1′H-[1,3′-bipyrazole]-5′-carboxylic acid (Intermediate A-02, 12 mg, 0.060 mmol), ((3H-[1,2,3]triazolo[4,5-b]pyridin-3-yl)oxy)tris(dimethylamino)phosphonium hexafluorophosphate(V) (33 mg, 0.074 mmol), DCM (1.0 mL), and DIPEA (18 mg, 0.14 mmol) was stirred at 25° C. for 10 min. 5-(1-amino-2-hydroxypropan-2-yl)-7-(4-fluorophenyl)-3-methyl-2,3-dihydrofuro[2,3-c]pyridine-3-carboxamide (Intermediate J-01-diastereomer-1, 16 mg, 0.046 mmol) was added and the mixture was stirred for 1 h. The mixture was concentrated and purified by preparative HPLC (C18; 27-47% MeCN/water (0.1% TFA)) to afford the title compound. MS m/z=(M+H)⁺: calculated 520.2, observed 520.2. ¹H NMR (400 MHz, CD₃OD) δ 8.27-8.17 (m, 2H), 8.09 (d, J=2.5 Hz, 1H), 7.75 (s, 1H), 7.70 (d, J=1.8 Hz, 1H), 7.18-7.08 (m, 2H), 6.79 (s, 1H), 6.48 (t, J=2.1 Hz, 1H), 5.12 (d, J=9.2 Hz, 1H), 4.48 (d, J=9.2 Hz, 1H), 3.91 (s, 3H), 3.78 (s, 2H), 1.70 (s, 3H), 1.67 (s, 3H) ppm.

### Example 29-B

### 7-(4-fluorophenyl)-5-(2-hydroxy-1-(1′-methyl-1′H-[1,3′-bipyrazole]-5′-carboxamido)propan-2-yl)-3-methyl-2,3-dihydrofuro[2,3-c]pyridine-3-carboxamide (diastereomer-2)

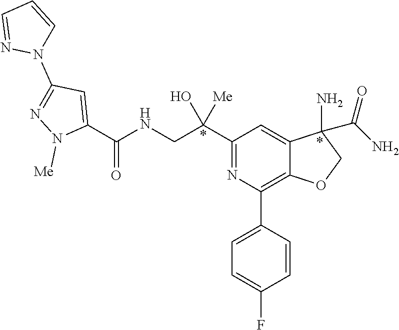 [SMILES: Cn1nc(-n2cccn2)cc1C(=O)NCC(C)(O)c1cc2c(c(-c3ccc(F)cc3)n1)OCC2(N)C(N)=O]

A mixture of 1′-methyl-1′H-[1,3′-bipyrazole]-5′-carboxylic acid (Intermediate A-02, 12 mg, 0.060 mmol), ((3H-[1,2,3]triazolo[4,5-b]pyridin-3-yl)oxy)tris(dimethylamino)phosphonium hexafluorophosphate(V) (33 mg, 0.074 mmol), DCM (1.0 mL), and DIPEA (18 mg, 0.14 mmol) was stirred at 25° C. for 10 min. 5-(1-amino-2-hydroxypropan-2-yl)-7-(4-fluorophenyl)-3-methyl-2,3-dihydrofuro[2,3-c]pyridine-3-carboxamide (Intermediate J-01-diastereomer-2, 16 mg, 0.046 mmol) was added and the mixture was stirred for 1 h. The mixture was concentrated and purified by preparative HPLC (C18; 27-47% MeCN/water (0.1% TFA)) to afford the title compound. MS m/z=(M+H)⁺: calculated 520.2, observed 520.2. ¹H NMR (400 MHz, CD₃OD) δ 8.27-8.16 (m, 2H), 8.09 (d, J=2.4 Hz, 1H), 7.77 (s, 1H), 7.70 (d, J=1.5 Hz, 1H), 7.22-7.09 (m, 2H), 6.79 (s, 1H), 6.48 (t, J=2.1 Hz, 1H), 5.12 (d, J=9.2 Hz, 1H), 4.49 (d, J=9.2 Hz, 1H), 3.91 (s, 3H), 3.78 (s, 2H), 1.70 (s, 3H), 1.67 (s, 3H) ppm.

### Example 29-C

### 7-(4-fluorophenyl)-5-(2-hydroxy-1-(1′-methyl-1′H-[1,3′-bipyrazole]-5′-carboxamido)propan-2-yl)-3-methyl-2,3-dihydrofuro[2,3-c]pyridine-3-carboxamide (diastereomer-3)

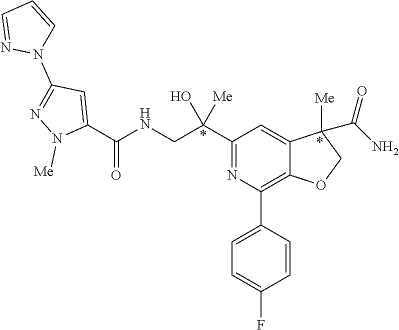 [SMILES: Cn1nc(-n2cccn2)cc1C(=O)NCC(C)(O)c1cc2c(c(-c3ccc(F)cc3)n1)OCC2(C)C(N)=O]

A mixture of 1′-methyl-1′H-[1,3′-bipyrazole]-5′-carboxylic acid (Intermediate A-02, 12 mg, 0.060 mmol), ((3H-[1,2,3]triazolo[4,5-b]pyridin-3-yl)oxy)tris(dimethylamino)phosphonium hexafluorophosphate(V) (33 mg, 0.074 mmol), DCM (1.0 mL), and DIPEA (18 mg, 0.14 mmol) was stirred at 25° C. for 10 min. 5-(1-amino-2-hydroxypropan-2-yl)-7-(4-fluorophenyl)-3-methyl-2,3-dihydrofuro[2,3-c]pyridine-3-carboxamide (Intermediate J-01-diastereomer-3, 16 mg, 0.046 mmol) was added and the mixture was stirred for 1 h. The mixture was concentrated and purified by preparative HPLC (C18; 27-47% MeCN/water (0.1% TFA)) to afford the title compound. MS m/z=(M+H)⁺: calculated 520.2, observed 520.2. ¹H NMR (400 MHz, CD₃OD) δ 8.27-8.14 (m, 2H), 8.07 (d, J=2.4 Hz, 1H), 7.78 (s, 1H), 7.69 (d, J=1.5 Hz, 1H), 7.23-7.06 (m, 2H), 6.80 (s, 1H), 6.47 (t, J=2.1 Hz, 1H), 5.12 (d, J=9.2 Hz, 1H), 4.47 (d, J=9.2 Hz, 1H), 3.98 (d, J=13.5 Hz, 1H), 3.87 (s, 3H), 3.58 (d, J=13.6 Hz, 1H), 1.70 (s, 3H), 1.67 (s, 3H) ppm.

### Example 29-D

### 7-(4-fluorophenyl)-5-(2-hydroxy-1-(1′-methyl-1′H-[1,3′-bipyrazole]-5′-carboxamido)propan-2-yl)-3-methyl-2,3-dihydrofuro[2,3-c]pyridine-3-carboxamide (diastereomer-4)

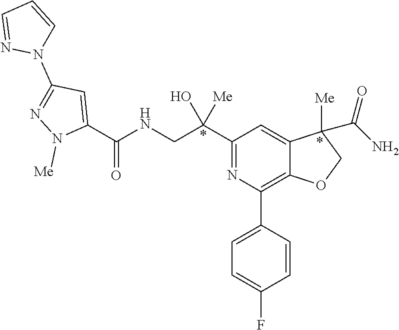 [SMILES: Cn1nc(-n2cccn2)cc1C(=O)NCC(C)(O)c1cc2c(c(-c3ccc(F)cc3)n1)OCC2(C)C(N)=O]

A mixture of 1′-methyl-1′H-[1,3′-bipyrazole]-5′-carboxylic acid (Intermediate A-02, 12 mg, 0.060 mmol), ((3H-[1,2,3]triazolo[4,5-b]pyridin-3-yl)oxy)tris(dimethylamino)phosphonium hexafluorophosphate(V) (33 mg, 0.074 mmol), DCM (1.0 mL), and DIPEA (18 mg, 0.14 mmol) was stirred at 25° C. for 10 min. 5-(1-amino-2-hydroxypropan-2-yl)-7-(4-fluorophenyl)-3-methyl-2,3-dihydrofuro[2,3-c]pyridine-3-carboxamide (Intermediate J-01-diastereomer-4, 16 mg, 0.046 mmol) was added and the mixture was stirred for 1 h. The mixture was concentrated and purified by preparative HPLC (C18; 27-47% MeCN/water (0.1% TFA)) to afford the title compound. MS m/z=(M+H)⁺: calculated 520.2, observed 520.2. ¹H NMR (400 MHz, CD₃OD) δ 8.25-8.15 (m, 2H), 8.07 (d, J=2.4 Hz, 1H), 7.77 (s, 1H), 7.69 (d, J=1.7 Hz, 1H), 7.21-7.06 (m, 2H), 6.80 (s, 1H), 6.47 (t, J=2.1 Hz, 1H), 5.12 (d, J=9.2 Hz, 1H), 4.47 (d, J=9.2 Hz, 1H), 3.98 (d, J=13.6 Hz, 1H), 3.87 (s, 3H), 3.58 (d, J=13.6 Hz, 1H), 1.70 (s, 3H), 1.67 (s, 3H) ppm.

### Example 30-B

### 7-(4-fluorophenyl)-3-methyl-5-(1,1,1-trifluoro-2-hydroxy-3-(1-methyl-3-(thiazol-4-yl)-1H-pyrazole-5-carboxamido)propan-2-yl)-2,3-dihydrofuro[2,3-c]pyridine-3-carboxamide (diastereomer-2)

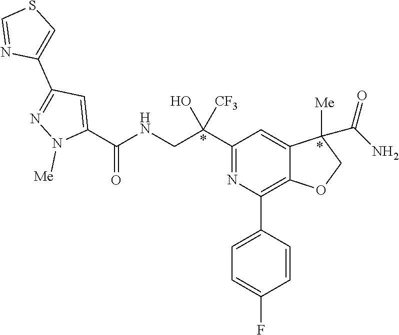 [SMILES: Cn1nc(-c2cscn2)cc1C(=O)NCC(O)(c1cc2c(c(-c3ccc(F)cc3)n1)OCC2(C)C(N)=O)C(F)(F)F]

A mixture of 1-methyl-3-(thiazol-4-yl)-1H-pyrazole-5-carboxylic acid (Intermediate A-05, 0.020 g, 0.096 mmol), 5-(3-amino-1,1,1-trifluoro-2-hydroxypropan-2-yl)-7-(4-fluorophenyl)-3-methyl-2,3-dihydrofuro[2,3-c]pyridine-3-carboxamide (Intermediate K-01-diastereomer-2, 16 mg, 0.046 mmol), HATU (46 mg, 0.12 mmol), DMF (8.0 mL), and DIPEA (0.10 mL, 0.40 mmol) was stirred at 25° C. for 1 h. Water (20 mL) was added and the mixture was extracted with EtOAc (20 mL×3). The combined organic extracts were dried with anhydrous sodium sulfate, filtered, and concentrated in vacuo. The residue was purified by preparative HPLC (C18; 45-65% MeCN/water (0.1% TFA)) to afford the title compound. MS m/z=(M+H)⁺: calculated 591.1, observed 591.1. ¹H NMR (500 MHz, CD₃OD) δ 9.02 (d, J=2.0 Hz, 1H), 8.32-8.18 (m, 2H), 7.83 (s, 1H), 7.76 (d, J=2.0 Hz, 1H), 7.19-7.08 (m, 2H), 7.02-6.91 (m, 1H), 5.15 (d, J=9.2 Hz, 1H), 4.55-4.48 (m, 2H), 3.92 (s, 3H), 3.88 (dd, J=13.9, 4.7 Hz, 1H), 1.67 (s, 3H) ppm.

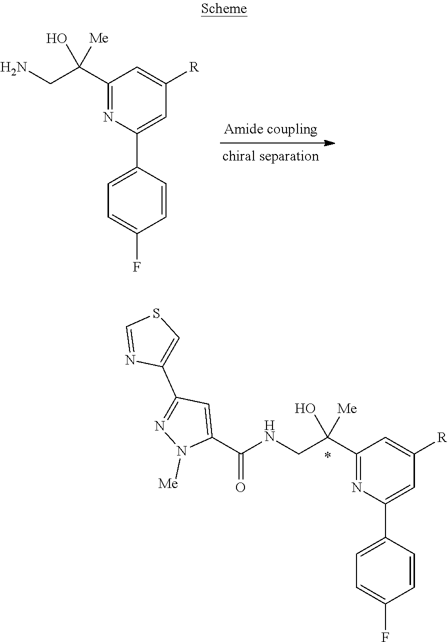

### Examples 31-A and 31-B

### N-(2-(4-(1-amino-2-methyl-1-oxopropan-2-yl)-6-(4-fluorophenyl)pyridin-2-yl)-2-hydroxypropyl)-1-methyl-3-(thiazol-4-yl)-1H-pyrazole-5-carboxamide (enantiomer-1 and enantiomer-2)

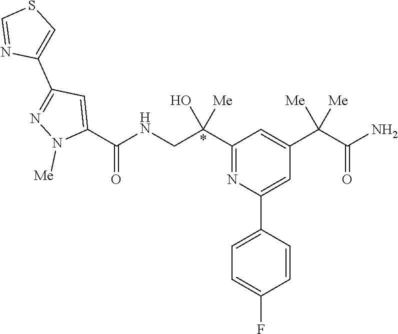 [SMILES: Cn1nc(-c2cscn2)cc1C(=O)NCC(C)(O)c1cc(C(C)(C)C(N)=O)cc(-c2ccc(F)cc2)n1]

A mixture of 2-(2-(1-amino-2-hydroxypropan-2-yl)-6-(4-fluorophenyl)pyridin-4-yl)-2-methylpropanamide (Intermediate L-01, 0.070 g, 0.21 mmol), 1-methyl-3-(thiazol-4-yl)-1H-pyrazole-5-carboxylic acid (Intermediate A-05, 44 mg, 0.21 mmol), DMF (8.0 mL), DIPEA (0.10 mL, 0.63 mmol), and a solution of propylphosphonic anhydride (50% in EtOAc; 0.20 g, 0.32 mmol) was stirred at 25° C. for 1 h. Water (5 mL) was added and the mixture was extracted with EtOAc (10 mL×3). The combined organic extracts were washed with brine (10 mL), dried with anhydrous sodium sulfate, filtered, and concentrated in vacuo. The residue was purified by preparative thin-layer chromatography (50% EtOAc/petroleum ether). The residue was purified by SFC (DAICEL CHIRALPAK AD, 250×30 mm, 10 μm; 45% EtOH (with 0.1% NH₄OH)/CO₂) to afford the title compounds. The first eluting isomer was designated enantiomer-1 (EXAMPLE 31-A). MS m/z=(M+H)⁺: calculated 523.1, observed 523.2. ¹H NMR (500 MHz, CD₃OD) δ 9.06 (d, J=2.5 Hz, 1H), 8.23-8.04 (m, 2H), 7.80 (d, J=2.5 Hz, 1H), 7.73-7.64 (m, 2H), 7.24-7.11 (m, 2H), 7.02 (s, 1H), 4.00 (s, 3H), 3.93 (d, J=13.4 Hz, 1H), 3.74 (d, J=14.0 Hz, 1H), 1.69 (s, 3H), 1.63 (d, J=3.0 Hz, 6H) ppm. The second eluting isomer was designated enantiomer-2 (EXAMPLE 31-B). MS m/z=(M+H)⁺: calculated 523.1, observed 523.2. ¹H NMR (500 MHz, CD₃OD) δ 8.94 (d, J=1.5 Hz, 1H), 8.00-7.92 (m, 2H), 7.68 (d, J=1.5 Hz, 1H), 7.61-7.55 (m, 2H), 7.04 (t, J=9.0 Hz, 2H), 6.90 (s, 1H), 3.88 (s, 3H), 3.81 (d, J=13.5 Hz, 1H), 3.62 (d, J=14.0 Hz, 1H), 1.57 (s, 3H), 1.51 (d, J=3.0 Hz, 6H) ppm.

### Examples 32-A and 32-B

### N-(2-(6-(4-fluorophenyl)-4-(2-hydroxypropan-2-yl)pyridin-2-yl)-2-hydroxypropyl)-1-methyl-3-(thiazol-4-yl)-1H-pyrazole-5-carboxamide (enantiomer-1 and enantiomer-2)

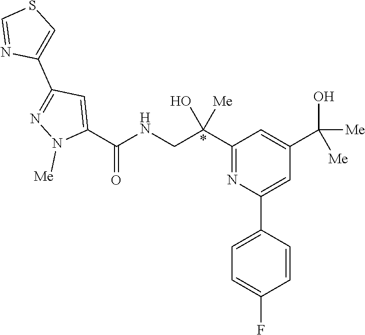 [SMILES: Cn1nc(-c2cscn2)cc1C(=O)NCC(C)(O)c1cc(C(C)(C)O)cc(-c2ccc(F)cc2)n1]

A mixture of 1-methyl-3-(thiazol-4-yl)-1H-pyrazole-5-carboxylic acid (Intermediate A-05, 0.050 g, 0.24 mmol), HATU (0.11 g, 0.29 mmol), DMF (3.0 mL), and DIPEA (0.10 mL, 0.72 mmol) was stirred at 25° C. for 30 min. 1-Amino-2-(6-(4-fluorophenyl)-4-(2-hydroxypropan-2-yl)pyridin-2-yl)propan-2-ol (Intermediate M-01, 73 mg, 0.24 mmol) was added and the mixture was stirred for 1 h. Water (10 mL) was added and the mixture was extracted with EtOAc (3 mL×3). The combined organic extracts were washed with brine (10 mL×2), dried with anhydrous sodium sulfate, filtered, and concentrated in vacuo. The residue was purified by preparative thin-layer chromatography (EtOAc). The residue was purified by SFC (CHIRALPAK AD-3, 50×4.6 mm, 3 μm; 5-40% EtOH (with 0.05% diethylamine)/CO₂) to afford the title compounds. The first eluting isomer was designated enantiomer-1 (EXAMPLE 32-A). MS m/z=(M+H)⁺: calculated 496.2, observed 496.2. ¹H NMR (400 MHz, CD₃OD) 9.03 (d, J=1.8 Hz, 1H), 8.14-8.04 (m, 2H), 7.82 (d, J=1.2 Hz, 1H), 7.77 (d, J=1.6 Hz, 1H), 7.73 (d, J=1.2 Hz, 1H), 7.18-7.10 (m, 2H), 7.00 (s, 1H), 4.00 (s, 3H), 3.93 (d, J=13.6 Hz, 1H), 3.74 (d, J=13.6 Hz, 1H), 1.66 (s, 3H), 1.55 (m, 6H) ppm. The second eluting isomer was designated enantiomer-2 (EXAMPLE 32-B). MS m/z=(M+H)⁺: calculated 496.2, observed 496.2. ¹H NMR (500 MHz, CD₃OD) δ 9.03 (s, 1H), 8.10 (dd, J=5.6, 8.7 Hz, 2H), 7.82 (d, J=1.2 Hz, 1H), 7.77 (d, J=1.6 Hz, 1H), 7.73 (d, J=1.2 Hz, 1H), 7.20-7.10 (m, 2H), 7.00 (s, 1H), 4.07-3.97 (m, 3H), 3.93 (d, J=13.6 Hz, 1H), 3.74 (d, J=13.6 Hz, 1H), 1.69-1.63 (m, 3H), 1.55 (m, 6H) ppm.

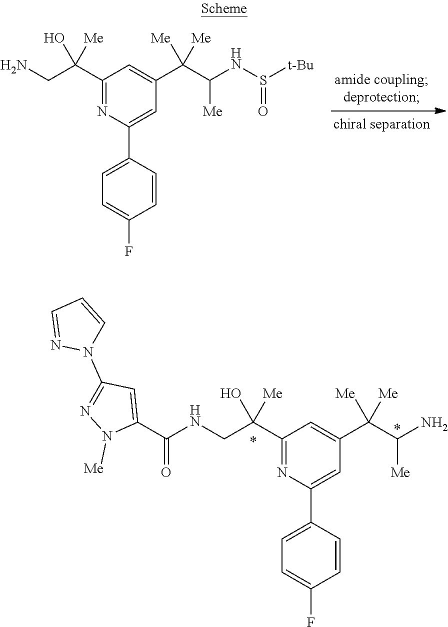 [SMILES: CC(NS(=O)C(C)(C)C)C(C)(C)c1cc(-c2ccc(F)cc2)nc(C(C)(O)CN)c1 ; CC(N)C(C)(C)c1cc(-c2ccc(F)cc2)nc(C(C)(O)CNC(=O)c2cc(-n3cccn3)nn2C)c1]

### Examples 33-A, 33-B, 33-C, and 33-D

### N-(2-(4-(3-amino-2-methylbutan-2-yl)-6-(4-fluorophenyl)pyridin-2-yl)-2-hydroxypropyl)-1′-methyl-1′H-[1,3′-bipyrazole]-5′-carboxamide (diastereomer-1, diastereomer-2, diastereomer-3, and diastereomer-4)

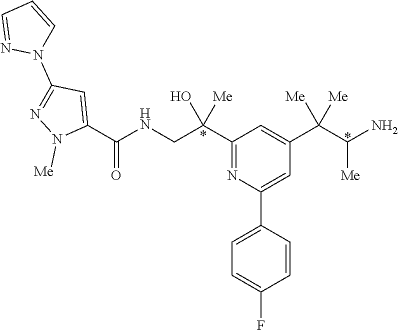 [SMILES: CC(N)C(C)(C)c1cc(-c2ccc(F)cc2)nc(C(C)(O)CNC(=O)c2cc(-n3cccn3)nn2C)c1]

### Step 1: N-(2-(4-(3-((tert-butylsulfinyl)amino)-2-methylbutan-2-yl)-6-(4-fluorophenyl)pyridin-2-yl)-2-hydroxypropyl)-1′-methyl-1′H-[1,3′-bipyrazole]-5′-carboxamide

A mixture of 1′-methyl-1′H-[1,3′-bipyrazole]-5′-carboxylic acid (Intermediate A-02, 54 mg, 0.28 mmol), ((3H-[1,2,3]triazolo[4,5-b]pyridin-3-yl)oxy)tris(dimethylamino)phosphonium hexafluorophosphate(V) (0.16 g, 0.35 mmol), DCM (3.0 mL), and DIPEA (0.20 mL, 1.1 mmol) was stirred at 25° C. for 3 min. N-(3-(2-(1-amino-2-hydroxypropan-2-yl)-6-(4-fluorophenyl)pyridin-4-yl)-3-methylbutan-2-yl)-2-methylpropane-2-sulfinamide (Intermediate N-01, 0.12 g, 0.23 mmol) was added and the mixture was stirred for 1 h. The mixture was concentrated and purified by preparative HPLC (C18; 53-73% MeCN/water (0.1% TFA)) to afford the title compound.

### Step 2: N-(2-(4-(3-amino-2-methylbutan-2-yl)-6-(4-fluorophenyl)pyridin-2-yl)-2-hydroxypropyl)-1′-methyl-1′H-[1,3′-bipyrazole]-5′-carboxamide (diastereomer-1, diastereomer-2, diastereomer-3, and diastereomer-4)

A mixture of N-(2-(4-(3-((tert-butylsulfinyl)amino)-2-methylbutan-2-yl)-6-(4-fluorophenyl)pyridin-2-yl)-2-hydroxypropyl)-1′-methyl-1′H-[1,3′-bipyrazole]-5′-carboxamide (95 mg, 0.16 mmol) and methanolic HCl (2.0 M, 2.0 mL, 4.0 mmol) was stirred at 25° C. for 2 h. The mixture was concentrated and purified by SFC run 1 (CHIRALPAK AD-3, 100×4.6 mm, 3 μm; 5-40% EtOH (with 0.05% diethylamine)/CO₂) to afford two mixtures each containing 2 isomers. The first eluting mixture from SFC run 1 was purified by SFC run 2 (CHIRALPAK AD-3, 100×4.6 mm, 3 μm; 5-40% 2-PrOH (with 0.05% diethylamine)/CO₂). The first eluting isomer from SFC run 2 was purified by preparative HPLC (C18; 25-45% MeCN/water (0.1% TFA)) to afford diastereomer-1 of the title compound (EXAMPLE 33-A). MS m/z=(M+H)⁺: calculated 506.2, observed 506.2. ¹H NMR (400 MHz, CD₃OD) δ 8.18-8.00 (m, 3H), 7.76-7.69 (m, 3H), 7.14 (t, J=8.4 Hz, 2H), 6.78 (s, 1H), 6.54-6.41 (m, 1H), 3.95-3.80 (m, 4H), 3.78-3.66 (m, 2H), 1.72 (s, 3H), 1.50-1.46 (m, 6H), 1.20 (d, J=6.6 Hz, 3H) ppm. The second eluting isomer from SFC run 2 was purified by preparative HPLC (C18; 25-45% MeCN/water (0.1% TFA)) to afford diastereomer-2 of the title compound (EXAMPLE 33-B). MS m/z=(M+H)⁺: calculated 506.2, observed 506.2. ¹H NMR (400 MHz, CD₃OD) δ 8.18-8.00 (m, 3H), 7.76-7.66 (m, 3H), 7.14 (t, J=8.4 Hz, 2H), 6.78 (s, 1H), 6.54-6.41 (m, 1H), 3.95-3.80 (m, 4H), 3.78-3.66 (m, 2H), 1.72 (s, 3H), 1.53-1.44 (m, 6H), 1.20 (d, J=6.6 Hz, 3H) ppm. The second eluting mixture from SFC run 1 was purified by SFC run 3 (CHIRALPAK IG-3, 100×4.6 mm, 3 μm; 40% 2-PrOH (with 0.05% diethylamine)/CO₂). The first eluting isomer from SFC run 3 was purified by preparative HPLC (C18; 25-45% MeCN/water (0.1% TFA)) to afford diastereomer-3 of the title compound (EXAMPLE 33-C). MS m/z=(M+H)⁺: calculated 506.2, observed 506.2. ¹H NMR (400 MHz, CD₃OD) δ 8.13-8.04 (m, 3H), 7.77-7.68 (m, 3H), 7.14 (t, J=8.4 Hz, 2H), 6.78 (s, 1H), 6.49 (s, 1H), 3.92-3.84 (m, 4H), 3.78-3.66 (m, 2H), 1.72 (s, 3H), 1.53-1.44 (m, 6H), 1.20 (d, J=6.8 Hz, 3H) ppm. The second eluting isomer from SFC run 3 was purified by preparative HPLC (C18; 25-45% MeCN/water (0.1% TFA)) to afford diastereomer-4 of the title compound (EXAMPLE 33-D). MS m/z=(M+H)⁺: calculated 506.2, observed 506.2. ¹H NMR (400 MHz, CD₃OD) δ 8.12-8.04 (m, 3H), 7.75-7.68 (m, 3H), 7.13 (t, J=8.4 Hz, 2H), 6.78 (s, 1H), 6.48 (s, 1H), 3.95-3.88 (m, 1H), 3.85 (s, 3H), 3.81-3.73 (m, 1H), 3.70-3.60 (m, 1H), 1.72 (s, 3H), 1.53-1.44 (m, 6H), 1.22 (d, J=6.8 Hz, 3H) ppm.

### Examples 34-A and 34-B

### N-(2-(4-(2-aminopropan-2-yl)-6-(4-fluorophenyl)pyridin-2-yl)-2-hydroxyethyl)-1′-methyl-1′H-[1,3′-bipyrazole]-5′-carboxamide (enantiomer-1 and enantiomer-2)

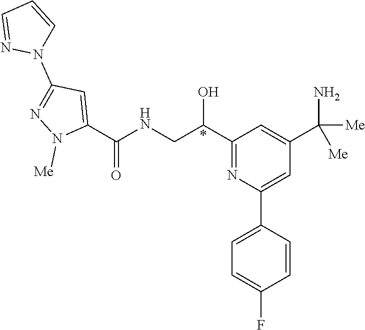 [SMILES: Cn1nc(-n2cccn2)cc1C(=O)NCC(O)c1cc(C(C)(C)N)cc(-c2ccc(F)cc2)n1]

### Step 1: benzyl (2-(2-(4-fluorophenyl)-6-(1-hydroxy-2-(1′-methyl-1′H-[1,3′-bipyrazole]-5′-carboxamido)ethyl)pyridin-4-yl)propan-2-yl)carbamate (enantiomer-1 and enantiomer-2)

A mixture of benzyl (2-(2-(2-amino-1-hydroxyethyl)-6-(4-fluorophenyl)pyridin-4-yl)propan-2-yl)carbamate (Intermediate 0-01, 0.60 g, 0.14 mmol), 1′-methyl-1′H-[1,3′-bipyrazole]-5′-carboxylic acid (Intermediate A-02, 27 mg, 0.14 mmol), ((3H-[1,2,3]triazolo[4,5-b]pyridin-3-yl)oxy)tris(dimethylamino)phosphonium hexafluorophosphate(V) (25 mg, 0.18 mmol), DCM (1.0 mL), and DIPEA (0.10 mL, 0.43 mmol) was stirred at 20° C. for 1 h. Brine (5 mL) was added and the mixture was extracted with EtOAc (5 mL×2). The combined organic extracts were washed with brine (5 mL×2), dried with anhydrous sodium sulfate, filtered, and concentrated in vacuo. The residue was purified by preparative HPLC (C18; 52-72% MeCN/water (0.1% TFA)). The residue was purified by SFC (CHIRALPAK AY-3, 100×4.6 mm, 3 μm; 5-40% 2-PrOH (with 0.05% diethylamine)/CO₂) to afford the title compounds. The first eluting enantiomer was designated enantiomer-1 and the second eluting enantiomer was designated enantiomer-2.

### Step 2-A: N-(2-(4-(2-aminopropan-2-yl)-6-(4-fluorophenyl)pyridin-2-yl)-2-hydroxyethyl)-1′-methyl-1′H-[1,3′-bipyrazole]-5′-carboxamide (enantiomer-1)

A mixture of benzyl (2-(2-(4-fluorophenyl)-6-(1-hydroxy-2-(1′-methyl-1′H-[1,3′-bipyrazole]-5′-carboxamido)ethyl)pyridin-4-yl)propan-2-yl)carbamate (enantiomer-1, 0.020 g, 0.033 mmol), palladium(II) chloride (0.60 mg, 3.4 μmol), triethylsilane (12 mg, 0.10 mmol), triethylamine (0.10 mL, 0.10 mmol), and THF (0.50 mL) was stirred at 20° C. for 30 min. The mixture was filtered and concentrated in vacuo. The residue was purified by preparative HPLC (C18; 20-40% MeCN/water (0.1% TFA)) to afford the title compound (EXAMPLE 34-A). MS m/z=(M+H)⁺: calculated 464.2, observed 464.2. ¹H NMR (400 MHz, CD₃OD) δ 8.16-8.08 (m, 3H), 7.81 (d, J=1.6 Hz, 1H), 7.71 (d, J=1.6 Hz, 1H), 7.65 (d, J=1.6 Hz, 1H), 7.22-7.13 (m, 2H), 6.87 (s, 1H), 6.50 (t, J=2.0 Hz, 1H), 5.05 (dd, J=5.4, 6.7 Hz, 1H), 4.01 (s, 3H), 3.92-3.84 (m, 1H), 3.80-3.69 (m, 1H), 1.78 (s, 6H) ppm.

### Step 2-B: N-(2-(4-(2-aminopropan-2-yl)-6-(4-fluorophenyl)pyridin-2-yl)-2-hydroxyethyl)-1′-methyl-1′H-[1,3′-bipyrazole]-5′-carboxamide (enantiomer-2)

A mixture of benzyl (2-(2-(4-fluorophenyl)-6-(1-hydroxy-2-(1′-methyl-1′H-[1,3′-bipyrazole]-5′-carboxamido)ethyl)pyridin-4-yl)propan-2-yl)carbamate (enantiomer-2, 0.030 g, 0.050 mmol), palladium(II) chloride (0.90 mg, 5.0 μmol), triethylsilane (18 mg, 0.15 mmol), triethylamine (0.10 mL, 0.15 mmol), and THF (0.50 mL) was stirred at 20° C. for 30 min. The mixture was filtered and concentrated in vacuo. The residue was purified by preparative HPLC (C18; 20-40% MeCN/water (0.1% TFA)) to afford the title compound (EXAMPLE 34-B). MS m/z=(M+H)⁺: calculated 464.2, observed 464.2. ¹H NMR (400 MHz, CD₃OD) δ 8.16-8.08 (m, 3H), 7.81 (d, J=1.6 Hz, 1H), 7.71 (d, J=1.6 Hz, 1H), 7.65 (d, J=1.6 Hz, 1H), 7.22-7.13 (m, 2H), 6.87 (s, 1H), 6.50 (t, J=2.0 Hz, 1H), 5.05 (dd, J=5.4, 6.7 Hz, 1H), 4.01 (s, 3H), 3.92-3.84 (m, 1H), 3.80-3.69 (m, 1H), 1.78 (s, 6H) ppm.

### Example 35-B

### N-(2-(4-(2-aminopropan-2-yl)-6-(4-fluorophenyl)pyridin-2-yl)-2-cyclopropyl-2-hydroxyethyl)-1′-methyl-1′H-[1,3′-bipyrazole]-5′-carboxamide (enantiomer-2)

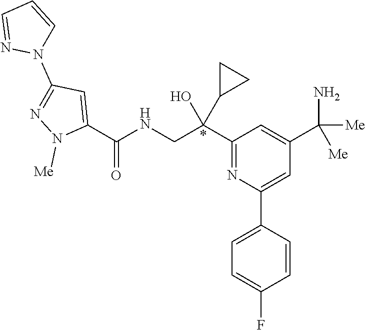 [SMILES: Cn1nc(-n2cccn2)cc1C(=O)NCC(O)(c1cc(C(C)(C)N)cc(-c2ccc(F)cc2)n1)C1CC1]

A mixture of 2-amino-1-(4-(2-aminopropan-2-yl)-6-(4-fluorophenyl)pyridin-2-yl)-1-cyclopropylethan-1-ol (Intermediate E-04-enantiomer-2, 0.020 g, 0.046 mmol), 1′-methyl-1′H-[1,3′-bipyrazole]-5′-carboxylic acid (Intermediate A-02, 8.8 mg, 0.046 mmol), HATU (18 mg, 0.048 mmol), DIPEA (0.040 mL, 0.23 mmol), and DMF (0.46 mL) was stirred at 23° C. for 16 h. The mixture was purified by preparative HPLC (C18; 5-100% MeCN/water (0.1% TFA)) to afford the title compound. MS m/z=(M+H)⁺: calculated 504.3, observed 504.4. ¹H NMR (500 MHz, CD₃OD) δ 8.18-8.11 (m, 2H), 8.10-8.05 (m, 1H), 7.80 (s, 1H), 7.75 (s, 1H), 7.67 (s, 1H), 7.19 (t, J=8.7 Hz, 2H), 6.73 (s, 1H), 6.49-6.43 (m, 1H), 4.19 (d, J=13.7 Hz, 1H), 3.94 (s, 3H), 3.83 (d, J=13.7 Hz, 1H), 1.62-1.52 (m, 1H), 0.73-0.65 (m, 1H), 0.61-0.49 (m, 1H), 0.43-0.33 (m, 1H), 0.29-0.20 (m, 1H) ppm.

### Example 36-A

### N-(2-(4-(2-aminopropan-2-yl)-6-(4-fluorophenyl)pyridin-2-yl)-3,3,3-trifluoro-2-hydroxypropyl)-1′-methyl-1′H-[1,3′-bipyrazole]-5′-carboxamide (enantiomer-1)

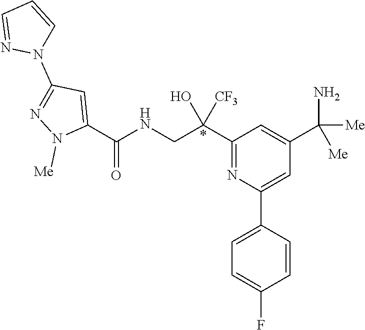 [SMILES: Cn1nc(-n2cccn2)cc1C(=O)NCC(O)(c1cc(C(C)(C)N)cc(-c2ccc(F)cc2)n1)C(F)(F)F]

A mixture of 3-amino-2-(4-(2-aminopropan-2-yl)-6-(4-fluorophenyl)pyridin-2-yl)-1,1,1-trifluoropropan-2-ol (Intermediate G-02-enantiomer-1, 0.020 g, 0.043 mmol), 1′-methyl-1′H-[1,3′-bipyrazole]-5′-carboxylic acid (Intermediate A-02, 8.2 mg, 0.043 mmol), HATU (17 mg, 0.045 mmol), DIPEA (0.037 mL, 0.21 mmol), and DMF (0.43 mL) was stirred at 23° C. for 16 h. The mixture was purified by preparative HPLC (C18; 5-100% MeCN/water (0.1% TFA)) to afford the title compound. MS m/z=(M+H)⁺: calculated 532.2, observed 532.3. ¹H NMR (500 MHz, CD₃OD) δ 8.23-8.15 (m, 2H), 8.11-8.06 (m, 1H), 7.95 (s, 1H), 7.92 (s, 1H), 7.67 (s, 1H), 7.28-7.20 (m, 2H), 6.74-6.69 (m, 1H), 6.50-6.44 (m, 1H), 4.55 (d, J=14.1 Hz, 1H), 4.04 (d, J=14.1 Hz, 1H), 3.95 (s, 3H), 1.79 (s, 6H) ppm.

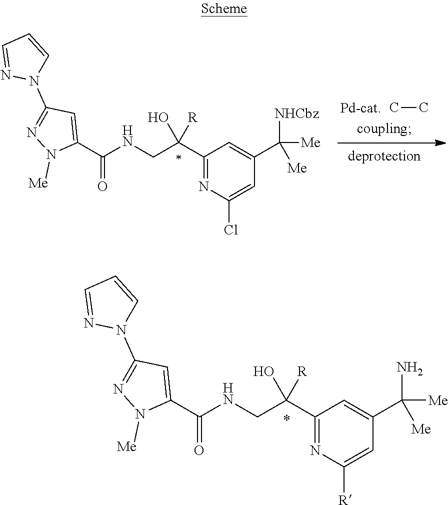

### Example 37-B

### N-(2-(4-(2-aminopropan-2-yl)-6-(4-(trifluoromethyl)phenyl)pyridin-2-yl)-2-hydroxypropyl)-1′-methyl-1′H-[1,3′-bipyrazole]-5′-carboxamide (enantiomer-2)

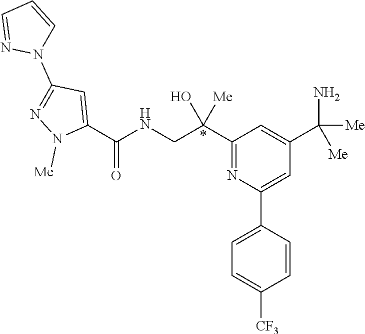 [SMILES: Cn1nc(-n2cccn2)cc1C(=O)NCC(C)(O)c1cc(C(C)(C)N)cc(-c2ccc(C(F)(F)F)cc2)n1]

A mixture of benzyl (2-(2-chloro-6-(2-hydroxy-1-(1′-methyl-1′H-[1,3′-bipyrazole]-5′-carboxamido)propan-2-yl)pyridin-4-yl)propan-2-yl)carbamate (Intermediate C-06-enantiomer-2, 0.050 g, 0.091 mmol), 4,4,5,5-tetramethyl-2-(4-(trifluoromethyl)phenyl)-1,3,2-dioxaborolane (0.030 g, 0.11 mmol), chloro(2-dicyclohexylphosphino-2′,4′,6′-triisopropyl-1,1′-biphenyl)[2-(2′-amino-1,1′-biphenyl)]palladium(II) (3.6 mg, 4.5 μmol), an aqueous solution of potassium carbonate (2.0 M, 0.14 mL, 0.27 mmol), and 1,4-dioxane (0.87 mL) was stirred in a 60° C. heating block for 16 h. The mixture was concentrated, suspended in EtOAc (2 mL), filtered, and concentrated. Hydrochloric acid (37% aqueous, 1.0 mL) was added and the mixture was stirred in an 80° C. heating block for 10 min. The mixture was concentrated and purified by preparative HPLC (C18; 5-100% MeCN/water (0.1% TFA)) to afford the title compound. MS m/z=(M+H)⁺: calculated 528.2, observed 528.3. ¹H NMR (500 MHz, CD₃OD) δ 8.34-8.27 (m, 2H), 8.09 (t, J=2.3 Hz, 1H), 7.93-7.91 (m, 1H), 7.89-7.87 (m, 1H), 7.79-7.73 (m, 2H), 7.70-7.68 (m, 1H), 6.83-6.81 (m, 1H), 6.49-6.46 (m, 1H), 3.98-3.92 (m, 4H), 3.79 (d, J=13.6 Hz, 1H), 1.79 (s, 6H), 1.71 (s, 3H) ppm.

### Example 38-B

### N-(2-(4-(2-aminopropan-2-yl)-6-(3,4-difluorophenyl)pyridin-2-yl)-2-hydroxypropyl)-1′-methyl-1′H-[1,3′-bipyrazole]-5′-carboxamide (enantiomer-2)

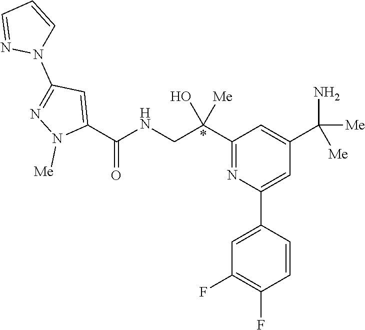 [SMILES: Cn1nc(-n2cccn2)cc1C(=O)NCC(C)(O)c1cc(C(C)(C)N)cc(-c2ccc(F)c(F)c2)n1]

A mixture of benzyl (2-(2-chloro-6-(2-hydroxy-1-(1′-methyl-1′H-[1,3′-bipyrazole]-5′-carboxamido)propan-2-yl)pyridin-4-yl)propan-2-yl)carbamate (Intermediate C-06-enantiomer-2, 0.050 g, 0.091 mmol), (3,4-difluorophenyl)boronic acid (17 mg, 0.11 mmol), chloro(2-dicyclohexylphosphino-2′,4′,6′-triisopropyl-1,1′-biphenyl)[2-(2′-amino-1,1′-biphenyl)]palladium(II) (3.6 mg, 4.5 μmol), an aqueous solution of potassium carbonate (2.0 M, 0.14 mL, 0.27 mmol), and 1,4-dioxane (0.87 mL) was stirred in a 60° C. heating block for 16 h. The mixture was concentrated, suspended in EtOAc (2 mL), filtered, and concentrated. Hydrochloric acid (37% aqueous, 1.0 mL) was added and the mixture was stirred in an 80° C. heating block for 10 min. The mixture was concentrated and purified by preparative HPLC (C18; 5-100% MeCN/water (0.1% TFA)) to afford the title compound. MS m/z=(M+H)⁺: calculated 496.2, observed 496.3. ¹H NMR (500 MHz, CD₃OD) δ 8.10 (d, J=2.5 Hz, 1H), 8.06 (ddd, J=12.0, 7.8, 2.1 Hz, 1H), 7.96-7.91 (m, 1H), 7.84-7.80 (m, 2H), 7.69 (d, J=1.8 Hz, 1H), 7.34 (dt, J=10.1, 8.5 Hz, 1H), 6.79 (s, 1H), 6.48 (t, J=2.2 Hz, 1H), 3.96-3.90 (m, 4H), 3.77 (d, J=13.6 Hz, 1H), 1.77 (s, 6H), 1.69 (s, 3H) ppm.

### Example 39-B

### N-(2-(4-(2-aminopropan-2-yl)-6-(4-chloro-3-fluorophenyl)pyridin-2-yl)-2-hydroxypropyl)-1′-methyl-1′H-[1,3′-bipyrazole]-5′-carboxamide (enantiomer-2)

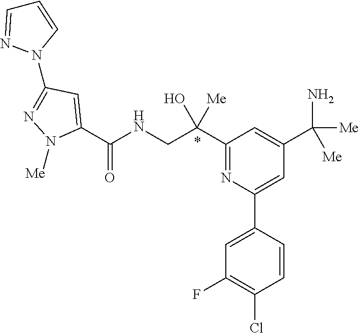 [SMILES: Cn1nc(-n2cccn2)cc1C(=O)NCC(C)(O)c1cc(C(C)(C)N)cc(-c2ccc(Cl)c(F)c2)n1]

A mixture of benzyl (2-(2-chloro-6-(2-hydroxy-1-(1′-methyl-1¹H-[1,3′-bipyrazole]-5′-carboxamido)propan-2-yl)pyridin-4-yl)propan-2-yl)carbamate (Intermediate C-06-enantiomer-2, 0.050 g, 0.091 mmol), (4-chloro-3-fluorophenyl)boronic acid (19 mg, 0.11 mmol), tetrakis(triphenylphosphine)palladium(0) (5.2 mg, 4.5 μmol), an aqueous solution of potassium carbonate (2.0 M, 0.14 mL, 0.27 mmol), and 1,4-dioxane (0.87 mL) was stirred in a 60° C. heating block for 16 h. The mixture was concentrated, suspended in EtOAc (2 mL), filtered, and concentrated. Hydrochloric acid (37% aqueous, 1.0 mL) was added and the mixture was stirred in an 80° C. heating block for 10 min. The mixture was concentrated and purified by preparative HPLC (C18; 5-100% MeCN/water (0.1% TFA)) to afford the title compound. MS m/z=(M+H)⁺: calculated 512.2, observed 512.3. ¹H NMR (500 MHz, CD₃OD) δ 8.13-8.09 (m, 1H), 8.03 (d, J=10.7 Hz, 1H), 7.94 (d, J=8.4 Hz, 1H), 7.88-7.83 (m, 2H), 7.69 (s, 1H), 7.55 (t, J=8.0 Hz, 1H), 6.81-6.78 (m, 1H), 6.50-6.47 (m, 1H), 3.99-3.89 (m, 4H), 3.77 (d, J=13.5 Hz, 1H), 1.77 (s, 6H), 1.69 (s, 3H) ppm.

### Example 40-B

### N-(2-(4-(2-aminopropan-2-yl)-6-(4-fluorobenzyl)pyridin-2-yl)-2-hydroxypropyl)-1′-methyl-1′H-[1,3′-bipyrazole]-5′-carboxamide (enantiomer-2)

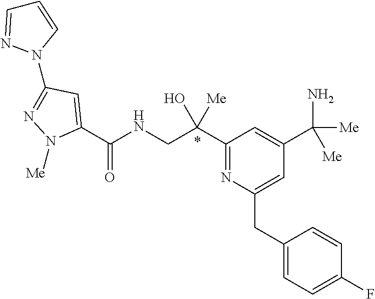 [SMILES: Cn1nc(-n2cccn2)cc1C(=O)NCC(C)(O)c1cc(C(C)(C)N)cc(Cc2ccc(F)cc2)n1]

### Step 1: tert-butyl (2-(4-(2-(((benzyloxy)carbonyl)amino)propan-2-yl)-6-(4-fluorobenzyl)pyridin-2-yl)-2-hydroxypropyl)carbamate (enantiomer-2)

A mixture of tert-butyl (2-(4-(2-(((benzyloxy)carbonyl)amino)propan-2-yl)-6-chloropyridin-2-yl)-2-hydroxypropyl)carbamate (Intermediate C-01-enantiomer-2, 0.15 g, 0.31 mmol), potassium trifluoro(4-fluorobenzyl)borate (0.14 g, 0.63 mmol), [1,1′-bis(diphenylphosphino)ferrocene]dichloropalladium(II) (46 mg, 0.063 mmol), cesium carbonate (0.31 g, 0.94 mmol), 1,4-dioxane (5.0 mL), and water (0.50 mL) was stirred in a 100° C. heating block for 12 h. The mixture was cooled to room temperature, water (5 mL) was added, and the mixture was extracted with EtOAc (10 mL×2). The combined organic extracts were washed with brine (10 mL×2), dried with anhydrous sodium sulfate, filtered, and concentrated in vacuo. The residue was purified by silica gel chromatography (0-30% EtOAc/petroleum ether) to afford the title compound.

### Step 2: benzyl (2-(2-(1-amino-2-hydroxypropan-2-yl)-6-(4-fluorobenzyl)pyridin-4-yl)propan-2-yl)carbamate (enantiomer-2)

A mixture of tert-butyl (2-(4-(2-(((benzyloxy)carbonyl)amino)propan-2-yl)-6-(4-fluorobenzyl)pyridin-2-yl)-2-hydroxypropyl)carbamate (enantiomer-2, 0.17 g, 0.23 mmol), DCM (3.0 mL), and TFA (1.0 mL) was stirred at 25° C. for 1 h. The mixture was concentrated to afford the title compound.

### Step 3: benzyl (2-(2-(4-fluorobenzyl)-6-(2-hydroxy-1-(1′-methyl-1′H-[1,3′-bipyrazole]-5′-carboxamido)propan-2-yl)pyridin-4-yl)propan-2-yl)carbamate (enantiomer-2)

A mixture of 1′-methyl-1′H-[1,3′-bipyrazole]-5′-carboxylic acid (Intermediate A-02, 0.030 g, 0.16 mmol), ((3H-[1,2,3]triazolo[4,5-b]pyridin-3-yl)oxy)tris(dimethylamino) phosphonium hexafluorophosphate(V) (69 mg, 0.16 mmol), DCM (3.0 mL), and DIPEA (0.082 mL, 0.47 mmol) was stirred at 25° C. for 10 min. Benzyl (2-(2-(1-amino-2-hydroxypropan-2-yl)-6-(4-fluorobenzyl)pyridin-4-yl)propan-2-yl)carbamate (enantiomer-2, 71 mg, 0.16 mmol) was added and the mixture was stirred for 1 h. The mixture was concentrated and purified by preparative HPLC (C18; 45-65% MeCN/water (0.1% TFA)) to afford the title compound.

### Step 4: N-(2-(4-(2-aminopropan-2-yl)-6-(4-fluorobenzyl)pyridin-2-yl)-2-hydroxypropyl)-1′-methyl-1′H-[1,3′-bipyrazole]-5′-carboxamide (enantiomer-2)

A mixture of benzyl (2-(2-(4-fluorobenzyl)-6-(2-hydroxy-1-(1′-methyl-1′H-[1,3′-bipyrazole]-5′-carboxamido)propan-2-yl)pyridin-4-yl)propan-2-yl)carbamate (enantiomer-2, 0.050 g, 0.080 mmol), palladium(II) chloride (2.8 mg, 0.016 mmol), triethylamine (0.033 mL, 0.24 mmol), triethylsilane (28 mg, 0.24 mmol), and THF (2.0 mL) was stirred at 20° C. for 30 min. The mixture was concentrated and purified by HPLC (C-18; 24-44% MeCN (with 0.1% TFA)/water (with 0.1% TFA)) to afford the title compound. MS m/z=(M+H)⁺: calculated 492.3, observed 492.2. ¹H NMR (400 MHz, CD₃OD) δ 8.16-8.12 (m, 1H), 7.77-7.70 (m, 2H), 7.32 (s, 1H), 7.30-7.25 (m, 2H), 6.96-6.87 (m, 2H), 6.79 (s, 1H), 6.51-6.48 (m, 1H), 4.21 (s, 2H), 4.00-3.91 (m, 4H), 3.65 (d, J=13.5 Hz, 1H), 1.69 (s, 6H), 1.64 (s, 3H) ppm.

### Example 41-A

### N-(2-(4-(2-aminopropan-2-yl)-6-(4-(difluoromethyl)phenyl)pyridin-2-yl)-3,3,3-trifluoro-2-hydroxypropyl)-1′-methyl-1′H-[1,3′-bipyrazole]-5′-carboxamide (enantiomer-1)

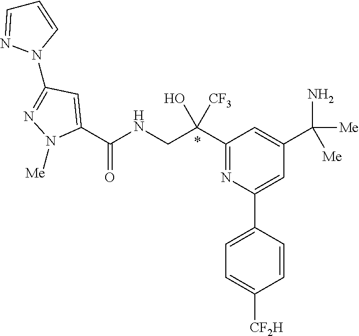 [SMILES: Cn1nc(-n2cccn2)cc1C(=O)NCC(O)(c1cc(C(C)(C)N)cc(-c2ccc(C(F)F)cc2)n1)C(F)(F)F]

### Step 1: benzyl (2-(2-(4-(difluoromethyl)phenyl)-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (enantiomer-1)

A mixture of benzyl (2-(2-chloro-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (Intermediate G-03-enantiomer-1, 0.040 g, 0.066 mmol), (4-(difluoromethyl)phenyl)boronic acid (14 mg, 0.079 mmol), chloro(2-dicyclohexylphosphino-2′,4′,6′-triisopropyl-1,1′-biphenyl)[2-(2′-amino-1,1′-biphenyl)]palladium(II) (5.2 mg, 6.6 μmol), an aqueous solution of K₃PO₄ (1.0 M, 0.13 mL, 0.13 mmol), and THF (0.33 mL) was stirred in a 100° C. heating block for 2 h. The mixture was cooled to room temperature, diluted with EtOAc, washed with water, dried with anhydrous sodium sulfate, filtered, and concentrated in vacuo. The residue was purified by silica gel chromatography (0-100% EtOAc/hexanes) to afford the title compound.

### Step 2: N-(2-(4-(2-aminopropan-2-yl)-6-(4-(difluoromethyl)phenyl)pyridin-2-yl)-3,3,3-trifluoro-2-hydroxypropyl)-1′-methyl-1′H-[1,3′-bipyrazole]-5′-carboxamide (enantiomer-1)

A mixture of benzyl (2-(2-(4-(difluoromethyl)phenyl)-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (enantiomer-1, 0.030 g, 0.043 mmol), palladium(II) chloride (2.3 mg, 0.013 mmol), triethylamine (0.024 mL, 0.17 mmol), triethylsilane (0.034 mL, 0.22 mmol), and DCM (0.22 mL) was stirred at 25° C. for 30 min. The mixture was diluted with EtOH, filtered, and concentrated in vacuo. The residue was purified by HPLC (C-18; 25-50% MeCN (with 0.1% TFA)/water (with 0.1% TFA)) to afford the title compound. MS m/z=(M+H)⁺: calculated 564.2, observed 564.4. ¹H NMR (500 MHz, CDCl₃) δ 8.05 (d, J=8.4 Hz, 2H), 8.00-7.97 (m, 2H), 7.80 (s, 1H), 7.63 (d, J=8.2 Hz, 2H), 7.61 (d, J=1.3 Hz, 1H), 6.71 (t, J=56.4 Hz, 1H), 6.64 (s, 1H), 6.54 (s, 1H), 6.37 (dd, J=2.4, 1.8 Hz, 1H), 6.33 (dd, J=8.5, 3.4 Hz, 1H), 4.66 (dd, J=14.1, 8.7 Hz, 1H), 4.01 (s, 3H), 3.90 (dd, J=14.1, 3.7 Hz, 1H), 1.59 (s, 2H), 1.53 (s, 6H) ppm.

### Example 42-A

### N-(2-(4-(2-aminopropan-2-yl)-6-(4-(trifluoromethyl)phenyl)pyridin-2-yl)-3,3,3-trifluoro-2-hydroxypropyl)-1′-methyl-1′H-[1,3′-bipyrazole]-5′-carboxamide (enantiomer-1)

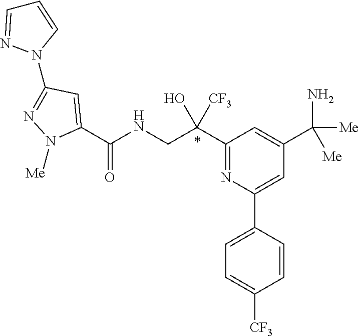 [SMILES: Cn1nc(-n2cccn2)cc1C(=O)NCC(O)(c1cc(C(C)(C)N)cc(-c2ccc(C(F)(F)F)cc2)n1)C(F)(F)F]

### Step 1: benzyl (2-(2-(1,1,1-trifluoro-2-hydroxy-3-(1′-methyl-1′H-[1,3′-bipyrazole]-5′-carboxamido)propan-2-yl)-6-(4-(trifluoromethyl)phenyl)pyridin-4-yl)propan-2-yl)carbamate (enantiomer-1)

A mixture of benzyl (2-(2-chloro-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (Intermediate G-03-enantiomer-1, 0.040 g, 0.066 mmol), (4-(trifluoromethyl)phenyl)boronic acid (15 mg, 0.079 mmol), chloro(2-dicyclohexylphosphino-2′,4′,6′-triisopropyl-1,1′-biphenyl)[2-(2′-amino-1,1′-biphenyl)]palladium(II) (5.2 mg, 6.6 μmol), an aqueous solution of K₃PO₄ (1.0 M, 0.13 mL, 0.13 mmol), and THF (0.33 mL) was stirred in a 100° C. heating block for 2 h. The mixture was cooled to room temperature, diluted with EtOAc, washed with water, dried with anhydrous sodium sulfate, filtered, and concentrated in vacuo. The residue was purified by silica gel chromatography (0-50% EtOAc/hexanes) to afford the title compound.

### Step 2: N-(2-(4-(2-aminopropan-2-yl)-6-(4-(trifluoromethyl)phenyl)pyridin-2-yl)-3,3,3-trifluoro-2-hydroxypropyl)-1′-methyl-1′H-[1,3′-bipyrazole]-5′-carboxamide (enantiomer-1)

A mixture of benzyl (2-(2-(1,1,1-trifluoro-2-hydroxy-3-(1′-methyl-1′H-[1,3′-bipyrazole]-5′-carboxamido)propan-2-yl)-6-(4-(trifluoromethyl)phenyl)pyridin-4-yl)propan-2-yl)carbamate (enantiomer-1, 26 mg, 0.036 mmol), palladium(II) chloride (1.9 mg, 0.011 mmol), triethylamine (0.020 mL, 0.15 mmol), triethylsilane (0.029 mL, 0.18 mmol), and DCM (0.18 mL) was stirred at 25° C. for 30 min. The mixture was diluted with EtOH, filtered, and concentrated in vacuo. The residue was purified by HPLC (C-18; 30-55% MeCN (with 0.1% TFA)/water (with 0.1% TFA)) to afford the title compound. MS m/z=(M+H)⁺: calculated 582.2, observed 582.4. ¹H NMR (500 MHz, CDCl₃) δ 8.08 (d, J=8.1 Hz, 2H), 8.01-7.97 (m, 2H), 7.83 (s, 1H), 7.75 (d, J=8.2 Hz, 2H), 7.61 (d, J=1.3 Hz, 1H), 6.64-6.49 (m, 2H), 6.41-6.29 (m, 2H), 4.66 (dd, J=14.0, 8.6 Hz, 1H), 4.02 (s, 3H), 3.91 (dd, J=14.1, 3.6 Hz, 1H), 1.66 (s, 2H), 1.54 (s, 6H) ppm.

### Example 43-A

### N-(2-(4-(2-aminopropan-2-yl)-6-(3,4-difluorophenyl)pyridin-2-yl)-3,3,3-trifluoro-2-hydroxypropyl)-1′-methyl-1′H-[1,3′-bipyrazole]-5′-carboxamide (enantiomer-1)

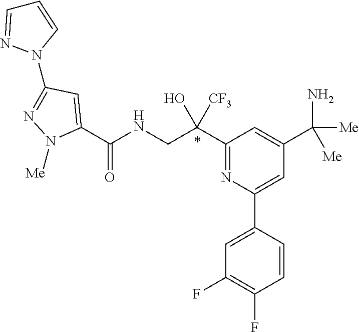 [SMILES: Cn1nc(-n2cccn2)cc1C(=O)NCC(O)(c1cc(C(C)(C)N)cc(-c2ccc(F)c(F)c2)n1)C(F)(F)F]

### Step 1: benzyl (2-(2-(3,4-difluorophenyl)-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (enantiomer-1)

A mixture of benzyl (2-(2-chloro-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (Intermediate G-03-enantiomer-1, 0.040 g, 0.066 mmol), (3,4-difluorophenyl)boronic acid (13 mg, 0.079 mmol), chloro(2-dicyclohexylphosphino-2′,4′,6′-triisopropyl-1,1′-biphenyl)[2-(2′-amino-1,1′-biphenyl)]palladium(II) (5.2 mg, 6.6 μmol), an aqueous solution of K₃PO₄ (1.0 M, 0.13 mL, 0.13 mmol), and THF (0.33 mL) was stirred in a 100° C. heating block for 2 h. The mixture was cooled to room temperature, diluted with EtOAc, washed with water, dried with anhydrous sodium sulfate, filtered, and concentrated in vacuo. The residue was purified by silica gel chromatography (0-50% EtOAc/hexanes) to afford the title compound.

### Step 2: N-(2-(4-(2-aminopropan-2-yl)-6-(3,4-difluorophenyl)pyridin-2-yl)-3,3,3-trifluoro-2-hydroxypropyl)-1′-methyl-1′H-[1,3′-bipyrazole]-5′-carboxamide (enantiomer-1)

A mixture of benzyl (2-(2-(3,4-difluorophenyl)-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (enantiomer-1, 26 mg, 0.038 mmol), palladium(II) chloride (2.0 mg, 0.011 mmol), triethylamine (0.021 mL, 0.15 mmol), triethylsilane (0.030 mL, 0.19 mmol), and DCM (0.19 mL) was stirred at 25° C. for 30 min. The mixture was diluted with EtOH, filtered, and concentrated in vacuo. The residue was purified by HPLC (C-18; 25-50% MeCN (with 0.1% TFA)/water (with 0.1% TFA)) to afford the title compound. MS m/z=(M+H)⁺: calculated 550.2, observed 550.4. ¹H NMR (500 MHz, CDCl₃) δ 8.00 (d, J=2.5 Hz, 1H), 7.90 (d, J=1.2 Hz, 1H), 7.81 (ddd, J=11.3, 7.6, 2.2 Hz, 1H), 7.77 (s, 1H), 7.74-7.69 (m, 1H), 7.62 (d, J=1.6 Hz, 1H), 7.31-7.24 (m, 1H), 6.57-6.49 (m, 2H), 6.39-6.36 (m, 1H), 6.32 (dd, J=8.3, 3.2 Hz, 1H), 4.66 (dd, J=14.1, 8.7 Hz, 1H), 4.02 (s, 3H), 3.89 (dd, J=14.2, 3.6 Hz, 1H), 1.61 (s, 2H), 1.52 (s, 6H) ppm.

### Example 44-A

### N-(2-(4-(2-aminopropan-2-yl)-6-(2,4-difluorophenyl)pyridin-2-yl)-3,3,3-trifluoro-2-hydroxypropyl)-1′-methyl-1′H-[1,3′-bipyrazole]-5′-carboxamide (enantiomer-1)

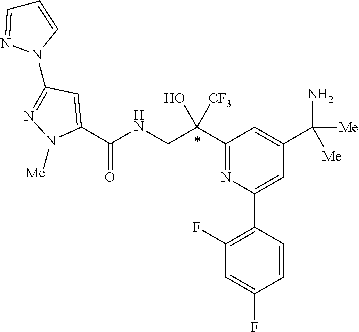 [SMILES: Cn1nc(-n2cccn2)cc1C(=O)NCC(O)(c1cc(C(C)(C)N)cc(-c2ccc(F)cc2F)n1)C(F)(F)F]

### Step 1: benzyl (2-(2-(2,4-difluorophenyl)-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (enantiomer-1)

A mixture of benzyl (2-(2-chloro-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (Intermediate G-03-enantiomer-1, 0.040 g, 0.066 mmol), (2,4-difluorophenyl)boronic acid (13 mg, 0.079 mmol), chloro(2-dicyclohexylphosphino-2′,4′,6′-triisopropyl-1,1′-biphenyl)[2-(2′-amino-1,1′-biphenyl)]palladium(II) (5.2 mg, 6.6 μmol), an aqueous solution of K₃PO₄ (1.0 M, 0.13 mL, 0.13 mmol), and THF (0.33 mL) was stirred in a 100° C. heating block for 2 h. The mixture was cooled to room temperature, diluted with EtOAc, washed with water, dried with anhydrous sodium sulfate, filtered, and concentrated in vacuo. The residue was purified by silica gel chromatography (0-50% EtOAc/hexanes) to afford the title compound.

### Step 2: N-(2-(4-(2-aminopropan-2-yl)-6-(2,4-difluorophenyl)pyridin-2-yl)-3,3,3-trifluoro-2-hydroxypropyl)-1′-methyl-1′H-[1,3′-bipyrazole]-5′-carboxamide (enantiomer-1)

A mixture of benzyl (2-(2-(2,4-difluorophenyl)-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (enantiomer-1, 25 mg, 0.036 mmol), palladium(II) chloride (1.9 mg, 0.011 mmol), triethylamine (0.020 mL, 0.15 mmol), triethylsilane (0.029 mL, 0.18 mmol), and DCM (0.18 mL) was stirred at 25° C. for 30 min. The mixture was diluted with EtOH, filtered, and concentrated in vacuo. The residue was purified by HPLC (C-18; 25-50% MeCN (with 0.1% TFA)/water (with 0.1% TFA)) to afford the title compound. MS m/z=(M+H)⁺: calculated 550.2, observed 550.4. ¹H NMR (500 MHz, CDCl₃) δ 1H NMR (500 MHz, Chloroform-d) δ 8.01 (d, J=2.2 Hz, 1H), 7.92 (t, J=1.4 Hz, 1H), 7.88 (td, J=8.8, 6.6 Hz, 1H), 7.81 (s, 1H), 7.63 (d, J=1.4 Hz, 1H), 7.05-7.00 (m, 1H), 6.93 (ddd, J=11.2, 8.7, 2.5 Hz, 1H), 6.57 (s, 1H), 6.53 (s, 1H), 6.39-6.37 (m, 1H), 6.36 (dd, J=8.3, 3.3 Hz, 1H), 4.63 (dd, J=14.0, 8.5 Hz, 1H), 4.01 (s, 3H), 3.91 (dd, J=14.1, 3.7 Hz, 1H), 1.61 (s, 2H), 1.52 (s, 3H), 1.52 (s, 3H) ppm.

### Example 45-A

### N-(2-(4-(2-aminopropan-2-yl)-6-(4-fluoro-2-methylphenyl)pyridin-2-yl)-3,3,3-trifluoro-2-hydroxypropyl)-1′-methyl-1′H-[1,3′-bipyrazole]-5′-carboxamide (enantiomer-1)

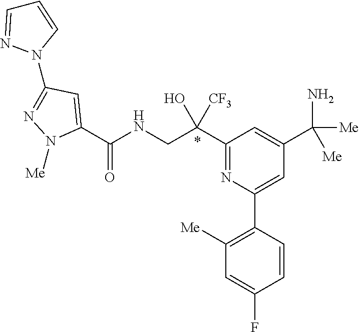 [SMILES: Cc1cc(F)ccc1-c1cc(C(C)(C)N)cc(C(O)(CNC(=O)c2cc(-n3cccn3)nn2C)C(F)(F)F)n1]

### Step 1: benzyl (2-(2-(4-fluoro-2-methylphenyl)-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (enantiomer-1)

A mixture of benzyl (2-(2-chloro-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (Intermediate G-03-enantiomer-1, 0.040 g, 0.066 mmol), (4-fluoro-2-methylphenyl)boronic acid (12 mg, 0.079 mmol), chloro(2-dicyclohexylphosphino-2′,4′,6′-triisopropyl-1,1′-biphenyl)[2-(2′-amino-1,1-biphenyl)]palladium(II) (5.2 mg, 6.6 μmol), an aqueous solution of K₃PO₄ (1.0 M, 0.13 mL, 0.13 mmol), and THF (0.33 mL) was stirred in a 100° C. heating block for 2 h. The mixture was cooled to room temperature, diluted with EtOAc, washed with water, dried with anhydrous sodium sulfate, filtered, and concentrated in vacuo. The residue was purified by silica gel chromatography (0-50% EtOAc/hexanes) to afford the title compound.

### Step 2: N-(2-(4-(2-aminopropan-2-yl)-6-(4-fluoro-2-methylphenyl)pyridin-2-yl)-3,3,3-trifluoro-2-hydroxypropyl)-1′-methyl-1′H-[1,3′-bipyrazole]-5′-carboxamide (enantiomer-1)

A mixture of benzyl (2-(2-(4-fluoro-2-methylphenyl)-6-(1,1,1-trifluoro-2-hydroxy-3-(1′-methyl-1′H-[1,3′-bipyrazole]-5′-carboxamido)propan-2-yl)pyridin-4-yl)propan-2-yl)carbamate (enantiomer-1, 17 mg, 0.025 mmol), palladium(II) chloride (1.3 mg, 7.4 μmol), triethylamine (0.014 mL, 98 μmol), triethylsilane (0.020 mL, 0.12 mmol), and DCM (0.12 mL) was stirred at 25° C. for 30 min. The mixture was diluted with EtOH, filtered, and concentrated in vacuo. The residue was purified by HPLC (C-18; 25-50% MeCN (with 0.1% TFA)/water (with 0.1% TFA)) to afford the title compound. MS m/z=(M+H)⁺: calculated 546.2, observed 546.4. ¹H NMR (500 MHz, CDCl₃) δ 8.01 (d, J=2.2 Hz, 1H), 7.77 (s, 1H), 7.64 (d, J=1.4 Hz, 1H), 7.57 (d, J=1.4 Hz, 1H), 7.35 (dd, J=8.4, 5.9 Hz, 1H), 7.02-6.94 (m, 2H), 6.61 (s, 1H), 6.51 (s, 1H), 6.40-6.39 (m, 1H), 6.32 (dd, J=8.4, 3.3 Hz, 1H), 4.64 (dd, J=14.0, 8.7 Hz, 1H), 4.02 (s, 3H), 3.87 (dd, J=14.0, 3.6 Hz, 1H), 2.29 (s, 3H), 1.58 (s, 2H), 1.51 (s, 3H), 1.50 (s, 3H) ppm.

### Assays Description

### Generation and Propagation of Recombinant RSV-A2-GFP Virus:

GFP sequence was derived from pJTI™ R4 Dest CMV N-EmGFP pA Vector (Invitrogen) and was cloned into the intergenic sequence between wild-type RSV-A2 P and M genes using standard recombineering techniques. Recombinant RSV-A2-GFP was propagated in Hep2 cells (human cells contains HeLa marker chromosomes and were derived via HeLa contamination, ATCC CCL-23) with infection Multiplicity of Infection (MOI) of 0.1. Virus was harvested 3 days after infection by collecting all culture material and freeze-thaw the viral culture in liquid nitrogen for at least 5 times. To generating virus working stocks, thaw frozen samples in 37° C. water bath then centrifuge at 218 g for 15 min at 4° C. Add 1/10 of 10×SPG (Biological Industries 06-3061-01-5A), mix, aliquot supernatant, and quick-freeze in liquid nitrogen then transfer to −80° C. freezer for storage. Virus titer was determined by automated plaque assay in HEp-2 cells following methods described previously (Wen Z. et al.; J Virol Methods. 2019 January; 263:88-95).

### RSV-A2-GFP Viral Replication Assay:

Calu-1 cells (human lung epidermoid carcinoma, ATCC HTB-54) assay ready freezedown (ARF generation described separately) were used in assays. Compound plates were prepared by dispensing compounds dissolved in DMSO into wells of 384 well coming 3985 polystyrene flat clear bottom optical imaging microplates (202.5 nL/well) using an ECHO acoustic dispenser. Each compound was tested in a 10-point serial 3-fold dilution (typical final concentrations: 403 nM-0.02 nM and 40.3 nM-0.002 nM). Wells with DMSO (final concentration of 0.4%) or a compound at a concentration at which viral replication was completely inhibited relative to a control compound were used as viral replication assay Min_E and Max_E control, respectively. To set up the assay, thaw ARF vial(s) in 37° C. water bath until small ice crystal remain and resuspend into assay media (DMEM containing 2% FBS, 100 U/ml Penicillin-Streptomycin). Count cells using default parameters on ViCell and dilute to 20,000 cells per ml in assay media. Add RSV GFP to cells at 24,000 pfu/ml (MOI=1.2). Mix by gentle inversion. 10 μl/well of 100% DMSO was dispensed to CellTiter-Glo (CTG) assay Max_E controls wells. Dispense cells at 50 μl/well using Bravo and 50 μl filtered tips into compound plates. Cover plates with MicroClime lids loaded with 7.5 ml of assay media to minimize evaporation. Incubate at 37 degrees C. and 5% CO₂ for 96 hrs. Following incubation, distinct, GFP-expressing cells are counted using an Acumen imaging system with appropriate settings. A same-well CTG assay is performed by adding 10 μl/well reconstituted CellTiter-Glo reagent (Promega G7573) and reading plates on PerkinElmer Envision according to manufacturer's instructions. Raw data were loaded and analyzed in ActivityBase. Antiviral IC50 and cytotoxicity CC50 values were determined using a 4 parameter logistic fit based on the Levenberg-Marquardt algorithm. Model: 205-4 Parameter Logistic.

### Generating Calu-1 Assay Ready Freezedown (ARF):

Calu-1 cells were purchased from ATCC (Cat #HTB-54) and are expanded in growth media (DMEM containing 10% FBS, 100 U/ml Penicillin-Streptomycin). To make ARF, remove and discard cell culture media, briefly rinse the cell layer with PBS to remove all traces of serum. Add 2.5 ml of TrypLE Express solution and incubate until cells dislodge, then add growth media and resuspend the cells by gentle pipetting. Count cells for concentration and determine viability using a ViCell. Centrifuge at RT, 300 g for 5 min to pellet cells. Gently aspirate supernatant, flick pellet to loosen cells. Resuspend cells in an appropriate volume of freezing medium (DMEM containing 10% DMSO, 10% FBS, 100 U/ml Penicillin-Streptomycin) to achieve 5×10\hat{ }6/ml. Transfer 1 ml of cell suspension to freezing vials. Put vials to CoolCell cryopreservation container and place CoolCell container upright in a −80° C. freezer overnight before transferring vials to liquid nitrogen vapor for storage.

Generation and Propagation of hMPV-GFP Virus:

GFP expressing rgHMPV #3 p3 was generated in Dr. Buchholz's lab (Biacchesi S. et al., J Virol. 2007 June; 81(11): 6057-6067.) and propagated in VERO cells (kidney tissue derived from a normal, adult African green monkey. ATCC Cat #CCL-81) with infection Multiplicity of Infection (MOI) of 0.1. Virus was harvested 4 days after infection by collecting all culture material and freeze-thaw the viral culture in liquid nitrogen twice. To generating virus working stocks, thaw frozen samples in 37° C. water bath then centrifuge at 218 g for 15 min at 4° C. Add 1/10 of 10×SPG (Biological Industries 06-3061-01-5A), mix, aliquot supernatant, and quick-freeze in liquid nitrogen then transfer to −80° C. freezer for storage. Virus titer was determined by performing titration test in 96-well plates and calculate approximate virus titer using GFP event/well data.

hMPV-GFP Viral Replication Assay:

Compound plates were prepared by dispensing compounds dissolved in DMSO into wells of 384 well coming 3985 polystyrene flat clear bottom optical imaging microplates (202.5 nL/well) using an ECHO acoustic dispenser. Each compound was tested in a 10-point serial 3-fold dilution (typical final concentrations: 40,300 nM-2 nM). Wells with DMSO (final concentration of 0.4%) or a compound at a concentration at which viral replication was completely inhibited relative to a control compound were used as viral replication assay Min_E and Max_E control, respectively. Continuous culture of VERO cells were maintained in complete culture media (OptiMEM supplemented with 2 mM GlutaMAX™ and 100 U/ml Penicillin-Streptomycin). To set up the assay, trypsinize VERO cells with 0.25% Trypsin-EDTA until cells dislodge, then re-suspend cells with 1 mL FBS. Spin down cells at 300 g for 5 min and wash cells twice in culture media, then count cells using default parameters on ViCell. Cells were then diluted to 100,000 cells/ml (5,000 cells/50 μl) in complete culture media+TrypLE Select (80 μl/ml). Add hMPV-GFP virus to cells at 125,000 pfu/ml (MOI=1.25). Mix by gentle inversion. 10 μl/well of 100% DMSO was dispensed to CellTiter-Glo (CTG) assay Max_E controls wells. Dispense cells at 50 μl/well using Bravo and 50 μl filtered tips into compound plates. Cover plates with MicroClime lids loaded with 7.5 ml of assay media to minimize evaporation. Lightly shake plate for 10 minutes at room temperature then incubate at 37 degrees C. and 5% CO₂ for 48 hrs. Following incubation, distinct, GFP-expressing cells are counted using an Acumen imaging system with appropriate settings. A same-well CTG assay is performed by adding 10 μl/well reconstituted CellTiter-Glo reagent (Promega G7573) and reading plates on PerkinElmer Envision according to manufacturer's instructions. Raw data were loaded and analyzed in ActivityBase. Antiviral IC50 and cytotoxicity CC50 values were determined using a 4 parameter logistic fit based on the Levenberg-Marquardt algorithm. Model: 205-4 Parameter Logistic.

### REFERENCES

Wen Z, Citron M, Bett A J, Espeseth A S, Vora K A, Zhang L, DiStefano D J. Development and application of a higher throughput RSV plaque assay by immunofluorescent imaging. J Virol Methods. 2019 January; 263:88-95. doi: 10.1016/j.jviromet.2018.10.022. Epub 2018 Oct. 28. PMD: 30381239.
Biacchesi S, Murphy B R, Collins P L, Buchholz U J. Frequent frameshift and point mutations in the SH gene of human metapneumovirus passaged in vitro. J Virol. 2007 June; 81(11):6057-67. doi: 10.1128/JVL.00128-07. Epub 2007 Mar. 21. PMID: 17376897; PMCID: PMC1900297.

### Assay Data

The EC₅₀ of each compound is listed in Table I and EC₅₀ ranges are as follows: A≤0.010 μM; B>0.010 μM-≤0.10 μM; C>0.10 μM; ND=Not Determined.

TABLE I
Summary of Activities for RSV

| EXAMPLE NO. | RSV EC₅₀ |
|---|---|
| 01-A | C |
| 01-B | A |
| 02-B | A |
| 03-B | A |
| 04-B | A |
| 05-B | A |
| 06-B | A |
| 07-B | A |
| 08-B | A |
| 09-B | A |
| 10-B | A |
| 11-B | A |
| 12-B | A |
| 13-B | A |
| 14-B | A |
| 15-B | A |
| 16-B | A |
| 17-B | A |
| 18-B | A |
| 19-B | A |
| 20-B | A |
| 21-A | A |
| 21-B | B |
| 22-A | C |
| 22-B | A |
| 23-B | A |
| 24-A | A |
| 24-B | B |
| 25-A | B |
| 25-B | C |
| 26-A | A |
| 27-A | C |
| 27-B | C |
| 27-C | A |
| 27-D | A |
| 28-A | A |
| 28-B | B |
| 28-C | C |
| 28-D | B |
| 29-A | B |
| 29-B | C |
| 29-C | A |
| 29-D | B |
| 30-B | A |
| 31-A | C |
| 31-B | A |
| 32-A | C |
| 32-B | A |
| 33-A | C |
| 33-B | C |
| 33-C | A |
| 33-D | A |
| 34-A | A |
| 34-B | C |
| 35-B | A |
| 36-A | A |
| 37-B | A |
| 38-B | A |
| 39-B | A |
| 40-B | A |
| 41-A | A |
| 42-A | A |
| 43-A | A |
| 44-A | A |
| 45-A | A |

The EC₅₀ of each compound is listed in Table II and EC₅₀ ranges are as follows: A≤0.010 μM; B>0.010 μM-≤0.10 μM; C>0.10 μM; ND=Not Determined.

TABLE II
Summary of Activities for hMPV

| EXAMPLE NO. | hMPV EC₅₀ |
|---|---|
| 01-A | C |
| 01-B | C |
| 02-B | C |
| 03-B | C |
| 04-B | C |
| 05-B | C |
| 06-B | C |
| 07-B | C |
| 08-B | C |
| 09-B | C |
| 10-B | C |
| 11-B | C |
| 12-B | C |
| 13-B | C |
| 14-B | C |
| 15-B | C |
| 16-B | C |
| 17-B | C |
| 18-B | C |
| 19-B | C |
| 20-B | C |
| 21-A | C |
| 21-B | C |
| 22-A | C |
| 22-B | C |
| 23-B | C |
| 24-A | C |
| 24-B | C |
| 25-A | C |
| 25-B | C |
| 26-A | C |
| 27-A | C |
| 27-B | C |
| 27-C | C |
| 27-D | C |
| 28-A | C |
| 28-B | C |
| 28-C | C |
| 28-D | C |
| 29-A | ND |
| 29-B | ND |
| 29-C | ND |
| 29-D | ND |
| 30-B | ND |
| 31-A | ND |
| 31-B | ND |
| 32-A | ND |
| 32-B | ND |
| 33-A | ND |
| 33-B | ND |
| 33-C | ND |
| 33-D | ND |
| 34-A | ND |
| 34-B | ND |
| 35-B | ND |
| 36-A | ND |
| 37-B | ND |
| 38-B | ND |
| 39-B | ND |
| 40-B | ND |
| 41-A | ND |
| 42-A | ND |
| 43-A | ND |
| 44-A | ND |
| 45-A | ND |

What is claimed is:

1. A compound of Formula I, 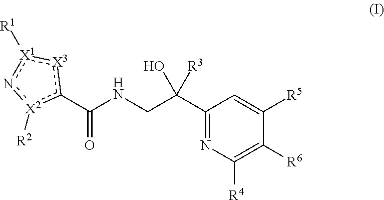
or a pharmaceutically acceptable salt thereof, wherein:
R¹ is selected from:
(a) —OC₁₋₃ alkyl,
(b) a 5-member heteroaryl ring comprised of three carbon atoms and (i) two of N, (ii) N and S, or (iii) N and O, and
(c) a 6-member heteroaryl ring comprised of four carbon atoms and two of N;
X¹ is selected from C and N;
X² is selected from C, S and N,
wherein X² is C when X¹ is N, or when X³ is S;
X³ is selected from C, S and N,
wherein X³ is C when X² is S or N, or when X¹ is N;
R² is selected from:
(a) —H,
(b) —C₁₋₃alkyl,
(c) —C₃₋₅cycloalkyl, and
(d) —OC₁₋₃alkyl;
R³ is selected from:
(a) —C₁₋₃alkyl,
(b) —C₃₋₅cycloalkyl,
(c) —CF₃,
(d) —CF₂H, and
(e) —H;
R⁴ is selected from:
(1) phenyl substituted with 1 to 3 substituents independently selected at each occurrence from:
(a) halo, and
(b) —C₁₋₃alkyl, or
(2) 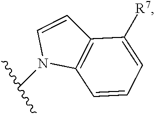
wherein R⁷ is selected from —H and halo, or
(3) 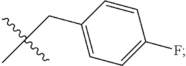 [SMILES: *Cc1ccc(F)cc1]
R⁵ is selected from:
(1) a —C₁₋₅alkyl, unsubstituted or substituted with (i) —NH₂, (ii) —(C═O)NH₂, or (iii) —OH,
(2) pyrrolidinyl, unsubstituted or substituted with —C₁₋₃ alkyl; and
R⁶ is selected from:
(3) —H, and
(4) —O—, wherein R⁶ and any alkyl moiety of R⁵ are taken together with their intervening atoms to form a cyclic group.

2. The compound of claim 1, or a pharmaceutically acceptable salt thereof, wherein:
R¹ is selected from:
ethoxy, 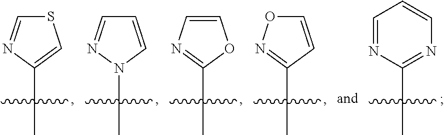
X¹ and X³ are C;
X² is N;
R² is methyl;
R³ is methyl;
R⁴ is phenyl substituted with 1 or 2 substituents independently selected at each occurrence from fluoro and chloro; and
R⁵ is a —C₃alkyl substituted with —NH₂; and
R⁶ is —H.

3. The compound of claim 1, or a pharmaceutically acceptable salt thereof, wherein R¹ is

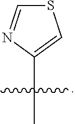 [SMILES: *c1cscn1]

4. The compound of claim 1, or a pharmaceutically acceptable salt thereof, wherein X¹ is C and X² is N.

5. The compound of claim 1, or a pharmaceutically acceptable salt thereof, wherein R⁵ is 2-aminopropan-2-yl.

6. The compound of claim 1, or a pharmaceutically acceptable salt thereof, wherein R⁴ is

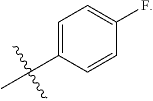 [SMILES: *c1ccc(F)cc1]

7. The compound of claim 1, or a pharmaceutically acceptable salt thereof, wherein the compound is of Formula II: 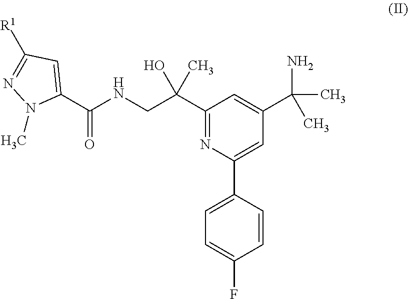
or a pharmaceutically acceptable salt thereof, wherein R¹ is defined as in claim 1.

8. The compound of claim 1, or a pharmaceutically acceptable salt thereof, wherein the compound is of Formula III: 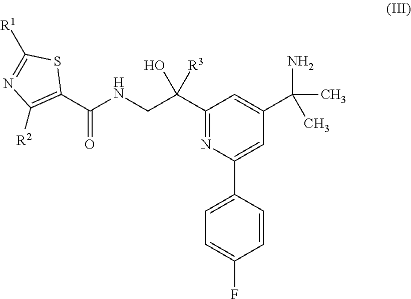
or a pharmaceutically acceptable salt thereof, wherein R¹, R² and R³ are defined as in claim 1.

9. The compound of claim 1 that is:
N-(2-(4-(2-aminopropan-2-yl)-6-(4-fluorophenyl)pyridin-2-yl)-2-hydroxypropyl)-3-ethoxy-1-
methyl-1H-pyrazole-5-carboxamide (enantiomer-1)
N-(2-(4-(2-aminopropan-2-yl)-6-(4-fluorophenyl)pyridin-2-yl)-2-hydroxypropyl)-3-ethoxy-1-
methyl-1H-pyrazole-5-carboxamide (enantiomer-2)
N-(2-(4-(2-aminopropan-2-yl)-6-(4-fluorophenyl)pyridin-2-yl)-2-hydroxypropyl)-1'-methyl-1'H-
[1,3'-bipyrazole]-5'-carboxamide (enantiomer-2)
N-(2-(4-(2-aminopropan-2-yl)-6-(4-fluorophenyl)pyridin-2-yl)-2-hydroxypropyl)-3-(isoxazol-3-
yl)-1-methyl-1H-pyrazole-5-carboxamide (enantiomer-2)
N-(2-(4-(2-aminopropan-2-yl)-6-(4-fluorophenyl)pyridin-2-yl)-2-hydroxypropyl)-1-methyl-3-
(oxazol-2-yl)-1H-pyrazole-5-carboxamide (enantiomer-2)
N-(2-(4-(2-aminopropan-2-yl)-6-(4-fluorophenyl)pyridin-2-yl)-2-hydroxypropyl)-1-methyl-3-
(thiazol-4-yl)-1H-pyrazole-5-carboxamide (enantiomer-2)
N-(2-(4-(2-aminopropan-2-yl)-6-(4-fluorophenyl)pyridin-2-yl)-2-hydroxypropyl)-1-ethyl-3-
(thiazol-4-yl)-1H-pyrazole-5-carboxamide (enantiomer-2)
N-(2-(4-(2-aminopropan-2-yl)-6-(4-fluorophenyl)pyridin-2-yl)-2-hydroxypropyl)-4-methyl-[2,4'-
bithiazole]-5-carboxamide (enantiomer-2)
N-(2-(4-(2-aminopropan-2-yl)-6-(4-fluorophenyl)pyridin-2-yl)-2-hydroxypropyl)-4-cyclopropyl-
[2,4'-bithiazole]-5-carboxamide (enantiomer-2)
N-(2-(4-(2-aminopropan-2-yl)-6-(4-fluorophenyl)pyridin-2-yl)-2-hydroxypropyl)-3-(thiazol-4-
yl)isothiazole-5-carboxamide (enantiomer-2)
N-(2-(4-(2-aminopropan-2-yl)-6-(4-fluorophenyl)pyridin-2-yl)-2-hydroxypropyl)-3-(pyrimidin-2-
yl)isothiazole-5-carboxamide (enantiomer-2)
N-(2-(4-(2-aminopropan-2-yl)-6-(4-fluorophenyl)pyridin-2-yl)-2-hydroxypropyl)-3-methoxy-1-
(pyrimidin-2-yl)-1H-pyrazole-4-carboxamide (enantiomer-2)
N-(2-(4-(2-aminopropan-2-yl)-6-(4-fluoro-1H-indol-1-yl)pyridin-2-yl)-2-hydroxypropyl)-1-
methyl-3-(thiazol-4-yl)-1H-pyrazole-5-carboxamide (enantiomer-2)
N-(2-(4-(2-aminopropan-2-yl)-6-(4-chlorophenyl)pyridin-2-yl)-2-hydroxypropyl)-1-methyl-3-
(thiazol-4-yl)-1H-pyrazole-5-carboxamide (enantiomer-2)
N-(2-(4-(2-aminopropan-2-yl)-6-(p-tolyl)pyridin-2-yl)-2-hydroxypropyl)-1-methyl-3-(thiazol-4-
yl)-1H-pyrazole-5-carboxamide (enantiomer-2)
N-(2-(4-(2-aminopropan-2-yl)-6-(3,4-difluorophenyl)pyridin-2-yl)-2-hydroxypropyl)-1-methyl-3-
(thiazol-4-yl)-1H-pyrazole-5-carboxamide (enantiomer-2)
N-(2-(4-(2-aminopropan-2-yl)-6-(3-chloro-4-fluorophenyl)pyridin-2-yl)-2-hydroxypropyl)-1-
methyl-3-(thiazol-4-yl)-1H-pyrazole-5-carboxamide (enantiomer-2)
N-(2-(4-(2-aminopropan-2-yl)-6-(4-chloro-3-fluorophenyl)pyridin-2-yl)-2-hydroxypropyl)-1-
methyl-3-(thiazol-4-yl)-1H-pyrazole-5-carboxamide (enantiomer-2)
N-(2-(4-(2-aminopropan-2-yl)-6-(2,4-difluorophenyl)pyridin-2-yl)-2-hydroxypropyl)-1-methyl-3-
(thiazol-4-yl)-1H-pyrazole-5-carboxamide (enantiomer-2)
N-(2-(4-(2-aminopropan-2-yl)-6-(4-fluoro-2-methylphenyl)pyridin-2-yl)-2-hydroxypropyl)-1-
methyl-3-(thiazol-4-yl)-1H-pyrazole-5-carboxamide (enantiomer-2)
N-(2-(4-(2-aminopropan-2-yl)-6-(2,3,4-trifluorophenyl)pyridin-2-yl)-2-hydroxypropyl)-1-methyl-
3-(thiazol-4-yl)-1H-pyrazole-5-carboxamide (enantiomer-2)
N-(2-(4-(2-aminopropan-2-yl)-6-(4-fluorophenyl)pyridin-2-yl)-2-hydroxybutyl)-1-methyl-3-
(thiazol-4-yl)-1H-pyrazole-5-carboxamide (enantiomer-1 and enantiomer-2)
N-(2-(4-(2-aminopropan-2-yl)-6-(4-fluorophenyl)pyridin-2-yl)-2-cyclopropyl-2-hydroxyethyl)-4-
methyl-2-(pyrimidin-2-yl)thiazole-5-carboxamide (enantiomer-1)
N-(2-(4-(2-aminopropan-2-yl)-6-(4-fluorophenyl)pyridin-2-yl)-2-cyclopropyl-2-hydroxyethyl)-4-
methyl-2-(pyrimidin-2-yl)thiazole-5-carboxamide (enantiomer-2)
N-(2-(4-(2-aminopropan-2-yl)-6-(4-fluorophenyl)pyridin-2-yl)-2-cyclopropyl-2-hydroxyethyl)-1-
methyl-3-(thiazol-4-yl)-1H-pyrazole-5-carboxamide (enantiomer-2)
N-(2-(4-(2-aminopropan-2-yl)-6-(4-fluorophenyl)pyridin-2-yl)-3,3-difluoro-2-hydroxypropyl)-1-
methyl-3-(thiazol-4-yl)-1H-pyrazole-5-carboxamide (enantiomer-1)
N-(2-(4-(2-aminopropan-2-yl)-6-(4-fluorophenyl)pyridin-2-yl)-3,3-difluoro-2-hydroxypropyl)-1-
methyl-3-(thiazol-4-yl)-1H-pyrazole-5-carboxamide (enantiomer-2)
N-(2-(4-(2-aminopropan-2-yl)-6-(4-fluorophenyl)pyridin-2-yl)-3,3,3-trifluoro-2-hydroxypropyl)-
4-methyl-2-(pyrimidin-2-yl)thiazole-5-carboxamide (enantiomer-1)
N-(2-(4-(2-aminopropan-2-yl)-6-(4-fluorophenyl)pyridin-2-yl)-3,3,3-trifluoro-2-hydroxypropyl)-
4-methyl-2-(pyrimidin-2-yl)thiazole-5-carboxamide (enantiomer-2)
N-(2-(4-(2-aminopropan-2-yl)-6-(4-fluorophenyl)pyridin-2-yl)-3,3,3-trifluoro-2-hydroxypropyl)-
1-methyl-3-(thiazol-4-yl)-1H-pyrazole-5-carboxamide (enantiomer-1)
N-(2-(6-(4-fluorophenyl)-4-(3-methylpyrrolidin-3-yl)pyridin-2-yl)-2-hydroxypropyl)-1-methyl-3-
(thiazol-4-yl)-1H-pyrazole-5-carboxamide
N-(2-(3-amino-7-(4-fluorophenyl)-3-methyl-2,3-dihydrofuro[2,3-c]pyridin-5-yl)-2-
hydroxypropyl)-1-methyl-3-(thiazol-4-yl)-1H-pyrazole-5-carboxamide (diastereoisomer-1)
N-(2-(3-amino-7-(4-fluorophenyl)-3-methyl-2,3-dihydrofuro[2,3-c]pyridin-5-yl)-2-
hydroxypropyl)-1-methyl-3-(thiazol-4-yl)-1H-pyrazole-5-carboxamide (diastereoisomer-2)
N-(2-(3-amino-7-(4-fluorophenyl)-3-methyl-2,3-dihydrofuro[2,3-c]pyridin-5-yl)-2-
hydroxypropyl)-1-methyl-3-(thiazol-4-yl)-1H-pyrazole-5-carboxamide (diastereoisomer-3)
N-(2-(3-amino-7-(4-fluorophenyl)-3-methyl-2,3-dihydrofuro[2,3-c]pyridin-5-yl)-2-
hydroxypropyl)-1-methyl-3-(thiazol-4-yl)-1H-pyrazole-5-carboxamide (diastereoisomer-4)
7-(4-fluorophenyl)-5-(2-hydroxy-1-(1'-methyl-1'H-[1,3'-bipyrazole]-5'-carboxamido)propan-2-yl)-
3-methyl-2,3-dihydrofuro[2,3-c]pyridine-3-carboxamide (diastereoisomer-1)
7-(4-fluorophenyl)-5-(2-hydroxy-1-(1'-methyl-1'/-[1,3'-bipyrazole]-5'-carboxamido)propan-2-yl)-
3-methyl-2,3-dihydrofuro[2,3-c]pyridine-3-carboxamide (diastereoisomer-2)
7-(4-fluorophenyl)-5-(2-hydroxy-1-(1'-methyl-l'H-[1,3'-bipyrazole]-5'-carboxamido)propan-2-yl)-
3-methyl-2,3-dihydrofuro[2,3-c]pyridine-3-carboxamide (diastereoisomer-3)
7-(4-fluorophenyl)-5-(2-hydroxy-1-(1'-methyl-1'W-[1,3'-bipyrazole]-5'-carboxamido)propan-2-yl)-
3-methyl-2,3-dihydrofuro[2,3-c]pyridine-3-carboxamide (diastereoisomer-4)
7-(4-fluorophenyl)-3-methyl-5-(1,1,1-trifluoro-2-hydroxy-3-(1-methyl-3-(thiazol-4-yl)-1H-
pyrazole-5-carboxamido)propan-2-yl)-2,3-dihydrofuro[2,3-c]pyridine-3-carboxamide
(diastereoisomer-2)
N-(2-(4-(1-amino-2-methyl-1-oxopropan-2-yl)-6-(4-fluorophenyl)pyridin-2-yl)-2-hydroxypropyl)-
1-methyl-3-(thiazol-4-yl)-1H-pyrazole-5-carboxamide (enantiomer-1)
N-(2-(4-(1-amino-2-methyl-1-oxopropan-2-yl)-6-(4-fluorophenyl)pyridin-2-yl)-2-hydroxypropyl)-
1-methyl-3-(thiazol-4-yl)-1H-pyrazole-5-carboxamide (enantiomer-2)
N-(2-(6-(4-fluorophenyl)-4-(2-hydroxypropan-2-yl)pyridin-2-yl)-2-hydroxypropyl)-1-methyl-3-
(thiazol-4-yl)-1H-pyrazole-5-carboxamide (enantiomer-1)
N-(2-(6-(4-fluorophenyl)-4-(2-hydroxypropan-2-yl)pyridin-2-yl)-2-hydroxypropyl)-1-methyl-3-
(thiazol-4-yl)-1H-pyrazole-5-carboxamide (enantiomer-2)
N-(2-(4-(3-amino-2-methylbutan-2-yl)-6-(4-fluorophenyl)pyridin-2-yl)-2-hydroxypropyl)-1'-
methyl-1'H-[1,3'-bipyrazole]-5'-carboxamide (diastereoisomer-1)
N-(2-(4-(3-amino-2-methylbutan-2-yl)-6-(4-fluorophenyl)pyridin-2-yl)-2-hydroxypropyl)-1'-
methyl-1'H-[1,3'-bipyrazole]-5'-carboxamide (diastereoisomer-2)
N-(2-(4-(3-amino-2-methylbutan-2-yl)-6-(4-fluorophenyl)pyridin-2-yl)-2-hydroxypropyl)-1'-
methyl-1'H-[1,3'-bipyrazole]-5'-carboxamide (diastereoisomer-3)
N-(2-(4-(3-amino-2-methylbutan-2-yl)-6-(4-fluorophenyl)pyridin-2-yl)-2-hydroxypropyl)-1'-
methyl-1'H-[1,3'-bipyrazole]-5'-carboxamide (diastereoisomer-4)
N-(2-(4-(2-aminopropan-2-yl)-6-(4-fluorophenyl)pyridin-2-yl)-2-hydroxyethyl)-1'-methyl-1'H-
[1,3'-bipyrazole]-5'-carboxamide (enantiomer-1)
N-(2-(4-(2-aminopropan-2-yl)-6-(4-fluorophenyl)pyridin-2-yl)-2-hydroxyethyl)-1'-methyl-1'H-
[1,3'-bipyrazole]-5'-carboxamide (enantiomer-2)
N-(2-(4-(2-aminopropan-2-yl)-6-(4-fluorophenyl)pyridin-2-yl)-2-cyclopropyl-2-hydroxyethyl)-1'-
methyl-1'H-[1,3'-bipyrazole]-5'-carboxamide (enantiomer-2)
N-(2-(4-(2-aminopropan-2-yl)-6-(4-fluorophenyl)pyridin-2-yl)-3,3,3-trifluoro-2-hydroxypropyl)-
l'-methyl-1'H-[1,3'-bipyrazole]-5'-carboxamide (enantiomer-1)
N-(2-(4-(2-aminopropan-2-yl)-6-(4-(trifluoromethyl)phenyl)pyridin-2-yl)-2-hydroxypropyl)-1'-
methyl-1'H-[1,3'-bipyrazole]-5'-carboxamide (enantiomer-2)
N-(2-(4-(2-aminopropan-2-yl)-6-(3,4-difluorophenyl)pyridin-2-yl)-2-hydroxypropyl)-1'-methyl-
1'H-[1,3'-bipyrazole]-5'-carboxamide (enantiomer-2)
N-(2-(4-(2-aminopropan-2-yl)-6-(4-chloro-3-fluorophenyl)pyridin-2-yl)-2-hydroxypropyl)-1'-
methyl-1'H-[1,3'-bipyrazole]-5'-carboxamide (enantiomer-2)
N-(2-(4-(2-aminopropan-2-yl)-6-(4-fluorobenzyl)pyridin-2-yl)-2-hydroxypropyl)-1'-methyl-1'H-
[1,3'-bipyrazole]-5'-carboxamide (enantiomer-2)
N-(2-(4-(2-aminopropan-2-yl)-6-(4-(difluoromethyl)phenyl)pyridin-2-yl)-3,3,3-trifluoro-2-
hydroxypropyl)-1'-methyl-1'H-[1,3'-bipyrazole]-5'-carboxamide (enantiomer-1)
N-(2-(4-(2-aminopropan-2-yl)-6-(4-(trifluoromethyl)phenyl)pyridin-2-yl)-3,3,3-trifluoro-2-
hydroxypropyl)-1'-methyl-1'H-[1,3'-bipyrazole]-5'-carboxamide (enantiomer-1)
N-(2-(4-(2-aminopropan-2-yl)-6-(3,4-difluorophenyl)pyridin-2-yl)-3,3,3-trifluoro-2-
hydroxypropyl)-1'-methyl-1'H-[1,3'-bipyrazole]-5'-carboxamide (enantiomer-1)
N-(2-(4-(2-aminopropan-2-yl)-6-(2,4-difluorophenyl)pyridin-2-yl)-3,3,3-trifluoro-2-
hydroxypropyl)-1'-methyl-1'H-[1,3'-bipyrazole]-5'-carboxamide (enantiomer-1)
N-(2-(4-(2-aminopropan-2-yl)-6-(4-fluoro-2-methylphenyl)pyridin-2-yl)-3,3,3-trifluoro-2-
hydroxypropyl)-1'-methyl-1'H-[1,3'-bipyrazole]-5'-carboxamide (enantiomer-1)
or a pharmaceutically acceptable salt thereof.

10. A pharmaceutical composition comprising an effective amount of the compound according to claim 1, or a pharmaceutically acceptable salt thereof, and a pharmaceutically acceptable carrier.

11. A method for the inhibition of the replication of hRSV in a human subject in need thereof which comprises administering to the subject an effective amount of the compound according to claim 1, or a pharmaceutically acceptable salt thereof.

12. A method for the treatment of hRSV infection in a human subject in need thereof which comprises administering to the subject an effective amount of the compound according to claim 1, or a pharmaceutically acceptable salt thereof.

13. A method for the prophylaxis of hRSV infection in a human subject which comprises administering to the subject an effective amount of the compound according to claim 1, or a pharmaceutically acceptable salt thereof.

14. A method for the inhibition of the replication of hMPV in a human subject in need thereof which comprises administering to the subject an effective amount of the compound according to claim 1, or a pharmaceutically acceptable salt thereof.

15. A method for the treatment of hMPV infection in a human subject in need thereof which comprises administering to the subject an effective amount of the compound according to claim 1, or a pharmaceutically acceptable salt thereof.

16. A method for prophylaxis of hMPV infection in a human subject which comprises administering to the subject an effective amount of the compound according to claim 1, or a pharmaceutically acceptable salt thereof.
